(19) United States
(12) Patent Application Publication
(10) Pub. No.: US 2025/0179585 A1
(43) Pub. Date: Jun. 5, 2025

(54) METHODS AND COMPOSITIONS FOR IDENTIFYING STRUCTURAL VARIANTS

(71) Applicant: Arima Genomics, Inc., Carlsbad, CA (US)

(72) Inventors: Anthony Schmitt, Apex, NC (US); Kristin Sikkink, Fallbrook, CA (US); Bret Derek Reid, San Diego, CA (US)

(73) Assignee: Arima Genomics, Inc., Carlsbad, CA (US)

(21) Appl. No.: 18/844,316

(22) PCT Filed: Mar. 6, 2023
(86) PCT No.: PCT/US2023/063807

§ 371 Date: Sep. 5, 2024

Related U.S. Application Data
(60) Provisional application No. 63/418,416, filed on Oct. 21, 2022.
(60) Provisional application No. 63/400,861, filed on Aug. 25, 2022.
(60) Provisional application No. 63/400,862, filed on Aug. 25, 2022.
(60) Provisional application No. 63/400,872, filed on Aug. 25, 2022.
(60) Provisional application No. 63/400,869, filed on Aug. 25, 2022.
(60) Provisional application No. 63/400,877, filed on Aug. 25, 2022.
(60) Provisional application No. 63/400,878, filed on Aug. 25, 2022.
(60) Provisional application No. 63/400,865, filed on Aug. 25, 2022.
(60) Provisional application No. 63/322,745, filed on Mar. 23, 2022.
(60) Provisional application No. 63/322,748, filed on Mar. 23, 2022.
(60) Provisional application No. 63/317,390, filed on Mar. 7, 2022.
(60) Provisional application No. 63/317,396, filed on Mar. 7, 2022.
(60) Provisional application No. 63/317,399, filed on Mar. 7, 2022.
(60) Provisional application No. 63/317,404, filed on Mar. 7, 2022.

(51) Int. Cl. C12Q 1/6886; C12Q 1/6813

(52) U.S. Cl. CPC C12Q 1/6886; C12Q 1/6813; C12Q 2600/156; C12Q 2600/16

(57) ABSTRACT

The technology relates in part to methods and compositions for detecting oncogenic structural variants.

### CROSS REFERENCE TO RELATED APPLICATIONS

This application claims the benefit under 35 U.S.C. § 119(e) of U.S. provisional application No. 63/317,390, filed Mar. 7, 2022, U.S. provisional application No. 63/400,861, filed Aug. 25, 2022, U.S. provisional application No. 63/317,396, filed Mar. 7, 2022, U.S. provisional application No. 63/400,862, filed Aug. 25, 2022, U.S. provisional application No. 63/317,399, filed Mar. 7, 2022, U.S. provisional application No. 63/322,745, filed Mar. 23, 2022, U.S. provisional application No. 63/400,865, filed Aug. 25, 2022, U.S. provisional application No. 63/317,404, filed Mar. 7, 2022, U.S. provisional application No. 63/322,748, filed Mar. 23, 2022, U.S. provisional application No. 63/400,869, filed Aug. 25, 2022, U.S. provisional application No. 63/418,416, filed Oct. 21, 2022, U.S. provisional application No. 63/400,877, filed Aug. 25, 2022, U.S. provisional application No. 63/400,878, filed Aug. 25, 2022 and U.S. provisional application No. 63/400,872, filed Aug. 25, 2022. The entire contents of each of these referenced applications is incorporated by reference herein.

### FIELD

The technology relates in part to methods and compositions for identifying structural variants.

### BACKGROUND

Cancers are often caused by genetic alterations, which include mutations (e.g., point mutations) and structural variations (e.g., translocations, inversions, insertions, deletions, and duplications). Genetic alterations can prevent certain genes from working properly. Genes that have mutations and/or structural variations that are linked to cancer may be referred to as cancer genes or oncogenes. Certain types of cancers have been linked to particular genetic alterations. However, there are cancers for which specific genetic alterations have not yet been identified.

A subject may acquire cancer-causing genetic alterations in a number of ways. In certain instances, a subject is born with a genetic alteration that is either inherited from a parent or arises during gestation. In certain instances, a subject is exposed to one or more factors that damage genetic material (e.g., UV light, cigarette smoke). In certain instances, genetic alterations arise as the subject ages.

Accurate and sensitive identification of genetic alterations is useful for understanding mechanisms of various cancers and for the development and selection of optimal treatment regimens for cancer patients. For structural variants, these typically are detected using RNA sequencing approaches, low-resolution karyotyping, and/or low throughput and biased FISH assays. Using such approaches, the accuracy and sensitivity of structural variant detection can be limited by factors such as low transcript abundance, transcript length, RNA degradation (e.g., in formalin fixed paraffin embedded (FFPE) tissues), and/or limited availability of fresh biopsy samples for RNA extraction. Provided herein are methods for accurate and sensitive identification of structural variants. Also provided herein are structural variants identified by methods described herein.

### SUMMARY

Provided in certain aspects are methods for detecting the presence or absence of a structural variant in a sample by a) performing a nucleic acid analysis on the selected sample, and the analysis may include a method that preserves spatial-proximal contiguity information; and b) detecting whether a structural variant is present or absent in the selected sample according to the nucleic acid analysis in (b), and where a breakpoint of the structural variant is not within the one or more cancer genes analyzed in (a).

Provided in certain aspects are methods for detecting the presence or absence of a structural variant in a sample by a) selecting a sample from a subject, wherein one or more oncogenes in the sample were analyzed for one or more genetic variations associated with cancer, and the one or more oncogenes have no detectable genetic variation associated with cancer; b) performing a nucleic acid analysis on the selected sample, where the analysis may include a method that preserves spatial-proximal contiguity information; and c) detecting whether a structural variant is present or absent in the selected sample according to the nucleic acid analysis in (b), with a breakpoint of the structural variant is not within the one or more cancer genes analyzed in (a).

Provided in certain aspects are methods for detecting the presence or absence of a structural variant in a sample by: a) performing a nucleic acid analysis on a sample from a subject, wherein the analysis includes i) generating proximity ligated nucleic acid molecules and ii) contacting the proximity ligated nucleic acid molecules with one or more capture probe species, thereby generating enriched proximity ligated nucleic acid molecules, wherein the one or more capture probe species each comprise a polynucleotide identical to or complementary to a subsequence of a cancer gene; and b) detecting whether a structural variant is present or absent in the selected sample according to the nucleic acid analysis in (a).

Provided in certain aspects are compositions of a set of synthetic oligonucleotide species, wherein:
a) each oligonucleotide species is 10 to 500 consecutive nucleotides in length; b) each oligonucleotide species has a polynucleotide identical to or complementary to a subsequence in an exon of an oncogene; and c) the polynucleotide maps to coordinates that are within 300-400 bp of one or more sites targeted by one or more restriction enzymes.

Provided in certain aspects are methods for detecting the presence or absence of a structural variant in a sample by: a) obtaining a sample from a subject over a plurality of time points; b) for the sample obtained at each of the time points, performing a nucleic acid analysis on the sample, where the analysis comprises a method that preserves spatial-proximal contiguity information; and
c) detecting whether a structural variant is present or absent in the selected sample according to the nucleic acid analysis in (b).

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1A shows a schematic of Capture-HiC data using target enrichment probes targeted to cancer genes in order to identify a structural variant (SV) that results in a gene fusion. FIG. 1B shows a schematic of Capture-HiC data using target enrichment probes targeted to cancer genes in order to identify an SV that results in a breakpoint outside of the targeted gene body.

FIG. 2A shows a schematic of an exemplary HiC and formalin-fixed, paraffin-embedded (FFPE) sample workflow. FIG. 2B shows a schematic of an exemplary workflow for detection of gene fusions in FFPE using Capture HiC. FIG. 2C shows a schematic of an exemplary workflow for identification of gene fusions.

FIGS. 3A-3E shows a representative HiC analysis showing the detection of an SV that results in a gene fusion, which can resolve complex SVs involving multiple genes. FIG. 3A shows a heatmap from 3D genome analysis identifying a MYBL1-CHD7 gene fusion and a MYBL1-CDH17 gene fusion. FIG. 3B shows a heatmap from 3D genome analysis identifying a MYBL1-AGTPBP1 gene fusion. FIG. 3C is a zoomed-in view around the approximate breakpoints in MYBL1 and CHD7. FIG. 3D shows a zoomed-in view around the approximate breakpoints in MYBL1 and CDH17. FIG. 3E shows a zoomed-in view around the approximate breakpoints in MYBL1 and CHD7.

FIG. 4 shows a representative Capture-HiC genome-scan analysis used to identify sequences with high spatial proximity to a targeted gene where the SV results in a gene fusion which can resolve complex SVs involving multiple genes. FIG. 4A depicts a quantification of the observed Capture-HiC read-pairs where at least 1 read-end aligns to MYBL1 and the other ends aligns to anywhere along chr8. FIG. 4B is the sample type of analysis as FIG. 4A, expect the x axis is the entire human genome rather than just chr8. FIG. 4C shows a depicted analogous to FIG. 4A, except here a quantification of the observed Capture-HiC read-pairs where at least 1 read-end aligns to CHD7 and the other ends aligns to anywhere along chr8. FIG. 4D is analogous to FIG. 4B where a quantification of the observed Capture-HiC read-pairs where at least 1 read-end aligns to CHD7 and the other ends aligns to anywhere along the human genome

FIG. 5 shows representative Capture-HiC Integrative Genomics Viewer (IGV) Browser analyses. FIG. 5A shows an IGV browser view of reads where one read-end aligns to MYBL1, and the other read end aligns around the CHD7 gene. FIG. 5B shows an IGV browser view of reads where one read-end aligns to MYBL1, and the other read end aligns around the AGTPBP1 gene on chr9. FIG. 5C shows an IGV browser view of reads where one read-end aligns to CHD7 and the other read end aligns around the MYBL1 gene. FIG. 5D shows an IGV browser view of reads where one read-end aligns to CHD7, and the other read end aligns around the CDH17 gene on chr8.

FIG. 6 shows a representative HiC analysis showing the detection of a SV that results in a breakpoint outside of a cancer-associated gene(s), but within a certain linear proximity to the cancer-associated gene(s). FIG. 6A shows a HiC contact matrix showing all inter-chromosomal contacts between chr5 and chr7. FIG. 6B shows a zoomed-in view around the approximate breakpoints on chr5 and chr7.

FIG. 7 shows representative Capture-HiC genome-scan analysis used to identify sequences with high spatial proximity to a targeted gene, where the SV breakpoint is outside of a targeted cancer-associated gene. FIG. 7A depicts a quantification of the observed Capture-HiC read-pairs where at least 1 read-end aligns to TERT and the other ends aligns to anywhere along the entire human genome. FIG. 7B depicts a quantification of the observed Capture-HiC read-pairs where at least 1 read-end aligns to MET and the other ends aligns to anywhere along the entire human genome.

FIG. 8 shows a representative Capture-HiC IGV Browser analyses, used for analyzing the breakpoint coordinates and genes involved in a particular SV where the SV comprises a breakpoint outside of a targeted cancer-associated gene. FIG. 8A shows an IGV browser view of reads where one read-end aligns to TERT, and the other read end aligns in and around the CAV1 gene. FIG. 8B shows an IGV browser view of reads where one read-end aligns to MET, and the other read end aligns around the TERT gene.

FIG. 9 shows examples of inter-chromosomal and intra-chromosomal gene fusions detected using methods described herein. FIG. 9A shows a Manhattan plot representation of an EWSR1-FLI1 gene fusion detected with probes targeting EWSR1. FIG. 9B shows a Manhattan plot representation of an ETV6-NTRK3 gene fusion detected with probes targeting NTRK3. FIG. 9C shows a Manhattan plot representation of a DYCN112-ALK gene fusion detected with probes targeting ALK. FIG. 9D shows a Manhattan plot representation of an NCOA4-RET gene fusion detected with probes targeting RET in a sample.

FIG. 10 shows the result of an exemplary process in which 3D genome analysis described herein was used to alter the course of patient management in a prospective glioma patient. FIG. 10A shows a plot of copy number variation profile lacking any detectable diagnostic MYB or MYBL1 gene fusion. FIG. 10B shows heatmaps from 3D genome analysis identifying a MYBL1-MAML2 gene fusion.

FIG. 11 shows detection of an NTRK1 proximity fusion in a subependymal giant cell astrocytoma sample using the methods described herein. FIG. 11A shows a HiC heatmap showing the TFE3-PRCC gene fusion with NTRK1 in proximity to the fusion breakpoint (hence, defining this fusion as an NTRK1 proximity fusion) and HiC signal showing NTRK1 interacting with genomic sequences across the breakpoint, which may influence changes in its expression levels. FIG. 11B shows a schematic of the same NTRK1 proximity fusion, showing a gene fusion event between PRCC chromosome 1 (chr1) and TFE3 on chromosome X (chrX). Importantly, NTRK1 (also on chr1) is located ˜66 kb away from the breakpoint on chr1, and so with respect to NTRK1 is a proximity fusion. Depicted is full length (non-chimeric) NTRK1 transcripts being expressed. FIG. 11C shows a micrograph of positive immunohistochemical staining of NTRK (using a pan-TRK antibody). FIG. 11D shows a micrograph of negative immunohistochemical staining of NTRK in normal tissue adjacent to the tumor tissue in FIG. 11C.

FIG. 12 shows detection of a PLAG1 proximity fusion in a myxoid leiomyosarcoma sample using the methods described herein. FIG. 12A shows a HiC heatmap showing the RAD51B-LYN gene fusion with PLAG1 in proximity to the fusion breakpoint (hence, defining this fusion as a PLAG1 proximity fusion) and HiC signal showing PLAG1 interacting with with genomic sequences across the breakpoint, which may influence changes in its expression levels. FIG. 12B shows a schematic of the same PLAG1 proximity fusion, showing a gene fusion event between LYN on chromosome 8 (chr8) and RAD51B on chromosome 14 (chr14). Importantly, PLAG1 (also on chr8) is located ˜170 kb away from the breakpoint on chr8, and so with respect to PLAG1 is a proximity fusion. Depicted is full length (non-chimeric) PLAG1 transcripts being expressed. FIG. 12C shows a micrograph of positive immunohistochemical staining of PLAG1 using anti-PLAG1 antibody.

FIG. 13 shows an immunohistochemistry stain using anti-CCND1 (Cyclin D1) antibody. FIG. 13A is a positive control. FIG. 13B shows the anti-CCND1 stain in epithelioid mesenchymal tumor with SMD cells.

FIG. 14 shows an immunohistochemistry stain using anti-CDK4 antibody. FIG. 14A is a positive control. FIG. 14B shows the anti-CDK4 stain in an adenosarcoma with sarcoma overgrowth (ASSO) tumor.

FIG. 15 shows an immunohistochemistry stain using anti-CCND1 (Cyclin D1) antibody. FIG. 15A is a positive control. FIG. 15B shows the anti-CCND1 stain in low grade (LG) epithelioid neoplasm with myomelanocytic differentiation tumor cells.

FIG. 16 shows an immunohistochemistry stain using anti-MyoD1 antibody. FIG. 16A is a positive control. FIG. 16B shows the anti-MyoD1 antibody staining of HG spindle cell sarcoma tumor cells.

FIG. 17 shows an immunohistochemistry stain using anti-ESR1 antibody. FIG. 17A is a positive control. FIG. 17B shows the anti-ESR1 stain in uterine tumor resembling ovarian sex cord tumor (UTROSCT) cells.

FIG. 18 shows an immunohistochemistry stain using anti-EGFR antibody. FIG. 18A is a positive control. FIG. 18B shows the anti-EGFR stain in colorectal carcinoma cells.

FIG. 19 shows an immunohistochemistry stain using anti-MDM2 antibody. FIG. 19A is a positive control. FIG. 19B shows the anti-MDM2 antibody in high-grade endometrial stromal sarcoma (HGESS) (uterine) tumor cells.

FIG. 20 shows an immunohistochemistry stain using anti-RB1 antibody. FIG. 20A is a positive control. FIG. 20B shows the anti-RB1 stain in leiomyosarcoma tumor cells.

FIG. 21 shows an immunohistochemistry stain using anti-ESR1 antibody. FIG. 21A is a positive control. FIG. 21B shows the anti-ESR1 stain in high grade sarcoma (recurrent tumor) tumor cells.

FIG. 22 shows immunohistochemistry stains in tumor cells. FIG. 22A shows an immunohistochemistry stain using anti-MDM2 antibody in adenosarcoma with sarcoma overgrowth (ASSO) tissue. FIG. 22B shows an immunohistochemistry stain using anti-CDK42 antibody in adenosarcoma with sarcoma overgrowth (ASSO) tissue. FIG. 22C shows an immunohistochemistry stain using anti-AR antibody in adenosarcoma with sarcoma overgrowth (ASSO) tissue.

FIG. 23 shows an immunohistochemistry stain using anti-PD-L1 antibody in glioblastoma tumor cells.

### DETAILED DESCRIPTION

Provided herein are methods and compositions for identifying structural variants. Also provided herein are methods and compositions for identifying oncogenic structural variants. Provided herein are methods and compositions for detecting structural variants. Also provided herein are methods and compositions for detecting oncogenic structural variants.

### Structural Variants

Provided herein are methods for detecting the presence or absence of a structural variant in a sample. Presence of a structural variant may refer to a detectable level or amount in a sample (e.g., by a detection method described herein). Absence of a structural variant may refer to an undetectable level or amount in a sample (e.g., by a detection method described herein). A structural variant may be referred to as a structural variation and/or a chromosomal rearrangement. A structural variant may comprise one or more of a translocation, inversion, insertion, deletion, and duplication. In some embodiments, a structural variant comprises a microduplication and/or a microdeletion. In some embodiments, a structural variant comprises a fusion (e.g., a gene fusion where a portion of a first gene is inserted into a portion of a second gene). Any type of structural variant, whether it be translocation, inversion, insertion, deletion, and/or duplication as described below, can be of any length, and in some embodiments, is about 1 base or base pair (bp) to about 250 megabases (Mb) in length. In some embodiments, a structural variation is about 1 base or base pair (bp) to about 50,000 kilobases (kb) in length (e.g., about 10 bp, 50 bp, 100 bp, 500 bp, 1 kb, 5 kb, 10 kb, 50 kb, 100 kb, 500 kb, 1000 kb, 5000 kb or 10,000 kb in length). A structural variant may be intra-chromosomal (rearrangement of genomic material within a chromosome) or inter-chromosomal (rearrangement of genomic material between two or more chromosomes).

A structural variant may comprise a translocation. A translocation is a genetic event that results in a rearrangement of chromosomal material. Translocations may include reciprocal translocations and Robertsonian translocations. A reciprocal translocation is a chromosome abnormality caused by exchange of parts between non-homologous chromosomes-two detached fragments of two different chromosomes are switched. A Robertsonian translocation occurs when two non-homologous chromosomes become attached, meaning that given two healthy pairs of chromosomes, one of each pair sticks and blends together homogeneously. A gene fusion may be created when a translocation joins two genes that are normally separate. Translocations may be balanced (i.e., in an even exchange of material with no genetic information extra or missing, sometimes with full functionality) or unbalanced (i.e., where the exchange of chromosome material is unequal resulting in extra or missing genes or fragments thereof).

A structural variant may comprise an inversion. An inversion is a chromosome rearrangement in which a segment of a chromosome is reversed end-to-end. An inversion may occur when a single chromosome undergoes breakage and rearrangement within itself. Inversions may be of two types: paracentric and pericentric. Paracentric inversions do not include the centromere, and both breaks occur in one arm of the chromosome. Pericentric inversions include the centromere, and there is a break point in each arm.

A structural variant may comprise an insertion. An insertion may be the addition of one or more nucleotide base pairs into a nucleic acid sequence. An insertion may be a microinsertion (generally a submicroscopic insertion of any length ranging from 1 base to about 10 megabases (e.g., about 1 megabase to about 3 megabases)). In certain embodiments, an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (e.g., insertion) of nucleic acid of unknown origin into a genome, chromosome, or segment thereof. In certain embodiments an insertion comprises the addition (e.g., insertion) of a single base.

A structural variant may comprise a deletion. In certain embodiments, a deletion is a genetic aberration in which a part of a chromosome or a sequence of DNA is missing. A deletion can, in certain embodiments, result in the loss of genetic material. In embodiments, a deletion can be translocated to another portion of the genome (balanced translocation or unbalanced translocation), such as on the same chromosome (same arm of the chromosome or other arm of the chromosome) or on a different chromosome. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion (generally a submicroscopic deletion of any length ranging from 1 base to about 10 megabases (e.g., about 1 megabase to about 3 megabases)). A deletion can comprise the deletion of a single base.

A structural variant may comprise a duplication. In certain embodiments, a duplication is a genetic aberration in which a part of a chromosome or a sequence of DNA is copied and inserted back into the genome. In certain embodiments, a duplication is any duplication of a region of DNA. In some embodiments, a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication (generally a submicroscopic duplication of any length ranging from 1 base to about 10 megabases (e.g., about 1 megabase to about 3 megabases)). A duplication sometimes comprises one or more copies of a duplicated nucleic acid. A duplication may be characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications may occur as the result of an error in homologous recombination or due to a retrotransposon event.

A structural variant may include one or more chromosomal rearrangements (e.g., translocations, inversions, insertions, deletions, duplications). For example, a structural variant may include one or more intra-chromosomal rearrangements. In certain instances, a structural variant may include one or more inter-chromosomal rearrangements. In certain instances, a structural variant may include one or more intra-chromosomal rearrangements and/or one or more inter-chromosomal rearrangements. Such a structural variant may be used as a marker for cancer. In some embodiments, such a structural variant may be used as a marker for cancer of any of the types listed in row 3 of Table 10. Accordingly, provided herein are methods for detecting the presence or absence of one or more intra-chromosomal rearrangements and/or one or more inter-chromosomal rearrangements. Also provided herein are methods for providing a diagnosis of cancer in a subject when the presence of one or more intra-chromosomal rearrangements and/or one or more inter-chromosomal rearrangements is present. In some embodiments, methods for providing a diagnosis of cancer in a subject when the presence of one or more intra-chromosomal rearrangements and/or one or more inter-chromosomal rearrangements is present, where the cancer is of any of the types listed in row 3 of Table 10.

### Breakpoints and Donor/Receiver Sites

A structural variant may be defined according to one or more breakpoints. A breakpoint generally refers to a genomic position (i.e., genomic coordinate) where a structural variant occurs (e.g., translocation, inversion, insertion, deletion, or duplication). A breakpoint may refer to a genomic position where an ectopic portion of genomic material is inserted (e.g., a recipient site for an insertion or a translocation). A breakpoint may refer to a genomic position where a portion of genomic material is deleted (e.g., a donor site for an insertion or a translocation). A breakpoint may refer to a pair of genomic positions (i.e., genomic coordinates) that have become flanking (i.e., adjacent) to one another as a result of a structural variant (e.g., translocation, inversion, insertion, deletion, or duplication). A breakpoint may be defined in terms of a position or positions in a reference genome. A breakpoint may be defined in terms of a position or positions in a human reference genome (e.g., HG38 human reference genome). Generally, genomic positions discussed herein are in reference to an HG38 human reference genome, and corresponding and/or equivalent positions in any other human reference genome are contemplated herein.

A breakpoint may be defined in terms mapping to a position or positions in a reference genome. A breakpoint may be defined in terms of mapping to a position or positions in a human reference genome (e.g., HG38 human reference genome). A breakpoint may map to a position in a reference genome when a nucleic acid sequence located upstream, downstream, or spanning the breakpoint aligns with a corresponding sequence in a reference genome. Any suitable mapping method (e.g., process, algorithm, program, software, module, the like or combination thereof) can be used and certain aspects of mapping processes are described hereafter.

Mapping a nucleic acid sequence may comprise mapping one or more nucleic acid sequence reads (e.g., sequence information from a fragment whose physical genomic position is unknown), which can be performed in a number of ways, and often comprises alignment of the obtained sequence reads with a matching sequence in a reference genome. In such alignments, sequence reads generally are aligned to a reference sequence and those that align are designated as being “mapped”, “a mapped sequence read” or “a mapped read”.

The terms “aligned”, “alignment”, or “aligning” generally refer to two or more nucleic acid sequences that can be identified as a match (e.g., 100% identity) or partial match. Alignments can be done manually or by a computer (e.g., a software, program, module, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (e.g., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch (i.e., a base not correctly paired with its canonical Watson-Crick base partner (e.g., A or T incorrectly paired with C or G). In some embodiments, an alignment comprises 1, 2, 3, 4 or 5 mismatches. Two or more sequences can be aligned using either strand. In certain embodiments a nucleic acid sequence is aligned with the reverse complement of another nucleic acid sequence. In certain instances, extra or missing bases within a sequence are expressed as gaps in an alignment and may or may not be factored into a percent identity calculation. For example, a percent identity calculation may include a number of mismatches and gaps or may include a number of mismatches only.

Various computational methods can be used to map and/or align sequence reads to a reference genome. Non-limiting examples of computer algorithms that can be used to align sequences include, without limitation, BLAST, BLITZ, FASTA, BOWTIE 1, BOWTIE 2, BWA, ELAND, MAQ, PROBEMATCH, SOAP or SEQMAP, or variations thereof or combinations thereof. In some embodiments, sequence reads can be aligned with reference sequences and/or sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database.

In some embodiments, a breakpoint (e.g., donor site) of a structural variant (e.g., insertion, translocation) maps to a particular location within a range of positions on a particular chromosome. A breakpoint for a donor site may map to a particular location within a range of positions that is different from the location of a receiving site. A breakpoint for a donor site may map to a particular location that is on the same chromosome as a receiving site or may map to a particular location that is on a different chromosome than a receiving site. In some embodiments, a breakpoint of a structural variant maps to a location between positions selected from the group consisting of: a position in Row 22 Table 10. In some embodiments, a breakpoint of a structural variant maps to a location between positions selected from the group consisting of: a position in Row 23 Table 10.

In some embodiments, a breakpoint of a structural variant maps to a particular location within a range of positions on a particular chromosome. In some embodiments, a breakpoint (e.g., receiving site) of a structural variant (e.g., insertion, translocation) maps to a particular location within a range of positions on a particular chromosome. In some embodiments, a breakpoint (e.g., donor site) of a structural variant (e.g., insertion, translocation) maps to a particular location within a range of positions on a particular chromosome. A breakpoint for a donor site may map to a particular location within a range of positions that is different from the location of a receiving site. A breakpoint for a donor site may map to a particular location that is on the same chromosome as a receiving site or may map to a particular location that is on a different chromosome than a receiving site. A structural variant may be defined in terms of a receiving site and a donor site. A receiving site may be referred to as a first partner or “partner 1” and a donor site may be referred to as a second partner or “partner 2.” In some embodiments, a structural variant may be defined in terms of comprising an ectopic portion of genomic DNA (i.e., a portion of genomic DNA at a receiving site from a different region of a chromosome or from a different chromosome). The ectopic portion may be referred to as a donor portion.

In some embodiments, a receiving site of a structural variant maps to a location between positions selected from the group consisting of: a position in Row 22 Table 10. In some embodiments, a receiving site of a structural variant maps to a location between positions selected from the group consisting of: a position in Row 23 Table 10. In some embodiments, a receiving site of a structural variant maps to a location between positions selected from the group consisting of: a position in Row 5 Table 10. In some embodiments, a receiving site of a structural variant maps to a location between positions selected from the group consisting of: a position in Row 6 Table 10.

In some embodiments, a structural variant may comprise an ectopic portion of genomic DNA (i.e., a portion of genomic DNA at a receiving site from a different region of a chromosome or from a different chromosome). The ectopic portion may be referred to as a donor portion. If the ectopic portion (donor portion) is from the same chromosome as the structural variant, the ectopic portion may be from a location outside of the position ranges provided above for certain structural variants. The ectopic portion may comprise genomic DNA from a genomic coordinate window provided herein, or part thereof. The ectopic portion may comprise genomic DNA from a genomic coordinate window provided herein, or part thereof, and may further comprise genomic DNA from a region outside of a genomic coordinate window provided herein.

In some embodiments, an ectopic portion of genomic DNA is characterized by its location (e.g., observed location for a given sample or samples) at a receiving site (e.g., at a structural variant site). In some embodiments, an ectopic portion is characterized by its location (e.g., observed location for a given sample samples) relative to the gene body of a gene and/or cancer gene. A gene body of a gene and/or cancer gene generally refers to a part of the gene and/or cancer gene that is transcribed. In some embodiments, an ectopic portion is within the gene body of a gene and/or cancer gene. In some embodiments, an ectopic portion is not within a gene body of a gene and/or cancer gene. For example, an ectopic portion may be located in an an intergenic region adjacent to a cancer gene, or within another gene adjacent to a cancer gene. In some embodiments, an ectopic portion is located at a position in proximity to the gene body for a gene and/or cancer gene. The term “in proximity” may refer to spatial proximity and/or linear proximity.

Spatial proximity generally refers to 3-dimensional chromatin proximity, which may be assessed according to a method that preserves spatial-proximal relationships, such as a method described herein or any suitable method known in the art. An ectopic portion may be located at a position in spatial proximity to the gene body for a gene and/or cancer gene when an ectopic portion and a gene and/or cancer gene (or a fragment thereof) are ligated in a proximity ligation assay or are bound by a common solid phase in a solid substrate-mediated proximity capture (SSPC) assay, for example.

Linear proximity generally refers to a linear base-pair distance, which may be assessed according to mapped distances in a reference genome, for example. Linear proximity distance may be provided as a distance between a 5′ or 3′ end of an ectopic portion and a 5′ or 3′ end of a gene and/or exon. An ectopic portion may be located at a position in linear proximity to the gene body of a gene, cancer gene, and/or oncogene when the ectopic portion is within about 1,000 base pairs, about 2,000 base pairs, about 3,000 base pairs, about 4,000 base pairs, about 5,000 base pairs, about 10,000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500,000 base pairs, about 600,000 base pairs, about 700,000 base pairs, about 800,000 base pairs, about 900,000 base pairs, or about 1,000,000 base pairs of a coding region of a gene, cancer gene, and/or oncogene. Sometimes the ectopic portion, while in proximity to a cancer gene or cancer gene, as described above, also happens to be within a non-cancer gene/cancer gene. Sometimes the ectopic portion, while in proximity to a cancer gene or oncogene, as described above, is not within a gene and is positioned in an intergenic region.

In some embodiments, a structural variant comprises an ectopic portion of genomic DNA from a chromosome selected from the group consisting of: a chromosome listed in rows 16, 17, 22, and 23 of Table 10 (donor site). In some embodiments, an ectopic portion is located at a position in a chromosome selected from the group consisting of: a chromosome listed in rows 5, 6, 8, and 9 of Table 10 (receiver site) in proximity to a coding region for a corresponding cancer gene selected from the group consisting of: a cancer gene listed in row 7 of Table 10. In some embodiments, an ectopic portion is located at a position in a chromosome selected from the group consisting of: a chromosome listed in rows 5, 6, 8, and 9 of Table 10 (receiver site) in spatial proximity to a coding region for a corresponding cancer gene selected from the group consisting of: a cancer gene listed in row 7 of Table 10. In some embodiments, an ectopic portion is located at a position in a chromosome selected from the group consisting of: a chromosome listed in rows 5, 6, 8, and 9 of Table 10 (receiver site) in linear proximity to a coding region for a corresponding cancer gene selected from the group consisting of: a cancer gene listed in row 7 of Table 10.

In some embodiments, an ectopic portion is located at a position in a chromosome selected from the group consisting of: a chromosome listed in rows 5, 6, 8, and 9 of Table 10 (receiver site) within about 1,000 base pairs, about 2,000 base pairs, about 3,000 base pairs, about 4,000 base pairs, about 5,000 base pairs, about 10,000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500,000 base pairs, about 600,000 base pairs, about 700,000 base pairs, about 800,000 base pairs, about 900,000 base pairs, or about 1,000,000 base pairs of a coding region of the corresponding cancer gene selected from the group consisting of: a cancer gene listed in row 7 of Table 10. In some embodiments, an ectopic portion is located at a position in a chromosome selected from the group consisting of: a chromosome listed in rows 5, 6, 8, and 9 of Table 10 within a linear distance of the 5′ end of a corresponding cancer gene selected from the group consisting of: a cancer gene listed in row 7 of Table 10. The linear distance from the 5′ end for cancer gene is shown in row 12 of Table 10. In some embodiments the linear distance from the 5′ end can be about +/−10 bp, +/−50 bp, +/−100 bp, +/−500 bp, +/−1 kb, +/−5 kb, +/−10 kb, +/−50 kb, +/−100 kb or +/−500 kb what is listed in row 12 of Table 10. In some embodiments, an ectopic portion is located at a position in a chromosome selected from the group consisting of: a chromosome listed in rows 5, 6, 8, and 9 of Table 10 within a linear distance of the 3′ end of a corresponding cancer gene selected from the group consisting of: a cancer gene listed in row 7 of Table 10. Row 13 of Table 10 shows the closest distance to the gene body of the corresponding cancer gene from row 7 of Table 10. If value in row 13 of Table 10 matches the value in row 12 of Table 10, the ectopic portion is nearer the 5′ of the corresponding cancer gene from row 7 of Table 10. If the value in row 13 of Table 10 does not match the value in row 12 of Table 10, the ectopic portion is nearer the 3′ of the corresponding cancer gene from row 7 of Table 10. If relevant (i.e. the values in row 12 and row 13 of Table 10 do not match), the linear distance from the 3′ end for cancer gene is shown in row 13 of Table 10. In some embodiments the linear distance from the 3′ end can be about +/−10 bp, +/−50 bp, +/−100 bp, +/−500 bp, +/−1 kb, +/−5 kb, +/−10 kb, +/−50 kb, +/−100 kb or +/−500 kb what is listed in row 13 of Table 10.

In some embodiments, a structural variant comprises an ectopic portion of genomic DNA from a chromosome selected from the group consisting of: a chromosome listed in rows 5, 6, 8, and 9 of Table 10 (donor site). In some embodiments, an ectopic portion is located at a position in a chromosome selected from the group consisting of: a chromosome listed in rows 16, 17, 22, and 23 of Table 10 (receiver site) in proximity to a coding region for a corresponding cancer gene selected from the group consisting of: a cancer gene listed in row 15 of Table 10. In some embodiments, an ectopic portion is located at a position in a chromosome selected from the group consisting of: a chromosome listed in rows 16, 17, 22, and 23 of Table 10 (receiver site) in spatial proximity to a coding region for a corresponding cancer gene selected from the group consisting of: a cancer gene listed in row 15 of Table 10. In some embodiments, an ectopic portion is located at a position in a chromosome selected from the group consisting of: a chromosome listed in rows 16, 17, 22, and 23 of Table 10 (receiver site) in linear proximity to a coding region for a corresponding cancer gene selected from the group consisting of: a cancer gene listed in row 15 of Table 10.

In some embodiments, an ectopic portion is located at a position in a chromosome selected from the group consisting of: a chromosome listed in rows 16, 17, 22, and 23 of Table 10 (receiver site) within about 1,000 base pairs, about 2,000 base pairs, about 3,000 base pairs, about 4,000 base pairs, about 5,000 base pairs, about 10,000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500,000 base pairs, about 600,000 base pairs, about 700,000 base pairs, about 800,000 base pairs, about 900,000 base pairs, or about 1,000,000 base pairs of a coding region of the corresponding cancer gene selected from the group consisting of: a cancer gene listed in row 15 of Table 10. In some embodiments, an ectopic portion is located at a position in a chromosome selected from the group consisting of: a chromosome listed in rows 16, 17, 22, and 23 of Table 10 within a linear distance of the 5′ end of a corresponding cancer gene selected from the group consisting of: a cancer gene listed in row 15 of Table 10. The linear distance from the 5′ end for cancer gene is shown in row 20 of Table 10. In some embodiments the linear distance from the 5′ end can be about +/−10 bp, +/−50 bp, +/−100 bp, +/−500 bp, +/−1 kb, +/−5 kb, +/−10 kb, +/−50 kb, +/−100 kb or +/−500 kb what is listed in row 20 of Table 10.

In some embodiments, an ectopic portion is located at a position in a chromosome selected from the group consisting of: a chromosome listed in rows 16, 17, 22, and 23 of Table 10 within a linear distance of the 3′ end of a corresponding cancer gene selected from the group consisting of: a cancer gene listed in row 15 of Table 10. Row 21 of Table 10 shows the closest distance to the gene body of the corresponding cancer gene from row 15 of Table 10. If value in row 21 of Table 10 matches the value in row 20 of Table 10, the ectopic portion is nearer the 5′ of the corresponding cancer gene from row 15 of Table 10. If the value in row 21 of Table 10 does not match the value in row 20 of Table 10, the ectopic portion is nearer the 3′ of the corresponding cancer gene from row 15 of Table 10. If relevant (i.e. the values in row 20 and row 21 of Table 10 do not match), the linear distance from the 3′ end for cancer gene is shown in row 21 of Table 10. In some embodiments the linear distance from the 3′ end can be about +/−10 bp, +/−50 bp, +/−100 bp, +/−500 bp, +/−1 kb, +/−5 kb, +/−10 kb, +/−50 kb, +/−100 kb or +/−500 kb what is listed in row 21 of Table 10.

### Oncogenes/Cancer Genes

A structural variant may be associated with one or more genes. For example, a structural variant may be associated with one or more cancer genes. A cancer gene is a gene that, when altered, is associated with cancer. Alterations may include mutations, structural variants, copy number variations, and the like and combinations thereof. With respect to cancer genes, alterations may be located within a cancer gene (i.e., intragenic with respect to the cancer gene) or outside of/adjacent to a cancer gene (i.e., extragenic with respect to the cancer gene). For structural variants, the terms “outside of” and “adjacent to,” as used herein in reference to a structural variant being outside of or adjacent to a cancer gene generally means that a breakpoint of a structural variant is not within the cancer gene. When the breakpoint of a structural variant is not within the cancer gene, it may be intergenic, or, within an adjacent gene. The structural variant can contain the gene, such as an inversion of the gene, an insertion of the gene, a duplication of the gene, or the like, or can contain a portion of the gene. In certain aspects, the structural variant may not include the gene, i.e., the structural variant does not contain the gene, insertion, inversion, duplication or any portion thereof.

In certain instances, alterations and/or structural variant breakpoints may be located within a different gene adjacent to a cancer gene. The gene may a non-cancer gene adjacent to a cancer gene or may not be a cancer gene adjacent to another cancer gene. The term “cancer gene” as used herein means a gene associated with cancer (for example, but not limited to, a tumor suppressor and oncogene). Alterations and/or structural variant breakpoints may be located in a portion of genomic DNA that is proximal to a cancer gene (e.g., within a certain linear proximity and/or within a certain spatial proximity). Alterations and/or structural variant breakpoints may affect expression of a cancer gene (e.g., increased expression, decreased expression, no expression, constitutive expression). Alterations and/or structural variant breakpoints may affect the function of a protein encoded by a cancer gene (e.g., increased function, decreased function, loss-of-function, gain-of-function, constitutive function, change in function). Non-limiting examples of cancer genes are provided in Table 7.

In some embodiments, a structural variant is associated with one or more genes selected from the group consisting of: genes in row 7 and row 15 of Table 10. In some embodiments, a structural variant associated with a one or more genes selected from the group consisting of: genes in row 7 and row 15 of Table 10 is detected in a sample from a subject, where the subject has cancer. Such structural variants may be used as markers for cancer. In embodiments, such structural variants may be used as markers for cancer of the types listed in row 3 of Table 10. In embodiments, such structural variants may be used as markers for the corresponding cancer disclosed in row 3 of Table 10 for that particular variant.

Accordingly, provided herein are methods for detecting the presence or absence of a structural variant is associated with one or more genes selected from the group consisting of: genes in row 7 and row 15 of Table 10. Also provided herein are methods for providing a diagnosis of cancer in a subject when the presence of a structural variant is associated with one or more genes selected from the group consisting of: genes in row 7 and row 15 of Table 10 is detected. Provided herein are methods for providing a diagnosis of cancer in a subject when the presence of a structural variant associated with one or more genes selected from the group consisting of: genes in row 7 and row 15 of Table 10) is detected, where the type of cancer is one listed in row 3 Table 10. In embodiments are methods for providing a diagnosis of cancer in a subject when the presence of a structural variant associated with one or more genes selected from the group consisting of: genes in row 7 and row 15 of Table 10) is detected, where the type of cancer is one listed in row 3 Table 10 row 3 of Table 10 for that particular variant.

In some embodiments, a structural variant and/or breakpoint of a structural variant is within a gene (e.g., within an intron and/or exon of a gene (e.g., an oncogene)). In some embodiments, a structural variant and/or breakpoint of a structural variant is outside of a gene (e.g., within an intergenic region or within a different nearby gene). In some embodiments, a structural variant and/or breakpoint of a structural variant is adjacent to a gene (e.g., within an intergenic region or within a different nearby gene). Thus, in some embodiments, a structural variant and/or breakpoint of a structural variant is not within a gene (e.g., an oncogene). In certain instances, a structural variant and/or breakpoint of a structural variant (e.g., an intergenic structural variant) may be defined in terms of linear distance to a gene (e.g., an oncogene). Linear distance may be measured from the 5′ end of a gene and/or a 3′ end of a gene. In some embodiments, a structural variant and/or breakpoint of a structural variant may be located at least about 1 kb to about 500 kb from the 5′ end or 3′ end of a gene. For example, a structural variant and/or breakpoint of a structural variant may be located at least about 1 kb, 2 kb, 3 kb, 4 kb, 5 kb, 10 kb, 20 kb, 30 kb, 40 kb, 50 kb, 60 kb, 70 kb, 80 kb, 90 kb, 100 kb, 200 kb, 300 kb, 400 kb, or 500 kb from the 5′ end or 3′ end of a gene. In some embodiments, a structural variant and/or breakpoint of a structural variant may be located at least about 1 kb to about 200,000 kb from the 5′ end or 3′ end of a gene. For example, a structural variant and/or breakpoint of a structural variant may be located at least about 1 kb, 2 kb, 3 kb, 4 kb, 5 kb, 10 kb, 20 kb, 30 kb, 40 kb, 50 kb, 60 kb, 70 kb, 80 kb, 90 kb, 100 kb, 200 kb, 300 kb, 400 kb, 500 kb, 1000 kb, 10,000 kb, 100,000 kb, 150,000 kb, or 200,000 kb from the 5′ end or 3′ end of a gene. In some embodiments, a structural variant and/or breakpoint of a structural variant is located at least about 10 base pairs from an oncogene terminus. In some embodiments, a structural variant and/or breakpoint of a structural variant is located at least about 100 base pairs from an oncogene terminus. In some embodiments, a structural variant and/or breakpoint of a structural variant is located at least about 500 base pairs from an oncogene terminus. In some embodiments, a structural variant and/or breakpoint of a structural variant is located at least about 1,000 base pairs from an oncogene terminus. In some embodiments, a structural variant and/or breakpoint of a structural variant is located at least about 4,000 base pairs from an oncogene terminus. In some embodiments, a structural variant and/or breakpoint of a structural variant is located at about 10 base pairs to about 700,000 base pairs from an oncogene terminus. In some embodiments, a structural variant and/or breakpoint of a structural variant is located at about 4,000 base pairs to about 700,000 base pairs from an oncogene terminus. In some embodiments, a structural variant and/or breakpoint of a structural variant is located at about 10 base pairs to about 100,000 base pairs from an oncogene terminus. In some embodiments, a structural variant and/or breakpoint of a structural variant is located at about 4,000 base pairs to about 100,000 base pairs from an oncogene terminus. In some embodiments, a structural variant and/or breakpoint of a structural variant is located at about 500 base pairs to about 1,630,000 base pairs from an oncogene terminus. In some embodiments, a structural variant and/or breakpoint of a structural variant is located at about 500 base pairs to about 650,000 base pairs from an oncogene terminus. In some embodiments, a structural variant and/or breakpoint of a structural variant is located at about 500 base pairs to about 100,000 base pairs from an oncogene terminus. An oncogene terminus may be a 5′ terminus or a 3′ terminus.

### Nucleic Acid

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), small interfering RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil (U). Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single-stranded or double-stranded. Single-stranded DNA (ssDNA), for example, can be generated by denaturing double-stranded DNA by heating or by treatment with alkali, for example. Accordingly, in some embodiments, ssDNA is derived from double-stranded DNA (dsDNA).

Nucleic acid (e.g., genomic DNA, nucleic acid targets, oligonucleotides, probes, primers) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions. In certain instances, a nucleotide sequence may be partially complementary to a target, in which not all nucleotides are complementary to every nucleotide in the target nucleic acid in all the corresponding positions.

The percent identity of two nucleotide sequences can be determined by aligning the sequences for optimal comparison purposes. When the total number of positions is different between the two nucleotide sequences, gaps may be introduced in the sequence of one or both sequences for optimal alignment. The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. In certain instances, extra or missing bases within a sequence are expressed as gaps in an alignment and may or may not be factored into a percent identity calculation. For example, a percent identity calculation may include a number of mismatches and gaps or may include a number of mismatches only.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, oligonucleotide, or probe, to a nucleic acid molecule having a sequence complementary to the primer, oligonucleotide, or probe compared to hybridization to a nucleic acid molecule not having a complementary sequence. For example, specific hybridization includes the hybridization of a primer, oligonucleotide, or probe to a target nucleic acid sequence that is complementary to the primer, oligonucleotide, or probe.

Primer, oligonucleotide, or probe sequences and length can affect hybridization to target nucleic acid sequences. Depending on the degree of mismatch between the primer, oligonucleotide, or probe and target nucleic acid, low, medium or high stringency conditions may be used to effect primer/target, oligonucleotide/target, or probe/target annealing. As used herein, the term “stringent conditions” refers to conditions for hybridization and washing. Methods for hybridization reaction temperature condition optimization are known, and can be found. e.g., in Current Protocols in Molecular Biology, John Wiley & Sons, N.Y., 6.3.1-6.3.6 (1989). Aqueous and non-aqueous methods are described in the aforementioned reference and either can be used. Non-limiting examples of stringent hybridization conditions include, for example, hybridization in 6× sodium chloride/sodium citrate (SSC) at about 45° C., followed by one or more washes in 0.2×SSC, 0.1% SDS at 50° C. Another example of stringent hybridization conditions includes hybridization in 6× sodium chloride/sodium citrate (SSC) at about 45° C., followed by one or more washes in 0.2×SSC, 0.1% SDS at 55° C. A further example of stringent hybridization conditions includes hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions can include 0.5 M sodium phosphate, 7% SDS at 65° C., followed by one or more washes at 0.2×SSC, 1% SDS at 65° C. Stringent hybridization temperatures also can be altered (generally, lowered) with the addition of certain organic solvents, such as formamide for example. Organic solvents such as formamide can reduce the thermal stability of double-stranded polynucleotides, so that hybridization can be performed at lower temperatures, while still maintaining stringent conditions and extending the useful life of heat labile nucleic acids.

In some embodiments, target nucleic acids comprise degraded DNA. Degraded DNA may be referred to as low-quality DNA or highly degraded DNA. Degraded DNA may be highly fragmented and may include damage such as base analogs and abasic sites subject to miscoding lesions and/or intermolecular crosslinking. For example, sequencing errors resulting from deamination of cytosine residues may be present in certain sequences obtained from degraded DNA (e.g., miscoding of C to T and G to A).

Nucleic acid may be derived from one or more sources (e.g., a biological sample described herein) by methods known in the art. Any suitable method can be used for isolating, extracting and/or purifying DNA from a biological sample (e.g., from blood or a blood product, tissue, tumor), non-limiting examples of which include methods of DNA preparation, various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N.J.); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccfDNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In certain aspects, cell-free nucleic acid comprises circulating cancer nucleic acid (e.g., cancer DNA). In certain aspects, cell-free nucleic acid comprises circulating tumor nucleic acid (e.g., tumor DNA).

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In some instances, cancer nucleic acid and/or tumor nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, or tumor nucleic acid).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

### Nucleic Acid Analysis

A method herein may comprise one or more nucleic acid analyses. For example, nucleic acid obtained from a sample from a subject may be analyzed for the presence or absence of a structural variant. Any suitable process for detecting a structural variant in a nucleic acid sample may be used. Non-limiting examples of processes for analyzing nucleic acid include amplification (e.g., polymerase chain reaction (PCR)), targeted sequencing, microarray, and fluorescence in situ hybridization (FISH), methods that preserve spatial-proximal contiguity information, methods that preserve spatial-proximity relationships, and methods that generate proximity ligated nucleic acid molecules.

In some embodiments, a nucleic acid analysis comprises nucleic acid amplification. For example, nucleic acids may be amplified under amplification conditions. The term “amplified” or “amplification” or “amplification conditions” generally refer to subjecting a target nucleic acid in a sample to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). Detecting a structural variant (SV) described herein using amplification (e.g., PCR) may include use of primers designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of PCR primers useful for identifying a structural variant are provided herein.

In some embodiments, a nucleic acid analysis comprises fluorescence in situ hybridization (FISH). Fluorescence in situ hybridization (FISH) is a technique that uses fluorescent probes that bind to a nucleic acid sequence with a high degree of sequence complementarity. In certain configurations, fluorescence microscopy may be used to observe where the fluorescent probe is bound to a chromosome. Detecting a structural variant (SV) described herein using fluorescence in situ hybridization (FISH) may include use of probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of probes useful for identifying a structural variant are provided herein.

In some embodiments, a nucleic acid analysis comprises a microarray (e.g., a DNA microarray, DNA chip, biochip). A DNA microarray is a collection of DNA probes attached to a solid surface. Probes can be short sections of a gene or other genomic DNA element that can hybridize to target nucleic acids in a sample (e.g., under high-stringency conditions). Probe-target hybridization is usually detected and quantified by detection of fluorophore-, silver-, or chemiluminescence-labeled targets to determine presence, absence, and/or relative abundance of target nucleic acid sequences in the sample. Detecting a structural variant (SV) described herein using DNA microarrays may include use of array probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of array probes useful for identifying a structural variant are provided herein.

In some embodiments, a nucleic acid analysis comprises sequencing (e.g., genome-wide sequencing, targeted sequencing). For targeted sequencing, a target nucleic acid may be amplified (e.g., by PCR with primers specific to the target), enriched using a probe-based approach, where one or more probes hybridize to a target nucleic acid prior to sequencing, or enriched using Cas9-mediated approaches, such as Cas9-guided adapter ligation, as described in Gilpatrick, T. et al., Targeted nanopore sequencing with Cas9-guided adapter ligation, Nature Biotechnology, volume 38, pages 433-438 (2020). Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides.

In some embodiments, a nucleic acid analysis comprises a method that preserves spatial-proximal relationships and/or spatial-proximal contiguity information (see e.g., International PCT Application Publication No. WO2019/104034; International PCT Application Publication No. WO2020/106776; International PCT Application Publication No. WO2020236851; Kempfer, R., & Pombo, A. (2019). Methods for mapping 3D chromosome architecture. Nature Reviews Genetics. doi: 10.1038/s41576-019-0195-2; and Schmitt, Anthony D.; Hu, Ming; Ren, Bing (2016). Genome-wide mapping and analysis of chromosome architecture. Nature Reviews Molecular Cell Biology. doi: 10.1038/nrm.2016.104; each of which is incorporated by reference in its entirety, to the extent permitted by law).

Methods that preserve spatial-proximal relationships and/or spatial-proximal contiguity information generally refer to methods that capture and preserve the native spatial conformation exhibited by nucleic acids when associated with proteins as in chromatin and/or as part of a nuclear matrix. Spatial-proximal contiguity information and/or spatial-proximity relationships can be preserved by proximity ligation, by solid substrate-mediated proximity capture (SSPC), by compartmentalization with or without a solid substrate or by use of a Tn5 tetramer. Methods that preserve spatial-proximal contiguity information and/or preserve spatial-proximity relationships may be based on proximity ligation or may be based on a different principle where special proximity is inferred. Methods based on proximity ligation may include, for example, 3C, 4C, 5C, Hi-C, TCC, GCC, TLA, PLAC-seq, HiChIP, ChIA-PET, Capture-C, Capture-HiC, single-cell HiC, sciHiC, single-cell 3C, single-cell methyl-3C, DNAase HiC, Micro-C, Tiled-C, and Low-C. Methods where special proximity is inferred based on a principle other than proximity ligation may include, for example, SPRITE, scSPRITE, Genome Architecture Mapping (GAM), ChIA-Drop, imaging-based approaches using labeled probes and visualization of DNA, and plus/minus sequencing of an imaged sample (e.g. in situ Genome Sequencing (IGS)). In some embodiments, a nucleic acid analysis comprises generating proximity ligated nucleic acid molecules (e.g., using a method described herein). In some embodiments, a nucleic acid analysis comprises sequencing the proximity ligated nucleic acid molecules, e.g., by a suitable sequencing process known in the art or described herein.

### Non-Spatial Proximal Contiguity DNA Sequencing Methodologies:

Non-spatial proximal contiguity sequencing methodologies, including but not limited to Shotgun WGS, Linked-Read WGS and other forms of synthetic long-read sequencing, Mate-pair WGS and similar techniques (Fosmids, BACs), Long-read WGS, and other known or anticipated non-spatial proximal contiguity DNA sequencing methodologies, either sequenced “in bulk” or with single-cell and/or spatial resolution, either in “genome-wide” or “targeted” format (“targeted” meaning, for example, by using known or anticipated target enrichment methodologies (e.g. probe based enrichment or PCR), or depletion methodologies (e.g. using CRISPR), or other targeted sequencing techniques (e.g. adaptive sampling), and either sequenced on any known or anticipated short or long-read sequencing platform.

### Spatial Proximal Contiguity DNA Sequencing Methodologies:

### Proximity Ligation DNA Sequencing:

Genome-wide proximity ligation sequencing techniques, including but not limited to: 3C-seq, Hi-C, DNAase HiC, Micro-C, Low-C, TCC, GCC, single-cell HiC, sciHiC, single-cell 3C, single-cell methyl-3C and other genome-wide bulk or single-cell and/or spatial derivatives, sequenced on any known or anticipated short or long-read sequencing platforms.

Targeted proximity ligation sequencing techniques, including but not limited to 3C-(q) PCR, 4C, 5C, Targeted Locus Amplification (TLA), PLAC-seq, HiChIP, ChIA-PET, Capture-C, Capture-HiC, Tiled-C and other genome-wide bulk or single-cell or spatial derivatives, including additional “targeted” techniques (“targeted” meaning, for example, by using known or anticipated target enrichment methodologies (e.g. probe based enrichment or PCR, or protein enrichment), or depletion methodologies (e.g. using CRISPR), or other targeted sequencing techniques (e.g. adaptive sampling), and sequenced on any known or anticipated short or long-read sequencing platforms.

### Non-Proximity Ligation DNA Sequencing:

Non-proximity ligation sequencing techniques, including but not limited to: SPRITE, scSPRITE, other SPRITE derivatives or related techniques involving barcoding of chromatin aggregates, ChIA-Drop or other droplet-based chromatin aggregate barcoding and sequencing techniques, and Genome Architecture Mapping or related techniques where spatial proximal contiguity is inferred from co-occurrence in cryosections. In addition, it is anticipated that additional derivatives of the above may be suitable for proximity fusion detection (i.e adjacent to a cancer gene), including “targeted” versions (“targeted” meaning, for example, by using known or anticipated target enrichment methodologies (e.g. probe based enrichment or PCR), or depletion methodologies (e.g. using CRISPR), or other targeted sequencing techniques (e.g. adaptive sampling), and sequenced on any known or anticipated short or long-read sequencing platforms.

### Imaging Methodologies:

Classic DNA FISH analysis, with one probe on either side of a breakpoint, can detect proximity fusions. However, recent derivatives thereof, including but not limited to SeqFISH, MERFISH, and OligoFISSEQ, could also detect proximity fusions, and due to their high plexity capability could be more tolerant to heterogeneous breakpoint locations and be able to detect proximity fusions involving more than one gene per experiment (possibly hundreds of genes or someday genome-scale).

### Imaging Plus Sequencing Methodologies:

In situ Genome Sequencing (IGS), or related techniques that sequence DNA molecules “in situ”, measuring the location in the nucleus of each sequenced DNA molecule.

### Optical Genome Mapping

PCR—As an example, breakpoint-crossing PCR could be used to detect proximity fusions, so long as the breakpoint is flanked by PCR primers.

Methodologies that infer breakpoints based on genomic coverage—in the absence of identifying a sequence fragment that contains a genomic breakpoint of a proximity (or gene) fusion, techniques may be used to infer structural variant breakpoints based on genomic coverage alone. For example, cytogenic microarrays (e.g. including but not limited to array-based CGH, SNP microarrays, or DNA methylation arrays) can be used to identify copy number gains and losses (i.e. unbalanced chromosomal rearrangements), and the genomic positions where the copy number gain or loss starts/ends can be inferred to be a structural variant breakpoint. One then may be able to look for cancer genes near those breakpoints to identify proximity fusions. While the description here uses microarrays as an example methodology for generating genomic coverage data, it is anticipated that essentially any of the above described sequencing-based methodologies (Non-spatial proximal contiguity DNA Sequencing Methodologies, Spatial proximal contiguity DNA Sequencing Methodologies, Imaging plus Sequencing Methodologies), or Optical Genome Mapping, or any technique that reliably quantifies genome coverage could potentially be used to infer breakpoints based on coverage, and potentially enable the detection of proximity fusions in the absence of a analyzed DNA fragment containing a breakpoint.

In some embodiments, a nucleic acid analysis comprises a method for preparing nucleic acids from particular types of samples that preserves spatial-proximal contiguity information in the sequence of the nucleic acids. Nucleic acid molecules that preserve spatial-proximal contiguity information can fragmented and sequenced using short-read sequencing methods (e.g., Illumina, nucleic acid fragments of lengths approximately 500 bp) or intact molecules that preserve spatial-proximal contiguity information can be sequenced using long-read sequencing (e.g., Illumina, Oxford Nanopore, or others, nucleic acid fragments of lengths approximately 10 Kbp or greater). Similarly, Nucleic acid molecules that preserve spatial-proximal contiguity information can be subject to “synthetic” long-reads, where intact molecules are fragmented and sequenced using short-read sequencing methods (e.g., Illumina, nucleic acid fragments of lengths approximately 500 bp), but where the contiguity of the intact molecules is preserved before or during fragmentation.

In certain embodiments, a sample can be a fixed sample that is embedded in a material such as paraffin (wax). In some embodiments, a sample can be a formalin fixed sample. In certain embodiments, a sample is formalin-fixed paraffin-embedded (FFPE) sample. In some embodiments, a formalin-fixed paraffin-embedded sample can be a tissue sample or a cell culture sample. In some embodiments, a tissue sample has been excised from a patient and can be diseased or damaged. In some embodiments, a tissue sample is not known to be diseased or damaged. In certain embodiments, a formalin-fixed paraffin-embedded sample can be a formalin-fixed paraffin-embedded section, block, scroll or slide. In certain embodiments, a sample can be a deeply formalin-fixed sample, as described below.

In certain embodiments, a formalin-fixed paraffin-embedded sample is provided on a solid surface and a method of preparing nucleic acid that preserves spatial-proximal contiguity information and/or spatial-proximity relationships is performed on the solid surface. In some embodiments, a solid surface is a pathology slide. In some embodiments, additional downstream reactions are also performed on the solid surface.

Those of skill in the art are familiar with methods that can be substituted for steps requiring centrifugation and that achieve a comparable result but are performed on a solid surface.

In some embodiments, methods that preserve spatial-proximal contiguity information and/or spatial-proximity relationships comprise methods that generate proximity ligated nucleic acid molecules (e.g., using proximity ligation). A proximity ligation method is one in which natively occurring spatially proximal nucleic acid molecules are captured by ligation to generate ligated products. Proximity ligation methods generally capture spatial-proximal contiguity information in the form of ligation products, whereby a ligation junction is formed between two natively spatially proximal nucleic acids. Once the ligation products are formed, the spatial-proximal contiguity information is detected using next generation sequencing, whereby one or more ligation junctions (either from an entire ligation product or fragment of a ligation product) are sequenced (as described herein). With this sequence information, one is informed that the nucleic acid molecules from a given ligation product (or ligation junction) are natively spatially proximal nucleic acids. In some embodiments, reagents that generate proximity ligated nucleic acid molecules can include a restriction endonuclease, a DNA polymerase, a plurality of nucleotides comprising at least one biotinylated nucleotide, and a ligase. In certain embodiments, two or more restriction endonucleases are used.

Any suitable method for carrying out proximity ligation may be used. For example, a HiC method typically includes the following steps: (1) digestion of chromatin of a solubilized and decompacted FFPE sample with a restriction enzyme (or fragmentation); (2) labelling the digested ends by filling in the 5′-overhangs with biotinylated nucleotides; and (3) ligating the spatially proximal digested ends, thus preserving spatial-proximal contiguity information. Once spatial-proximal contiguity information is preserved, further steps in a HiC method may include: purifying and enriching biotin-labelled ligation junction fragments, preparing a library from the enriched fragments and sequencing the library. Another example of a proximity ligation method may include the following steps: (1) digestion of chromatin of the solubilized and decompacted sample with a restriction enzyme (or fragmentation); (2) blunting the digested or fragmented ends or omission of the blunting procedure; and (3) ligating the spatially proximal ends, thus preserving spatial-proximal contiguity information. Once spatial-proximal contiguity information is preserved, further steps can include: using size selection to purify and enrich ligated fragments, which represent ligation junction fragments, preparing a library from the enriched fragments and sequencing the library. In some embodiments, proximity ligated nucleic acid molecules are generated in situ (i.e., within a nucleus). For methods that include Capture HiC, a further step is included where ligation products containing certain nucleic acid sequences are enriched using one or more capture probes (see e.g., International Patent Application Publication No. WO 2014/168575). A capture probe generally comprises a short sequence of nucleotides or oligonucleotide (e.g., 10-500 bases in length) capable of hybridizing to another nucleotide sequence. In some embodiments, a capture probe comprises a label (e.g., a label for selectively purifying specific nucleic acid sequences of interest). Labels are discussed herein and may include, for example, a biotin or digoxigenin label. In some embodiments, capture probes are designed according to a panel of sequences and/or genes of interest (e.g., an oncopanel provided herein).

In some embodiments, a nucleic acid analysis herein comprises generating proximity ligated nucleic acid molecules. In some embodiments, a nucleic acid analysis herein comprises contacting the proximity ligated nucleic acid molecules with one or more capture probe species, thereby generating enriched proximity ligated nucleic acid molecules. A capture probe species may comprise a polynucleotide identical to or complementary to a subsequence in a gene (e.g., an oncogene). A capture probe species may comprise a polynucleotide identical to or complementary to a subsequence in an exon of a gene (e.g., an oncogene). A capture probe species may further comprise one or more bases or a polynucleotide identical to or complementary to a subsequence in an intron of a gene (e.g., an oncogene). Thus, a capture probe species may comprise a first polynucleotide identical to or complementary to a subsequence in an exon of a gene (e.g., an oncogene), and may further comprise a one or more bases or second polynucleotide identical to or complementary to a subsequence in an intron of a gene (e.g., an oncogene). A capture probe species may comprise a polynucleotide identical to or complementary to a subsequence in an exon of a gene (e.g., an oncogene) listed in Table 7. A capture probe species may further comprise a polynucleotide identical to or complementary to a subsequence in an intron of a gene (e.g., an oncogene) listed in Table 7. In some embodiments, a polynucleotide (i.e., a polynucleotide in a capture probe) maps to coordinates that are proximal to a site targeted by a restriction enzyme (i.e., a restriction enzyme recognition site). In some embodiments, a polynucleotide (i.e., a polynucleotide in a capture probe) maps to coordinates that are within 300-400 bp of a site targeted by a restriction enzyme. In some embodiments, a polynucleotide (i.e., a polynucleotide in a capture probe) maps to coordinates that are within 350 bp of a site targeted by a restriction enzyme. A site targeted by a restriction enzyme may be selected according to one or more corresponding restriction enzymes used to generate proximity ligated nucleic acid molecules. For example, a restriction enzyme (and/or a corresponding restriction enzyme recognition site) may be chosen from type I, II or III restriction enzymes (i.e., restriction endonucleases) such as AccI, AciI, AflIII, AluI, Alw44I, ApaI, AsnI, AvaI, AvaII, BamHI, BanlI, BclI, BglI, BglII, BlnI, BsmI, BssHII, BstEII, BstUI, CfoI, ClaI, DdeI, DpnI, DpnII, DraI, EclXI, EcoRI, EcoRI, EcoRII, EcoRV, HaelI, HaeII, HhaI, HindII, HindIII, HpaI, HpaII, KpnI, KspI, MaeII, McrBC, MluI, MluNI, MspI, NciI, NcoI, NdeI, NdeII, NheI, NotI, NruI, NsiI, PstI, PvuI, PvuII, RsaI, SacI, SalI, Sau3AI, ScaI, ScrFI, SfiI, SmaI, SpeI, SphI, SspI, StuI, StyI, SwaI, TaqI, XbaI, and XhoI. In some embodiments, a restriction enzyme (and/or a corresponding restriction enzyme recognition site) is chosen from one or more of MboI, HinfI, MseI and DdeI. In some embodiments, a restriction enzyme (and/or a corresponding restriction enzyme recognition site) is chosen from one or more of HpyCH4IV, HinfI, HinP1I and MseI. In some embodiments, a restriction enzyme (and/or a corresponding restriction enzyme recognition site) is NlaIII. In some embodiments, a restriction enzyme (and/or a corresponding restriction enzyme recognition site) is chosen from one or more of AciI, HinP1I, HpaII, HpyCH4IV, MspI, and TaqI. In some embodiments, a restriction enzyme (and/or a corresponding restriction enzyme recognition site) is chosen from one or more of BfaI, MseI, and CviQI. In some embodiments, a restriction enzyme (and/or a corresponding restriction enzyme recognition site) is chosen from one or more of LlaAI, MboI, MgoI, MkrAI, NdeII, NlaII, NmeCI, NphI, Sau3AI, Kzo9I, DpnII, BstMBI, BssMI, and Bsp143I. In some embodiments, a restriction enzyme (and/or a corresponding restriction enzyme recognition site) is DpnII. In some embodiments, a restriction enzyme (and/or a corresponding restriction enzyme recognition site) is HinfI. In some embodiments, a restriction enzyme recognition site comprises GATC. In some embodiments, a restriction enzyme recognition site comprises \hat{ }GATC (where \hat{ } is the cut site on the positive strand). In some embodiments, a restriction enzyme recognition site comprises GANTC (where “N” can be any of the 4 DNA nucleotide bases: A, C, G, T). In some embodiments, a restriction enzyme recognition site comprises G\hat{ }ANTC (where \hat{ } is the cut site on the positive strand, and “N” can be any of the 4 DNA nucleotide bases: A, C, G, T).

In some embodiments, a method herein comprises contacting proximity ligated nucleic acid molecules with a plurality of capture probe species. A plurality of capture probe species may each comprise a polynucleotide identical to or complementary to a subsequence in a gene (e.g., an oncogene). A plurality of capture probe species may each comprise a polynucleotide identical to or complementary to a subsequence in a subsequence in an exon of a gene (e.g., an oncogene). A plurality of capture probe species may each comprise a polynucleotide identical to or complementary to a subsequence in an exon of gene (e.g., an oncogene) listed in Table 1. In some embodiments, a plurality of capture probe species comprises about 10 or more capture probe species. In some embodiments, a plurality of capture probe species comprises about 20 or more capture probe species. In some embodiments, a plurality of capture probe species comprises about 50 or more capture probe species. In some embodiments, a plurality of capture probe species comprises about 100 or more capture probe species. In some embodiments, a plurality of capture probe species comprises about 500 or more capture probe species. In some embodiments, a plurality of capture probe species comprises about 1,000 or more capture probe species. In some embodiments, a plurality of capture probe species comprises about 10,000 or more capture probe species. In some embodiments, a plurality of capture probe species comprises about 100,000 or more capture probe species. In some embodiments, a plurality of capture probe species comprises about 300,000 or more capture probe species.

In some embodiments, a method herein comprises sequencing proximity ligated nucleic acid molecules. In some embodiments, a method herein comprises sequencing enriched proximity ligated nucleic acid molecules. Any suitable sequencing process may be used (e.g., a sequencing process described herein). In some embodiments, a sequencing process generates hundreds of sequence reads. In some embodiments, a sequencing process generates thousands of sequence reads. In some embodiments, a sequencing process generates tens of thousands of sequence reads. In some embodiments, a sequencing process generates hundreds of thousands of sequence reads. In some embodiments, a sequencing process generates millions of sequence reads. In some embodiments, a sequencing process generates hundreds of millions of sequence reads.

### Samples

Provided herein are methods and compositions for processing and/or analyzing nucleic acid. Nucleic acid utilized in methods and compositions described herein may be isolated from a sample obtained from a subject (e.g., a test subject). A subject can be any living or non-living organism, including but not limited to a human and a non-human animal. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a human. A subject may be a male or female. A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. In some embodiments, a subject is an adult patient. In some embodiments, a subject is a pediatric patient.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism. In some embodiments, a nucleic acid sample is isolated or obtained from a cell(s), tissue, organ, and/or the like of an animal (e.g., an animal subject). In some instances, a nucleic acid sample may be obtained as part of a diagnostic analysis.

A sample or test sample may be any specimen that is isolated or obtained from a subject or part thereof (e.g., a human subject, a cancer patient, a tumor). Non-limiting examples of specimens include fluid or tissue from a subject, including, without limitation, blood or a blood product (e.g., whole blood, serum, plasma, blood spot, blood smear, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebrospinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of tumor or cancer DNA found in a patient's blood, for example, may be performed using, e.g., whole blood, serum, or plasma. Methods for preparing serum or plasma from blood obtained from a subject (e.g., patient; cancer patient) are known. For example, a subject's blood (e.g., patient's blood; cancer patient's blood) can be placed in a tube containing EDTA or a specialized commercial product such as Cell-Free DNA BCT (Streck, Omaha, NE) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation.

In some embodiments, a sample is a tissue sample, a cell sample, a blood sample, or a urine sample. In some embodiments, a sample comprises formalin-fixed, paraffin-embedded (FFPE) tissue. In some embodiments, a sample comprises frozen tissue. In some embodiments, a sample comprises peripheral blood. In some embodiments, a sample comprises blood obtained from bone marrow. In some embodiments, a sample comprises cells obtained from urine. In some embodiments, a sample comprises cell-free nucleic acid. In some embodiments, a sample comprises one or more tumor cells. In some embodiments, a sample comprises one or more circulating tumor cells. In some embodiments, a sample comprises a solid tumor. In some embodiments, a sample comprises a blood tumor.

### Cancers

In some embodiments, a subject has, or is suspected of having, a disease. In some embodiments, a subject has, or is suspected of having, cancer. In some embodiments, a subject has, or is suspected of having, a cancer associated with one or more genes and/or cancer genes described herein. For example, in some embodiments, a subject has, or is suspected of having, a cancer associated with one or more genes and/or cancer genes selected from the group consisting of: the cancer genes listed in row 7, row 15 of Table 10 and any combinations thereof. In some embodiments, a subject has, or is suspected of having, a cancer associated with one or more structural variants described herein.

Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like. In some embodiments, a cancer is a rare cancer. In some embodiments, a cancer is glioma. In some embodiments, a cancer is glioblastoma. In some embodiments, a cancer is pediatric glioblastoma. In some embodiments, a cancer is glioblastoma multiforme/anaplastic astrocytoma with piloid features (ANA PA). In some embodiments, a cancer is a sarcoma. In some embodiments, a cancer is leiomyosarcoma (LMS). In some embodiments, a cancer is myxoid leiomyosarcoma. In some embodiments, a cancer is uterine cancer. In some embodiments, a cancer is uterine leiomyosarcoma. In some embodiments, a cancer is uterine myxoid leiomyosarcoma. In some embodiments, a cancer is metastatic high-grade sarcoma, uterine origin. In some embodiments, a cancer is a brain tumor. In some embodiments, a cancer is a benign brain tumor. In some embodiments, a cancer is an astrocytic brain tumor. In some embodiments, a cancer is subependymal giant cell astrocytoma (SEGA). In some embodiments, a cancer is pleomorphic xanthoastrocytoma (PXA). In some embodiments, a cancer is a malignant brain tumor. In some embodiments, a cancer is a bone cancer. In some embodiments, a cancer is chordoma. In some embodiments, a cancer is a central nervous system (CNS) tumor. In some embodiments, a cancer is meningioma. In some embodiments, a cancer is an embryonal tumor. In some embodiments, a cancer is an embryonal central nervous system tumor. In some embodiments, a cancer is embryonal tumors with multilayered rosettes (ETMR). In some embodiments, a cancer is a kidney/renal cancer. In some embodiments, a cancer is a primitive neuroectodermal tumor (PNET). In some embodiments, a cancer is a kidney primitive neuroectodermal tumor (PNET). In some embodiments, a cancer is lymphoma. In some embodiments, a cancer is Burkitt lymphoma. In some embodiments, a cancer is Burkitt lymphoma (human immunodeficiency virus (HIV)+ and/or Epstein-Barr Virus (EBV)+). In some embodiments, a cancer is Hodgkins lymphoma. In some embodiments, a cancer is classic Hodgkins lymphoma. In some embodiments, a cancer is B cell lymphoma. In some embodiments, a cancer is diffuse large B cell lymphoma. In some embodiments, a cancer is a cytoma. In some embodiments, a cancer is plasmacytoma. In some embodiments, a cancer is osseous plasmacytoma. In some embodiments, a cancer is an adenoma. In some embodiments, a cancer is pituitary adenoma.

### Diagnosis and Treatment

In some embodiments, a method herein comprises providing a diagnosis and/or a likelihood of cancer in a subject. A diagnosis and/or likelihood of cancer may be provided when the presence of a structural variant described herein is detected. In some embodiments, a method herein comprises performing a further test (e.g., biopsy, blood test, imaging, surgery) to confirm a cancer diagnosis.

In some embodiments, a method herein comprises selecting a sample from a subject. In some embodiments, one or more oncogenes in a selected sample are or were previously analyzed for one or more genetic variations associated with cancer. Genetic variations associated with cancer may comprise one or more genetic variations chosen from mutations, translocations, inversions, insertions, deletions, duplications, microdeletions, and microduplications. In some embodiments, one or more oncogenes may be analyzed for the one or more genetic variations associated with cancer according to one or more methods chosen from RNA-Seq (transcriptome analysis), chromosomal karyotyping, FISH panel, microarray, targeted sequencing, cancer NGS panel, and methylation array. In some embodiments, one or more oncogenes comprise no detectable genetic variation associated with cancer (e.g., as analyzed by one or more of the aforementioned methods).

In some embodiments, a selected sample is or was previously analyzed for one or more druggable targets. Druggable targets means clinically actionable targets. In some embodiments, one or more oncogenes in a selected sample are or were previously analyzed for one or more druggable targets associated with cancer. Druggable targets may include genes and/or cancer genes and/or oncogenes (i.e., genes, cancer genes and/or oncogenes encoding druggable targets) provided in a database containing druggable targets (e.g., ONCOKB (Memorial Sloan Kettering's Precision Oncology Knowledge Base)). ONCOKB is a precision oncology knowledge base developed at Memorial Sloan Kettering Cancer Center that contains biological and clinical information about genomic alterations in cancer. In some embodiments, druggable targets include genes and/or oncogenes categorized under one or more therapeutic levels, diagnostic levels, and/or prognostic levels (e.g., in the ONCOKB database). In some embodiments, druggable targets include genes and/or oncogenes categorized under therapeutic level 1 (FDA-approved drugs; 43 genes), therapeutic level 2 (standard care; 24 genes), therapeutic level 3 (clinical evidence; 33 genes) and/or therapeutic level R1/R2 (resistance; 11 genes). In some embodiments, druggable targets include genes and/or oncogenes categorized under diagnostic level Dx1 (required for diagnosis; 22 genes) and/or diagnostic level Dx2 (supports diagnosis; 53 genes). In some embodiments, druggable targets include genes and/or oncogenes categorized under prognostic level Px1 (guideline-recognized with well-powered data; 25 genes) and/or prognostic level Px2 (guideline-recognized with limited data; 15 genes).

Tier 1 is either a Therapeutic Level 1, 2, 3 or R1 gene from OncoKB. Tier 1 also includes NCCN Biomarker compendium genes where the “Test Purpose” for the gene is “Predictive”, “Treatment”, or “Therapy Determination”.

Tier 2 is genes involved in fusions where that gene is the direct target of a drug from an ongoing clinical trial according to clinicaltrials.gov

Tier 3 is either a Diagnostic Level 1 or 2, or a Prognostic Level 1 or 2 gene according to OncoKb. Tier 3 also includes NCCN Biomarker compendium genes where the “Test Purpose” for the gene must contain (at a minimum) either “Diagnostic”, “Prognostic”, “Essential Diagnostic”, “Workup”, “Risk Stratification”, or “Risk Assessment”. The additional criteria for Tier 3 is that the gene must be found in the disease for which it is diagnostic or prognostic.

Tier 4 is none of the above.

In some embodiments, a method comprises (a) selecting a sample from a subject, where the selected sample is or was previously analyzed for one or more druggable targets, and no detectable druggable target is or was identified; (b) performing a nucleic acid analysis on the selected sample, wherein the analysis comprises a method that preserves spatial-proximal contiguity information; and (c) detecting whether a structural variant is present or absent in the selected sample according to the nucleic acid analysis in (b), wherein a breakpoint of the structural variant is not within one or more genes and/or oncogenes encoding the one or more druggable targets analyzed in (a). In some embodiments, a method comprises identifying a new druggable target according to the genomic location of the structural variant (e.g., a druggable target not analyzed in (a) and/or a druggable target not listed in ONCOKB).

In some embodiments, a method comprises (a) selecting a sample from a subject, where the selected sample is or was previously analyzed for one or more druggable targets, and no detectable druggable target is or was identified; (b) performing a nucleic acid analysis on the selected sample, wherein the analysis comprises a method that preserves spatial-proximal contiguity information; and (c) detecting whether a structural variant is present or absent in the selected sample according to the nucleic acid analysis in (b), wherein a breakpoint of the structural variant is not in proximity (linear proximity and/or spatial proximity) to one or more genes and/or oncogenes encoding the one or more druggable targets analyzed in (a). In some embodiments, a method comprises identifying a new druggable target according to the genomic location of the structural variant (e.g., a druggable target not analyzed in (a) and/or a druggable target not listed in ONCOKB).

The term “in proximity” may refer to spatial proximity and/or linear proximity. Spatial proximity generally refers to 3-dimensional chromatin proximity, which may be assessed according to a method that preserves spatial-proximal relationships, such as a method described herein or any suitable method known in the art. A structural variant may be located at a position in spatial proximity to a gene and/or oncogene when a structural variant and a gene and/or oncogene (or a fragment thereof) are ligated in a proximity ligation assay or are bound by a common solid phase in a solid substrate-mediated proximity capture (SSPC) assay, for example. Linear proximity generally refers to a linear base-pair distance, which may be assessed according to mapped distances in a reference genome, for example. Linear proximity distance may be provided as a distance between a 5′ or 3′ end of a structural variant and a 5′ or 3′ end of a gene and/or oncogene encoding a druggable target.

In some embodiments, a method herein comprises administering a treatment to a subject. A treatment may be administered to a subject when the presence of a structural variant described herein is detected. Suitable treatments may be determined by a physician and may include one or more modulators (e.g., activators, blockers) of one or more genes, proteins, oncogenes, oncoproteins (proteins encoded by oncogenes), and/or oncogene-related components associated with a detected structural variant.

An oncogene-related component generally refers to one or more components chosen from (i) an oncogene, including exons, introns, and 5′ (upstream), e.g. promoter regions, or 3′ (downstream) regulatory elements; (ii) transcription products, mRNA, or cDNA; (iii) translation products, protein, gene products, or gene expression products, or homologs of, synthetic versions of, analogs of, receptors of, agonists to receptors of, antagonists to receptors of, upstream pathway regulators of, or downstream pathway targets of translation products, protein, gene products, or gene expression products; and (iv) any component that could be considered by one skilled in the art as a target for a modulator (e.g., activator, blocker, drug, medicament).

A modulator generally refers to an agent that is capable of changing an activity (e.g., change in level and/or nature of an activity) of a component in a system compared to a component's activity under otherwise comparable conditions when the modulator is absent. A modulator herein may refer to an agent that is capable of changing an activity (e.g., change in level and/or nature of an activity) of a gene, protein, oncogene, oncoprotein, and/or oncogene-related component in a system compared to a gene's, protein's, oncogene's, oncoprotein's, and/or oncogene-related component's activity under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target component of interest. In some embodiments, a modulator interacts indirectly (e.g., directly with an intermediate agent that interacts with the target component) with a target component of interest. In some embodiments, a modulator affects the level of a target component of interest, as one non-limiting example by impacting an upstream signaling pathway associated with the target component of interest. In some embodiments, a modulator affects an activity of a target component of interest without affecting a level of the target component, as one non-limiting example by impacting a downstream signaling pathway associated with the target component of interest. In some embodiments, a modulator affects both level and activity of a target component of interest, such that an observed difference in activity is not entirely explained by or commensurate with an observed difference in level.

The term “modulator of [cancer gene]” or “[cancer gene] modulator” means “modulator of [cancer gene], modulator of [cancer gene] protein, and/or [cancer gene]-related components” or “[cancer gene], [cancer gene] protein, and/or [cancer gene]-related components modulator,” respectively, where [cancer gene] can mean any cancer gene identified herein.

In some embodiments, a treatment comprises a modulator of a cancer gene, where the cancer gene is selected from the group consisting of: cancer genes listed in row 7, row 15 of Table 10 and any combinations thereof.

In some embodiments, a method herein comprises predicting an outcome of a cancer treatment. An outcome of a cancer treatment may be predicted when the presence of a structural variant described herein is detected. For example, an outcome of a cancer treatment that includes a gene-specific modulator and/or an oncogene-specific modulator may be predicted when the presence of a structural variant associated with the gene and/or oncogene is detected.

In some embodiments, a method comprises predicting an outcome of a modulator treatment of a cancer gene, where the cancer gene is selected from the group consisting of: cancer genes listed in row 7, row 15 of Table 10, and any combinations thereof when the presence of a structural variant described herein is detected (e.g., a structural variant associated with a cancer gene listed in row 7 and row 15 of Table 10).

In some embodiments, a sample from a subject is obtained over a plurality of time points. A plurality of time points may include time point over a number of days, weeks, months, and/or years. In some embodiments, a disease state is monitored over a plurality of time points. For example, a method to detect the presence, absence, or amount of a structural variant described herein may be performed over a plurality of time points to monitor the status of a disease (e.g., a disease (e.g., cancer) associated with the structural variant detected). In some embodiments, minimal residual disease (MRD) is monitored in a subject. Minimal residual disease (MRD) generally refers to cancer cells remaining after treatment that often cannot be detected by standard scans (e.g., X-ray, mammogram, computerized tomography (CT) scan, bone scan, magnetic resonance imaging (MRI), positron emission tomography (PET) scan, ultrasound) or tests (blood test, tissue biopsy, needle biopsy, liquid biopsy, endoscopic exam). Such cells have the potential to cause a relapse of cancer in a subject. In some embodiments, a method herein comprises detecting a presence of minimal residual disease (MRD) in a subject when a structural variant described herein is present. In some embodiments, a method herein comprises detecting a presence of minimal residual disease (MRD) in a subject when a structural variant described herein is present at a detectable level or amount (e.g., detectable by a method described herein). In some embodiments, a method herein comprises detecting an absence of minimal residual disease (MRD) in a subject when a structural variant described herein is absent. In some embodiments, a method herein comprises detecting an absence of minimal residual disease (MRD) in a subject when a structural variant described herein is present at an undetectable level or amount (e.g., undetectable by a method described herein). In some embodiments, a method herein comprises detecting an amount of a structural variant described herein in a sample. A level of minimal residual disease (MRD) in a subject may be determined according to an amount of structural variant detected in a sample. In some embodiments, a method herein comprises administering a treatment, or continuing to administer a treatment, to the subject when a structural variant is present. In some embodiments, a method herein comprises stopping a treatment for the subject when a structural variant is absent.

### Compositions

Provided in certain embodiments are compositions. A composition may comprise a nucleic acid. A composition may comprise an isolated nucleic acid. The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components.

In some embodiments, a composition comprises a nucleic acid comprising a structural variant, or portion thereof. Examples of structural variant types are described herein. In some embodiments, a composition comprises an isolated nucleic acid comprising a structural variant, or portion thereof. In some embodiments, a structural variant or part thereof maps to a location at, near, or between particular positions in a human reference genome. In some embodiments, a breakpoint of a structural variant maps to a location at, near, or between particular positions in a human reference genome. In some embodiments, the positions are in an HG38 human reference genome.

In some embodiments, a breakpoint of a structural variant maps to a location between positions selected from the group consisting of: positions listed in row 5, row 6, row 22, and row 23 of Table 10.

In some embodiments, a structural variant may comprise an ectopic portion of genomic DNA (i.e., a portion of genomic DNA at a receiving site from a different region of a chromosome or from a different chromosome). The ectopic portion may be referred to as a donor portion. If the ectopic portion (donor portion) is from the same chromosome as the structural variant, the ectopic portion may be from a location outside of the position ranges provided above for certain structural variants. The ectopic portion may comprise genomic DNA from a genomic coordinate window provided below, or part thereof. The ectopic portion may comprise genomic DNA from a genomic coordinate window provided below, or part thereof, and may further comprise genomic DNA from a region outside of a genomic coordinate window provided below.

In some embodiments, a structural variant comprises an ectopic portion of genomic DNA from positions selected from the group consisting of: positions listed in row 5, row 6, row 22, and row 23 of Table 10. In some embodiments, a nucleic acid or isolated nucleic acid comprises a label. In some embodiments, a nucleic acid or isolated nucleic acid comprises a detectable label. In some embodiments, a nucleic acid or isolated nucleic acid comprises a fluorescent label. In some embodiments, a nucleic acid or isolated nucleic acid comprises a colorimetric label. Examples of labels include radiolabels such as ³²P, ³³P, ¹²⁵I, or ³⁵S; enzyme labels such as alkaline phosphatase: fluorescent labels such as fluorescein isothiocyanate (FITC): or other labels such as biotin, avidin, digoxigenin, antigens, haptens, or fluorochromes. Labels and detectable labels typically are not associated with the nucleic acid in vivo and thereby do not naturally occur with the nucleic acid.

In some embodiments, a nucleic acid or isolated nucleic acid comprises one or more chemical moieties, biomolecules, and/or member of a binding pair (e.g., configured for immobilization of nucleic acids to a solid support). In some embodiments, a nucleic acid or isolated nucleic acid comprises one or more of thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof. Chemical moieties, biomolecules, and members of a binding pair typically are not associated with the nucleic acid in vivo and thereby do not naturally occur with the nucleic acid.

In some embodiments, a nucleic acid or isolated nucleic acid is modified to comprise one or more polynucleotide components, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI), the like or combinations thereof. In some embodiments, a nucleic acid or isolated nucleic acid comprises one or more adapters (e.g., sequencing adapters). Sequencing adapters may comprise sequences complementary to flow-cell anchors, and sometimes are utilized to immobilize a nucleic acid to a solid support, such as the inside surface of a flow cell, for example. Adapters and other polynucleotide components described above typically are not associated with the nucleic acid in vivo and thereby do not naturally occur with the nucleic acid.

In some embodiments, a composition herein comprises a nucleic acid or isolated nucleic acid and one or more enzymes. In some embodiments, a composition herein comprises a nucleic acid or isolated nucleic acid and one or more isolated enzymes. In some embodiments, a composition herein comprises a nucleic acid or isolated nucleic acid and one or more recombinant enzymes. In some embodiments, a composition herein comprises a nucleic acid or isolated nucleic acid and one or more isolated recombinant enzymes. Enzymes may include one or more enzymes useful for performing a method described herein (e.g., a nucleic acid analysis described herein). In some embodiments, one or more enzymes comprise one or more ligases. In some embodiments, one or more enzymes comprise one or more endonucleases (e.g., one or more restriction enzymes). In some embodiments, one or more enzymes comprise one or more polymerases. Certain enzymes described above typically are not associated with the nucleic acid in vivo and thereby do not naturally occur with the nucleic acid.

In some embodiments, a composition herein comprises a nucleic acid or isolated nucleic acid and one or more synthetic oligonucleotides. In some embodiments, a composition herein comprises a nucleic acid or isolated nucleic acid and one or more primers (e.g., amplification primers, PCR primers). Primers may be capable of hybridizing to the nucleic acid or isolated nucleic acid. In some embodiments, a composition herein comprises a nucleic acid or isolated nucleic acid and one or more probes. Probes may be capable of hybridizing to the nucleic acid or isolated nucleic acid. Probes may include capture probes and/or labeled probes. In some embodiments, one or more probes are fluorescently labeled probes. Synthetic oligonucleotides, primers, and probes described herein typically are not associated with the nucleic acid in vivo and thereby do not naturally occur with the nucleic acid.

In some embodiments, a nucleic acid or isolated nucleic acid is in a vector. A vector is any vehicle used to house a fragment of DNA sequence. Vectors may be useful for ferrying DNA into a host cell (e.g., as part of a molecular cloning procedure), and may assist in multiplying, isolating, or expressing the DNA fragment. Non-limiting examples of vectors include DNA vectors, viral vectors, plasmids, phage vectors, autonomously replicating sequence (ARS), artificial chromosome, yeast artificial chromosome (e.g., YAC), and the like. In some embodiments, a vector is an expression vector. In some embodiments, a vector is a cloning vector. Vectors typically are not associated with the nucleic acid in vivo and thereby do not naturally occur with the nucleic acid.

### Oligonucleotides

Provided herein are oligonucleotides. Oligonucleotides may be artificially synthesized. Accordingly, provided herein in certain embodiments are synthetic oligonucleotides. An oligonucleotide generally refers to a nucleic acid (e.g., DNA, RNA) polymer that is distinct from a target nucleic acid (e.g., a target nucleic acid comprising one or more structural variants described herein), and may be referred to as oligos, probes, and/or primers. Oligonucleotides may be short in length (e.g., less than 50 bp, less than 40 bp, less than 30 bp, less than 20 bp, less than 10 bp). In some embodiments, oligonucleotides are between about 10 to about 500 consecutive nucleotides in length. For example, an oligonucleotide may be about 10, 20, 30, 40, 50, 60, 70, 80, 90, 100, 150, 200, 250, 300, 350, 400, 450, or 500 consecutive nucleotides in length.

Oligonucleotides may be designed to hybridize to a region of a sample nucleic acid that is proximal to, adjacent to, and/or spanning a structural variant described herein, or portion thereof. Oligonucleotides may be designed to hybridize to a portion or portions of a genome that is/are proximal to, adjacent to, overlapping, partially overlapping, or spanning a structural variant or portion thereof. Oligonucleotides may be designed to hybridize to a region of a sample nucleic acid that comprises a receiving site, a donor site, or a combination of a receiving site and a donor site.

Oligonucleotides may include probes and/or primers useful for detecting presence, absence, or amount of a structural variant in a nucleic acid sample. Probes and/or primers may be used in conjunction with any suitable nucleic acid analysis (e.g., a nucleic acid analysis method described herein). For example, probes and/or primers may be used in an amplification process (e.g., PCR, quantitative PCR), FISH (e.g., labeled FISH probes, labeled FISH probe pairs (e.g., with fluorophore and quencher)), microarray, nucleic acid capture, nucleic acid enrichment, nucleic acid sequencing, and the like. In some embodiments, oligonucleotides include a capture probe described herein. In some embodiments, oligonucleotides include a plurality of capture probes described herein.

Oligonucleotides may include a probe or primer capable of hybridizing to a region of a first breakpoint and a region of a second breakpoint of a structural variant described herein. Accordingly, such probes and primers comprise a first sequence complementary to a receiving site in a structural variant and a second sequence complementary to a donor site in a structural variant. Such probes and primers are useful for detecting the presence, absence, or amount of a structural variant in a sample, for example, by way of hybridizing to the sample nucleic acid when the structural variant is present and not hybridizing to the sample nucleic acid when the structural variant is absent.

In some embodiments, an oligonucleotide comprises (i) a first polynucleotide identical to or complementary to a subsequence (e.g., of 5 or more consecutive nucleotides in length) within a region of a chromosome comprising a receiving site for a structural variant described herein, and (ii) a second polynucleotide identical to or complementary to a subsequence (e.g., of 5 or more consecutive nucleotides in length) within a region of a chromosome comprising a donor site for a structural variant described herein. Such oligonucleotide can specifically hybridize (e.g., under stringent hybridization conditions) to a target sequence comprising the subsequence of (i) and the subsequence of (ii).

Oligonucleotides may include a pair of probes or primers capable of hybridizing to a region of a first breakpoint and a region of a second breakpoint of a structural variant described herein. Accordingly, such probe and primer pairs comprise a first member complementary to a receiving site in a structural variant and a second member complementary to a donor site in a structural variant. Such probes and primers may be useful for detecting the presence or absence of a structural variant in a sample, for example, by way of hybridizing to the sample nucleic acid at specific locations when the structural variant is present and hybridizing to the sample nucleic acid at different locations when the structural variant is absent.

In some embodiments, a composition comprises (a) a first oligonucleotide comprising a first polynucleotide identical to or complementary to a subsequence (e.g., of 5 or more consecutive nucleotides in length) within a region of a chromosome comprising a receiving site for a structural variant described herein; and (b) a second oligonucleotide comprising a second polynucleotide identical to or complementary to a subsequence (e.g., of 5 or more consecutive nucleotides in length) within a region of a chromosome comprising a donor site for a structural variant described herein. Such oligonucleotides may specifically hybridize (e.g., under stringent hybridization conditions) to a target nucleic acid comprising the subsequences of (a) and (b). In some embodiments, the first oligonucleotide specifically hybridizes (e.g., under stringent hybridization conditions) to a target nucleic acid comprising the subsequence of (a) and does not specifically hybridize to a target nucleic acid comprising the subsequence of (b). In some embodiments, the second oligonucleotide specifically hybridizes (e.g., under stringent hybridization conditions) to a target nucleic acid comprising the subsequence of (b) and does not specifically hybridize to a target nucleic acid comprising the subsequence of (a).

In some embodiments, a composition comprises (a) a first oligonucleotide comprising a first polynucleotide identical to or complementary to a subsequence of 5 or more consecutive nucleotides in length within a region of a chromosome, where the region spans positions selected from the group consisting of: positions listed in row 5 and row 6 of Table 10; and (b) a second oligonucleotide comprising a second polynucleotide identical to or complementary to a subsequence of about 5 or more consecutive nucleotides in length within a region of a chromosome, where the region spans positions selected from the group consisting of: positions listed in row 22 and row 23 of Table 10. The first oligonucleotide may specifically hybridize (e.g., under stringent hybridization conditions) to a target nucleic acid comprising the subsequence of the corresponding chromosome in (a). The second oligonucleotide may specifically hybridize (e.g., under stringent hybridization conditions) to a target nucleic acid comprising the subsequence of the corresponding chromosome in (b). In some embodiments, the first oligonucleotide specifically hybridizes (e.g., under stringent hybridization conditions) to a target nucleic acid comprising the subsequence of the corresponding chromosome in (a) and does not specifically hybridize to a target nucleic acid comprising the subsequence of the corresponding chromosome in (b). In some embodiments, the second oligonucleotide specifically hybridizes (e.g., under stringent hybridization conditions) to a target nucleic acid comprising the subsequence of the corresponding chromosome in (b) and does not specifically hybridize to a target nucleic acid comprising the subsequence of the corresponding chromosome in (a).

### Kits

Provided in certain embodiments are kits. The kits may include any components and compositions described herein (e.g., nucleic acids, oligonucleotides, primers, probes (e.g., capture probes), vectors, enzymes) useful for performing any of the methods described herein, in any suitable combination. Kits may further include any reagents, buffers, or other components useful for carrying out any of the methods described herein.

Components of a kit may be present in separate containers, or multiple components may be present in a single container. Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for using oligonucleotides, primers, and/or probes described herein. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### Certain Implementations

Following are non-limiting examples of certain implementations of the technology.

### Certain Implementations

Following are non-limiting examples of certain implementations of the technology.
A1. A method for detecting the presence or absence of a structural variant in a sample, the method comprising:
   a) selecting a sample from a subject, wherein one or more cancer genes in the sample were analyzed for one or more genetic variations associated with cancer, and the one or more cancer genes comprise no detectable genetic variation associated with cancer;
   b) performing a nucleic acid analysis on the selected sample, wherein the analysis comprises a method that preserves spatial-proximal contiguity information; and
   c) detecting whether a structural variant is present or absent in the selected sample according to the nucleic acid analysis in (b), wherein a breakpoint of the structural variant is not within the one or more cancer genes analyzed in (a).
A1.1 The method of embodiment A1, wherein the breakpoint of the structural variant is within an intergenic region.
A1.2 The method of embodiment A1, wherein the breakpoint of the structural variant is within a gene other than the cancer gene.
A2. The method of any one of embodiments A1-A1.2, wherein the one or more cancer genes are chosen from one or more of any cancer gene found in row 7 and 15 of Table 10.
A3. The method of any one of embodiments A1-A1.2, wherein the one or more cancer genes are selected from the groups consisting of: cancer genes found in row 7 and 15 of Table 10.
A4. The method of any one of embodiments A1-A3, wherein the one or more cancer genes were analyzed for the one or more genetic variations associated with cancer according to one or more methods chosen from RNA-Seq, chromosomal karyotyping, FISH panel, microarray, cancer NGS panel, and methylation array.
A5. The method of any one of embodiments A1-A4, wherein the one or more genetic variations associated with cancer comprise one or more genetic variations chosen from mutations, translocations, inversions, insertions, deletions, duplications, microdeletions, and microduplications.
A6. The method of any one of embodiments A1-A5, wherein the structural variant comprises one or more of a translocation, inversion, insertion, deletion, and duplication.
A7. The method of any one of embodiments A1-A6, wherein the structural variant comprises a microduplication and/or a microdeletion.
A8. The method of embodiment A6, wherein the translocation or the insertion comprises nucleic acid from a chromosome that is the same chromosome on which a cancer gene of the one or more cancer genes is located.
A9. The method of embodiment A6, wherein the translocation or the insertion comprises nucleic acid from a chromosome that is a different chromosome from which a cancer gene of the one or more cancer genes is located.
A10. The method of any one of embodiments A1-A9, wherein a breakpoint for the structural variant is located on the same chromosome as the cancer gene.
A11. The method of any one of embodiments A1-A10, wherein a breakpoint for the structural variant is located at least about 10 base pairs from a cancer gene terminus.
A11.1 The method of any one of embodiments A1-A10, wherein a breakpoint for the structural variant is located at least about 500 base pairs from a cancer gene terminus.
A12. The method of any one of embodiments A1-A10, wherein a breakpoint for the structural variant is located at least about 4,000 base pairs from a cancer gene terminus.
A13. The method of any one of embodiments A1-A10, wherein a breakpoint for the structural variant is located at about 4,000 base pairs to about 700,000 base pairs from a cancer gene terminus.
A14. The method of any one of embodiments A1-A10, wherein a breakpoint for the structural variant is located at about 4,000 base pairs to about 100,000 base pairs from a cancer gene terminus.
A14.1 The method of any one of embodiments A1-A10, wherein a breakpoint for the structural variant is located at about 500 base pairs to about 650,000 base pairs from a cancer gene terminus.
A14.2 The method of any one of embodiments A1-A10, wherein a breakpoint for the structural variant is located at about 500 base pairs to about 1,630,000 base pairs from a cancer gene terminus.
A14.3 The method of any one of embodiments A11-A14.2, wherein the cancer gene terminus is a 5′ terminus.
A14.4 The method of any one of embodiments A11-A14.2, wherein the cancer gene terminus is a 3′ terminus.
A15. The method of any one of embodiments A1-A14.4, wherein the nucleic acid analysis in (b) comprises generating proximity ligated nucleic acid molecules.
A16. The method of embodiment A15, wherein the nucleic acid analysis in (b) further comprises sequencing the proximity ligated nucleic acid molecules.
A17. The method of embodiment A15 or A16, wherein the nucleic acid analysis in (b) further comprises contacting the proximity ligated nucleic acid molecules with one or more capture probe species, thereby generating enriched proximity ligated nucleic acid molecules.
A18. The method of embodiment A17, wherein the nucleic acid analysis in (b) further comprises sequencing the enriched proximity ligated nucleic acid molecules.
A19. The method of embodiment A16 or A18, wherein the sequencing generates hundreds of sequence reads.
A20. The method of embodiment A16 or A18, wherein the sequencing generates thousands of sequence reads.
A21. The method of embodiment A16 or A18, wherein the sequencing generates tens of thousands of sequence reads.
A22. The method of embodiment A16 or A18, wherein the sequencing generates hundreds of thousands of sequence reads.
A23. The method of embodiment A16 or A18, wherein the sequencing generates millions of sequence reads.
A23.1 The method of embodiment A16 or A18, wherein the sequencing generates hundreds of millions of sequence reads.
A24. The method of any one of embodiments A17-A23.1, wherein the one or more capture probe species each comprise a polynucleotide identical to or complementary to a subsequence in an exon of a gene listed in Table 7.
A24.1 The method of embodiment A24, wherein the one or more capture probe species each further comprise a polynucleotide identical to or complementary to a subsequence in an intron of a gene listed in Table 7.
A25. The method of embodiment A24 or A24.1, wherein the polynucleotide maps to coordinates that are within 300-400 bp of one or more sites targeted by one or more restriction enzymes.
A26. The method of embodiment A24 or A24.1, wherein the polynucleotide maps to coordinates that are within 350 bp of one or more sites targeted by one or more restriction enzymes.
A26.1 The method of embodiment A25 or A26, wherein the one or more sites targeted by the one or more restriction enzymes comprise \hat{ }GATC, wherein \hat{ } is a cut site.
A26.2 The method of any one of embodiments A25-A26.1, wherein the one or more sites targeted by the one or more restriction enzymes comprise G\hat{ }ANTC, wherein \hat{ } is a cut site and N is A, C, G, or T.
A27. The method of any one of embodiments A25-A26.2, wherein generating proximity ligated nucleic acid molecules comprises use of the one or more restriction enzymes.
A28. The method of any one of embodiments A1-A27, wherein the subject is a human.
A29. The method of embodiment A28, wherein the subject is an adult patient.
A30. The method of embodiment A28, wherein the subject is a pediatric patient.
A31. The method of any one of embodiments A1-A30, wherein the subject has, or is suspected of having, a disease.
A32. The method of any one of embodiments A1-A31, wherein the subject has, or is suspected of having, cancer.
A32.1. The method of embodiment A32, wherein the cancer is selected from the cancers listed in row 3 of Table 10.
A32.2 The method of embodiment A32 or A32.1, wherein the cancer is selected from the blood cancers.
A32.2.1 The method of embodiment A32 or A32.2, wherein the cancer is selected from cancers that are not a blood cancers.
A32.3 The method of embodiment A32 to A32.2, wherein the cancer is a solid heme type.
A32.4 The method of embodiment A32 to A323, wherein the cancer is a liquid heme type.
A32.5 The method of embodiment A32 to A32.4, wherein the cancer is not a solid heme type.
A32.6 The method of embodiment A32 to A325, wherein the cancer is not a liquid heme type.
A33. The method of embodiment A32, wherein the cancer is a rare cancer.
A34. The method of embodiment A32 to A33, wherein the cancer is uterine myxoid leiomyosarcoma.
A35. The method of embodiment A32 to A33, wherein the cancer is subependymal giant cell astrocytoma (SEGA).
A36. The method of embodiment A32 to A33, wherein the cancer is a malignant brain tumor.
A37. The method of embodiment A32 to A33, wherein the cancer is chordoma.
A38. The method of embodiment A32 to A33, wherein the cancer is meningioma.
A39. The method of embodiment A32 to A33, wherein the cancer is embryonal tumors with multilayered rosettes (ETMR).
A40. The method of embodiment A32 to A33, wherein the cancer is metastatic high-grade sarcoma, uterine origin.
A41. The method of embodiment A32 to A33, wherein the cancer is pleomorphic xanthoastrocytoma (PXA).
A42. The method of embodiment A32 to A33, wherein the cancer is glioblastoma multiforme/anaplastic astrocytoma with piloid features (ANA PA).
A42.1 The method of embodiment A32 to A33, wherein the cancer is glioma.
A42.2 The method of embodiment A32 to A33, wherein the cancer is myxoid leiomyosarcoma (LMS).
A42.3 The method of embodiment A32 to A33, wherein the cancer is plasmacytoma.
A42.4 The method of embodiment A32 to A33, wherein the cancer is osseous plasmacytoma.
A42.5 The method of embodiment A32 to A33, wherein the cancer is classic Hodgkins lymphoma.
A42.6 The method of embodiment A32 to A33, wherein the cancer is diffuse large B cell lymphoma.
A42.7 The method of embodiment A32 to A33, wherein the cancer is leiomyosarcoma.
A43. The method of any one of embodiments A1-A42.7, wherein the sample is a tissue sample, a cell sample, a blood sample, or a urine sample.
A44. The method of any one of embodiments A1-A43, wherein the sample comprises FFPE tissue.
A45. The method of any one of embodiments A1-A43, wherein the sample comprises frozen tissue.
A46. The method of any one of embodiments A1-A43, wherein the sample comprises peripheral blood.
A47. The method of any one of embodiments A1-A43, wherein the sample comprises blood obtained from bone marrow.
A48. The method of any one of embodiments A1-A43, wherein the sample comprises cells obtained from urine.
A49. The method of any one of embodiments A1-A43, wherein the sample comprises cell-free nucleic acid.
A50. The method of any one of embodiments A1-A49, wherein the sample comprises one or more tumor cells.
A51. The method of any one of embodiments A1-A50, wherein the sample comprises one or more circulating tumor cells.
A52. The method of any one of embodiments A1-A49, wherein the sample comprises a solid tumor.
A53. The method of any one of embodiments A1-A49, wherein the sample comprises a blood tumor.
A54. The method of any one of embodiments A1-A53, further comprising administering a treatment to the subject when the structural variant is present.
A55. The method of any one of embodiments A1-A54, further comprising identifying a cancer gene spatially proximal to the structural variant.
A56. The method of embodiment A55, further comprising administering a cancer gene-specific treatment to the subject according to the identified cancer gene located spatially proximal to the structural variant.
A57. The method of any one of embodiments A54-A56, wherein the treatment comprises one or more treatments chosen from a modulator of a cancer gene.
A58. The method of any one of embodiments A54-A57, wherein the treatment comprises one or more treatments chosen from a modulator of a cancer gene, wherein the cancer gene is one of the cancer genes of row 7 and/or row 15 of Table 10.
B1. A method for detecting the presence or absence of a structural variant in a sample, the method comprising:
   a) performing a nucleic acid analysis on a sample from a subject, wherein the analysis comprises i) generating proximity ligated nucleic acid molecules and ii) contacting the proximity ligated nucleic acid molecules with one or more capture probe species, thereby generating enriched proximity ligated nucleic acid molecules, wherein the one or more capture probe species each comprise a polynucleotide identical to or complementary to a subsequence in an exon of an cancer gene; and
   b) detecting whether a structural variant is present or absent in the selected sample according to the nucleic acid analysis in (a).
B1.1 The method of embodiment B1, wherein the one or more capture probe species each further comprise a polynucleotide identical to or complementary to a subsequence in an intron of a cancer gene.
B2. The method of embodiment B1 or B1.1, wherein the one or more capture probe species each comprise a polynucleotide identical to or complementary to a subsequence in an exon of a cancer gene listed in Table 7.
B3. The method of any one of embodiments B1-B2, wherein (a) comprises contacting the proximity ligated nucleic acid molecules with a plurality of capture probe species.
B4. The method of embodiment B3, wherein the plurality of capture probe species each comprise a polynucleotide identical to or complementary to a subsequence in an exon of a cancer gene listed in Table 7.
B4.1 The method of embodiment B4, wherein the plurality of capture probe species each further comprise a polynucleotide identical to or complementary to a subsequence in an intron of a cancer gene listed in Table 7.
B5. The method of any one of embodiment B3 to B4.1, wherein the plurality of capture probe species comprises about 10 or more capture probe species.
B6. The method of any one of embodiment B3 to B4.1, wherein the plurality of capture probe species comprises about 20 or more capture probe species.
B7. The method of any one of embodiment B3 to B4.1, wherein the plurality of capture probe species comprises about 50 or more capture probe species.
B8. The method of any one of embodiment B3 to B4.1, wherein the plurality of capture probe species comprises about 100 or more capture probe species.
B9. The method of any one of embodiment B3 to B4.1, wherein the plurality of capture probe species comprises about 500 or more capture probe species.
B10. The method of any one of embodiment B3 to B4.1, wherein the plurality of capture probe species comprises about 1,000 or more capture probe species.
B11. The method of any one of embodiment B3 to B4.1, wherein the plurality of capture probe species comprises about 10,000 or more capture probe species.
B12. The method of any one of embodiment B3 to B4.1, wherein the plurality of capture probe species comprises about 100,000 or more capture probe species.
B13. The method of any one of embodiment B3 to B4.1, wherein the plurality of capture probe species comprises about 300,000 or more capture probe species.
B14. The method of any one of embodiments B1-B13, wherein the polynucleotide maps to coordinates that are within 300-400 bp of one or more sites targeted by one or more restriction enzymes.
B15. The method of any one of embodiments B1-B13, wherein the polynucleotide maps to coordinates that are within 350 bp of one or more sites targeted by one or more restriction enzymes.
B15.1 The method of embodiment B14 or B15, wherein the one or more sites targeted by the one or more restriction enzymes comprise \hat{ }GATC, wherein \hat{ } is a cut site.
B15.2 The method of any one of embodiments B14-B15.1, wherein the one or more sites targeted by the one or more restriction enzymes comprise G\hat{ }ANTC, wherein \hat{ } is a cut site and N is A, C, G, or T.
B16. The method of any one of embodiments B14-B15.2, wherein generating proximity ligated nucleic acid molecules comprises use of the one or more restriction enzymes.
B17. The method of any one of embodiments B1-B16, wherein the nucleic acid analysis in (a) further comprises sequencing the enriched proximity ligated nucleic acid molecules.
B18. The method of embodiment B17, wherein the sequencing generates hundreds of sequence reads.
B19. The method of embodiment B17, wherein the sequencing generates thousands of sequence reads.
B20. The method of embodiment B17, wherein the sequencing generates tens of thousands of sequence reads.
B21. The method of embodiment B17, wherein the sequencing generates hundreds of thousands of sequence reads.
B22. The method of embodiment B17, wherein the sequencing generates millions of sequence reads.
B22.1 The method of embodiment B17, wherein the sequencing generates hundreds of millions of sequence reads.
B23. The method of any one of embodiments B1-B22.1, wherein the structural variant comprises one or more of a translocation, inversion, insertion, deletion, and duplication.
B24. The method of any one of embodiments B1-B23, wherein the structural variant comprises a microduplication and/or a microdeletion.
B25. The method of any one of embodiments B1-B24, wherein a breakpoint of the structural variant is located within a cancer gene.
B26. The method of any one of embodiments B1-B24, wherein a breakpoint of the structural variant is located outside of a cancer gene.
B27. The method of embodiment B25 or B26, wherein the cancer gene is chosen from one of the cancer genes listed in row 7 and/or row 15 of Table 10.
B28. The method of embodiment B25 or B26, wherein the cancer gene is selected from the groups consisting of: cancer genes found in row 7 and 15 of Table 10.
B30. The method of any one of embodiments B1-B29, wherein the subject is a human.
B31. The method of embodiment B30, wherein the subject is an adult patient.
B32. The method of embodiment B30, wherein the subject is a pediatric patient.
B33. The method of any one of embodiments B1-B32, wherein the subject has, or is suspected of having, a disease.
B34. The method of any one of embodiments B1-B33, wherein the subject has, or is suspected of having, cancer.
B34.1 The method of embodiment B34, wherein the cancer is selected from the cancers listed in row 3 of Table 10.
B34.2 The method of embodiment B34-B34.1, wherein the cancer is a blood cancer.
B34.4 The method of embodiment B34-B34.2 wherein the cancer is a liquid heme type.
B34.5 The method of embodiment B34-B34.3, wherein the cancer is not a solid heme type.
B34.6 The method of embodiment B34-B34.4 wherein the cancer is not a liquid heme type.
B35. The method of embodiment B34 to B34.6 wherein the cancer is a rare cancer.
B36. The method of embodiment B34 to B35, wherein the cancer is uterine myxoid leiomyosarcoma.
B37. The method of embodiment B34 to B35, wherein the cancer is subependymal giant cell astrocytoma (SEGA).
B38. The method of embodiment B34 to B35, wherein the cancer is a malignant brain tumor.
B39. The method of embodiment B34 to B35, wherein the cancer is chordoma.
B40. The method of embodiment B34 to B35, wherein the cancer is meningioma.
B41. The method of embodiment B34 to B35, wherein the cancer is embryonal tumors with multilayered rosettes (ETMR).
B42. The method of embodiment B34 to B35, wherein the cancer is metastatic high-grade sarcoma, uterine origin.
B43. The method of embodiment B34 to B35, wherein the cancer is pleomorphic xanthoastrocytoma (PXA).
B44. The method of embodiment B34 to B35, wherein the cancer is glioblastoma multiforme/anaplastic astrocytoma with piloid features (ANA PA).
B45. The method of embodiment B34 to B35, wherein the cancer is glioma.
B46. The method of embodiment B34 to B35, wherein the cancer is kidney primitive neuroectodermal tumor (PNET).
B46.1 The method of embodiment B34 to B35, wherein the cancer is myxoid leiomyosarcoma (LMS).
B46.2 The method of embodiment B34 to B35, wherein the cancer is Burkitt lymphoma.
B46.3 The method of embodiment B34 to B35, wherein the cancer is plasmacytoma.
B46.4 The method of embodiment B34 to B35, wherein the cancer is osseous plasmacytoma.
B46.5 The method of embodiment B34 to B35, wherein the cancer is classic Hodgkins lymphoma.
B46.6 The method of embodiment B34 to B35, wherein the cancer is diffuse large B cell lymphoma.
B46.7 The method of embodiment B34 to B35, wherein the cancer is pituitary adenoma.
B46.8 The method of embodiment B34 to B35, wherein the cancer is leiomyosarcoma.
B47. The method of any one of embodiments B1-B46.8, wherein the sample is a tissue sample, a cell sample, a blood sample, or a urine sample.
B48. The method of any one of embodiments B1-B47, wherein the sample comprises FFPE tissue.
B49. The method of any one of embodiments B1-B47, wherein the sample comprises frozen tissue.
B50. The method of any one of embodiments B1-B47, wherein the sample comprises peripheral blood.
B51. The method of any one of embodiments B1-B47, wherein the sample comprises blood obtained from bone marrow.
B52. The method of any one of embodiments B1-B47, wherein the sample comprises cells obtained from urine.
B53. The method of any one of embodiments B1-B47, wherein the sample comprises cell-free nucleic acid.
B54. The method of any one of embodiments B1-B53, wherein the sample comprises one or more tumor cells.
B55. The method of any one of embodiments B1-B54, wherein the sample comprises one or more circulating tumor cells.
B56. The method of any one of embodiments B1-B53, wherein the sample comprises a solid tumor.
B57. The method of any one of embodiments B1-B53, wherein the sample comprises a blood tumor.
B58. The method of any one of embodiments B1-B57, further comprising detecting the presence of cancer in the subject when the structural variant is present.
B59. The method of any one of embodiments B1-B58, further comprising administering a treatment to the subject when the structural variant is present.
B60. The method of any one of embodiments B1-B59, further comprising identifying a cancer gene spatially proximal to the structural variant.
B61. The method of embodiment B60, further comprising administering a cancer gene-specific treatment to the subject according to the identified cancer gene located spatially proximal to the structural variant.
A56. The method of embodiment A55, further comprising administering a cancer gene-specific treatment to the subject according to the identified cancer gene located spatially proximal to the structural variant.
B62. The method of any one of embodiments B59-B61, wherein the treatment comprises one or more treatments chosen from a modulator of a cancer gene.
B63. The method of any one of embodiments B59-B61, wherein the treatment comprises one or more treatments chosen from a modulator of a cancer gene, wherein the cancer gene is one of the cancer genes of row 7 and/or row 15 of Table 10.
C1. A composition comprising a set of synthetic oligonucleotide species, wherein:
   a) each oligonucleotide species is 10 to 500 consecutive nucleotides in length;
   b) each oligonucleotide species comprises a polynucleotide identical to or complementary to a subsequence in an exon of a cancer gene; and
   c) the polynucleotide maps to coordinates that are within 300-400 bp of one or more sites targeted by one or more restriction enzymes.
C1.1 The composition of embodiment C1, wherein each oligonucleotide species further comprises a polynucleotide identical to or complementary to a subsequence a cancer gene.
C1.2 The composition of embodiment C1 or C1.1, wherein each oligonucleotide species further comprises a polynucleotide identical to or complementary to a subsequence in an intron of a cancer gene.
C2. The composition of embodiment C1 or C1.1, wherein the polynucleotide maps to coordinates that are within 350 bp of one or more sites targeted by one or more restriction enzymes.
C2.1 The composition of any one of embodiments C1 to C2, wherein the one or more sites targeted by the one or more restriction enzymes comprise \hat{ }GATC, wherein \hat{ } is a cut site.
C2.2 The composition of any one of embodiments C1 to C2.1, wherein the one or more sites targeted by the one or more restriction enzymes comprise G\hat{ }ANTC, wherein \hat{ } is a cut site and N is A, C, G, or T.
C3. The composition of any one of embodiments C1-C2.2, wherein each oligonucleotide species comprises a polynucleotide identical to or complementary to a subsequence in an exon of a cancer gene listed in Table 7.
C3.1 The composition of any one of embodiments C1-C2.2, wherein each oligonucleotide species further comprises a polynucleotide identical to or complementary to a subsequence in an intron of a cancer gene listed in Table 7.
C4. The composition of any one of embodiments C1-C3.1, wherein the set of synthetic oligonucleotide species comprises about 10 or more oligonucleotide species.
C5. The composition of any one of embodiments C1-C3.1, wherein the set of synthetic oligonucleotide species comprises about 20 or more oligonucleotide species.
C6. The composition of any one of embodiments C1-C3.1, wherein the set of synthetic oligonucleotide species comprises about 50 or more oligonucleotide species.
C7. The composition of any one of embodiments C1-C3.1, wherein the set of synthetic oligonucleotide species comprises about 100 or more oligonucleotide species.
C8. The composition of any one of embodiments C1-C3.1, wherein the set of synthetic oligonucleotide species comprises about 500 or more oligonucleotide species.
C9. The composition of any one of embodiments C1-C3.1, wherein the set of synthetic oligonucleotide species comprises about 1,000 or more oligonucleotide species.
C10. The composition of any one of embodiments C1-C3.1, wherein the set of synthetic oligonucleotide species comprises about 10,000 or more oligonucleotide species.
C11. The composition of any one of embodiments C1-C3.1, wherein the set of synthetic oligonucleotide species comprises about 100,000 or more oligonucleotide species.
C12. The composition of any one of embodiments C1-C3.1, wherein the set of synthetic oligonucleotide species comprises about 300,000 or more oligonucleotide species.
C13. A kit comprising a composition of any one of embodiments C1-C12 and instructions for use.
D1. A method for detecting the presence or absence of a structural variant in a sample, the method comprising:
   a) obtaining a sample from a subject over a plurality of time points;
   b) for the sample obtained at each of the time points, performing a nucleic acid analysis on the sample, wherein the analysis comprises a method that preserves spatial-proximal contiguity information; and
   c) detecting whether a structural variant is present or absent in the selected sample according to the nucleic acid analysis in (b).
D2. The method of embodiment D1, further comprising detecting presence of minimal residual disease (MRD) in the subject when the structural variant is present, or detecting absence of minimal residual disease (MRD) in the subject when the structural variant is absent.
D3. The method of embodiment D2, further comprising administering a treatment, or continuing to administer a treatment, to the subject when the structural variant is present.
D4. The method of embodiment D2, further comprising stopping a treatment for the subject when the structural variant is absent.
D5. The method of any one of embodiments D1-D4, further comprising detecting an amount of the structural variant in the sample.
D6. The method of embodiment D5, further comprising detecting a level of minimal residual disease (MRD) in the subject according to the amount of structural variant detected in the sample.
D7. The method of any one of embodiments D1-D6, wherein a breakpoint of the structural variant is located within a cancer gene.
D8. The method of any one of embodiments D1-D6, wherein a breakpoint of the structural variant is located outside of a cancer gene.
D9. The method of embodiment D7 or D8, wherein the cancer gene is chosen from a cancer gene in row 7 and/or row 15 of Table 10.
D10. The method of embodiment D7 or D8, wherein the cancer gene is selected from cancer genes in Table 7.
D11. The method of embodiment D7 or D8, wherein the cancer gene is chosen from a cancer gene in row 7 and/or row 15 of Table 10.
D12. The method of any one of embodiments D1-D11.1, wherein the structural variant comprises one or more of a translocation, inversion, insertion, deletion, and duplication.
D13. The method of any one of embodiments D1-D12, wherein the structural variant comprises a microduplication and/or a microdeletion.
D14. The method of embodiment D12, wherein the translocation or the insertion comprises nucleic acid from a chromosome that is the same chromosome on which a cancer gene is located.
D15. The method of embodiment D12, wherein the translocation or the insertion comprises nucleic acid from a chromosome that is a different chromosome from which a cancer gene is located.
D16. The method of any one of embodiments D1-D15, wherein a breakpoint for the structural variant is located on the same chromosome as a cancer gene.
D17. The method of any one of embodiments D1-D16, wherein the nucleic acid analysis in (b) comprises generating proximity ligated nucleic acid molecules.
D18. The method of embodiment D17, wherein the nucleic acid analysis in (b) further comprises sequencing the proximity ligated nucleic acid molecules.
D19. The method of embodiment D18, wherein the sequencing generates hundreds of sequence reads.
D20. The method of embodiment D18, wherein the sequencing generates thousands of sequence reads.
D21. The method of embodiment D18, wherein the sequencing generates tens of thousands of sequence reads.
D22. The method of embodiment D18, wherein the sequencing generates hundreds of thousands of sequence reads.
D23. The method of embodiment D18, wherein the sequencing generates millions of sequence reads.
D23.1 The method of embodiment D18, wherein the sequencing generates hundreds of millions of sequence reads.
D24. The method of any one of embodiments D1 to D23.1, wherein the subject is a human.
D25. The method of embodiment D24, wherein the subject is an adult patient.
D26. The method of embodiment D24, wherein the subject is a pediatric patient.
D27. The method of any one of embodiments D2-D26, wherein the disease is cancer.
D27.1 The method of any one of embodiments D2-D26, wherein the disease is cancer, where the cancer is of a type listed in row 3 of Table 10.
D27.2 The method of any one of embodiments D2-D26, wherein the disease is cancer, where the cancer is selected from the group of cancers consisting of: cancer types listed in row 3 of Table 10.
D28. The method of embodiment D27-D27.2, wherein the cancer is a rare cancer.
D28.1 The method of embodiment D27-D28, wherein the cancer is a blood cancer.
D28.2 The method of embodiment D27-D28.1 wherein the cancer is a liquid heme type.
D28.3 The method of embodiment D27-D28.2, wherein the cancer is not a solid heme type.
D28.4 The method of embodiment D27-D28.3 wherein the cancer is not a liquid heme type.
D29. The method of embodiment D27 to D28.4, wherein the cancer is uterine myxoid leiomyosarcoma.
D30. The method of embodiment D27 to D28.4, wherein the cancer is subependymal giant cell astrocytoma (SEGA).
D31. The method of embodiment D27 to D28.4, wherein the cancer is a malignant brain tumor.
D32. The method of embodiment D27 to D28.4, wherein the cancer is chordoma.
D33. The method of embodiment D27 to D28.4, wherein the cancer is meningioma.
D34. The method of embodiment D27 to D28.4, wherein the cancer is embryonal tumors with multilayered rosettes (ETMR).
D35. The method of embodiment D27 to D28.4, wherein the cancer is metastatic high-grade sarcoma, uterine origin.
D36. The method of embodiment D27 to D28.4, wherein the cancer is pleomorphic xanthoastrocytoma (PXA).
D37. The method of embodiment D27 to D28.4, wherein the cancer is glioblastoma multiforme/anaplastic astrocytoma with piloid features (ANA PA).
D38. The method of embodiment D27 to D28.4, wherein the cancer is glioma.
D39. The method of embodiment D27 to D28.4, wherein the cancer is kidney primitive neuroectodermal tumor (PNET).
D39.1 The method of embodiment D27 to D28.4, wherein the cancer is myxoid leiomyosarcoma (LMS).
D39.2 The method of embodiment D27 to D28.4, wherein the cancer is Burkitt lymphoma.
D39.3 The method of embodiment D27 to D2.48, wherein the cancer is plasmacytoma.
D39.4 The method of embodiment D27 to D28.4, wherein the cancer is osseous plasmacytoma.
D39.5 The method of embodiment D27 to D28.4, wherein the cancer is classic Hodgkins lymphoma.
D39.6 The method of embodiment D27 to D28.4, wherein the cancer is diffuse large B cell lymphoma.
D39.7 The method of embodiment D27 to D28.4, wherein the cancer is pituitary adenoma.
D39.8 The method of embodiment D27 to D28.4, wherein the cancer is leiomyosarcoma.
D40. The method of any one of embodiments D1-D39.8, wherein the sample is a tissue sample, a cell sample, a blood sample, or a urine sample.
D41. The method of any one of embodiments D1-D40, wherein the sample comprises FFPE tissue.
D42. The method of any one of embodiments D1-D40, wherein the sample comprises frozen tissue.
D43. The method of any one of embodiments D1-D40, wherein the sample comprises peripheral blood.
D44. The method of any one of embodiments D1-D40, wherein the sample comprises blood obtained from bone marrow.
D45. The method of any one of embodiments D1-D40, wherein the sample comprises cells obtained from urine.
D46. The method of any one of embodiments D1-D40, wherein the sample comprises cell-free nucleic acid.
D47. The method of any one of embodiments D1-D46, wherein the sample comprises one or more tumor cells.
D48. The method of any one of embodiments D1-D47, wherein the sample comprises one or more circulating tumor cells.
D49. The method of any one of embodiments D1-D46, wherein the sample comprises a solid tumor.
D50. The method of any one of embodiments D1-D46, wherein the sample comprises a blood tumor.

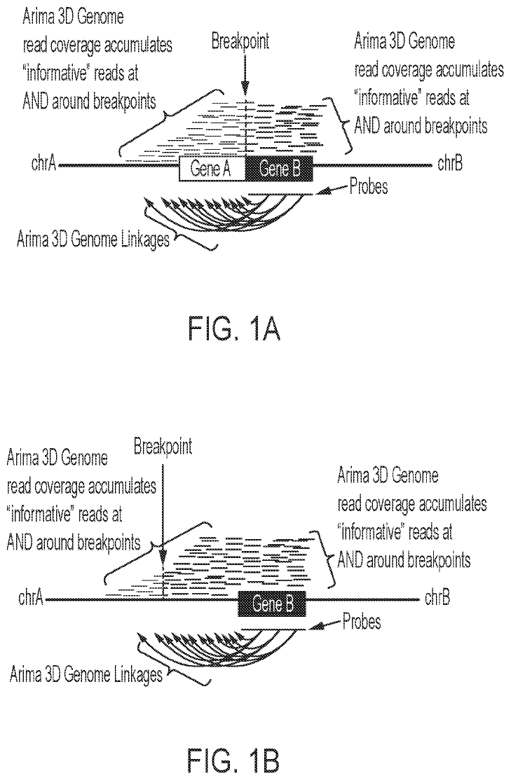

FIG. 1A shows a schematic of Capture-HiC data using target enrichment probes targeted to cancer genes, in order to identify a SV that results in a gene fusion. The schematic shows a SV between hypothetical chromosome A and hypothetical chromosome B, which creates a gene fusion between Gene A (on chromosome A) and Gene B (on chromosome B). The breakpoint is located in the center, where Gene A is fused to Gene B. The horizontal bar below Gene B depicts the targeting of probes to enrich for Gene B during the Capture-HiC workflow. The “arcs with arrows” at the bottom depict the concept that a captured HiC fragment containing Gene B may also contain a fragment from Gene A, or the genetic locus around Gene A, due to the nature of capturing 3D spatial proximity of DNA. This concept is portrayed in the figure as “3D Genome Linkages”-meaning fragments that are linked between Gene B and Gene A due to spatial proximity. There would also likely be a fragment between Gene B and Gene A or the locus around Gene B, but those are not depicted as they are not necessarily informative to detect a structural variant (SV) between chrA and chrB. Above the chromosome depicts dark gray and light gray sequence reads from this hypothetical Capture-HiC experiment. Dark gray fragments are derived from chrB and light gray fragments are derived from chrA. The intended depiction here is that each dark gray fragment (or sequence read) is linked to a light grray fragment and thus informative to detect an SV between chrA and chrB. An entirely dark gray fragment can be linked to an entirely light gray fragment, and still be informative despite neither fragment containing the breakpoint. Also depicted here is the notion that some sequence reads will contain the actual breakpoint, indicated by a black tick mark. Lastly, it is intentionally depicted here that the read coverage of reads linked to Gene B get lesser as one moves further away along the genome from Gene B. This is to reflect the property of the 3D genome that the spatial proximity between any two points along the genome is higher when they are linearly proximal, and further when they are linearly distal along a chromosome.

FIG. 1B shows a schematic of Capture-HiC data using target enrichment probes targeted to cancer genes, in order to identify a SV that results in a breakpoint outside of the targeted gene body. Shown here is a schematic similar to FIG. 1, but with the following differences. First, the breakpoint here is outside of the targeted gene body. Shown here the breakpoint does not lie within a gene, but the same principle would be true if the breakpoint lied within a non-targeted gene as the core concept of this figure is to illustrate the detection of SVs where the breakpoints lie outside of any targeted gene (or any targeted sequence/region). Because the breakpoint is outside of Gene B, the dark gray fragments/reads directly above the Gene B icon can be linked to either light gray fragments from chrA, or, dark gray fragments from chrB but outside of chrB between Gene B and chrA. Those reads where both linked fragments are dark gray are not particularly informative to SV and breakpoint detection, only those between gene B and chrA. Also note that it is intentionally depicted that some reads linked to Gene B are both dark gray and light gray and contain the breakpoint. This is intended to show that the sequence fragment containing the breakpoint may spatially interact with sequence elements from the targeted Gene B, making it possible for targeted HiC data to detect not only the SVs (light gray to dark gray linkages), but also the breakpoint itself (dark gray to light gray/dark gray linkages). The number of breakpoints containing fragments and the total number of linkages between Gene B and chrA would be influenced by the linear distance between the breakpoint and the enriched gene due to the property of the 3D genome that the spatial proximity between any two points along the genome is higher when they are linearly proximal, and further when they are linearly distal along a chromosome.

### EXAMPLES

The examples set forth below illustrate certain implementations and do not limit the technology.

### Example 1: Identification of Structural Variants in Cancer Samples

In this Example, the identification of structural variants in cancer samples is described.

### HiC for FFPE

For FFPE samples, 1-10 FFPE sections of 5-10 μm thickness were subject to a HiC protocol for FFPE tissues (Arima Genomics, San Diego, CA). The FFPE samples were deparaffinized and rehydrated using one incubation with Xylene, one incubation with 100% ethanol, and one incubation with water. Following the water incubation, the deparaffinized and rehydrated tissue was incubated in Lysis Buffer (formulation below in Table 1) on ice for 20 min.

TABLE 1
Lysis Buffer

| Reagent | Stock Conc. | Units | μL/rxn | Final Conc. | Units | Master Mix |
|---|---|---|---|---|---|---|
| Tris-HCl | 1000 | mM | 1.667 | 8.333 | mM | 62.333 |
| pH 8.0 | | | | | | |
| NaCl | 1000 | mM | 1.667 | 8.333 | mM | 62.333 |
| IGEPAL | 10 | % | 3.333 | 0.167 | % | 124.667 |
| Protease | 100 | % | 33.333 | 16.667 | % | 1246.667 |
| Inhibitor | | | | | | |
| Cocktail | | | | | | |
| DI Water | | | 160.00 | | | 5984.000 |
| | | Total/rxn | 200.00 | μL/rxn | | 7480 |

Following lysis incubation, samples were pelleted, decanted, and resuspended in 20 μl of 1× Tris Buffer pH 7.4.

Then, 24 μl of Conditioning Solution (formulation below in Table 2) was added and the samples were incubated at 74° C. for 40 min.

TABLE 2
Conditioning Solution

| Reagent | Stock Conc. | Units | μL/rxn | Final Conc. | Units | Master Mix |
|---|---|---|---|---|---|---|
| SDS | 20 | % | 1.104 | 0.920 | % | 41.290 |
| DI Water | | | 22.896 | | | 856.310 |
| | | Total/rxn | 24.000 | μL/rxn | | 897.6 |

20 μl of Stop Solution 2 (10.71% TritonX-100) was then added and the samples were incubated at 37° C. for 15 min.

After incubation in the Stop Solution, 12 μl of a Digestion Master Mix (formulation below in Table 3) was added and the samples were incubated for 1 hr at 37° C., followed by 20 min at 62° C.

TABLE 3
Digestion Master Mix

| | | | | | | |
|---|---|---|---|---|---|---|
| Reagent | Stock Conc. | Units | μL/rxn | Final Conc. | Units | Master Mix |

| | | | | | |
|---|---|---|---|---|---|
| NEB3.1 | 10 | x | 7.000 | | 261.800 |
| Dpnll | 50 | U/μL | 1 | | 37.400 |
| Hinfl | 50 | U/μL | 4 | | 149.6000 |
| | | Total/rxn | 12.000 | μL/rxn | 448.8 |

Then, 16 μl of a Fill-In Master Mix (formulation below in Table 4) was added and the samples were incubated for 45 min at 23° C. (room temperature).

TABLE 4
Fill-In Master Mix

| Reagent | Stock Conc. | Units | μL/rxn | Final Conc. | Units | Master Mix |
|---|---|---|---|---|---|---|
| dCTP | 10 | mM | 0.281 | 0.176 | mM | 10.509 |
| dGTP | 10 | mM | 0.281 | 0.176 | mM | 10.509 |
| dTTP | 10 | mM | 0.281 | 0.176 | mM | 10.509 |
| Biotin-dATP | 0.4 | mM | 7.013 | 0.175 | mM | 262.286 |
| 1X NEB3.1 | 1 | X | 4.144 | 0.259 | X | 154.986 |
| Klenow | 5 | U/μL | 4.000 | 1.250 | U/μL | 149.600 |
| | | Total/rxn | 16.000 | μL/rxn | | 598.4 |

82 μl of a Ligation Master Mix (formulation below in Table 5) was then added and the samples were incubated overnight at 23° C. (room temperature).

TABLE 5
Ligation Master Mix

| Reagent | Stock Conc. | Units | μL/rxn | Final Conc. | Units | Master Mix |
|---|---|---|---|---|---|---|
| 10% TritonX-100 | 10 | % | 13.580 | 1.656 | % | 507.892 |
| BSA | 100 | X | 1.650 | 2.012 | X | 61.710 |
| Ligase Buffer | 10 | X | 16.500 | 2.012 | X | 617.100 |
| T4 DNA Ligase | | | 12.00 | | | 448.800 |
| DI Water | | | 38.270 | | | 1431.298 |
| | | Total/rxn | 82.000 | μL/rxn | | 3066.8 |

Following the ligation incubation, 16.6 μl of 5 M NaCl was added and the samples were incubated overnight at 65° C.

Then, 35.5 μl of a Reverse Crosslinking Master Mix (formulation below in Table 6) was added and the samples were incubated overnight at 55° C.

TABLE 6
Reverse Crosslinking Master Mix

| | | | | | | Master |
|---|---|---|---|---|---|---|
| Reagent | Stock Conc. | Units | μL/rxn | Final Conc. | Units | Mix |
| SDS | 20 | % | 10.500 | 2.561 | % | 261.800 |
| Proteinase | | | 25.000 | | | 935.000 |
| K | | | | | | |
| | | Total/rxn | 35.000 | μL/rxn | | 1327.7 |

Following the reverse crosslinking incubation, DNA was purified using SPRI beads and then sonicated/sheared. DNA was size selected for fragments 200-600 bp in length using SPRI beads. Biotinylated DNA was enriched using Streptavidin beads, and on-bead DNA fragments were converted into adapter ligated Illumina sequencing libraries using reagents from the SWIFT ACCEL-NGS 2S Plus DNA Library Kit (Swift Biosciences/IDT).

Then, adapter ligated and bead-bound DNA was PCR amplified using reagents from KAPA, and the resulting PCR-amplified DNA was purified using SPRI beads. For samples subject to Capture-HiC, sufficient PCR cycles were used in order to obtain at least 500 ng (optimally 1500 ng) of DNA (the minimum amount of DNA used for probe hybridization in the Capture-HiC protocol). HiC libraries were subject to shallow sequencing QC on an Illumina MINISEQ. HiC libraries were subject to deep NGS on either Illumina HISEQ or NOVASEQ instruments.

### HiC for Blood

The HiC protocol for blood (Arima Genomics, San Diego, CA) matches that of FFPE protocol described above, except for the following differences.

Blood samples are not already fixed and then are not paraffin embedded. Therefore, the first step for blood is to crosslink blood cells using 2% formaldehyde for 10 min, quench crosslinking using a final concentration of 125 mM Glycine, and then begin HiC with the Lysis Step (see above).

The blood protocol differs from FFPE in the Conditioning Solution step, where Conditioning Solution for blood is added at 62° C. for 10 min. The blood protocol also differs from FFPE in the Ligation step, where Ligation reaction is 15 min instead of overnight. The blood protocol also differs from FFPE after Ligation but before DNA purification, in that a single Reverse Crosslinking master mix containing Proteinase K, NaCl, and SDS is added to the sample and it is incubated at 55° C. for 30 min, then 68° C. for 90 min, and then purified using SPRI beads.

The remainder of the protocol, including DNA shearing, size selection, library prep, PCR and Capture-HiC (below) is the same between blood and FFPE.

### Capture-HiC

First, 1500 ng of amplified HiC library was “pre-cleared” in order to remove residual biotinylated DNA. This was done by negative selection—the 1500 ng of amplified HiC library was combined with streptavidin beads, and the unbound DNA fraction was carried forward and the bound fraction was discarded.

The now pre-cleared amplified HiC library was then subject to Capture Enrichment, consisting of a) hybridization, b) capture; and c) amplification; according to the Agilent SURESELECT XTHS reagents and standard protocol. Capture targets/probes were custom-designed by Arima, using the Agilent SUREDESIGN software suite (details below). Following Capture Enrichment, Capture-HiC libraries were shallow sequenced on a MINISEQ or more deeply sequenced on an Illumina HISEQ.

### Capture Probe Design

A list of unique genes was compiled from the following sources:
NYU GenomePACT Panel
NYU Fusion SEQ′r Panel
ArcherDx VariantPlex Myeloid Panel
ArcherDx Pan Heme Panel
Stanford STAMP Heme Panel
ArcherDx Pan Solid Tumor
ArcherDx VariantPlex Solid Tumor
Childrens Hospital of Philadelphia (CHOP) Comprehensive Tumor and Fusion Panel
Agilent All-in-One Solid Tumor Panel
Agilent ClearSeq Comprehensive Cancer Panel
Foundation Medicine Foundation One CDx Panel
Stanford STAMP Solid Tumor Panel
Stanford STAMP Fusion Panel

These genes were then cross-referenced to the Ensembl data base, with 885 total genes collected (see Table 1 below). The exon coordinates were then located for all 885 genes, as well as the HiC restriction enzyme cut sites (Arima Genomics, San Diego, CA) within and directly flanking the exons. To define the target capture regions, the sequences within 350 bp from restriction enzyme cut sites were identified. For cut sites flanking the exons, the “inward” 350 bp (the 350 bp in the direction of the exon) was targeted. For this probe design, the cut sites were: \hat{ }GATC and G\hat{ }ANTC (where \hat{ } is the cut site on the positive strand, and “N” can be any of the 4 genomic bases, A, C, G, T). Collectively, this approach identified a set of coordinates in and around exons of genes of interest. These coordinates were then uploaded into the Agilent SUREDESIGN™ Software Suite for the design of individual probe sequences. Probe design was carried out using some custom parameters, including 1× tiling density, moderate stringency repeat masking, and optimized performance boosting. The probes were designed against the HG38 human reference genome. The total size of the target region was 12.075 Mb and following probe design 92.79449% (11.483 Mb) was covered by probes. In total, 335,242 probes were designed.

TABLE 7
Oncopanel genes

| | | | | |
|---|---|---|---|---|
| ABCB1 | CXCR4 | HOXA10 | NELL2 | RPS15 |
| ABCC2 | CXXC5 | HOXA9 | NF1 | RPS6KA2 |
| ABL1 | CYB5R2 | HOXB13 | NF2 | RPS6KB1 |
| ABL2 | CYLD | HRAS | NFATC2 | RPTOR |
| ABRAXAS1 | CYP17A1 | HSD3B1 | NFE2L2 | RRM1 |
| ACTG1 | CYP19A1 | HSP90AA1 | NFIB | RSPO2 |
| ACVR1 | CYP2A6 | HSP90AB1 | NFKB1 | RSPO3 |
| ACVR1B | CYP2B6 | ID3 | NFKB2 | RUNX1 |
| ACVR2A | CYP2C19 | ID4 | NFKBIA | RUNX1T1 |
| ADAMTS20 | CYP2C9 | IDH1 | NFKBIE | RXRA |
| ADGRA2 | CYP2D6 | IDH2 | NIN | RXRB |
| ADGRB3 | DAXX | IGF1R | NKX2-1 | RXRG |
| ADGRF5 | DCC | IGF2 | NLRP1 | S1PR2 |
| ADGRL3 | DCK | IGF2R | NME1 | SAMD9 |
| AFDN | DDB2 | IGHA1 | NOTCH1 | SBDS |
| AFF1 | DDIT3 | IGHA2 | NOTCH2 | SDC4 |
| AFF3 | DDR1 | IGHG1 | NOTCH3 | SDHA |
| AICDA | DDR2 | IGHG2 | NOTCH4 | SDHB |
| AKAP9 | DDX3X | IGHG3 | NPM1 | SDHC |
| AKT1 | DDX41 | IGHG4 | NR4A3 | SDHD |
| AKT2 | DEK | IGHJ1 | NRAS | SEMA6A |
| AKT3 | DENND3 | IGHJ2 | NRG1 | SERPINA9 |
| ALK | DHX15 | IGHJ3 | NSD1 | SETBP1 |
| ALOX12B | DICER1 | IGHJ4 | NSD2 | SETD2 |
| AMER1 | DIS3 | IGHJ5 | NSD3 | SETD5 |
| ANKRD24 | DLEU1 | IGHJ6 | NT5C2 | SF3B1 |
| ANKRD26 | DNAH9 | IGHM | NTRK1 | SGK1 |
| APC | DNAJB1 | IKBKB | NTRK2 | SH2B3 |
| APLNR | DNM2 | IKBKE | NTRK3 | SH2D1A |
| AR | DNMT3A | IKZF1 | NUMA1 | SH3BP5 |
| ARAF | DNMT3B | IKZF2 | NUMBL | SHH |
| ARFGAP3 | DNTT | IKZF3 | NUP214 | SHOC2 |
| ARFRP1 | DOT1L | IL16 | NUP93 | SLC22A1 |
| ARHGAP26 | DPH3 | IL2 | NUP98 | SLC22A2 |
| ARHGAP6 | DPYD | IL21R | NUTM1 | SLC29A1 |
| ARID1A | DROSHA | IL2RA | NUTM2A | SLC31A1 |
| ARID1B | DST | IL2RB | OGA | SLC34A2 |
| ARID2 | DUSP22 | IL2RG | P2RY8 | SLC45A3 |
| ARNT | E2F2 | IL3 | PAG1 | SLCO1B1 |
| ASB13 | EBF1 | IL3RA | PAICS | SMAD2 |
| ASH1L | EED | IL6ST | PAK3 | SMAD4 |
| ASPSCR1 | EGF | IL7R | PALB2 | SMARCA4 |
| ASXL1 | EGFR | ING4 | PARP1 | SMARCB1 |
| ATF1 | EGR1 | INHBA | PARP2 | SMARCE1 |
| ATM | EIF4A1 | INPP4B | PARP3 | SMC1A |
| ATR | EML4 | INSR | PAX3 | SMC3 |
| ATRX | EMSY | IRAG2 | PAX5 | SMO |
| AURKA | ENTPD1 | IRF2 | PAX7 | SMUG1 |
| AURKB | EP300 | IRF4 | PAX8 | SNCAIP |
| AURKC | EP400 | IRF8 | PBRM1 | SNX31 |
| AUTS2 | EPC1 | IRS2 | PBX1 | SOCS1 |
| AXIN1 | EPCAM | ITGA10 | PCBP1 | SOCS3 |
| AXL | EPHA2 | ITGA9 | PCDHAC2 | SOS1 |
| B2M | EPHA3 | ITGB2 | PCLAF | SOX10 |
| BAP1 | EPHA5 | ITGB3 | PDCD1 | SOX11 |
| BARD1 | EPHA7 | ITK | PDCD1LG2 | SOX2 |
| BATF3 | EPHB1 | ITPKB | PDE4DIP | SOX9 |
| BAX | EPHB4 | JAK1 | PDGFB | SP140 |
| BCL10 | EPHB6 | JAK2 | PDGFD | SPEN |
| BCL11A | EPOR | JAK3 | PDGFRA | SPI1 |
| BCL11B | ERBB2 | JARID2 | PDGFRB | SPOP |
| BCL2 | ERBB3 | JAZF1 | PDK1 | SPRED1 |
| BCL2A1 | ERBB4 | JMJD1C | PER1 | SPTA1 |
| BCL2L1 | ERCC1 | JUN | PGAP3 | SRC |
| BCL2L2 | ERCC2 | KAT6A | PHF1 | SRSF2 |
| BCL3 | ERCC3 | KAT6B | PHF6 | SS18 |
| BCL6 | ERCC4 | KDM5A | PHKB | SS18L1 |
| BCL9 | ERCC5 | KDM5C | PHLPP2 | SSX1 |
| BCOR | ERG | KDM6A | PHOX2B | SSX2 |
| BCORL1 | ERRFI1 | KDR | PICALM | SSX4 |
| BCR | ESR1 | KEAP1 | PIGA | STAG2 |
| BEND2 | ESR2 | KEL | PIK3C2B | STAT1 |
| BIRC2 | ESRRA | KIT | PIK3C2G | STAT3 |
| BIRC3 | ETNK1 | KLF2 | PIK3C3 | STAT4 |
| BIRC5 | ETS1 | KLF6 | PIK3CA | STAT5B |
| BLM | ETV1 | KLHL6 | PIK3CB | STAT6 |
| BLNK | ETV4 | KMT2A | PIK3CD | STIL |
| BMF | ETV5 | KMT2B | PIK3CG | STK11 |
| BMP7 | ETV6 | KMT2C | PIK3R1 | STK36 |
| BMPR1A | EWSR1 | KMT2D | PIK3R2 | STRBP |
| BOD1L1 | EXOC2 | KNL1 | PIM1 | STX11 |
| BRAF | EXT1 | KRAS | PIM2 | SUFU |
| BRCA1 | EXT2 | LAMA2 | PKD1L2 | SUZ12 |
| BRCA2 | EZH1 | LAMP1 | PKHD1 | SYK |
| BRD3 | EZH2 | LCK | PKN1 | SYNE1 |
| BRD4 | EZR | LIFR | PLAG1 | SYT1 |
| BRINP3 | FAM216A | LIMD1 | PLCG1 | TAF1 |
| BRIP1 | FANCA | LMO1 | PLCG2 | TAF15 |
| BTG1 | FANCC | LMO2 | PLEKHG5 | TAF1L |
| BTK | FANCD2 | LPP | PLEKHS1 | TAL1 |
| BUB1B | FANCE | LRP1B | PML | TAS2R38 |
| CACNA1E | FANCF | LTF | PMS1 | TBX22 |
| CALR | FANCG | LTK | PMS2 | TBX3 |
| CAMTA1 | FANCL | LUC7L2 | POLD1 | TCF12 |
| CARD11 | FAS | LYL1 | POLE | TCF3 |
| CASP8 | FBXW4 | LYN | POT1 | TCF7L1 |
| CBFA2T3 | FBXW7 | LZTR1 | POU5F1 | TCF7L2 |
| CBFB | FGF1 | LZTS1 | PPARG | TCL1A |
| CBL | FGF10 | MAF | PPAT | TEK |
| CBLB | FGF12 | MAFB | PPM1D | TENT5C |
| CBLC | FGF14 | MAGEA1 | PPP2R1A | TERC |
| CCDC170 | FGF19 | MAGI1 | PPP2R2A | TERT |
| CCDC50 | FGF23 | MAL | PPP6C | TET1 |
| CCN6 | FGF3 | MALT1 | PRCC | TET2 |
| CCNB3 | FGF4 | MAML2 | PRDM1 | TET3 |
| CCND1 | FGF6 | MAML3 | PRDM10 | TFE3 |
| CCND2 | FGFR1 | MAMLD1 | PRDM16 | TFEB |
| CCND3 | FGFR2 | MAP2K1 | PREX2 | TFG |
| CCNE1 | FGFR3 | MAP2K2 | PRKACA | TGFB1 |
| CCR4 | FGFR4 | MAP2K4 | PRKACB | TGFBR2 |
| CD22 | FGR | MAP3K1 | PRKAR1A | TGFBR3 |
| CD274 | FH | MAP3K13 | PRKAR2B | TGM7 |
| CD28 | FIP1L1 | MAP3K7 | PRKCA | THADA |
| CD44 | FLCN | MAPK1 | PRKCB | THBS1 |
| CD58 | FLI1 | MAPK8 | PRKCD | TIMP3 |
| CD70 | FLT1 | MARK1 | PRKCI | TIPARP |
| CD74 | FLT3 | MARK4 | PRKD1 | TLR2 |
| CD79A | FLT4 | MAST1 | PRKD2 | TLR4 |
| CD79B | FN1 | MAST2 | PRKD3 | TLX1 |
| CD83 | FOS | MBD1 | PRKDC | TLX3 |
| CDA | FOSB | MBTD1 | PRPF8 | TMEM216 |
| CDC25A | FOXA1 | MCL1 | PSIP1 | TMPRSS2 |
| CDC25C | FOXL2 | MDM2 | PSMB1 | TNFAIP3 |
| CDC73 | FOXO1 | MDM4 | PSMB2 | TNFRSF13B |
| CDH1 | FOXO3 | MEAF6 | PSMB5 | TNFRSF14 |
| CDH11 | FOXO4 | MECOM | PSMD1 | TNFRSF1A |
| CDH2 | FOXP1 | MED12 | PSMD2 | TNFRSF1B |
| CDH20 | FOXP4 | MED13 | PTCH1 | TNFSF4 |
| CDH23 | FOXR2 | MEF2B | PTEN | TNK2 |
| CDH5 | FSTL5 | MEN1 | PTGS2 | TOP1 |
| CDK12 | FUBP1 | MERTK | PTK2B | TP53 |
| CDK4 | FUS | MET | PTPN1 | TP63 |
| CDK6 | FUT8 | MITF | PTPN11 | TPM3 |
| CDK8 | FYN | MKNK1 | PTPRD | TPR |
| CDKN1A | FZR1 | MLC1 | PTPRO | TRAF3 |
| CDKN1B | G6PD | MLF1 | PTPRT | TRIM24 |
| CDKN2A | GABRA6 | MLH1 | PYCR1 | TRIM33 |
| CDKN2B | GATA1 | MLH3 | QKI | TRIP11 |
| CDKN2C | GATA2 | MLLT1 | RAB29 | TRRAP |
| CEBPA | GATA3 | MLLT10 | RAC1 | TSC1 |
| CEBPD | GATA6 | MME | RAD21 | TSC2 |
| CEBPE | GDNF | MMP2 | RAD50 | TSHR |
| CEBPG | GID4 | MN1 | RAD51 | TSLP |
| CHD1 | GLI1 | MNX1 | RAD51B | TYK2 |
| CHD2 | GLIS2 | MPL | RAD51C | TYRO3 |
| CHD4 | GNA11 | MRE11 | RAD51D | U2AF1 |
| CHD5 | GNA13 | MRTFA | RAD52 | U2AF2 |
| CHD7 | GNAI3 | MRTFB | RAD54L | UBR5 |
| CHEK1 | GNAQ | MSH2 | RAF1 | UGT1A1 |
| CHEK2 | GNAS | MSH3 | RAG1 | UMODL1 |
| CHIC2 | GNB1 | MSH6 | RAG2 | USP6 |
| CIC | GPS2 | MSMB | RALGDS | USP9X |
| CIITA | GRB7 | MST1R | RANBP1 | VAV1 |
| CILK1 | GRIN2A | MTAP | RARA | VEGFA |
| CKS1B | GRM3 | MTOR | RARB | VGLL2 |
| CMPK1 | GRM8 | MTR | RARG | VGLL3 |
| COL1A1 | GSK3B | MTRR | RB1 | VHL |
| CRBN | GSTP1 | MUC1 | RBBP6 | WAS |
| CREB1 | GUCY1A2 | MUSK | RBM10 | WRN |
| CREB3L2 | H1-2 | MUTYH | RBM15 | WT1 |
| CREBBP | H1-3 | MYB | RECQL4 | WWTR1 |
| CRKL | H1-4 | MYBL1 | REL | XPA |
| CRLF2 | H1-5 | MYC | RELA | XPC |
| CRTC1 | H2AC6 | MYCL | RET | XPO1 |
| CSF1 | H3-3A | MYCN | RHEB | XRCC2 |
| CSF1R | H3-3B | MYD88 | RHOA | YAP1 |
| CSF3R | H3C14 | MYH11 | RHOH | YES1 |
| CSMD3 | H3C2 | MYH9 | RICTOR | YWHAE |
| CSNK2B | HCAR1 | MYOD1 | RIT1 | ZCCHC7 |
| CTCF | HDAC1 | NAB2 | RNASEL | ZMYM2 |
| CTDNEP1 | HGF | NBN | RNF2 | ZMYM3 |
| CTLA4 | HIF1A | NCOA1 | RNF213 | ZNF217 |
| CTNNA1 | HLF | NCOA2 | RNF43 | ZNF384 |
| CTNNB1 | HMGA2 | NCOA3 | ROS1 | ZNF521 |
| CUL3 | HNF1A | NCOA4 | RPL22 | ZNF703 |
| CUL4A | HNRNPK | NCOR2 | RPN1 | ZRSR2 |
| CUX1 | HOOK3 | NEK6 | RPS14 | ZSWIM4 |

### HiC Data Analysis

To identify structural variants, raw HiC read-pairs were mapped to the human reference (hg38) and deduplicated. Mapped and deduplicated read pairs were then analyzed using the HiC-BREAKFINDER software (Dixon, Nature Genetics, 2018) to call structural variants.

For data visualization, HiC read-pairs were analyzed using the JUICER software, which outputs a “.hic” file that can be uploaded into the desktop JUICEBOX software for visualization of HiC heatmaps. Visual inspection, along with the structural variant calls from HiC-BREAKFINDER, were used to approximate the structural variant breakpoints from HiC analysis.

### Capture-HiC Data Preliminary Analysis

To identify structural variants, raw Capture-HiC read-pairs were mapped to the human reference (hg38) and deduplicated. Then, the genome was binned into different size genomic bins (e.g. 1 Mb, 50 kb, 1 kb), and then the total observed HiC read-pairs was summed between the gene of interest and every other bin in the genome. Each pair was tested (i.e., the number of counts between the gene of interest and Bin X) for statistical significance, modeled against a null distribution from non-tumor Capture-HiC data, and corrected for multiple testing. The output of this analysis are bins of the genome with statistically significant observed interactions with the gene of interest. The premise is that the gene within the bin(s) of highest statistical significance is involved in a structural variant with the gene of interest.

For data visualization, the observed read counts between a gene of interest and all other genomic bins can be represented as a “Manhattan Plot”. Data can also be visualized in the IGV browser, but portraying only the read-pairs with at least 1 end mapping to the gene of interest.

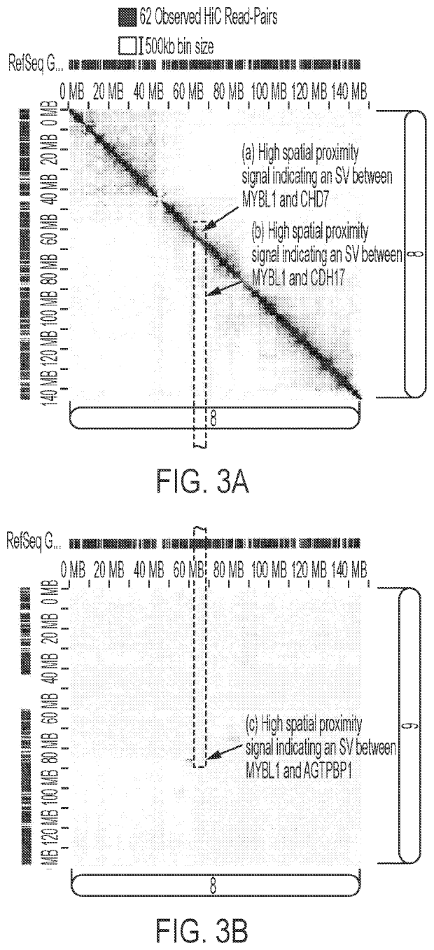

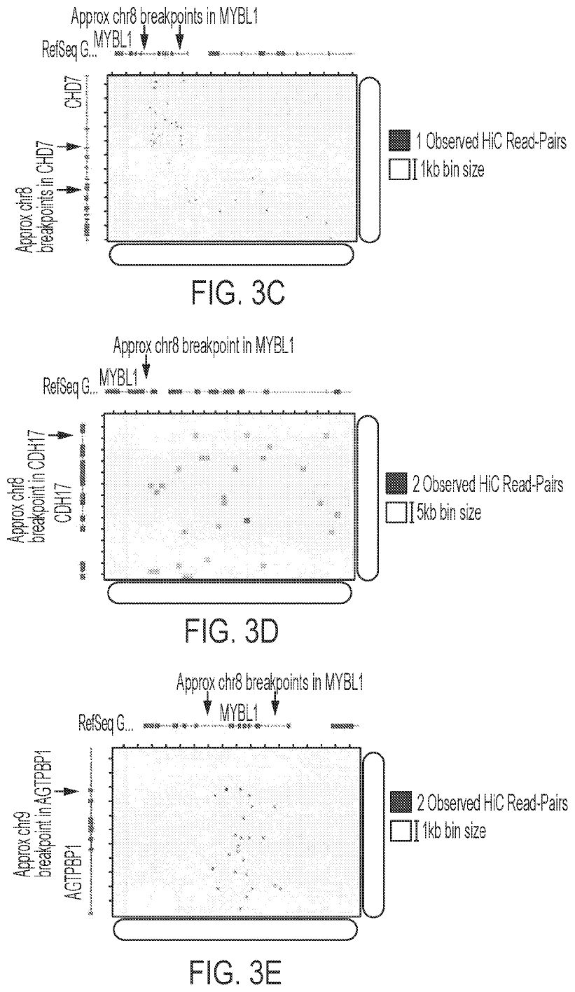

FIG. 3 shows a representative HiC analysis showing the detection of an SV that results in a gene fusion, which can resolve complex SVs involving multiple genes. FIG. 3A shows a HiC contact matrix showing all intra-chromosomal contacts within entire chr8. The tracks above and on the left side are gene positions. The bin size of this chromosome-wide analysis is 500 kb. The color darkness correlates with the number of observed HiC contacts between any pairs of genomic bins. The darkest color indicates 62 or greater observed HiC contacts. FIG. 3B shows a HiC contact matrix showing all inter-chromosomal contacts between chr8 and chr9. The track on the left are genes along the entire chr9, and the track across the top are all genes along the entire chr8. The two HiC heatmaps of FIG. 3A and FIG. 3B are directly stacked on top of one another so that the gene positions running left to right are the same between the two contact matrices. The dashed box encompasses the MYBL1 gene on chr8 and 3 SVs involving MYBL1. The top SV (indicated with the notation (a)), as indicated by a high spatial proximity (HiC) signal, is between MYBL1 and CHD7, albeit difficult to appreciate due to the close proximity of the gene-pair to the matrix diagonal. The middle SV (indicated with the notation (b)), as indicated by a high spatial proximity (HiC) signal, is between MYBL1 and CDH17. The bottom structural (indicated with the notation (c)), as indicated by a high spatial proximity (HiC) signal, is between MYBL1 and AGTPBP1. The first two (a+b) are intra-chromosomal SVs within chr8, and the last (c) is inter-chromosomal between chr8 and chr9. FIG. 3C is a zoomed-in view around the approximate breakpoints in MYBL1 and CHD7. The arrows show the approximate breakpoint locations inferred from the HiC analysis, with two breakpoints in MYBL1 and two breakpoints in CHD7. The HiC signal indicates that the sequence between the two MYBL1 breakpoints is in spatial proximity with the sequence that comprises the 5′ end of CHD7 up to the first breakpoint in CHD7. The HiC signal also indicates that the sequence from the 5′ end of MYBL1 up to the first breakpoint is in spatial proximity with the sequence in CHD7 from the second breakpoint to the 3′ end of the CHD7 gene body. FIG. 3D shows a zoomed-in view around the approximate breakpoints in MYBL1 and CDH17. The arrows indicate the approximate breakpoint locations inferred from the HiC analysis, with one breakpoint in MYBL1 and one breakpoint in CDH17. The HiC signal indicates that the sequence from the 5′ end of MYBL1 up to the breakpoint is in spatial proximity with the sequence in CDH17 from the 5′ end of the gene up to the breakpoint. FIG. 3E shows a zoomed-in view around the approximate breakpoints in MYBL1 and CHD7. The arrows indicate the approximate breakpoint locations inferred from the HiC analysis, with two breakpoints in MYBL1 and two breakpoints in AGTPBP1. The HiC signal indicates that the sequence between the two MYBL1 breakpoints is in spatial proximity with the sequence that comprises the 5′ end of AGTPBP1 up to the breakpoint in AGTPBP1.

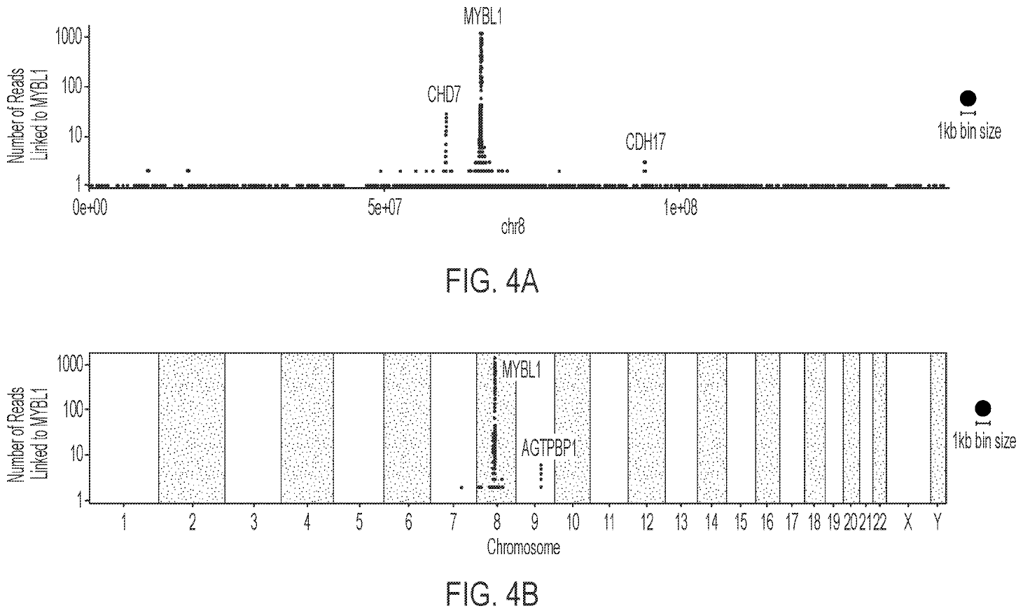

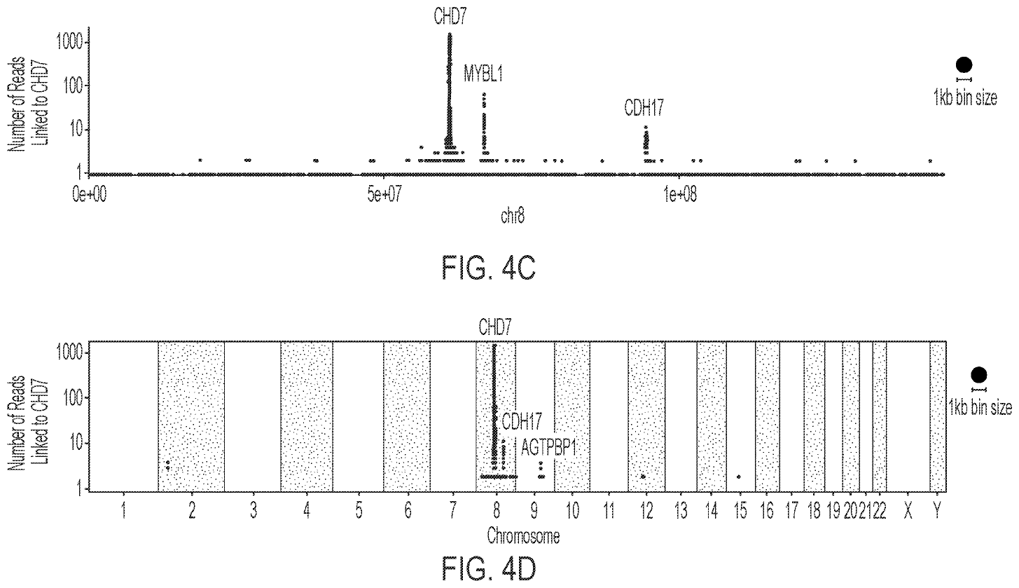

FIG. 4 shows a representative Capture-HiC genome-scan analysis used to identify sequences with high spatial proximity to a targeted gene where the SV results in a gene fusion which can resolve complex SVs involving multiple genes. FIG. 4A depicts a quantification of the observed Capture-HiC read-pairs where at least 1 read-end aligns to MYBL1 and the other ends aligns to anywhere along chr8. The plot is essentially a “scan” of how many Capture-HiC contacts are observed between MYBL1 and any bin of bin size 1 kb along chr8. One would then interpret that if there are high observed contacts, i.e., high spatial proximity, between MYBL1 and a linearly distal bin on chr8, that would be indicative of a SV that places MYBL1 into close linear proximity with that bin. The highest “peak” of signal is expectedly around MYBL1, as those segments linearly proximal to MYBL1 are also expected to be in highest spatial proximity. There is a “peak” upstream (to the left) of MYBL1 where the peak bin lies within CHD7, and then a lesser signal downstream where the peak bin lies within CDH17. This analysis broadly identifies that MYBL1 is in close spatial proximity to very distal genes CHD17 and CDH17, indicating SVs involving those 3 genes. FIG. 4B is the sample type of analysis as FIG. 4A, expect the x axis is the entire human genome rather than just chr8. The x-axis now has chromosome labels, and so the signal that was once spread across the entire plot in FIG. 4A is compressed into a single segment that comprises chr8 in FIG. 4B. The highest “peak” of signal is expectedly again around MYBL1, and the signal along chr8 is so compressed one cannot make out the peak at CHD7 or CDH17. However, there is a “peak” on chr9 within AGTPBP1. Taken together with FIG. 4A, these analyses broadly identify that MYBL1 is in close spatial proximity to very distal genes CHD17 and CDH17 on chr8, and AGTPBP1 on chr9, indicating SVs involving those 4 genes. Because the gene panel also targets the oncogene CHD7, FIG. 4C shows a depicted analogous to FIG. 4A, except here a quantification of the observed Capture-HiC read-pairs where at least 1 read-end aligns to CHD7 and the other ends aligns to anywhere along chr8. The genes MYBL1 and CDH17 shows “peaks” of high spatial proximity to CHD7. FIG. 4D is analogous to FIG. 4B where a quantification of the observed Capture-HiC read-pairs where at least 1 read-end aligns to CHD7 and the other ends aligns to anywhere along the human genome. Despite the compression along the x-axis, one can still visually appreciate the “peak” in CDH17, and then can also appreciate the “peak” at chr9 within AGTPBP1.

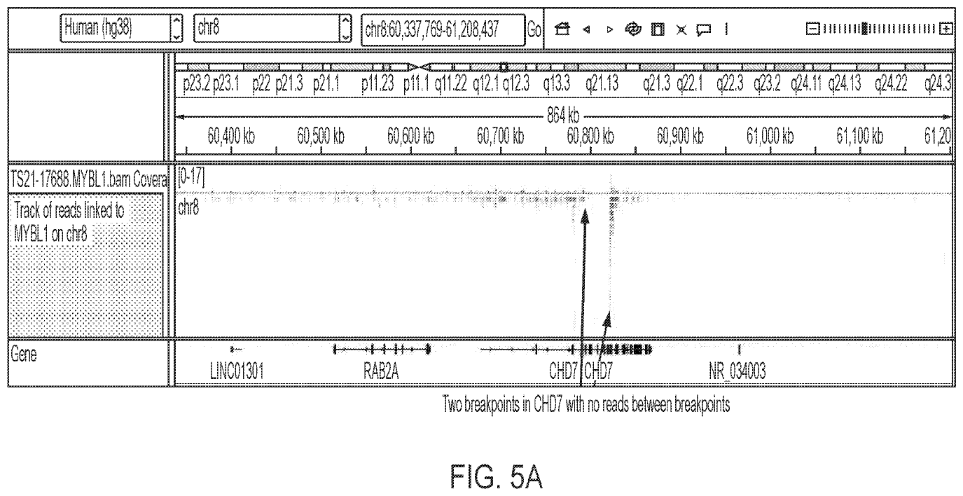

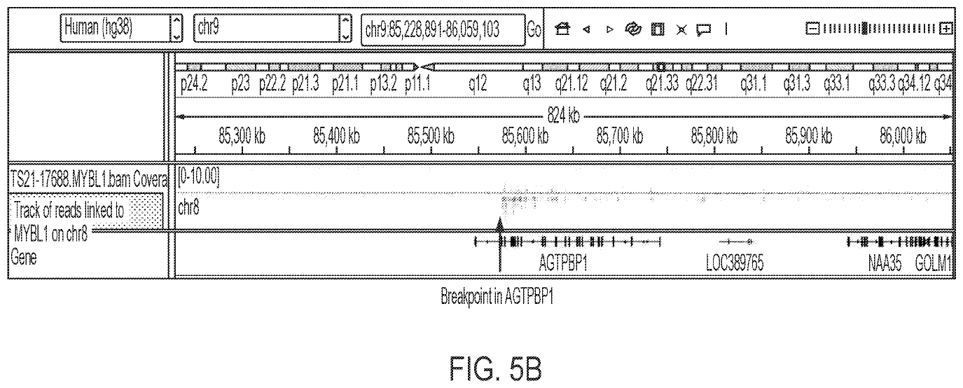

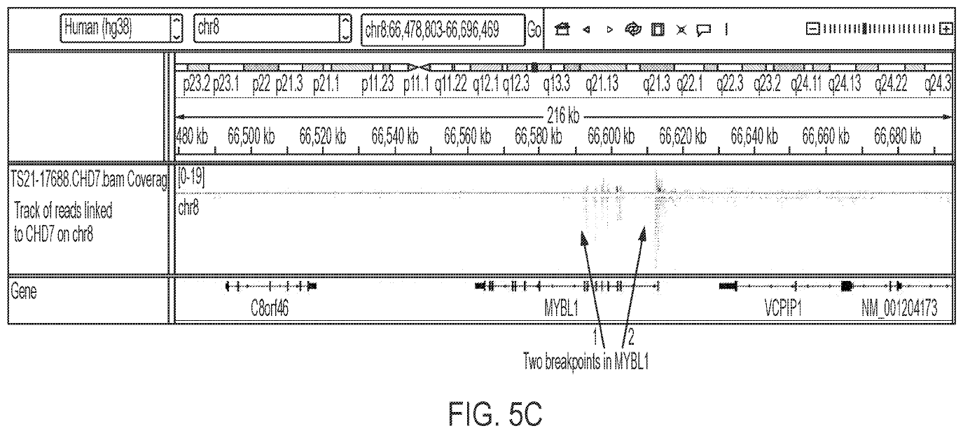

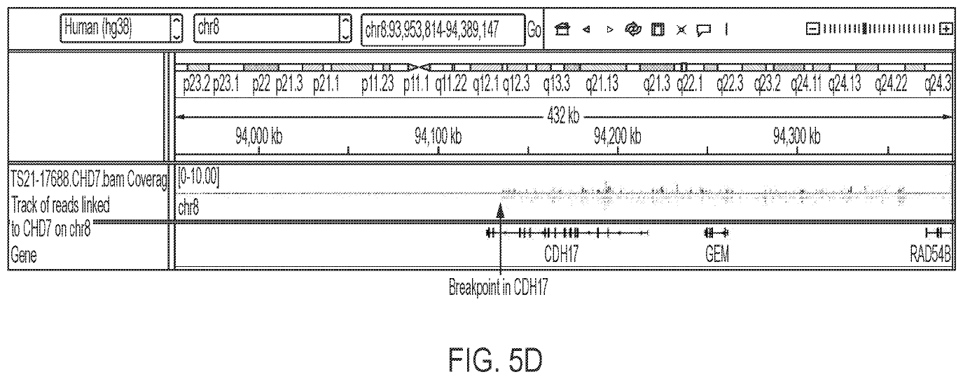

FIG. 5 shows representative Capture-HiC IGV Browser analyses, used for analyzing the breakpoint coordinates and genes involved in a particular SV that results in a gene fusion and which can resolve complex SVs involving multiple genes. The IGV is a publicly accessible tool for the visual exploration of genomic data (James T. Robinson, Helga Thorvaldsdóttir, Wendy Winckler, Mitchell Guttman, Eric S. Lander, Gad Getz, Jill P. Mesirov. Integrative Genomics Viewer. Nature Biotechnology 29, 24-26 (2011)). This figure is a “read-level” analysis version of FIG. 4. In particular, the way the data were processed was equivalent to FIG. 4, where all read-pairs that have one read-end aligning to the target gene, MYBL1, were extracted and then the raw reads were uploaded into the IGV browser for visualization. The processing of these reads was therefore equivalent to FIG. 4, except FIG. 4 then enumerates the total number of reads in a given window/bin size, and here individual reads are shown in the IGV browser. This browser view also facilitates the higher resolution read-level analysis of the “peaks” that were identified in the genome-scan analysis. Accordingly, FIG. 5A shows an IGV browser view of reads where one read-end aligns to MYBL1, and the other read end aligns around the CHD7 gene. The exact genome coordinates of the IGV view are shown as text towards the top of the IGV snapshot. The analysis indicates two breakpoints in CHD7 when involved in an SV with MYBL1 (arrows). Also of note is the absence of any reads between the two breakpoints, indicating the segment between those two breakpoints has been deleted in the context of the SV with MYBL1. Finally, one can appreciate at the read-level that the highest abundance of reads who's other-read end aligns to MYBL1 is at the breakpoints, and then the abundance of reads linked to MYBL1 decreases as one moves linearly distal to the breakpoints. This indicates the concept that the peak of read abundance is at the coordinates with greatest linear (and spatial) proximity to MYBL1, and then as one moves away linearly the breakpoint the abundance of spatial proximity signal with MYBL1 also decreases. FIG. 5B is similar to FIG. 5A, except shows an IGV browser view of reads where one read-end aligns to MYBL1, and the other read end aligns around the AGTPBP1 gene on chr9. The exact genome coordinates of the IGV view are shown as text towards the top of the IGV snapshot. Similar to FIG. 5A, one can appreciate the breakpoint at the “peak” of read abundance. One can also appreciate that there are only Capture-HiC reads between MYBL1 and the segment of AGTPBP1 from the 5′ end of the gene up to the breakpoint. There are 0 reads where one end aligns to MYBL1 and the other read end aligns to the segment of AGTPBP1 from the breakpoint to the 3′ end of the gene, indicating the structure of the SV involves MYBL1 and only the portion of AGTPBP1 from the breakpoint to the 5′ end of the gene. Together, FIGS. 5A and 5B demonstrate using the IGV browser how one can analyze breakpoints of the genes involved in the SV with MYBL1 and more detailed structural analysis of the portions of each gene involved in the SV with MYBL1. To get an understanding of the breakpoints and segments of MYBL1 involved in the SV, one can also do the “reverse analysis” and analyze an IGV browser view of reads where one read-end aligns to CHD7, and the other read end aligns around the MYBL1 gene, as shown in FIG. 5C. The exact genome coordinates of the IGV view are shown as text towards the top of the IGV snapshot. The analysis indicates two breakpoints in MYBL1 when involved in an SV with CHD7 (arrows). Also of note is the absence of any reads from breakpoint #1 to the 3′ end of MYBL1, indicating that the sequence segment from breakpoint #1 to the 3′ end of MYBL1 is not involved in the SV with CHD7. The IGV analysis also show a “peak” in spatial proximity signal around the 5′ end of MYBL1, labeled as breakpoint #2, with the expected Capture-HiC signal decay as one moves away (toward the right) from the breakpoint. FIG. 5D is similar to FIG. 5C except FIG. 5D shows an IGV browser view of reads where one read-end aligns to CHD7, and the other read end aligns around the CDH17 gene on chr8. The exact genome coordinates of the IGV view are shown as text towards the top of the IGV snapshot. One can appreciate the emergence of spatial proximity to CHD7 at the labeled breakpoint in CDH17, indicating that only the portion of CDH17 from the 5′ end of the gene up to the breakpoint is involved in an SV with CHD7. Together, FIGS. 5C and 5D demonstrate using the IGV browser how one can analyze breakpoints of the genes involved in the SV with CHD7, and, more detailed structural analysis of the portions of each gene involved in the SV with CHD7.

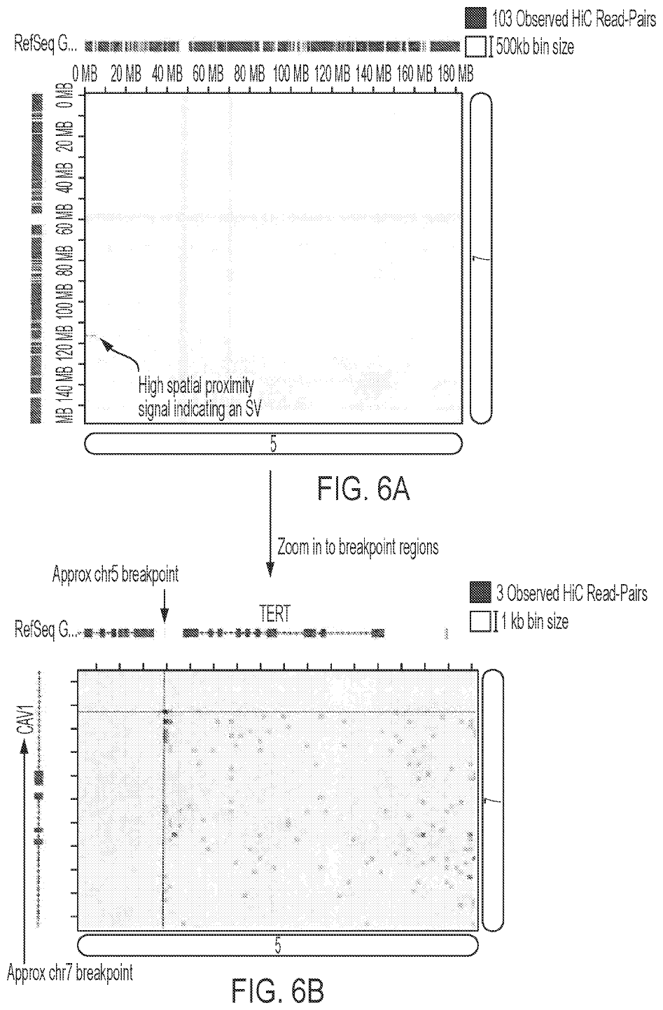

FIG. 6 shows a representative HiC analysis showing the detection of an SV that results in a breakpoint outside of a cancer-associated gene(s), but within a certain linear proximity to the cancer-associated gene(s). FIG. 6A shows a HiC contact matrix showing all inter-chromosomal contacts between chr5 and chr7. The tracks above and on the left side are gene positions. The bin size of this chromosome-wide analysis is 500 kb. The color darkness correlates with the number of observed HiC contacts between any pairs of genomic bins. The darkest color indicates 103 or greater observed HiC contacts. The arrow points to a segment of high spatial proximity between the two chromosomes, indicating the presence of an SV involving the respective segments on chr5 and chr7. FIG. 6B shows a zoomed-in view around the approximate breakpoints on chr5 and chr7. The tracks above and on the left side are gene positions. The bin size of this chromosome-wide analysis is 1 kb. The color darkness correlates with the number of observed HiC contacts between any pairs of genomic bins. The darkest color indicates 3 or greater observed HiC contacts. The approximate breakpoint locations inferred from the HiC analysis are shown with appropriately marked arrows, with one breakpoint on chr5 and one breakpoint on chr7. The breakpoint on chr5 is approximately 3,167 bp from the 3′ end of the gene body of the oncogene TERT (labeled in text, top). The breakpoint on chr5 is within the CAV1 gene (labeled in text, left), which is also 125,196 bp from the 5′ end of the gene body of the oncogene MET (out of view because this view is zoomed-in around the breakpoints).

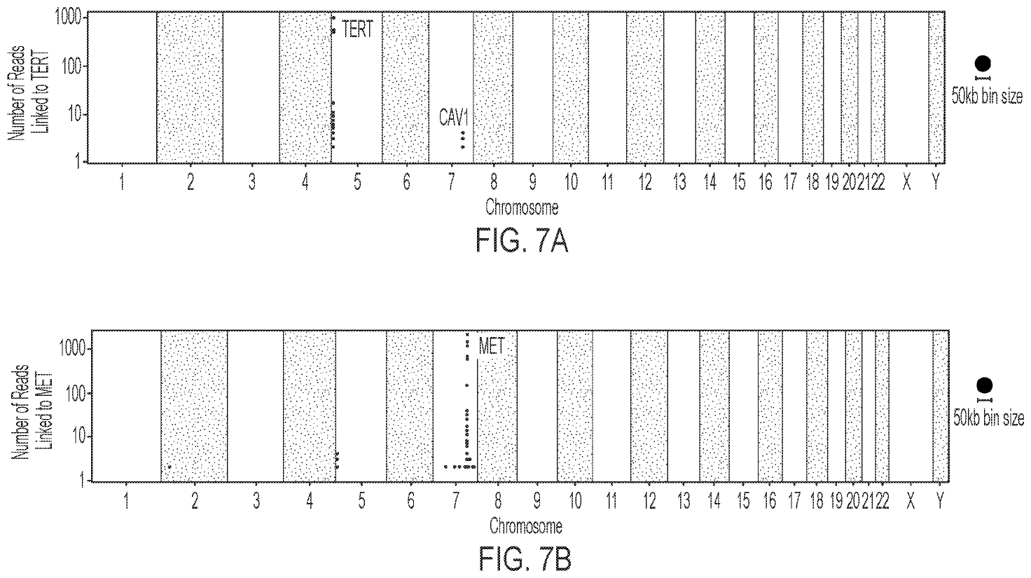

FIG. 7 shows representative Capture-HiC genome-scan analysis used to identify sequences with high spatial proximity to a targeted gene, where the SV breakpoint is outside of a targeted cancer-associated gene. FIG. 7A depicts a quantification of the observed Capture-HiC read-pairs where at least 1 read-end aligns to TERT and the other ends aligns to anywhere along the entire human genome. The x-axis has chromosome labels. The highest “peak” of signal is expectedly again around TERT, and there is also a “peak” on chr7 within CAV1. These data indicate that TERT is involved in a SV with a segment on chr7 and where the breakpoint may lie within the CAV1 gene. FIG. 7B depicts a quantification of the observed Capture-HiC read-pairs where at least 1 read-end aligns to MET and the other ends aligns to anywhere along the entire human genome. The x-axis has chromosome labels. The highest “peak” of signal is expectedly again around MET, and there is also a “peak” on chr5 near the TERT gene. These data indicate that MET is involved in an SV with a segment on chr5 and where the breakpoint may lie near the TERT gene. Note that in FIGS. 7A and 7B, the window/bin size for the genome-scan analysis is 50 kb, as labeled to the right of the genome-scan plots.

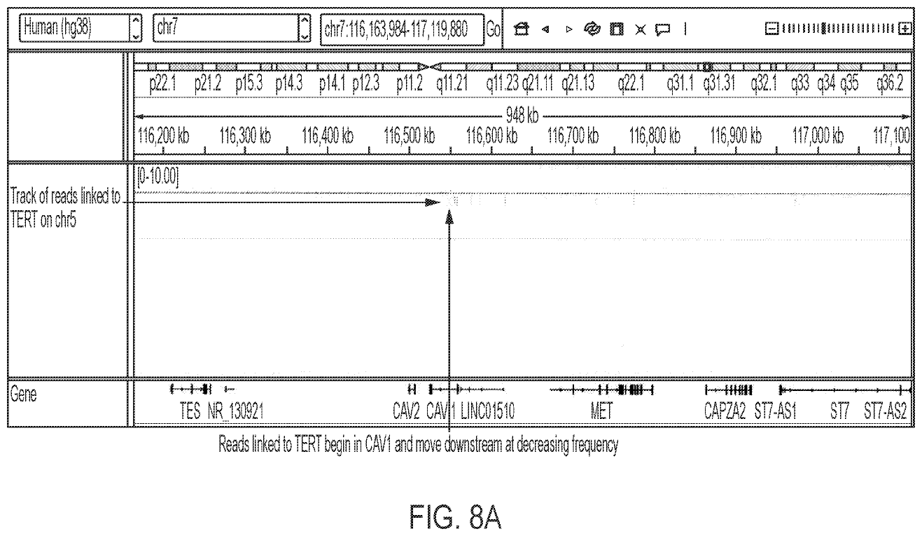

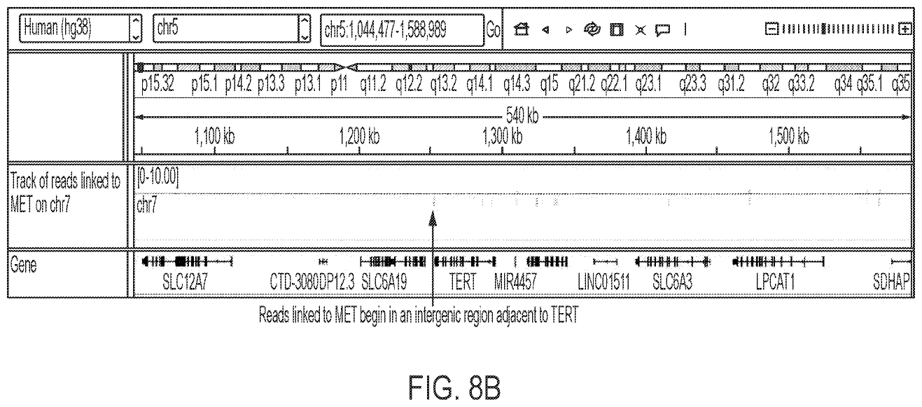

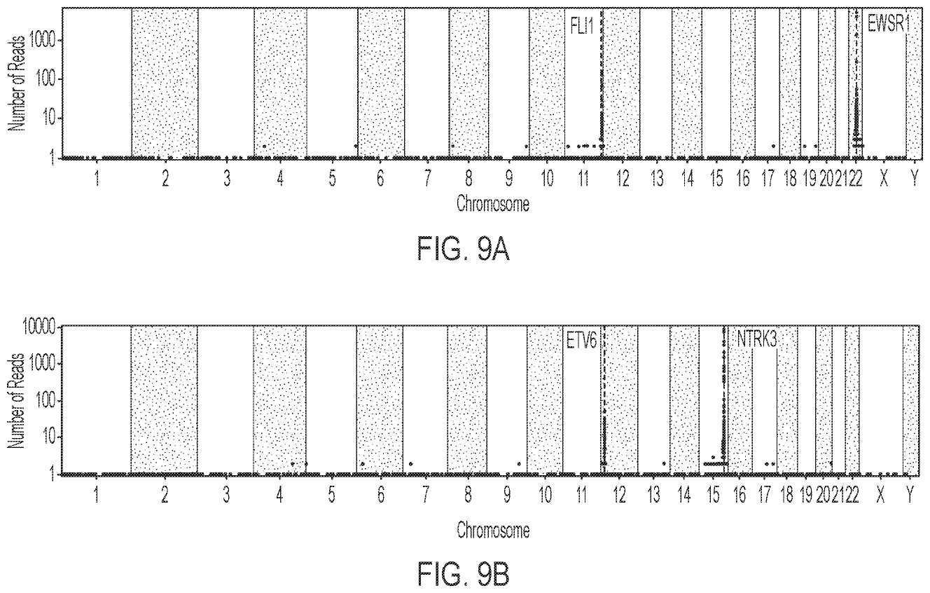

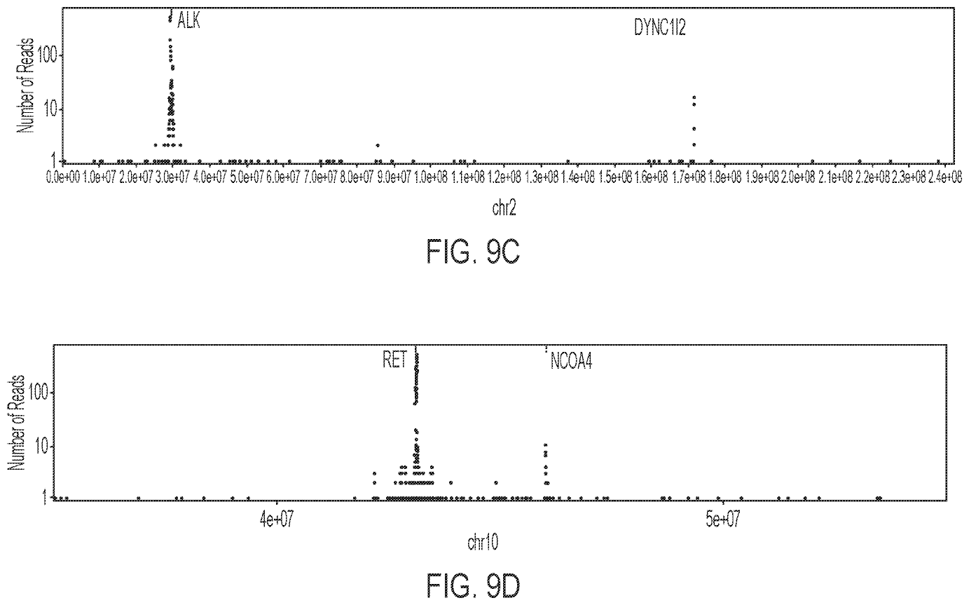

FIG. 8 shows a representative Capture-HiC IGV Browser analyses, used for analyzing the breakpoint coordinates and genes involved in a particular SV where the SV comprises a breakpoint outside of a targeted cancer-associated gene. This figure is a “read-level” analysis version of FIG. 7. The processing of these reads was equivalent to FIG. 7, except FIG. 7 then enumerates the total number of reads in a given window/bin size, and here individual reads are shown in the IGV browser. This browser view also facilitates the higher resolution read-level analysis of the “peaks” that were identified in the genome-scan analysis from FIG. 7. FIG. 8A shows an IGV browser view of reads where one read-end aligns to TERT, and the other read end aligns in and around the CAV1 gene. The exact genome coordinates of the IGV view are shown as text towards the top of the IGV snapshot. The analysis indicates the emergence of spatial proximity (Capture-HiC reads) signal starting in CAV1, indicating a breakpoint in CAV1. FIG. 8B shows an IGV browser view of reads where one read-end aligns to MET, and the other read end aligns around the TERT gene. The exact genome coordinates of the IGV view are shown as text towards the top of the IGV snapshot. The analysis indicates the emergence of spatial proximity (Capture-HiC reads) signal starting in an intergenic region adjacent to TERT, indicate a breakpoint at that intergenic region adjacent to TERT.

### Example 2: Uncovering Gene Fusions with 3D Genomics

Gene fusions as biomarkers have broad clinical utility in cancer patients. They may promote accurate diagnosis, early detection, prognosis, and selection of optimal treatment regimens. Identifying gene fusions in tumor biopsies is critical for understanding disease etiology. However, detecting gene fusions in tumor biopsies can be difficult for various reasons. For example, karyotyping may provide low-resolution; and fluorescence in situ hybridization (FISH) assays have low throughput and may be biased. RNA-seq does not perform well in formalin-fixed, paraffin-embedded (FFPE) tissue blocks due to RNA degradation, low transcript abundance, RNA panel design, or a combination of these issues. Clinical next generation sequencing (NGS) panels often fail to yield clear genetic drivers of disease as they predominantly focus on coding regions of the genome.

Profiling FFPE Tumors with 3D Genomics

A novel DNA-based partner-agnostic approach was developed for identifying fusions from formalin-fixed, paraffin-embedded (FFPE) tumor sample using 3D genomics based on Arima-HiC technology. In some instances, target enrichment (Capture-HiC) and NGS were also utilized.

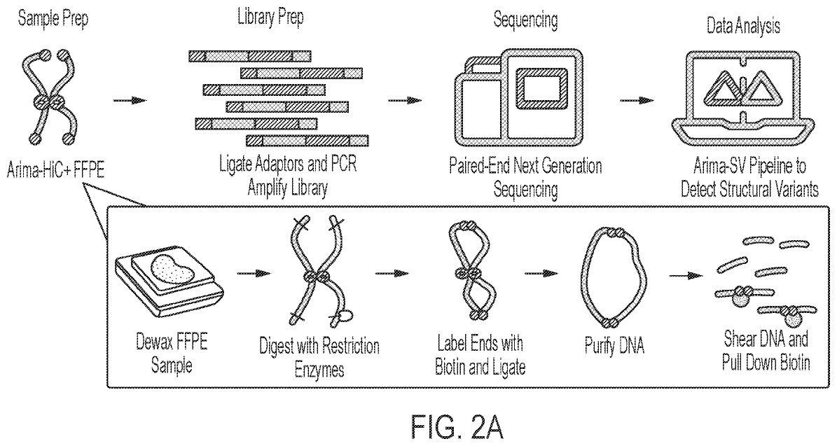

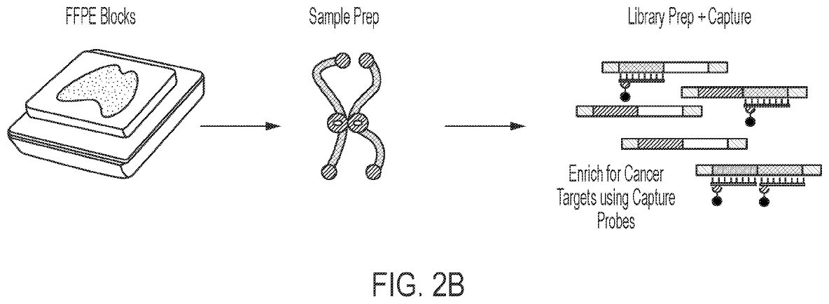

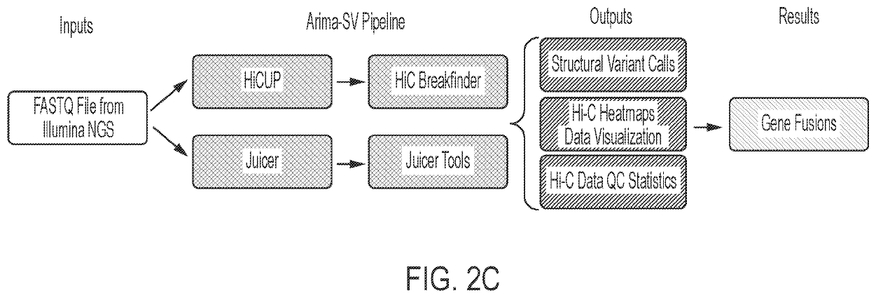

As shown in the workflows in FIGS. 2A and 2B, patient FFPE samples were subjected to Capture-HiC, using a custom panel design for 884 known cancer-related genes. Briefly, FFPE tissue scrolls were dewaxed and the tissue rehydrated. The samples were then subjected to chromatin digestion, end-labeling, and proximity ligation prior to DNA purification. Purified DNA was next prepared as a short-read sequencing library and sequenced on a NovaSeq System. FASTQ files input into the Arima-SV pipeline, shown in FIG. 2C, which enable the calling of variants, production of HiC heatmaps for identification of gene fusions.

### Results

184 FFPE tumors across tumor types were profiled. Clinical validation of the Capture-HiC approach was first performed by re-analyzing 33 FFPE tumors comprising actionable gene fusions detected by the RNA-based NYU FUSION SEQer CLIA assay. A 100% concordance (33/33) between Capture-HiC and RNA panels was observed.

151 driver-negative FFPE tumors were analyzed using genome-wide HiC, including 62 CNS tumors, 59 gynecological sarcomas, and 22 solid heme tumors, with no detectable genetic drivers from prior DNA and RNA panel CLIA assays. Amongst these, HiC analysis identified previously undetected fusions in 72% (109/151) of tumors. A summary of the results is shown in Table 8 below. In the table, patients are binned based on the clinical significance of their biomarker.

TABLE 8
151 Driver-negative Patients Analyzed

| | Findings with | |
|---|---|---|
| Sample Types | Arima Technology | Relevance |
| 66% Gynecological | 34% patients with | 53% Clinically |
| Sarcoma (n = 58) | biomarker targeted by | Actionable Genes |
| 63% Solid Heme | FDA-approved drugs | |
| (n = 22) | (n = 51) (TIER 1) | |
| 40% CNS (n = 65) | 4% patients with | |
| | biomarkers targeted by | |
| | ongoing clinical trials | |
| | (n = 6) (TIER 2) | |
| | 15% patients with biomarkers of | |
| | prognostic/diagnostic | |
| | significance (n = 22) | |
| | (TIER 3) | |

### Clinical Significance

### Clinical Significance

To attribute clinical significance to the fusions detected, the genes implicated in our fusion calls were compared with NCCN and WHO guidelines, and OncoKB, and assigned which tumors had a therapeutic level biomarker (TIER 1 and TIER 2) (e.g., PD-L1, NTRK, RAD51B), or a diagnostic/prognostic biomarker (TIER 3) (e.g., MYBL1 in glioma). Of the 63 FFPE tumors tested, 39.7% ( 25/63) of tumors were found to have fusions involving a therapeutic level biomarker (TIER 1 and TIER 2) and a further 12.7% ( 8/63) had fusions involving a diagnostic or prognostic biomarker (TIER 3), indicating an overall diagnostic yield of 52.4%. The remaining 19% ( 12/63) had fusions of potential clinical significance (TIER 4), according to OncoKB. Of the total 122 tumor driver-negative patients analyzed, 34% ( 41/122) of samples had fusions involving a therapeutic level biomarker (TIER 1), 4% ( 5/122) had fusions involving a biomarker targeted by ongoing clinical trials (TIER 2), and a further 14% ( 19/122) had fusions involving a diagnostic or prognostic biomarker (TIER 3), indicating an overall diagnostic yield of 53%. Additionally, 16% ( 19/122) had fusions of potential clinical significance (TIER 4), according to OncoKB.

### 3D Genome Analysis Assists Patient Management in Prospective Glioma Patient

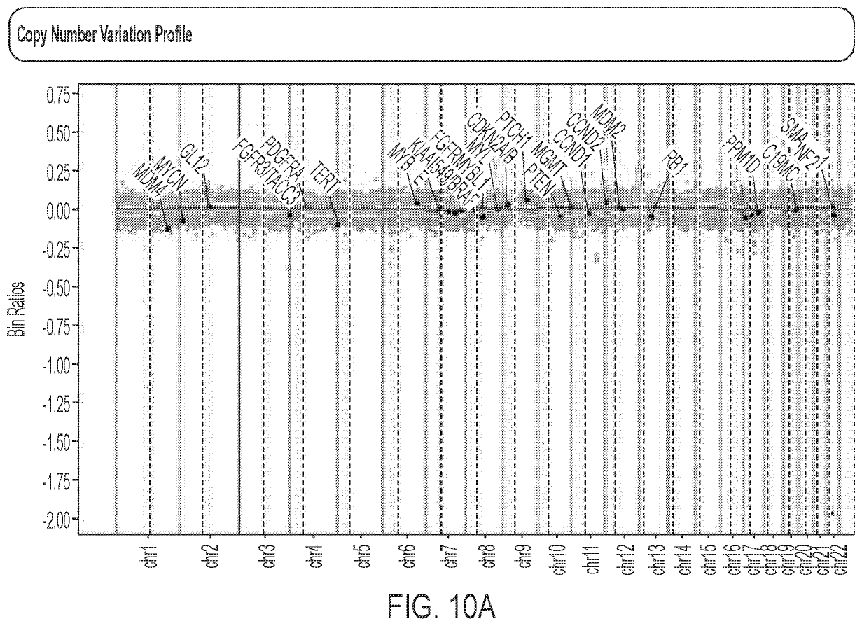

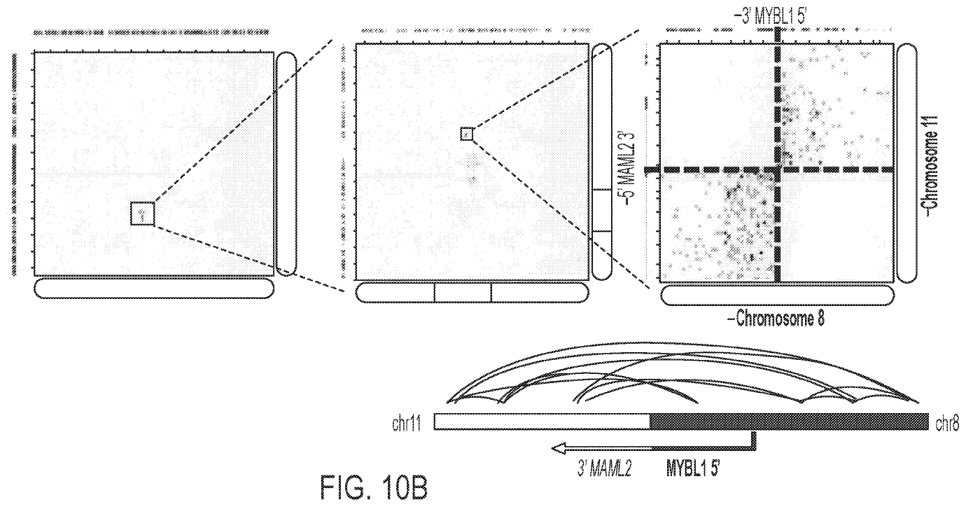

In another example, MYBL1 fusions were detected in two glioma cases that were previously missed by RNA panels. Tables 9A and 9B, and FIG. 10A show a summary of patient presentation, initial treatment, and pathologic workup. FIG. 10 shows the result of an exemplary process in which 3D genome analysis described herein was used to alter the course of patient management in a prospective glioma patient. These studies resulted in a brain tumor classification result of a probable MYB/MYBL1 low grade glioma. The studies also showed, however, a lack of any detectable diagnostic MYB or MYBL1 gene fusion.

TABLE 9A

| ASSAY | RESULT | TREATMENT |
|---|---|---|
| DNA Next Generation | Negative or IDH ½ | Unclear if |
| Sequencing | mutations | adjuvant therapy |
| RNA Fusion | Negative for | required |
| required SEQer | gene fusions | |

TABLE 9B
Brain Tumor Methylation Classifier

| Class Score | Methylation Family | Interpretation |
|---|---|---|
| 0.983 | LGG, MYB | Positive |
| 0.004 | MTGF_GBM | |
| 0.002 | MTGF_IDH_GLM | |
| 0.001 | SUBEPN, SPINE | |
| 0.001 | LGG, RGNT | |

TABLE 9C

| ASSAY | RESULT | TREATMENT |
|---|---|---|
| Arima Technology | Positive for MYBL1-MAML2 | No adjuvant therapy |
| | gene fusion | required |

As shown in FIG. 10B, 3D genome analysis identified a MYBL1-MAML2 gene fusion, which supported a diagnosis of a MYBL1 low grade glioma, ultimately sparing the patient from adjuvant chemotherapy post-resection. See also, Table 9C.

Gene Fusion Detected in Subependymal Giant Cell Astrocytoma with 3D Genomics

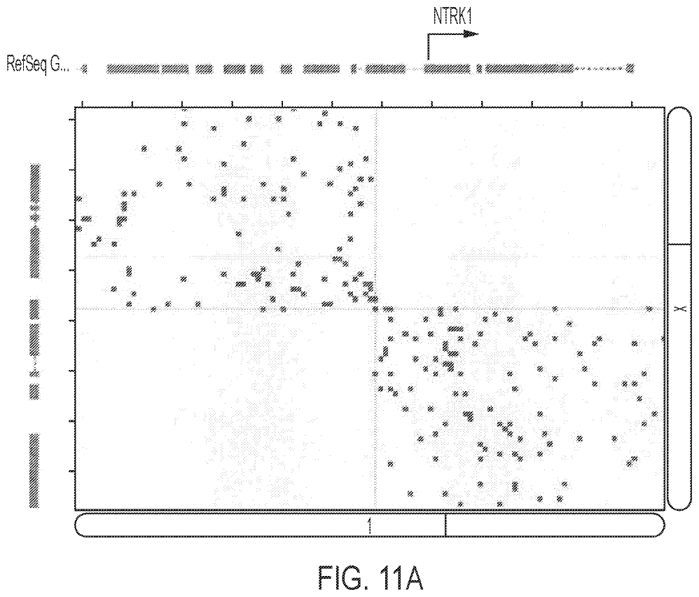

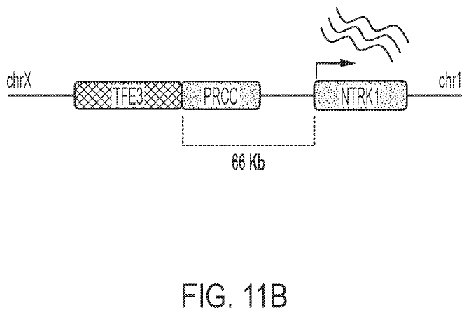

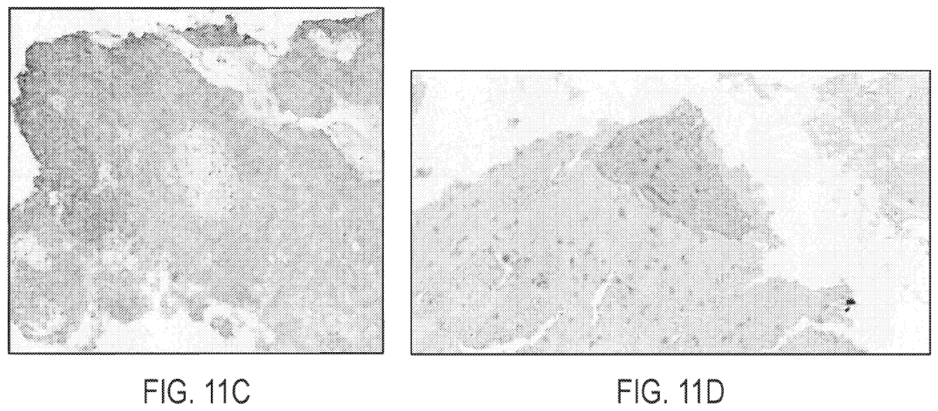

FIG. 11 shows detection of an NTRK1 proximity fusion in a subependymal giant cell astrocytoma sample using the methods described herein. FIG. 11A shows a HiC heatmap showing the TFE3-PRCC gene fusion with NTRK1 in proximity to the fusion breakpoint (hence, defining this fusion as an NTRK1 proximity fusion) and HiC signal showing NTRK1 interacting with genomic sequences across the breakpoint, which may influence changes in its expression levels. FIG. 11B shows a schematic of the same NTRK1 proximity fusion, showing a gene fusion event between PRCC chromosome 1 (chr1) and TFE3 on chromosome X (chrX). Importantly, NTRK1 (also on chr1) is located ˜66 kb away from the breakpoint on chr1, and so with respect to NTRK1 is a proximity fusion. Depicted is full length (non-chimeric) NTRK1 transcripts being expressed. FIG. 11C shows a micrograph of positive immunohistochemical staining of NTRK (using a pan-TRK antibody). FIG. 11D shows a micrograph of negative immunohistochemical staining of NTRK in normal tissue adjacent to the tumor tissue in FIG. 11C.

NTRK1 is the target of several therapies, such as larotrectonib.

### Gene Fusion Detected in Myxoid Leiomyosarcoma

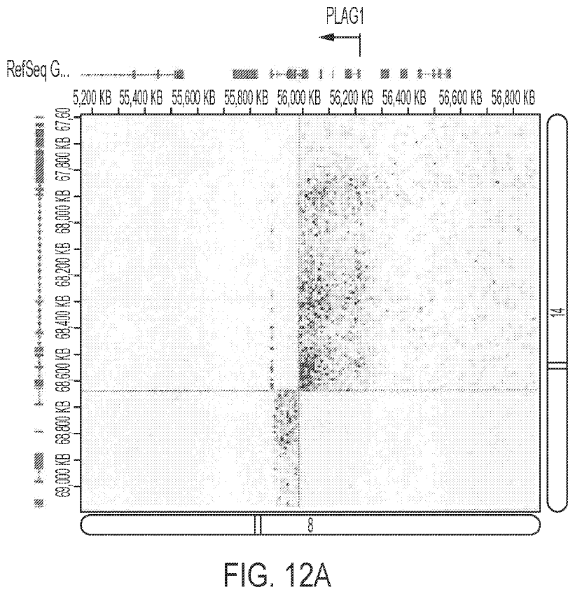

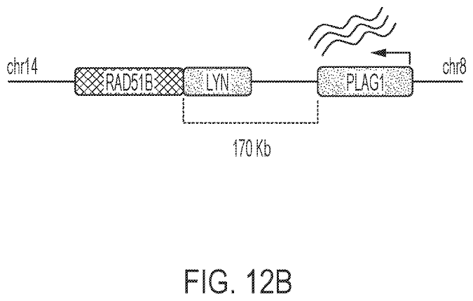

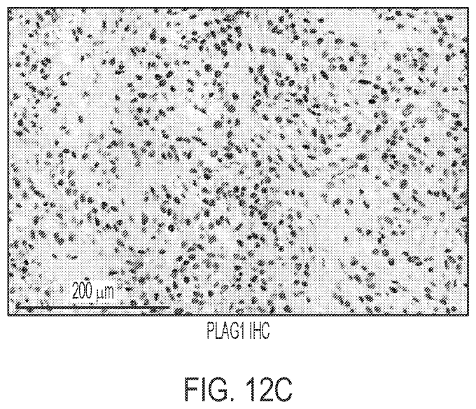

In another example, FIG. 12 shows detection of a PLAG1 proximity fusion in a myxoid leiomyosarcoma sample using the methods described herein. FIG. 12A shows a HiC heatmap showing the RAD51B-LYN gene fusion with PLAG1 in proximity to the fusion breakpoint (hence, defining this fusion as a PLAG1 proximity fusion) and HiC signal showing PLAG1 interacting with with genomic sequences across the breakpoint, which may influence changes in its expression levels. FIG. 12B shows a schematic of the same PLAG1 proximity fusion, showing a gene fusion event between LYN on chromosome 8 (chr8) and RAD51B on chromosome 14 (chr14). Importantly, PLAG1 (also on chr8) is located ˜170 kb away from the breakpoint on chr8, and so with respect to PLAG1 is a proximity fusion. Depicted is full length (non-chimeric) PLAG1 transcripts being expressed. FIG. 12C shows a micrograph of positive immunohistochemical staining of PLAG1 using anti-PLAG1 antibody.

PLAG1 is a NATIONAL COMPREHENSIVE CANCER NETWORK™ (“NCCN”) diagnostic biomarker in uterine sarcomas.

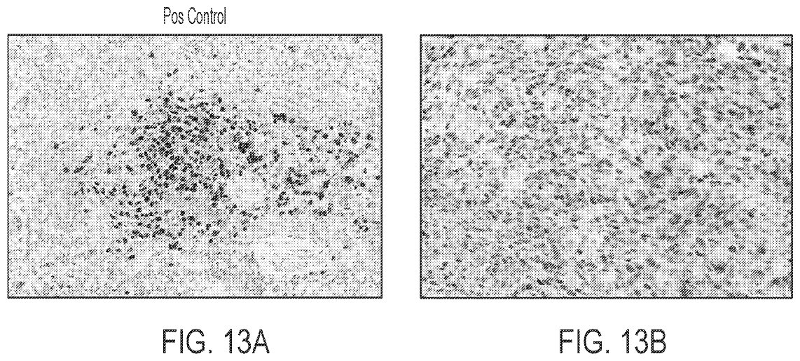

In an embodiment, a break in CCDN1 on chromosome 11 is described (S28). To confirm the gene fusion event affected CCND1 expression, immunohistochemistry (IHC) was performed according to known methods. FIG. 13 shows an IHC stain using anti-CCND1 (Cyclin D1) antibody where the diffusely positive signal demonstrates that there was an increased abundance of the CCND1 protein in the tumor sample. FIG. 13A is a positive control. FIG. 13B shows the anti-CCND1 stain in an epithelioid mesenchymal tumor with SMD cells. CCND1 is an NCCN diagnostic biomarker in uterine sarcomas.

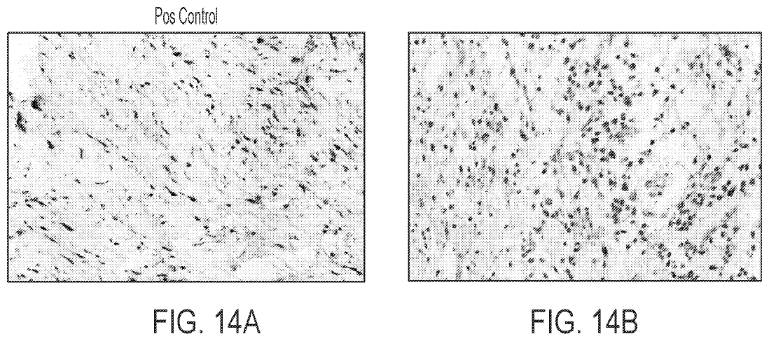

In an embodiment, an interaction was detected between CDK4 on chromosome 12 and KATNBL1 on chromosome 15 (S40). To confirm the gene fusion event affected CDK4 expression, immunohistochemistry (IHC) was performed according to known methods. FIG. 14 shows an IHC stain using anti-CDK4 antibody where the focally positive signal demonstrates that there was an increased abundance of the CDK4 protein in the tumor sample. FIG. 14A is a positive control. FIG. 14B shows the anti-CDK4 stain in an adenosarcoma with sarcoma overgrowth (ASSO) tumor. CDK4 is the target of on-trial drug narazaciclib.

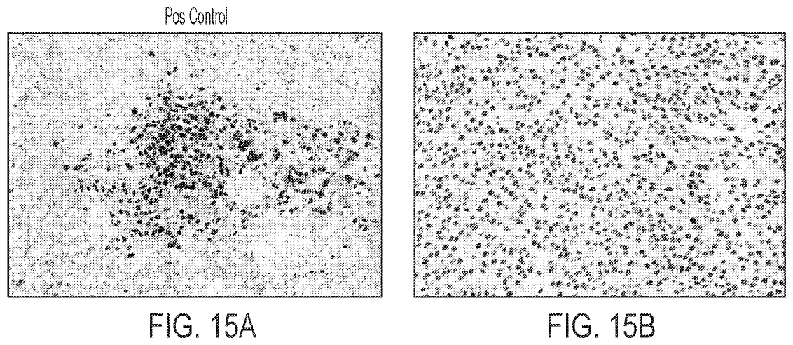

In an embodiment, an interaction was detected between CCND11 (Cyclin D1) on chromosome 11 and MRPL23 on chromosome 11 (S35). To confirm the gene fusion event affected CCND1 (Cyclin D1) expression, immunohistochemistry (IHC) was performed according to known methods. FIG. 15 shows an IHC stain using anti-CCND1 (Cyclin D1) antibody where the diffusely positive signal demonstrates that there was an increased abundance of the CCND1 (Cyclin D1) protein in the tumor sample FIG. 15A is a positive control. FIG. 15B shows the anti-CCND1 stain in low grade (LG) epithelioid neoplasm with myomelanocytic differentiation tumor cells. CCND1 is an NCCN diagnostic biomarker in uterine sarcomas.

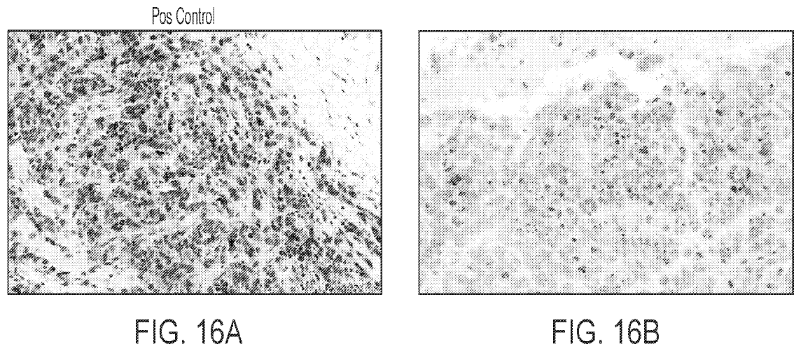

In an embodiment, an interaction was detected between MyoD1 on chromosome 11 and LMO2 on chromosome 11 (S50). To confirm the gene fusion event affected MyoD1 expression, immunohistochemistry (IHC) was performed according to known methods. FIG. 16 shows an IHC stain using anti-MyoD1 antibody where the diffusely positive signal demonstrates that there was an increased abundance of the MyoD1 protein in the tumor sample. FIG. 16A is a positive control. FIG. 16B shows the anti-MyoD1 antibody staining of HG spindle cell sarcoma tumor cells. MyoD1 is an NCCN diagnostic biomarker in uterine sarcomas.

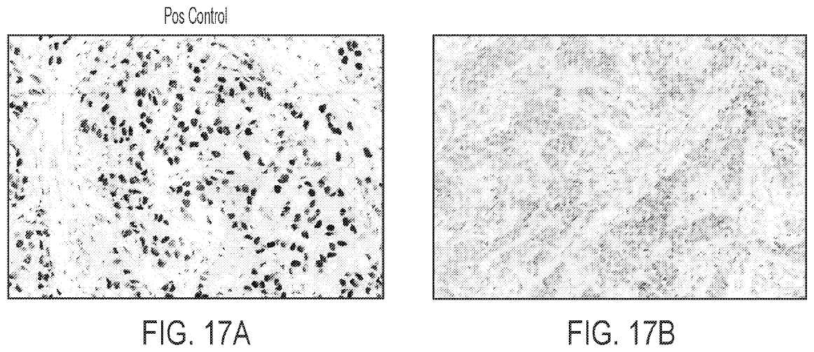

In an embodiment, an interaction was detected between ESR1 on chromosome 6 and NCOA3 on chromosome 20 (S41). To confirm the gene fusion event affected ESR1 expression, immunohistochemistry (IHC) was performed according to known methods. FIG. 17 shows an IHC stain using anti-ESR1 antibody where the diffusely positive signal demonstrates that there was an increased abundance of the ESR1 protein in the tumor sample. FIG. 17A is a positive control. FIG. 17B shows the anti-ESR1 stain in uterine tumor resembling ovarian sex cord tumor (UTROSCT) cells. ESR1 is the target of fulvestrant.

In an embodiment, an interaction was detected with EGFR on chromosome 7. To confirm the gene fusion event affected EGFR expression, immunohistochemistry (IHC) was performed according to known methods. FIG. 18 shows an IHC stain using anti-EGFR antibody where the diffusely positive signal demonstrates that there was an increased abundance of the EGFR protein in the tumor sample. FIG. 18A is a positive control. FIG. 18B shows the anti-EGFR stain in colorectal carcinoma cells. EGFR is the target of several therapies, such as cetuximab.

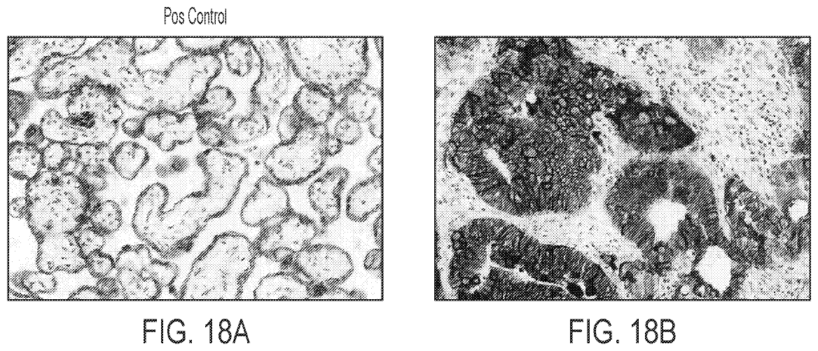

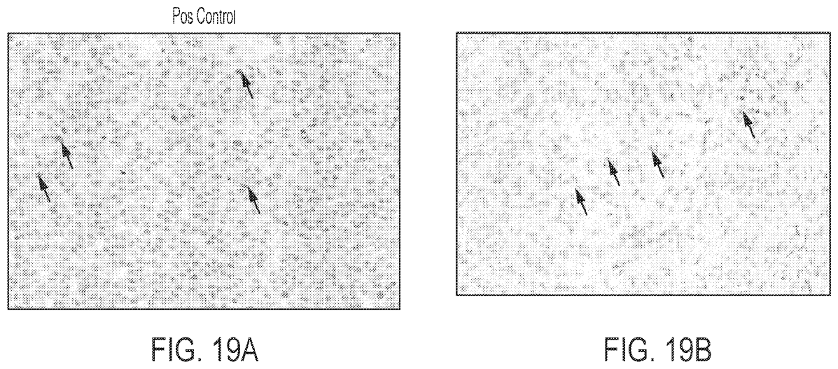

In an embodiment, a breakpoint was detected in MDM2 on chromosome 12 (S16). To confirm the gene fusion event affected MDM2 expression, immunohistochemistry (IHC) was performed according to known methods. FIG. 19 shows an IHC stain using anti-MDM2 antibody where the focally positive signal demonstrates that there was an increased abundance of the MDM2 protein in the tumor sample. FIG. 19A is a positive control. FIG. 19B shows the anti-MDM2 antibody in high-grade endometrial stromal sarcoma (HGESS) (uterine) tumor cells. MDM2 is the target of on-trial drug navtemadlin.

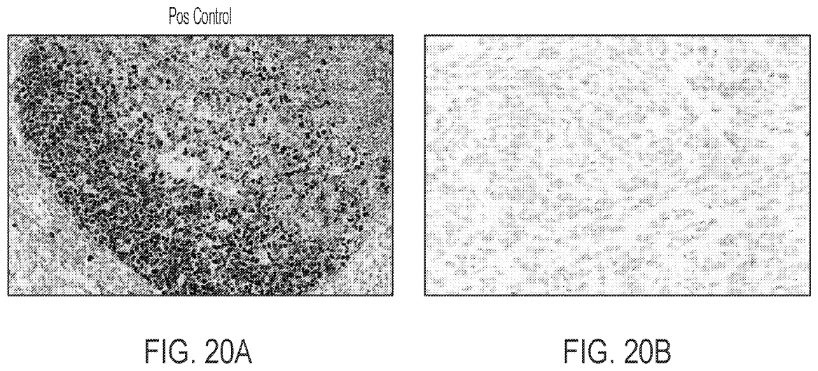

In an embodiment, a genomic interaction in S75 was discovered. To confirm the gene fusion event affected RB1 expression, immunohistochemistry (IHC) was performed according to known methods. FIG. 20 shows an IHC stain using anti-RB1 antibody that demonstrates that there was a decrease in the RB1 protein in the tumor sample. FIG. 20A is a positive control. FIG. 20B shows the anti-RB1 stain in leiomyosarcoma tumor cells.

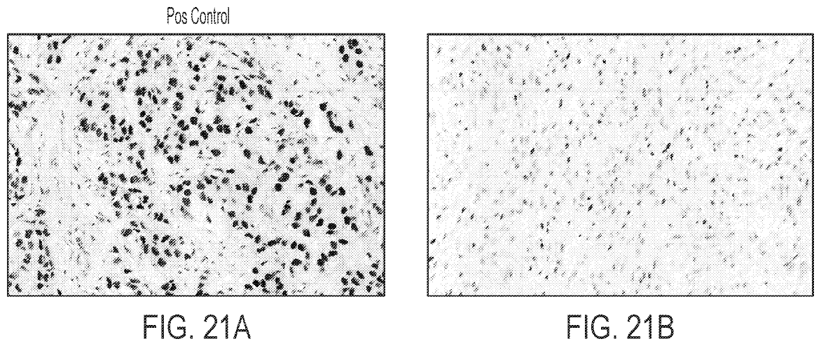

In an embodiment, at least one genomic interaction was detected involving ESR1 on chromosome 6 (S46). To confirm the gene fusion event affected ESR1 expression, immunohistochemistry (IHC) was performed according to known methods. FIG. 21 shows an IHC stain using anti-ESR1 antibody where the diffusely positive signal demonstrates that there was an increased abundance of the ESR1 protein in the tumor sample. FIG. 21A is a positive control. FIG. 21B shows the anti-ESR1 stain in high grade sarcoma (recurrent tumor) tumor cells. ESR1 is the target of fulvestrant

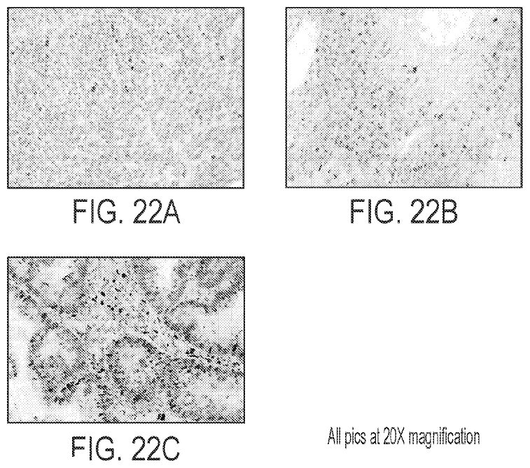

In an embodiment, at least one genomic interaction was detected involving MDM2 on chromosome 12 (S58). To confirm the gene fusion event affected MDM2 expression, immunohistochemistry (IHC) was performed according to known methods. FIG. 22A shows an IHC stain using anti-MDM2 antibody where the focally positive signal demonstrates that there was an increased abundance of the MDM2 protein in adenosarcoma with sarcoma overgrowth (ASSO) tissue. MDM2 is the target of on-trial drug navtemadlin.

In an embodiment, at least one genomic interaction was detected involving CDK4 on chromosome 12 (S58). To confirm the gene fusion event affected CDK4 expression, immunohistochemistry (IHC) was performed according to known methods. FIG. 22B shows an IHC stain using anti-CDK4 antibody where the slightly positive signal demonstrates that there was an increased abundance of the CDK4 protein in adenosarcoma with sarcoma overgrowth (ASSO) tissue. CDK4 is the target of on-trial drug narazaciclib.

In an embodiment, at least one genomic interaction was detected involving AR on chromosome X (S58). To confirm the gene fusion event affected AR expression, immunohistochemistry (IHC) was performed according to known methods. FIG. 22C shows an IHC stain using anti-AR antibody where the diffusely positive signal demonstrates that there was an increased abundance of the AR protein in adenosarcoma with sarcoma overgrowth (ASSO) tissue.

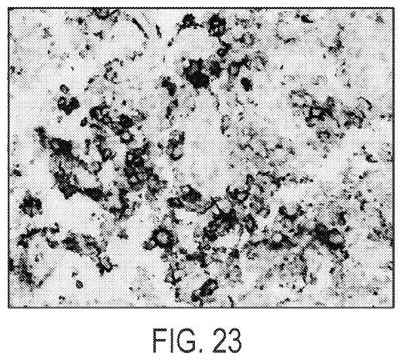

In an embodiment, at least one genomic interaction was detected involving PD-L1 on chromosome 9 (S65). A proximity fusion involving PD-L1 was discovered using one embodiment of the spatial-proximal contiguity assays described herein. To confirm the gene fusion event affected PD-L1 expression, immunohistochemistry (IHC) was performed according to known methods. FIG. 23 shows an IHC stain using anti-PD-L1 antibody where the positive signal demonstrates that there was an increased abundance of the PD-L1 protein in glioblastoma tumor tissue. The expression of PD-L1 in the tumor tissue shown by the antibody stain indicates that the tumor cells are not as susceptible to the immune system as tumor cells without PD-L1 expression would be. Treatment with drugs that block PD-L1 (or the broader PD-1 receptor-mediated pathway) would allow tumor cells to be susceptible to the patient's T-cells. Treatment options for PD-L1 mediated cancers are discussed further in commonly owned applications entitled “Methods of Selecting and Treating Cancer Subjects that are Candidates for Treatment Using Inhibitors of a PD-1 Pathway” and “Methods of Selecting and Treating Cancer Subjects Having a Genetic Structural Variant Associated with PTPRD,” both filed Mar. 6, 2023.

Together, these results demonstrate clinical validation of the structural variants identified herein, and highlight the utility for 3D genome profiling to increase diagnostic yield by finding clinically actionable fusions in tumors without available NGS fusion assays (e.g., solid hematological tumors). As described herein, the 3D genomic methods have identified “proximity fusions” with non-coding/intergenic breaks, which can lead to activation of druggable targets or diagnostic biomarkers as described herein.

### REFERENCES

Dixon, J. R., et al. (2018). “Integrative detection and analysis of structural variation in cancer genomes.” Nature Genetics. 50(10), 1388-1398.
Harewood, L., et al. (2017). “Hi-C as a tool for precise detection and characterisation of chromosomal rearrangements and copy number variation in human tumours.” Genome Biology, 18(1), 125.
Product Flyer: Arima-HiC FFPE. Arima Genomics Literature.
Bioinformatics User Guide: Arima Structural Variant Pipeline. Arima Genomics.

### Structural Variants Identified

Table 10 (encompassing all sub-tables) below shows certain structural variants identified by methods described herein. Certain samples were classified as having undiagnosed tumors/cancers with no clear with no known tumor driver (e.g., oncogene) as assessed by standard cytogenetic/molecular testing (i.e., chromosomal karyotyping, a FISH panel, DNA microarray, and a cancer next generation sequencing (NGS) panel). The choroid plexus carcinoma sample additionally was subjected to a methylation array.

TABLE 10

| Row | | | | | |
|---|---|---|---|---|---|
| 1 | VARIANT ID | 1 | 2 | 3 | 4 |
| 2 | SAMPLE | S1 | S2 | S2 | S2 |
| | NUMBER | | | | |
| 3 | Tumor type | Melanoma | Colorectal | Colorectal | Colorectal |
| | | | Carcinoma | Carcinoma | Carcinoma |
| 4 | Partner 1 | Break in FMN1 | break in SLFN12L | break in NRG1 | break in BCAT1 |
| | type | | | | |
| 5 | Approx. | chr15: | chr17: | chr8: | chr12: |
| | breakpoint | 32,935,001-32,940,000 | 35,530,001-35,535,000 | 32,120,001-32,125,000 | 24,854,001-24,855,000 |
| | coordinate | | | | |
| | window 1A | | | | |
| 6 | Approx. | chr15: | chr17: | chr8: | chr12: |
| | breakpoint | 32,930,001-32,945,000 | 35,525,001-35,540,000 | 32,115,001-32,130,000 | 24,852,001-24,857,000 |
| | coordinate | | | | |
| | window 1B | | | | |
| 7 | Relevant | N/A | RAD51D | NRG1 | KRAS |
| | cancer | | | | |
| | gene(s) | | | | |
| 8 | Gene 5′ | N/A | chr17: 35,119,860 | chr8: 32,548,267 | chr12: 25,250,929 |
| 9 | Gene 3′ | N/A | chr17: 35,092,221 | chr8: 32,767,959 | chr12: 25,205,246 |
| 10 | Cancer Gene | N/A | Tier 1 | Tier 1 | Tier 1 |
| | Tier | | | | |
| 11 | HRR GENE | N/A | YES | NO | NO |
| 12 | Linear | N/A Break in Gene | 410141 | N/A Break in Gene | 395929 |
| | distance to 5′ | | | | |
| | (bp) | | | | |
| 13 | Closest | N/A Break in Gene | 410141 | N/A Break in Gene | 350246 |
| | distance to | | | | |
| | gene body | | | | |
| | (bp) | | | | |
| 14 | Partner 2 | Break in BRAF | Intergenic break | Break in | Intergenic break |
| | gene or | | | ENSG00000253363 | |
| | intergenic | | | | |
| 15 | Relevant | BRAF | N/A | N/A | N/A |
| | cancer | | | | |
| | gene(s) | | | | |
| 16 | Gene 5′ | chr7: 140,924,929 | N/A | N/A | N/A |
| 17 | Gene 3′ | chr7: 140,730,665 | N/A | N/A | N/A |
| 18 | Cancer Gene | Tier 1 | N/A | N/A | N/A |
| | Tier | | | | |
| 19 | HRR GENE | NO | N/A | N/A | N/A |
| 20 | Linear | N/A Break in Gene | N/A | N/A Break in Gene | N/A |
| | distance to 5′ | | | | |
| | (bp) | | | | |
| 21 | Closest | N/A Break in Gene | N/A | N/A Break in Gene | N/A |
| | distance to | | | | |
| | gene body | | | | |
| | (bp) | | | | |
| 22 | Approx. | chr7: | chr4: | chr10: | chr12: |
| | partner | 140,790,001-140,795,000 | 40,280,001-40,285,000 | 112,060,001-112,065,000 | 27,509,001-27,510,000 |
| | breakpoint | | | | |
| | coordinate | | | | |
| | window 2A | | | | |
| 23 | Approx. | chr7: | chr4: | chr10: | chr12: |
| | partner | 140,785,001-140,800,000 | 40,275,001-40,290,000 | 112,055,001-112,070,000 | 27,507,001-27,512,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 5 | 6 | 7 | 8 |
| 2 | SAMPLE | S3 | S4 | S5 | S6 |
| | NUMBER | | | | |
| 3 | Tumor type | Colorectal | Colorectal | Colorectal | Colorectal |
| | | Carcinoma | Carcinoma | Carcinoma | Carcinoma |
| 4 | Partner 1 | break in ZNF710-AS1 | break in PAN3 | break in NRG1 | Intergenic break |
| | type | | | | |
| 5 | Approx. | chr15: | chr13: | chr8: | chr17: |
| | breakpoint | 90,075,001-90,080,000 | 28,211,001-28,212,000 | 32,645,001-32,650,000 | 42,640,001-42,645,000 |
| | coordinate | | | | |
| | window 1A | | | | |
| 6 | Approx. | chr15: | chr13: | chr8: | chr17: |
| | breakpoint | 90,070,001-90,085,000 | 28,210,001-28,213,000 | 32,640,001-32,655,000 | 42,635,001-42,650,000 |
| | coordinate | | | | |
| | window 1B | | | | |
| 7 | Relevant | IDH2 | FLT3 | NRG1 | EZH1 |
| | cancer | | | | BRCA1 |
| | gene(s) | | | | |
| 8 | Gene 5′ | | chr13: 28,100,576 | chr8: 32,548,267 | EZH1: |
| | | | | | chr17: 42,745,040 |
| | | | | | BRCA1: |
| | | | | | chr17: 43,125,364 |
| 9 | Gene 3′ | chr15: 90,083,045 | chr13: 28,003,274 | chr8: 32,767,959 | EZH1: |
| | | | | | chr17: 42,700,275 |
| | | | | | BRCA1: |
| | | | | | chr17: 43,044,295 |
| 10 | Cancer Gene | Tier 1 | Tier 1 | Tier 1 | EZH1: Tier 2 |
| | Tier | | | | BRCA1: Tier 1 |
| 11 | HRR GENE | NO | NO | NO | EZH1: NO |
| | | | | | BRCA1: YES |
| 12 | Linear | 22468 | 110425 | N/A Break in Gene | EZH1: 100,039 |
| | distance to 5′ | | | | BRCA1: 480,364 |
| | (bp) | | | | |
| 13 | Closest | 3045 | 110425 | N/A Break in Gene | EZH1: 55,274 |
| | distance to | | | | BRCA1: 399,295 |
| | gene body | | | | |
| | (bp) | | | | |
| 14 | Partner 2 | break in ENOX1 | break in N4BP2L2 | break in LINC01721 | break in SPTB |
| | gene or | | | | |
| | intergenic | | | | |
| 15 | Relevant | N/A | BRCA2 | N/A | N/A |
| | cancer | | | | |
| | gene(s) | | | | |
| 16 | Gene 5′ | N/A | chr13: 32,315,086 | N/A | N/A |
| 17 | Gene 3′ | N/A | chr13: 32,400,268 | N/A | N/A |
| 18 | Cancer Gene | N/A | Tier 1 | N/A | N/A |
| | Tier | | | | |
| 19 | HRR GENE | N/A | YES | N/A | N/A |
| 20 | Linear | N/A Break in Gene | 154915 | N/A Break in Gene | N/A |
| | distance to 5′ | | | | |
| | (bp) | | | | |
| 21 | Closest | N/A Break in Gene | 69733 | N/A Break in Gene | N/A |
| | distance to | | | | |
| | gene body | | | | |
| | (bp) | | | | |
| 22 | Approx. | chr13: | chr13: | chr20: | chr14: |
| | partner | 43,600,001-43,605,000 | 32,470,001-32,471,00 | 24,155,001-24,160,000 | 64,770,001-64,775,000 |
| | breakpoint | | | | |
| | coordinate | | | | |
| | window 2A | | | | |
| 23 | Approx. | chr13: | chr13: | chr20: | chr14: |
| | partner | 43,590,001-43,610,000 | 32,469,001-32,472,00 | 24,150,001-24,165,000 | 64,765,001-64,780,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 9 | 10 | 11 | 12 |
| 2 | SAMPLE | S7 | S7 | S8 | S9 |
| | NUMBER | | | | |
| 3 | Tumor type | Chordoma (PDx model) | Chordoma (PDx model) | Chordoma | Chordoma |
| 4 | Partner 1 | break in TIPIN | break in FAM157C | break in USP20 | break in NTRK2 |
| | type | | | | |
| 5 | Approx. | chr15: | chr16: | chr9: | chr9: |
| | breakpoint | 66,352,001-66,353,000 | 90,100,001-90,110,000 | 129,850,001-129,860,000 | 84,740,001-84,750,000 |
| | coordinate | | | | |
| | window 1A | | | | |
| 6 | Approx. | chr15: | chr16: | chr9: | chr9: |
| | breakpoint | 66,350,001-66,355,000 | 90,090,001-90,120,000 | 129,840,001-129,870,000 | 84,730,001-84,760,000 |
| | coordinate | | | | |
| | window 1B | | | | |
| 7 | Relevant | MAP2K1 | FANCA | ABL1 | NTRK2 |
| | cancer | | | | |
| | gene(s) | | | | |
| 8 | Gene 5′ | chr15: 66,386,912 | chr16: 89,816,647 | chr9: 130,713,016 | chr9: 84,669,131 |
| 9 | Gene 3′ | chr15: 66,491,544 | chr16: 89,737,549 | chr9: 130,887,670 | chr9: 85,027,050 |
| 10 | Cancer Gene | Tier 1 | Tier 1 | Tier 1 | Tier 1 |
| | Tier | | | | |
| 11 | HRR GENE | NO | YES | NO | NO |
| 12 | Linear | 33912 | 283354 | 853016 | N/A Break in Gene |
| | distance to 5′ | | | | |
| | (bp) | | | | |
| 13 | Closest | 33912 | 283354 | 853016 | N/A Break in Gene |
| | distance to | | | | |
| | gene body | | | | |
| | (bp) | | | | |
| 14 | Partner 2 | intergenic | break in BRSK2 | break in ABCC9 | break in CNTRL |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A | N/A Break in Gene | N/A Break in Gene |
| | distance to 5′ | | | | |
| | (bp) | | | | |
| 21 | Closest | N/A | N/A | N/A Break in Gene | N/A Break in Gene |
| | distance to | | | | |
| | gene body | | | | |
| | (bp) | | | | |
| 22 | Approx. | chr6: | chr11: | Chr9: | chr9: |
| | partner | 153,641,001-153,642,000 | 1,380,001-1,390,000 | 18,381,001-18,382,000 | 121,140,001-121,150,000 |
| | breakpoint | | | | |
| | coordinate | | | | |
| | window 2A | | | | |
| 23 | Approx. | chr6: | chr11: | Chr9: | chr9: |
| | partner | 153,639,001-153,644,000 | 1,370,001-1,400,000 | 18,377,000-18,386,000 | 121,130,001-121,160,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 13 | 14 | 15 | 16 |
| 2 | SAMPLE | S9 | S10 | S11 | S12 |
| | NUMBER | | | | |
| 3 | Tumor type | Chordoma | Chordoma | Chordoma (PDx model) | Meningioma |
| 4 | Partner 1 | break in NR_110931 | break in NR_136588 | intergenic | break in INS |
| | type | | | | |
| 5 | Approx. | chr16: | chr2: | chr10: | chr11: |
| | breakpoint | 89,460,001-89,470,000 | 208,650,001-208,655,000 | 121,035,001-121,040,000 | 2,155,001-2,160,000 |
| | coordinate | | | | |
| | window 1A | | | | |
| 6 | Approx. | chr16: | chr2: | chr10: | chr11: |
| | breakpoint | 89,450,001-89,480,000 | 208,645,001-208,660,000 | 121,030,001-121,045,000 | 2,150,001-2,165,000 |
| | coordinate | | | | |
| | window 1B | | | | |
| 7 | Relevant | FANCA | IDH1 | FGFR2 | IGF2 |
| | cancer | | | | |
| | gene(s) | | | | |
| 8 | Gene 5′ | chr16: 89,816,647 | chr2: 208,255,071 | chr10: 121,598,403 | chr11: 2,138,974 |
| 9 | Gene 3′ | chr16: 89,737,549 | chr2: 208,236,229 | chr10: 121,479,857 | chr11: 2,129,112 |
| 10 | Cancer Gene | Tier 1 | Tier 1 | Tier 1 | Tier 2 |
| | Tier | | | | |
| 11 | HRR GENE | YES | NO | NO | NO |
| 12 | Linear | 346647 | 394930 | 558403 | 16027 |
| | distance to 5′ | | | | |
| | (bp) | | | | |
| 13 | Closest | 267549 | 394930 | 439857 | 16027 |
| | distance to | | | | |
| | gene body | | | | |
| | (bp) | | | | |
| 14 | Partner 2 | break in WIPF3 | intergenic | intergenic | break in KCNMA1-AS3 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A | N/A | N/A Break in Gene |
| | distance to 5′ | | | | |
| | (bp) | | | | |
| 21 | Closest | N/A | N/A | N/A | N/A Break in Gene |
| | distance to | | | | |
| | gene body | | | | |
| | (bp) | | | | |
| 22 | Approx. | chr7: | chr2: | chr10: | chr10: |
| | partner | 29,910,001-29,920,000 | 218,100,001-218,105,000 | 123,565,001-123,570,000 | 77,375,001-77,380,000 |
| | breakpoint | | | | |
| | coordinate | | | | |
| | window 2A | | | | |
| 23 | Approx. | chr7: | chr2: | chr10: | chr10: |
| | partner | 29,900,001-29,930,000 | 218,095,001-218,110,000 | 123,560,001-123,575,000 | 77,370,001-77,385,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 17 | 18 | 19 | 20 |
| 2 | SAMPLE | S13 | S14 | S14 | S14 |
| | NUMBER | | | | |
| 3 | Tumor type | Colorectal Carcinoma | Leukemia (ALL) | Leukemia (ALL) | Leukemia (ALL) |
| 4 | Partner 1 | break in NR_134631 | break in CDK6 | break in CDK6 | break in EP300 |
| | type | | | | |
| 5 | Approx. | chr22: | chr7: | chr7: | chr22: |
| | breakpoint | 41,555,001-41,560,000 | 92,822,001-92,823,000 | 92,820,001-92,825,000 | 41,133,001-41,134,000 |
| | coordinate | | | | |
| | window 1A | | | | |
| 6 | Approx. | chr22: | chr7: | chr7: | chr22: |
| | breakpoint | 41,550,001-41,565,000 | 92,820,001-92,825,000 | 92,815,001-92,830,000 | 41,131,001-41,136,000 |
| | coordinate | | | | |
| | window 1B | | | | |
| 7 | Relevant | EP300 | CDK6 | CDK6 | EP300 |
| | cancer | | | SAMD9 | |
| | gene(s) | | | | |
| 8 | Gene 5′ | chr22: 41,092,592 | chr7: 92,836,573 | CDK6: | chr22: 41,092,592 |
| | | | | chr7: 92,836,573 | |
| | | | | SAMD9: | |
| | | | | chr7: 93,118,023 | |
| 9 | Gene 3′ | chr22: 41,180,077 | chr7: 92,604,921 | CDK6: | chr22: 41,180,077 |
| | | | | chr7: 92,604,921SA | |
| | | | | MD9: | |
| | | | | chr7: 93,099,513 | |
| 10 | Cancer Gene | Tier 2 | Tier 2 | CDK6: Tier 2 | Tier 2 |
| | Tier | | | SAMD9: Tier 3 | |
| 11 | HRR GENE | NO | NO | NO | NO |
| 12 | Linear | 462409 | N/A Break in Gene | CDK6: N/A Break in Gene | N/A Break in Gene |
| | distance to 5′ | | | | |
| | (bp) | | | SAMD9: 293,023 | |
| 13 | Closest | 374924 | N/A Break in Gene | CDK6: N/A Break in Gene | N/A Break in Gene |
| | distance to | | | | |
| | gene body | | | SAMD9: 274,513 | |
| | (bp) | | | | |
| 14 | Partner 2 | break in MYH9 | break in SKAP2 | intergenic | break in ZNF384 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A Break in Gene | N/A | N/A Break in Gene |
| | distance to 5′ | | | | |
| | (bp) | | | | |
| 21 | Closest | N/A Break in Gene | N/A Break in Gene | N/A | N/A Break in Gene |
| | distance to | | | | |
| | gene body | | | | |
| | (bp) | | | | |
| 22 | Approx. | chr22: | chr7: | chr5: | chr12: |
| | partner | 36,365,001-36,370,000 | 26,819,001-26,820,000 | 120,405,001-120,410,000 | 6,689,001-6,690,000 |
| | breakpoint | | | | |
| | coordinate | | | | |
| | window 2A | | | | |
| 23 | Approx. | chr22: | chr7: | chr5: | chr12: |
| | partner | 36,360,001-36,375,000 | 26,817,001-26,822,000 | 120,400,001-120,415,000 | 6,687,001-6,690,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 21 | 22 | 23 | 24 |
| 2 | SAMPLE | S15 | S16 | S16 | S17 |
| | NUMBER | | | | |
| 3 | Tumor type | Intermediate- | High-grade | High-grade | Leukemia (ALL) |
| | | high grade | endometrial | endometrial | |
| | | Fibrosarcoma | stromal sarcoma | stromal sarcoma | |
| | | NOS | (HGESS) - Uterine | (HGESS) - Uterine | |
| 4 | Partner 1 | break in SNU13 | break in CPM | break in BCOR | Intergenic break |
| | type | | | | |
| 5 | Approx. | chr22: | chr12: | chrX: | chr7: |
| | breakpoint | 41,680,001-41,685,000 | 68,930,001-68,935,000 | 40,065,001-40,070,000 | 54,005,001-54,010,000 |
| | coordinate | | | | |
| | window 1A | | | | |
| 6 | Approx. | chr22: | chr12: | chrX: | chr7: |
| | breakpoint | 41,675,001-41,690,000 | 68,925,001-68,940,000 | 40,060,001-40,075,000 | 54,000,001-54,015,000 |
| | coordinate | | | | |
| | window 1B | | | | |
| 7 | Relevant | EP300 | MDM2 | BCOR | EGFR |
| | cancer | | | | |
| | gene(s) | | | | |
| 8 | Gene 5′ | chr22: 41,092,592 | chr12: 68,809,002 | chrX: 40,177,213 | chr7: 55,019,017 |
| 9 | Gene 3′ | chr22: 41,180,077 | chr12: 68,840,807 | chrX: 40,051,254 | chr7: 55,211,628 |
| 10 | Cancer Gene | Tier 2 | Tier 2 | Tier 3 | Tier 1 |
| | Tier | | | | |
| 11 | HRR GENE | NO | NO | NO | NO |
| 12 | Linear | 587409 | 120999 | N/A Break in Gene | 1009017 |
| | distance to 5′ | | | | |
| | (bp) | | | | |
| 13 | Closest | 499924 | 89194 | N/A Break in Gene | 1009017 |
| | distance to | | | | |
| | gene body | | | | |
| | (bp) | | | | |
| 14 | Partner 2 | break in PPP1R16B | intergenic break | break in ZC3H7B | break in NUP205 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A | N/A Break in Gene | N/A Break in Gene |
| | distance to 5′ | | | | |
| | (bp) | | | | |
| 21 | Closest | N/A Break in Gene | N/A | N/A Break in Gene | N/A Break in Gene |
| | distance to | | | | |
| | gene body | | | | |
| | (bp) | | | | |
| 22 | Approx. | chr20: | chr12: | chr22: | chr7: |
| | partner | 38,810,001-38,815,000 | 52,735,001-52,740,000 | 41,340,001-41,345,000 | 135,565,001-135,570,000 |
| | breakpoint | | | | |
| | coordinate | | | | |
| | window 2A | | | | |
| 23 | Approx. | chr20: | chr12: | chr22: | chr7: |
| | partner | 38,805,001-38,820,000 | 52,730,001-52,745,000 | 41,335,001-41,350,000 | 135,560,001-135,575,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | FIG. 19 | N/A | N/A |
| 26 | NOTES | | | | |
| 1 | VARIANT ID | 25 | 26 | 27 | 28 |
| 2 | SAMPLE | S18 | S19 | S20 | S21 |
| | NUMBER | | | | |
| 3 | Tumor type | Leukemia (ALL) | Colorectal Carcinoma | Colorectal Carcinoma | Leukemia (ALL) |
| 4 | Partner 1 | break in LOC645177 | break in ZNF605 | break in NR_110559 | break in PTK2B |
| | type | | | | |
| 5 | Approx. | chr12: | chr12: | chr5: | chr8: |
| | breakpoint | 25,010,001-25,015,000 | 132,955,001-132,960,000 | 112,240,001- | 27,380,001-27,385,000 |
| | coordinate | | | 112,250,000 | |
| | window 1A | | | | |
| 6 | Approx. | chr12: | chr12: | chr5: | chr8: |
| | breakpoint | 25,005,001-25,020,000 | 132,950,001-132,965,000 | 112,230,001-112,260,000 | 27,370,001-27,395,000 |
| | coordinate | | | | |
| | window 1B | | | | |
| 7 | Relevant | KRAS | POLE | APC | PTK2B |
| | cancer | | | | |
| | gene(s) | | | | |
| 8 | Gene 5′ | chr12: 25,250,929 | chr12: 132,687,342 | chr5: 112,737,885 | chr8: 27,311,482 |
| 9 | Gene 3′ | chr12: 25,205,246 | chr12: 132,623,762 | chr5: 112,846,239 | chr8: 27,459,390 |
| 10 | Cancer Gene | Tier 1 | Tier 3 | Tier 3 | Tier 3 |
| | Tier | | | | |
| 11 | HRR GENE | NO | NO | NO | NO |
| 12 | Linear | 235929 | 267659 | 487885 | N/A Break in Gene |
| | distance to 5′ | | | | |
| | (bp) | | | | |
| 13 | Closest | 190246 | 267659 | 487885 | N/A Break in Gene |
| | distance to | | | | |
| | gene body | | | | |
| | (bp) | | | | |
| 14 | Partner 2 | intergenic | break in NAV1 | break in IDO2 | break in ABHD17B |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A Break in Gene | N/A Break in Gene | N/A Break in Gene |
| | distance to 5′ | | | | |
| | (bp) | | | | |
| 21 | Closest | N/A | N/A Break in Gene | N/A Break in Gene | N/A Break in Gene |
| | distance to | | | | |
| | gene body | | | | |
| | (bp) | | | | |
| 22 | Approx. | chr4: | chr1: | chr8: | chr9: |
| | partner | 35,080,001-35,085,000 | 201,815,001-201,820,000 | 40,000,001-40,010,000 | 71,875,001-71,880,000 |
| | breakpoint | | | | |
| | coordinate | | | | |
| | window 2A | | | | |
| 23 | Approx. | chr4: | chr1: | chr8: | chr9: |
| | partner | 35,075,001-35,090,000 | 201,810,001-201,825,000 | 39,990,001-40,020,000 | 71,870,001-71,885,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 29 | 30 | 31 | 32 |
| 2 | SAMPLE | S22 | S23 | S24 | S24 |
| | NUMBER | | | | |
| 3 | Tumor type | Undifferentiated/ | Ewings Sarcoma | Sex cord tumor with | HG malignant |
| | | poorly differentiated | | annular tubules | epithelioid and |
| | | malignant uterine | | (SCTAT) | spindled |
| | | neoplasm | | | neoplasm |
| 4 | Partner 1 | break in SLC44A2 | break in EWSR1 | intergenic | break in ABCC8 |
| | type | | | | |
| 5 | Approx. | chr19: | chr22: | chr8: | chr11: |
| | breakpoint | 10,625,001-10,630,000 | 29,285,001-29,290,000 | 66,470,001-66,475,000 | 17,420,001-17,425,000 |
| | coordinate | | | | |
| | window 1A | | | | |
| 6 | Approx. | chr19: | chr22: | chr8: | chr11: |
| | breakpoint | 10,620,001-10,635,000 | 29,280,001-29,295,000 | 66,465,001-66,480,000 | 17,415,001-17,430,000 |
| | coordinate | | | | |
| | window 1B | | | | |
| 7 | Relevant | SMARCA4 | EWSR1 | MYBL1 | MYOD1 |
| | cancer | | | | |
| | gene(s) | | | | |
| 8 | Gene 5′ | chr19: 10,961,001 | chr22: 29,268,268 | chr8: 66,613,218 | chr11: 17,719,571 |
| 9 | Gene 3′ | chr19: 11,062,256 | chr22: 29,300,521 | chr8: 66,562,175 | chr1117,722,136 |
| 10 | Cancer Gene | Tier 3 | Tier 3 | Tier 3 | Tier 3 |
| | Tier | | | | |
| 11 | HRR GENE | NO | NO | NO | NO |
| 12 | Linear | 331001 | N/A Break in Gene | 138218 | 294571 |
| | distance to 5′ | | | | |
| | (bp) | | | | |
| 13 | Closest | 331001 | N/A Break in Gene | 87175 | 294571 |
| | distance to | | | | |
| | gene body | | | | |
| | (bp) | | | | |
| 14 | Partner 2 | intergenic | break in ERG | break in STUB1 | break in LMO2 |
| | gene or | | | | |
| | intergenic | | | | |
| 15 | Relevant | N/A | ERG | N/A | V/A |
| | cancer | | | | |
| | gene(s) | | | | |
| 16 | Gene 5′ | N/A | chr21: 38,498,477 | N/A | N/A |
| 17 | Gene 3′ | N/A | chr21: 38,380,036 | N/A | N/A |
| 18 | Cancer Gene | N/A | Tier 3 | N/A | N/A |
| | Tier | | | | |
| 19 | HRR GENE | N/A | NO | N/A | N/A |
| 20 | Linear | N/A | N/A Break in Gene | N/A Break in Gene | N/A Break in Gene |
| | distance to 5′ | | | | |
| | (bp) | | | | |
| 21 | Closest | N/A | N/A Break in Gene | N/A Break in Gene | N/A Break in Gene |
| | distance to | | | | |
| | gene body | | | | |
| | (bp) | | | | |
| 22 | Approx. | chr20: | chr21: | chr16: | chr11: |
| | partner | 45,280,001-45,285,000 | 38,385,001-38,390,000 | 680,001-685,000 | 33,875,001-33,880,000 |
| | breakpoint | | | | |
| | coordinate | | | | |
| | window 2A | | | | |
| 23 | Approx. | chr20: | chr21: | chr16: | chr11: |
| | partner | 45,275,001-45,290,000 | 38,380,001-38,395,000 | 675,001-690,000 | 33,870,001-33,885,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 33 | 34 | 35 | 36 |
| 2 | SAMPLE | S25 | S26 | S27 | S28 |
| | NUMBER | | | | |
| 3 | Tumor type | Plasmacytoma | Plasma Cell | Osseous | Epithelioid |
| | | | Neoplasm | Plasmacytoma | mesenchymal |
| | | | | | tumor with SMD |
| 4 | Partner 1 | intergenic break | intergenic break | intergenic break | intergenic break |
| | type | | | | |
| 5 | Approx. | chr11: | chr11: | chr11: | chr11: |
| | breakpoint | 69,375,001-69,380,000 | 69,510,001-69,515,000 | 69,445,001-69,450,000 | 69,500,001-69,501,000 |
| | coordinate | | | | |
| | window 1A | | | | |
| 6 | Approx. | chr11: | chr11: | chr11: | chr11: |
| | breakpoint | 69,370,001-69,385,000 | 69,505,001-69,520,000 | 69,440,001-69,455,000 | 69,498,001-69,503,000 |
| | coordinate | | | | |
| | window 1B | | | | |
| 7 | Relevant | CCND1 | CCND1 | CCND1 | CCND1 |
| | cancer | | | | |
| | gene(s) | | | | |
| 8 | Gene 5′ | chr11: 69,641,156 | chr11: 69,641,156 | chr11: 69,641,156 | chr11: 69,641,156 |
| 9 | Gene 3′ | chr11: 69,654,474 | chr11: 69,654,474 | chr11: 69,654,474 | chr11: 69,654,474 |
| 10 | Cancer Gene | Tier 3 | Tier 3 | Tier 3 | Tier 3 |
| | Tier | | | | |
| 11 | HRR GENE | NO | NO | NO | NO |
| 12 | Linear | 261156 | 126156 | 191156 | 140156 |
| | distance to 5′ | | | | |
| | (bp) | | | | |
| 13 | Closest | 261156 | 126156 | 191156 | 140156 |
| | distance to | | | | |
| | gene body | | | | |
| | (bp) | | | | |
| 14 | Partner 2 | intergenic break | intergenic break | intergenic break | intergenic break |
| | gene or | | | | |
| | intergenic | | | | |
| 15 | Relevant | N/A | IgH locus | N/A | N/A |
| | cancer | | | | |
| | gene(s) | | | | |
| 16 | Gene 5 | N/A | IgH locus | N/A | N/A |
| 17 | Gene 3′ | N/A | IgH locus | N/A | N/A |
| 18 | Cancer Gene | N/A | Tier 4 | N/A | N/A |
| | Tier | | | | |
| 19 | HRR GENE | N/A | NO | N/A | N/A |
| 20 | Linear | N/A | IgH locus | N/A | N/A |
| | distance to 5′ | | | | |
| | (bp) | | | | |
| 21 | Closest | N/A | IgH locus | N/A | N/A |
| | distance to | | | | |
| | gene body | | | | |
| | (bp) | | | | |
| 22 | Approx. | chr14: | chr14: | chr14: | chr11: |
| | partner | 105,710,001-105,715,000 | 105,770,001-105,775,000 | 105,860,001-105,865,000 | 101,198,001-101,199,000 |
| | breakpoint | | | | |
| | coordinate | | | | |
| | window 2A | | | | |
| 23 | Approx. | chr14: | chr14: | chr14: | chr11: |
| | partner | 105,705,001-105,720,000 | 105,765,001-105,780,000 | 105,855,001-105,870,000 | 101,196,001-101,201,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | FIG. 13 |
| 26 | NOTES | | | | |
| 1 | VARIANT ID | 37 | 38 | 39 | 40 |
| 2 | SAMPLE | S29 | S30 | S30 | S30 |
| | NUMBER | | | | |
| 3 | Tumor type | Spindle cell sarcoma | Undifferentiated | Undifferentiated | Undifferentiated |
| | | with myogenic | Uterine Sarcoma | Uterine Sarcoma | Uterine Sarcoma |
| | | differentiation | (UUS) - Uterine | (UUS) - Uterine | (UUS) - Uterine |
| 4 | Partner 1 | break in KIAA2026 | break in LYN | break in RAD51B | break in KREMEN1 |
| | type | | | | |
| 5 | Approx. | chr9: | chr8: | chr14: | chr22: |
| | breakpoint | 5,990,001-6,000,000 | 55,930,001-55,940,000 | 68,678,001-68,679,000 | 29,130,001-29,135,000 |
| | coordinate | | | | |
| | window 1A | | | | |
| 6 | Approx. | chr9: | chr8: | chr14: | chr22: |
| | breakpoint | 5,990,001-6,010,000 | 55,920,001-55,950,000 | 68,676,001-68,681,000 | 29,125,001-29,140,000 |
| | coordinate | | | | |
| | window 1B | | | | |
| 7 | Relevant | PD-L1 (CD274) | PLAG1 | RAD51B | CHEK2 |
| | cancer | PD-L2 (CD273) | | | |
| | gene(s) | | | | |
| 8 | Gene 5′ | PD-L1 (CD274): | chr8: 56,211,273 | chr14: 67,865,032 | chr22: 28,741,820 |
| | | chr9: 5,450,542 | | | |
| | | PD-L2 (CD273): | | | |
| | | chr9: 5,510,531 | | | |
| 9 | Gene 3′ | PD-L1 (CD274): | chr8: 56,160,909 | chr14: 68,683,118 | chr22: 28,687,743 |
| | | chr9: 5,470,55 | | | |
| | | 4PD-L2 (CD273): | | | |
| | | chr9: 5,571,282 | | | |
| 10 | Cancer Gene | PD-L1 (CD274): Tier 1 | Tier 3 | Tier 1 | Tier 1 |
| | Tier | PD-L2 (CD273): Tier 4 | | | |
| 11 | HRR GENE | NO | NO | YES | YES |
| 12 | Linear | PD-L1 (CD274): 539,459 | 271273 | N/A break in gene | 388181 |
| | distance to 5′ | PD-L2 (CD273): 479,470 | | | |
| | (bp) | | | | |
| 13 | Closest | PD-L1 (CD274): 519,447 | 220909 | N/A break in gene | 388181 |
| | distance to | PD-L2 (CD273): 418,719 | | | |
| | gene body | | | | |
| | (bp) | | | | |
| 14 | Partner 2 | break in ADAMTS17 | break in CASC21 | break in RPSAP52 | intergenic break |
| | gene or | | | | |
| | intergenic | | | | |
| 15 | Relevant | N/A | MYC | N/A | SMARCA4 |
| | cancer | | | | |
| | gene(s) | | | | |
| 16 | Gene 5′ | N/A | chr8: 127,736,084 | N/A | chr19: 10,961,001 |
| 17 | Gene 3′ | N/A | chr8: 127,741,434 | N/A | chr19: 11,062,256 |
| 18 | Cancer Gene | N/A | Tier 4 | N/A | Tier 3 |
| | Tier | | | | |
| 19 | HRR GENE | N/A | NO | N/A | NO |
| 20 | Linear | N/A Break in Gene | 396084 | N/A Break in Gene | 569000 |
| | distance to 5′ | | | | |
| | (bp) | | | | |
| 21 | Closest | N/A Break in Gene | 396084 | N/A Break in Gene | 467745 |
| | distance to | | | | |
| | gene body | | | | |
| | (bp) | | | | |
| 22 | Approx. | chr15: | chr8: | chr12: | chr19: |
| | partner | 100,300,001-100,310,000 | 127,330,001-127,340,000 | 65,816,001-65,817,000 | 11,530,001-11,535,000 |
| | breakpoint | | | | |
| | coordinate | | | | |
| | window 2A | | | | |
| 23 | Approx. | chr15: | chr8: | chr12: | chr19: |
| | partner | 100,290,001-100,320,000 | 127,320,001-127,350,000 | 65,814,001-65,819,000 | 11,525,001-11,540,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 41 | 42 | 43 | 44 |
| 2 | SAMPLE | S30 | S31 | S31 | S31 |
| | NUMBER | | | | |
| 3 | Tumor type | Undifferentiated | Low-grade | Low-grade | Low-grade |
| | | Uterine Sarcoma | endometrial | endometrial | endometrial |
| | | (UUS) - Uterine | stromal sarcoma | stromal sarcoma | stromal sarcoma |
| | | | (LGESS) - Uterine | (LGESS) - Uterine | (LGESS) - Uterine |
| 4 | Partner 1 | break in BCAT1 | intergenic break | intergenic break | break in MEGF11 |
| | type | | | | |
| 5 | Approx. | chr12: | chr8: | chr8: | chr15: |
| | breakpoint | 24,930,001-24,935,000 | 56,140,001-56,150,000 | 89,850,001-89,855,000 | 66,065,001-66,070,000 |
| | coordinate | | | | |
| | window 1A | | | | |
| 6 | Approx. | chr12: | chr8: | chr8: | chr15: |
| | breakpoint | 24,925,001-24,940,000 | 56,130,001-56,160,000 | 89,845,001-89,860,000 | 66,060,001-66,075,000 |
| | coordinate | | | | |
| | window 1B | | | | |
| 7 | Relevant | KRAS | PLAG1 | NBN | MAP2K1 |
| | cancer | | | | |
| | gene(s) | | | | |
| 8 | Gene 5′ | chr12: 25,250,929 | chr8: 56,211,273 | chr8: 89,984,682 | chr15: 66,386,912 |
| 9 | Gene 3′ | chr12: 25,205,246 | chr8: 56,160,909 | chr8: 89,924,515 | chr15: 66,491,544 |
| 10 | Cancer Gene | Tier 1 | Tier 3 | Tier 1 | Tier 1 |
| | Tier | | | | |
| 11 | HRR GENE | NO | NO | YES | NO |
| 12 | Linear | 315929 | 61273 | 129682 | 316912 |
| | distance to 5′ | | | | |
| | (bp) | | | | |
| 13 | Closest | 270246 | 10909 | 69515 | 316912 |
| | distance to | | | | |
| | gene body | | | | |
| | (bp) | | | | |
| 14 | Partner 2 | intergenic break | break in VPS13B | break in TSNARE1 | break in TJP1 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A Break in Gene | N/A Break in Gene | N/A Break in Gene |
| | distance to 5′ | | | | |
| | (bp) | | | | |
| 21 | Closest | N/A | N/A Break in Gene | N/A Break in Gene | N/A Break in Gene |
| | distance to | | | | |
| | gene body | | | | |
| | (bp) | | | | |
| 22 | Approx. | chr12: | chr8: | chr8: | chr15: |
| | partner | 67,455,001-67,460,000 | 99,020,001-99,030,000 | 142,210,001-142,215,008 | 29,755,001-29,760,000 |
| | breakpoint | | | | |
| | coordinate | | | | |
| | window 2A | | | | |
| 23 | Approx. | chr12: | chr8: | chr8: | chr15: |
| | partner | 67,450,001-67,465,000 | 99,010,001-99,040,000 | 142,205,001-142,220,008 | 29,750,001-29,765,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 45 | 46 | 47 | 48 |
| 2 | SAMPLE | S32 | S32 | S32 | S33 |
| | NUMBER | | | | |
| 3 | Tumor type | Fibrosarcoma | Fibrosarcoma | Fibrosarcoma | Sarcoma with |
| | | | | | sex-cord like |
| | | | | | differentiation |
| 4 | Partner 1 | break in TRIM37 | break in RAPGEFL1 | intergenic break | break in CCT6B |
| | type | | | | |
| 5 | Approx. | chr17: | chr17: | chr17: | chr17: |
| | breakpoint | 58,982,001-58,983,000 | 40,185,001-40,190,000 | 31,720,001-31,725,000 | 34,940,001-34,945,000 |
| | coordinate | | | | |
| | window 1A | | | | |
| 6 | Approx. | chr17: | chr17: | chr17: | chr17: |
| | breakpoint | 58,980,001-58,985,000 | 40,180,001-40,195,000 | 31,715,001-31,730,000 | 34,935,001-34,950,000 |
| | coordinate | | | | |
| | window 1B | | | | |
| 7 | Relevant | RAD51C | CDK12 | NF1 | RAD51D |
| | cancer | | ERBB2 | | |
| | gene(s) | | | | |
| 8 | Gene 5′ | chr17: 58,692,602 | CDK12: | chr17: 31,094,977 | chr17: 35,119,860 |
| | | | chr17: 39,461,761 | | |
| | | | ERBB2: | | |
| | | | chr17: 39,700,064 | | |
| 9 | Gene 3′ | chr17: 58,735,611 | CDK12: | chr17: 31,377,675 | chr17: 35,092,221 |
| | | | chr17: 39,532,477E | | |
| | | | RBB2: | | |
| | | | chr17: 39,728,658 | | |
| 10 | Cancer Gene | Tier 1 | CDK12: Tier 1 | Tier 1 | Tier 1 |
| | Tier | | ERBB2: Tier 1 | | |
| 11 | HRR GENE | YES | CDK12: YES | NO | YES |
| | | | ERBB2: NO | | |
| 12 | Linear | 289399 | CDK12: 723,240 | 625024 | 174860 |
| | distance to 5′ | | ERBB2: 484,937 | | |
| | (bp) | | | | |
| 13 | Closest | 246390 | CDK12: 652,524 | 342326 | 147221 |
| | distance to | | ERBB2: 456,343 | | |
| | gene body | | | | |
| | (bp) | | | | |
| 14 | Partner 2 | break in PITPNC1 | intergenic break | intergenic break | break in PIMREG |
| | gene or | | | | |
| | intergenic | | | | |
| 15 | Relevant | N/A | SUZ12 | N/A | N/A |
| | cancer | | | | |
| | gene(s) | | | | |
| 16 | Gene 5′ | N/A | chr17: 31,937,007 | N/A | N/A |
| 17 | Gene 3′ | N/A | chr17: 32,001,038 | N/A | N/A |
| 18 | Cancer Gene | N/A | Tier 3 | N/A | N/A |
| | Tier | | | | |
| 19 | HRR GENE | N/A | NO | N/A | N/A |
| 20 | Linear | N/A Break in Gene | 112007 | N/A | N/A Break in Gene |
| | distance to 5′ | | | | |
| | (bp) | | | | |
| 21 | Closest | N/A Break in Gene | 112007 | N/A | N/A Break in Gene |
| | distance to | | | | |
| | gene body | | | | |
| | (bp) | | | | |
| 22 | Approx. | chr17: | chr17: | chr17: | chr17: |
| | partner | 67,659,001-67,660,000 | 31,820,001-31,825,000 | 37,885,001-37,890,000 | 6,445,001-6,450,000 |
| | breakpoint | | | | |
| | coordinate | | | | |
| | window 2A | | | | |
| 23 | Approx. | chr17: | chr17: | chr17: | chr17: |
| | partner | 67,657,001-67,662,000 | 31,815,001-31,830,000 | 37,880,001-37,895,000 | 6,440,001-6,455,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 49 | 50 | 51 | 52 |
| 2 | SAMPLE | S33 | S34 | S35 | S35 |
| | NUMBER | | | | |
| 3 | Tumor type | Sarcoma with | Low Grade | low grade (LG) | low grade (LG) |
| | | sex-cord like | Adenosarcoma | epithelioid | epithelioid |
| | | differentiation | | neoplasm with | neoplasm with |
| | | | | myomelanocytic | myomelanocytic |
| | | | | differentiation | differentiation |
| 4 | Partner 1 | break in ATRX | intergenic break | intergenic break | break in ELF1 |
| | type | | | | |
| 5 | Approx. | chrX: | chr14: | chr11: | chr13: |
| | breakpoint | 77,530,001-77,535,000 | 68,753,001-68,754,000 | 69,370,001-69,375,000 | 41,030,001-41,035,000 |
| | coordinate | | | | |
| | window 1A | | | | |
| 6 | Approx. | chrX: | chr14: | chr11: | chr13: |
| | breakpoint | 77,525,001-77,540,000 | 68,751,001-68,756,000 | 69,365,001-69,380,000 | 41,025,001-41,040,000 |
| | coordinate | | | | |
| | window 1B | | | | |
| 7 | Relevant | ATRX | RAD51B | CCND1 | FOXO1 |
| | cancer | | | | |
| | gene(s) | | | | |
| 8 | Gene 5′ | chrX: 77,786,216 | chr14: 67,865,032 | chr11: 69,641,156 | chr13: 40,666,641 |
| 9 | Gene 3′ | chrX: 77,504,880 | chr14: 68,683,118 | chr11: 69,654,474 | chr13: 40,555,667 |
| 10 | Cancer Gene | Tier 3 | Tier 1 | Tier 3 | Tier 3 |
| | Tier | | | | |
| 11 | HRR GENE | NO | YES | NO | NO |
| 12 | Linear | N/A break in gene | 887969 | 266156 | 363360 |
| | distance to 5′ | | | | |
| | (bp) | | | | |
| 13 | Closest | N/A break in gene | 69883 | 266156 | 363360 |
| | distance to | | | | |
| | gene body | | | | |
| | (bp) | | | | |
| 14 | Partner 2 | intergenic break | break in RPSAP52 | break in MRPL23 | break in OSBPL5 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A Break in Gene | N/A Break in Gene | N/A Break in Gene |
| | distance to 5′ | | | | |
| | (bp) | | | | |
| 21 | Closest | N/A | N/A Break in Gene | N/A Break in Gene | N/A Break in Gene |
| | distance to | | | | |
| | gene body | | | | |
| | (bp) | | | | |
| 22 | Approx. | chrX: | chr12: | chr11: | chr11: |
| | partner | 83,500,001-83,505,000 | 65,811,001-65,812,000 | 1,955,001-1,960,000 | 3,165,001-3,170,000 |
| | breakpoint | | | | |
| | coordinate | | | | |
| | window 2A | | | | |
| 23 | Approx. | chrX: | chr12: | chr11: | chr11: |
| | partner | 83,495,001-83,510,000 | 65,809,001-65,814,000 | 1,950,001-1,965,000 | 3,160,001-3,175,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | FIG. 15 | N/A |
| 26 | NOTES | | | | |
| 1 | VARIANT ID | 53 | 54 | 55 | 56 |
| 2 | SAMPLE | S36 | S36 | S37 | S37 |
| | NUMBER | | | | |
| 3 | Tumor type | Perivascular | Perivascular | Highly atypical | Highly atypical |
| | | epithelioid cell | epithelioid cell | spindled and | spindled and |
| | | tumour (PEComa) | tumour (PEComa) | epithelioid | epithelioid |
| | | | | neoplasm with | neoplasm with |
| | | | | myxoid features, | myxoid features, |
| | | | | c/w sarcoma | c/w sarcoma |
| 4 | Partner 1 | intergenic break | break in FGFR1 | intergenic break | break in RAD51B |
| | type | | | | |
| 5 | Approx. | chr8: | chr8: | chr1: | chr14: |
| | breakpoint | 31,380,001-31,390,000 | 38,410,001-38,415,000 | 157,263,001-157,264,000 | 68,324,001-68,325,000 |
| | coordinate | | | | |
| | window 1A | | | | |
| 6 | Approx. | chr8: | chr8: | chr1: | chr14: |
| | breakpoint | 31,370,001-31,400,000 | 38,405,001-38,420,000 | 157,261,001-157,266,000 | 68,322,001-68,327,000 |
| | coordinate | | | | |
| | window 1B | | | | |
| 7 | Relevant | NRG1 | FGFR1 | NTRK1 | RAD51B |
| | cancer | | | | |
| | gene(s) | | | | |
| 8 | Gene 5′ | chr8: 31,639,222 | chr8: 38,468,641 | chr1: 156,860,865 | chr14: 67,865,032 |
| 9 | Gene 3′ | chr8: 32,764,405 | chr8: 38,411,138 | chr1: 156,881,850 | chr14: 68,683,118 |
| 10 | Cancer Gene | Tier 1 | Tier 1 | Tier 1 | Tier 1 |
| | Tier | | | | |
| 11 | HRR GENE | NO | NO | NO | YES |
| 12 | Linear | 249222 | N/A break in gene | 403135 | N/A break in gene |
| | distance to 5′ | | | | |
| | (bp) | | | | |
| 13 | Closest | 249222 | N/A break in gene | 403135 | N/A break in gene |
| | distance to | | | | |
| | gene body | | | | |
| | (bp) | | | | |
| 14 | Partner 2 | break in NR_125425 | break in SDCBP | intergenic break | break in NRXN3 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A Break in Gene | N/A | N/A Break in Gene |
| | distance to 5′ | | | | |
| | (bp) | | | | |
| 21 | Closest | N/A Break in Gene | N/A Break in Gene | N/A | N/A Break in Gene |
| | distance to | | | | |
| | gene body | | | | |
| | (bp) | | | | |
| 22 | Approx. | chr8: | chr8: | chr1: | chr14: |
| | partner | 2,540,001-2,550,000 | 58,570,001-58,575,000 | 226,934,001-226,935,000 | 79,637,001-79,638,000 |
| | breakpoint | | | | |
| | coordinate | | | | |
| | window 2A | | | | |
| 23 | Approx. | chr8: | chr8: | chr1: | chr14: |
| | partner | 2,530,001-2,560,000 | 58,565,001-58,580,000 | 226,932,001-226,937,000 | 79,635,001-79,640,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 57 | 58 | 59 | 60 |
| 2 | SAMPLE | S38 | S38 | S39 | S39 |
| | NUMBER | | | | |
| 3 | Tumor type | Undifferentiated | Undifferentiated | High grade | High grade |
| | | Uterine Sarcoma | Uterine Sarcoma | Adenosarcoma | Adenosarcoma |
| | | (UUS) - Uterine | (UUS) - Uterine | with sarcoma | with sarcoma |
| | | | | overgrowth | overgrowth |
| | | | | (HG ASSO) | (HG ASSO) |
| 4 | Partner 1 | break in LCLAT1 | break in PLAG1 | intergenic break | intergenic break |
| | type | | | | |
| 5 | Approx. | chr2: | chr8: | chr8: | chr5: |
| | breakpoint | 30,640,001-30,645,000 | 56,160,001-56,165,000 | 56,137,001-56,138,000 | 1,308,001-1,309,000 |
| | coordinate | | | | |
| | window 1A | | | | |
| 6 | Approx. | chr2: | chr8: | chr8: | chr5: |
| | breakpoint | 30,635,001-30,650,000 | 56,155,001-56,170,000 | 56,135,001-56,140,000 | 1,306,001-1,3011,000 |
| | coordinate | | | | |
| | window 1B | | | | |
| 7 | Relevant | ALK | PLAG1 | PLAG1 | TERT |
| | cancer | | | | |
| | gene(s) | | | | |
| 8 | Gene 5′ | chr2: 29,921,586 | chr8: 56,211,273 | chr8: 56,211,273 | chr5: 1,295,068 |
| 9 | Gene 3′ | chr2: 29,192,774 | chr8: 56,160,909 | chr8: 56,160,909 | chr5: 1,253,167 |
| 10 | Cancer Gene | Tier 1 | Tier 3 | Tier 3 | Tier 3 |
| | Tier | | | | |
| 11 | HRR GENE | NO | NO | NO | NO |
| 12 | Linear | 718415 | N/A Break in Gene | 73273 | 12933 |
| | distance to 5′ | | | | |
| | (bp) | | | | |
| 13 | Closest | 718415 | N/A Break in Gene | 22909 | 12933 |
| | distance to | | | | |
| | gene body | | | | |
| | (bp) | | | | |
| 14 | Partner 2 | intergenic | break in PBX1 | break in RAD51B | intergenic break |
| | gene or | break | | | |
| | intergenic | | | | |
| 15 | Relevant | N/A | N/A | RAD51B | N/A |
| | cancer | | | | |
| | gene(s) | | | | |
| 16 | Gene 5′ | N/A | N/A | chr14: 67,865,032 | N/A |
| 17 | Gene 3′ | N/A | N/A | chr14: 68,683,118 | N/A |
| 18 | Cancer Gene | N/A | N/A | Tier 1 | N/A |
| | Tier | | | | |
| 19 | HRR GENE | N/A | N/A | YES | N/A |
| 20 | Linear | N/A | N/A Break in Gene | N/A Break in Gene | NA |
| | distance to 5′ | | | | |
| | (bp) | | | | |
| 21 | Closest | N/A | N/A Break in Gene | N/A Break in Gene | N/A |
| | distance to | | | | |
| | gene body | | | | |
| | (bp) | | | | |
| 22 | Approx. | chr12: | chr1: | chr14: | chr5: |
| | partner | 112,155,001-112,160,000 | 164,640,001-164,645,000 | 68,478,001-68,479,000 | 35,395,001-35,396,000 |
| | breakpoint | | | | |
| | coordinate | | | | |
| | window 2A | | | | |
| 23 | Approx. | chr12: | chr1: | chr14: | chr5: |
| | partner | 112,150,001-112,165,000 | 164,635,001-164,650,000 | 68,476,001-68,481,000 | 35,393,001-35,398,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 61 | 62 | 63 | 64 |
| 2 | SAMPLE | S39 | S39 | S39 | S40 |
| | NUMBER | | | | |
| 3 | Tumor type | High grade | High grade | High grade | Adenosarcoma |
| | | Adenosarcoma | Adenosarcoma | Adenosarcoma | with sarcoma |
| | | with sarcoma | with sarcoma | with sarcoma | overgrowth |
| | | overgrowth | overgrowth | overgrowth | (ASSO) |
| | | (HG ASSO) | (HG ASSO) | (HG ASSO) | |
| 4 | Partner 1 | intergenic break | break in FLT1 | break in GLYCTK-AS1 | breka in SYN2 |
| | type | | | | |
| 5 | Approx. | chr20: | chr13: | chr3: | chr3: |
| | breakpoint | 46,825,001-46,830,000 | 28,453,001-28,454,000 | 52,289,001-52,290,000 | 12,110,001-12,115,000 |
| | coordinate | | | | |
| | window 1A | | | | |
| 6 | Approx. | chr20: | chr13: | chr3: | chr3: |
| | breakpoint | 46,820,001-46,835,000 | 28,451,001-28,456,000 | 52,287,001-52,292,000 | 12,105,001-12,120,000 |
| | coordinate | | | | |
| | window 1B | | | | |
| 7 | Relevant | NCOA3 | FLT3 | PARP3 | RAF1 |
| | cancer | | | | |
| | gene(s) | | | | |
| 8 | Gene 5′ | chr20: 47,501,887 | chr13: 28,100,576 | chr3: 51,942,345 | chr3: 12,664,187 |
| 9 | Gene 3′ | chr20: 47,656,872 | chr13: 28,003,274 | chr3: 51,948,862 | chr3: 12,582,101 |
| 10 | Cancer Gene | Tier 3 | Tier 1 | Tier 1 | Tier 1 |
| | Tier | | | | |
| 11 | HRR GENE | NO | NO | YES | NO |
| 12 | Linear | 671887 | 352425 | 346656 | 549187 |
| | distance to 5′ | | | | |
| | (bp) | | | | |
| 13 | Closest | 671887 | 352425 | 340139 | 467101 |
| | distance to | | | | |
| | gene body | | | | |
| | (bp) | | | | |
| 14 | Partner 2 | break in ATPSCKMT | break in STARD13 | intergenic break | breka in TBX4 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A Break in Gene | NA | N/A Break in Gene |
| | distance to 5′ | | | | |
| | (bp) | | | | |
| 21 | Closest | N/A Break in Gene | N/A Break in Gene | N/A | N/A Break in Gene |
| | distance to | | | | |
| | gene body | | | | |
| | (bp) | | | | |
| 22 | Approx. | chr5: | chr13: | chr3: | chr17: |
| | partner | 10,235,001-10,240,000 | 33,176,001-33,177,000 | 42,036,001-42,037,000 | 61,465,001-61,470,000 |
| | breakpoint | | | | |
| | coordinate | | | | |
| | window 2A | | | | |
| 23 | Approx. | chr5: | chr13: | chr3: | chr17: |
| | partner | 10,230,001-10,245,000 | 33,174,001-33,179,000 | 42,034,001-42,039,000 | 61,460,001-61,475,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 65 | 66 | 67 | 68 |
| 2 | SAMPLE | S40 | S40 | S40 | S40 |
| | NUMBER | | | | |
| 3 | Tumor type | Adenosarcoma | Adenosarcoma | Adenosarcoma | Adenosarcoma |
| | | with sarcoma | with sarcoma | with sarcoma | with sarcoma |
| | | overgrowth | overgrowth | overgrowth | overgrowth |
| | | (ASSO) | (ASSO) | (ASSO) | (ASSO) |
| 4 | Partner 1 | intergenic break | break in RAB39A | break in LOC283387 | intergenic break |
| | type | | | | |
| 5 | Approx. | chr17: | chr11: | chr12: | chr12: |
| | breakpoint | 61,610,001-61,615,000 | 107,955,001-107,960,000 | 57,885,001-57,890,000 | 68,466,001-68,467,000 |
| | coordinate | | | | |
| | window 1A | | | | |
| 6 | Approx. | chr17: | chr11: | chr12: | chr12: |
| | breakpoint | 61,605,001-61,620,000 | 107,950,001-107,965,000 | 57,880,001-57,895,000 | 68,464,001-68,469,000 |
| | coordinate | | | | |
| | window 1B | | | | |
| 7 | Relevant | BRIP1 | ATM | CDK4 | MDM2 |
| | cancer | | | | |
| | gene(s) | | | | |
| 8 | Gene 5′ | chr17: 61,863,528 | chr11: 108,223,067 | chr12: 57,752,310 | chr12: 68,809,002 |
| 9 | Gene 3′ | chr17: 61,679,139 | chr11: 108,369,102 | chr12: 57,747,727 | chr12: 68,840,807 |
| 10 | Cancer Gene | Tier 1 | Tier 1 | Tier 2 | Tier 2 |
| | Tier | | | | |
| 11 | HRR GENE | YES | YES | NO | NO |
| 12 | Linear | 248528 | 263067 | 132691 | 342002 |
| | distance to 5′ | | | | |
| | (bp) | | | | |
| 13 | Closest | 64139 | 263067 | 132691 | 342002 |
| | distance to | | | | |
| | gene body | | | | |
| | (bp) | | | | |
| 14 | Partner 2 | break in VGLL4 | intergenic break | break in KATNBL1 | intergenic break |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A | N/A Break in Gene | N/A |
| | distance to 5′ | | | | |
| | (bp) | | | | |
| 21 | Closest | N/A Break in Gene | N/A | N/A Break in Gene | N/A |
| | distance to | | | | |
| | gene body | | | | |
| | (bp) | | | | |
| 22 | Approx. | chr3: | chr11: | chr15: | chr12: |
| | partner | 11,625,001-11,630,000 | 110,975,001-110,980,000 | 34,145,001-34,150,000 | 61,095,001-61,096,000 |
| | breakpoint | | | | |
| | coordinate | | | | |
| | window 2A | | | | |
| 23 | Approx. | chr3: | chr11: | chr15: | chr12: |
| | partner | 11,620,001-11,635,000 | 110,970,001-110,985,000 | 34,140,001-34,155,000 | 61,093,001-61,098,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | FIG. 14 | N/A |
| 26 | NOTES | | | | |
| 1 | VARIANT ID | 69 | 70 | 71 | 72 |
| 2 | SAMPLE | S40 | S41 | S42 | S42 |
| | NUMBER | | | | |
| 3 | Tumor type | Adenosarcoma | Uterine tumor | Uterine smooth | Uterine smooth |
| | | with sarcoma | resembling | muscle tumor of | muscle tumor of |
| | | overgrowth (ASSO) | ovarian sex cord | uncertain malignant | uncertain malignant |
| | | | tumor (UTROSCT) | potential (STUMP) | potential (STUMP) |
| 4 | Partner 1 | break in ESYT1 | break in ESR1 | break in FANCA | break in PLAG1 |
| | type | | | | |
| 5 | Approx. | chr12: | chr6: | chr16: | chr8: |
| | breakpoint | 56,132,001-56,133,000 | 151,890,001-151,895,000 | 89,791,001-89,792,000 | 56,205,001-56,210,000 |
| | coordinate | | | | |
| | window 1A | | | | |
| 6 | Approx. | chr12: | chr6: | chr16: | chr8: |
| | breakpoint | 56,130,001-56,135,000 | 151,885,001-151,900,000 | 89,789,001-89,794,000 | 56,200,001-56,215,000 |
| | coordinate | | | | |
| | window 1B | | | | |
| 7 | Relevant | ERBB3 | ESR1 | FANCA | PLAG1 |
| | cancer | | | | |
| | gene(s) | | | | |
| 8 | Gene 5′ | chr12: 56,080,165 | chr6: 151,690,496 | chr16: 89,816,647 | chr8: 56,211,273 |
| 9 | Gene 3′ | chr12: 56,103,505 | chr6: 152,103,274 | chr16: 89,737,549 | chr8: 56,160,909 |
| 10 | Cancer Gene | Tier 2 | Tier 1 | Tier 1 | Tier 3 |
| | Tier | | | | |
| 11 | HRR GENE | NO | NO | YES | NO |
| 12 | Linear | 51836 | N/A Break in Gene | N/A Break in Gene | N/A Break in Gene |
| | distance to 5′ | | | | |
| | (bp) | | | | |
| 13 | Closest | 28496 | N/A Break in Gene | N/A Break in Gene | N/A Break in Gene |
| | distance to | | | | |
| | gene body | | | | |
| | (bp) | | | | |
| 14 | Partner 2 | break in LINC02882 | break in NCOA3 | intergenic break | break in PRLR |
| | gene or | | | | |
| | intergenic | | | | |
| 15 | Relevant | N/A | NCOA3 | N/A | N/A |
| | cancer | | | | |
| | gene(s) | | | | |
| 16 | Gene 5′ | N/A | chr20: 47,501,887 | N/A | N/A |
| 17 | Gene 3′ | N/A | chr20: 47,656,872 | N/A | N/A |
| 18 | Cancer Gene | N/A | Tier 3 | N/A | N/A |
| | Tier | | | | |
| 19 | HRR GENE | N/A | NO | N/A | N/A |
| 20 | Linear | N/A Break in Gene | N/A Break in Gene | N/A | N/A Break in Gene |
| | distance to 5′ | | | | |
| | (bp) | | | | |
| 21 | Closest | N/A Break in Gene | N/A Break in Gene | N/A | N/A Break in Gene |
| | distance to | | | | |
| | gene body | | | | |
| | (bp) | | | | |
| 22 | Approx. | chr12: | chr20: | chr13: | chr5: |
| | partner | 74,138,001-74,139,000 | 47,635,001-47,640,000 | 44,810,001-44,811,000 | 35,225,001-35,230,000 |
| | breakpoint | | | | |
| | coordinate | | | | |
| | window 2A | | | | |
| 23 | Approx. | chr12: | chr20: | chr13: | chr5: |
| | partner | 74,136,001-74,141,000 | 47,630,001-47,645,000 | 44,808,001-44,813,000 | 35,220,001-35,235,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | FIG. 17 | N/A | N/A |
| 26 | NOTES | | | | |
| 1 | VARIANT ID | 73 | 74 | 75 | 76 |
| 2 | SAMPLE | S43 | S44 | S45 | S46 |
| | NUMBER | | | | |
| 3 | Tumor type | Uterine smooth | Plasmacytoma | High-grade | Atypical |
| | | muscle tumor of | | endometrial | leiomyosarcoma |
| | | uncertain malignant | | stromal sarcoma | (LM) with low |
| | | potential (STUMP) | | (HGESS) - Uterine | recurrence risk |
| 4 | Partner 1 | break in RAD51B | break in SPCS1 | intergenic break | break in RAD51B |
| | type | | | | |
| 5 | Approx. | chr14: | chr3: | chr3: | chr14: |
| | breakpoint | 68,650,001-68,655,000 | 52,700,001-52,710,000 | 10,140,001-10,145,000 | 68,660,001-68,665,000 |
| | coordinate | | | | |
| | window 1A | | | | |
| 6 | Approx. | chr14: | chr3: | chr3: | chr14: |
| | breakpoint | 68,645,001-68,660,000 | 52,690,001-52,720,000 | 10,135,001-10,150,000 | 68,655,001-68,670,000 |
| | coordinate | | | | |
| | window 1B | | | | |
| 7 | Relevant | RAD51B | BAP1 | FANCD2 | RAD51B |
| | cancer | | | | |
| | gene(s) | | | | |
| 8 | Gene 5′ | chr14: 67,865,032 | chr3: 52,410,008 | chr3: 10,026,437 | chr14: 67,865,032 |
| 9 | Gene 3′ | chr14: 68,683,118 | chr3: 52,401,008 | chr3: 10,101,932 | chr14: 68,683,118 |
| 10 | Cancer Gene | Tier 1 | Tier 1 | Tier 1 | Tier 1 |
| | Tier | | | | |
| 11 | HRR GENE | YES | NO | YES | YES |
| 12 | Linear | N/A Break in Gene | 289993 | 113564 | N/A Break in Gene |
| | distance to 5′ | | | | |
| | (bp) | | | | |
| 13 | Closest | N/A Break in Gene | 289993 | 38069 | N/A Break in Gene |
| | distance to | | | | |
| | gene body | | | | |
| | (bp) | | | | |
| 14 | Partner 2 | intergenic break | break in THRB | break in ADCY1 | break in NUDT3 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A Break in Gene | N/A Break in Gene | N/A Break in Gene |
| | distance to 5′ | | | | |
| | (bp) | | | | |
| 21 | Closest | N/A | N/A Break in Gene | N/A Break in Gene | N/A Break in Gene |
| | distance to | | | | |
| | gene body | | | | |
| | (bp) | | | | |
| 22 | Approx. | chr12: | chr3: | chr7: | chr6: |
| | partner | 65,725,001-65,730,000 | 24,240,001-24,250,000 | 45,680,001-45,685,000 | 34,365,001-34,370,000 |
| | breakpoint | | | | |
| | coordinate | | | | |
| | window 2A | | | | |
| 23 | Approx. | chr12: | chr3: | chr7: | chr6: |
| | partner | 65,720,001-65,735,000 | 24,230,001-24,260,000 | 45,675,001-45,690,000 | 34,360,001-34,375,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 77 | 78 | 79 | 80 |
| 2 | SAMPLE | S46 | S46 | S46 | S47 |
| | NUMBER | | | | |
| 3 | Tumor type | Atypical | high grade sarcoma | high grade sarcoma | HG spindle cell |
| | | leiomyosarcoma | (recurrent tumor) | (recurrent tumor) | sarcoma |
| | | (LM) with low | | | |
| | | recurrence risk | | | |
| 4 | Partner 1 | break in ARMT1 | break in ESR1 | intergenic break | break in NCOA2 |
| | type | | | | |
| 5 | Approx. | chr6: | chr6: | chr11: | chr8: |
| | breakpoint | 151,455,001-151,460,000 | 151,940,001-151,945,000 | 2,073,001-2,074,000 | 70,138,001-70,139,000 |
| | coordinate | | | | |
| | window 1A | | | | |
| 6 | Approx. | chr6: | chr6: | chr11: | chr8: |
| | breakpoint | 151,450,001-151,465,000 | 151,935,001-151,950,000 | 2,071,001-2,076,000 | 70,136,001-70,141,000 |
| | coordinate | | | | |
| | window 1B | | | | |
| 7 | Relevant | ESR1 | ESR1 | IGF2 | NCOA2 |
| | cancer | | | | |
| | gene(s) | | | | |
| 8 | Gene 5′ | chr6: 151,690,496 | chr6: 151,690,496 | chr11: 2,138,974 | chr8: 70,403,808 |
| 9 | Gene 3′ | chr6: 152,103,274 | chr6: 152,103,274 | chr11: 2,129,112 | chr8: 70,109,782 |
| 10 | Cancer Gene | Tier 1 | Tier 1 | Tier 2 | Tier 3 |
| | Tier | | | | |
| 11 | HRR GENE | NO | NO | NO | NO |
| 12 | Linear | 230496 | N/A Break in Gene | 64974 | N/A Break in Gene |
| | distance to 5′ | | | | |
| | (bp) | | | | |
| 13 | Closest | 230496 | N/A Break in Gene | 55112 | N/A Break in Gene |
| | distance to | | | | |
| | gene body | | | | |
| | (bp) | | | | |
| 14 | Partner 2 | break in SOD2 | break in NCOA3 | intergenic break | break in GREB1 |
| | gene or | | | | |
| | intergenic | | | | |
| 15 | Relevant | N/A | NCOA3 | N/A | N/A |
| | cancer | | | | |
| | gene(s) | | | | |
| 16 | Gene 5′ | N/A | chr20: 47,501,887 | N/A | N/A |
| 17 | Gene 3′ | N/A | chr20: 47,656,872 | N/A | N/A |
| 18 | Cancer Gene | N/A | Tier 3 | N/A | N/A |
| | Tier | | | | |
| 19 | HRR GENE | N/A | NO | N/A | N/A |
| 20 | Linear | N/A Break in Gene | N/A Break in Gene | N/A | N/A Break in Gene |
| | distance to 5′ | | | | |
| | (bp) | | | | |
| 21 | Closest | N/A Break in Gene | N/A Break in Gene | N/A | N/A Break in Gene |
| | distance to | | | | |
| | gene body | | | | |
| | (bp) | | | | |
| 22 | Approx. | chr6: | chr20: | chr19: | chr2: |
| | partner | 159,675,001-159,680,000 | 47,635,001-47,640,000 | 56,880,001-56,881,000 | 11,563,001-11,564,000 |
| | breakpoint | | | | |
| | coordinate | | | | |
| | window 2A | | | | |
| 23 | Approx. | chr6: | chr20: | chr19: | chr2: |
| | partner | 159,670,001-159,685,000 | 47,630,001-47,645,000 | 56,878,001-56,883,000 | 11,561,001-11,566,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | FIG. 21 | N/A | N/A | N/A |
| 26 | NOTES | | | | |
| 1 | VARIANT ID | 81 | 82 | 83 | 84 |
| 2 | SAMPLE | S48 | S49 | S50 | S51 |
| | NUMBER | | | | |
| 3 | Tumor type | Low-grade | High-grade | HG malignant | Osseous |
| | | endometrial | endometrial | epithelioid and | plasmcytoma |
| | | stromal sarcoma | stromal sarcoma | spindled neoplasm | |
| | | (LGESS) - Uterine | (HGESS) - Uterine | | |
| 4 | Partner 1 | break in PHF1 | break in EPC1 | break in ABCC8 | intergenic break |
| | type | | | | in IgL locus, |
| | | | | | about 60 kb |
| | | | | | downstream from |
| | | | | | IgL genes |
| 5 | Approx. | chr6: | chr10: | chr11: | chr22: |
| | breakpoint | 33,410,001-33,415,000 | 32,289,001-32,290,000 | 17,420,001-17,425,000 | 22,985,001-22,990,000 |
| | coordinate | | | | |
| | window 1A | | | | |
| 6 | Approx. | chr6: | chr10: | chr11: | chr22: |
| | breakpoint | 33,405,001-33,420,000 | 32,287,001-32,292,000 | 17,415,001-17,430,000 | 22,980,001-22,995,000 |
| | coordinate | | | | |
| | window 1B | | | | |
| 7 | Relevant | PHF1 | EPC1 | MyoD1 | IgL |
| | cancer | | | | |
| | gene(s) | | | | |
| 8 | Gene 5′ | chr6: 33,411,014 | chr10: 32,347,158 | chr11: 17,719,571 | |
| 9 | Gene 3′ | chr6: 33,416,439 | chr10: 32,267,751 | chr11: 17,722,136 | |
| 10 | Cancer Gene | Tier 3 | Tier 3 | Tier 3 | Tier 3 |
| | Tier | | | | |
| 11 | HRR GENE | NO | NO | NO | NO |
| 12 | Linear | N/A Break in Gene | N/A Break in Gene | 294571 | |
| | distance to 5′ | | | | |
| | (bp) | | | | |
| 13 | Closest | N/A Break in Gene | N/A Break in Gene | 294571 | |
| | distance to | | | | |
| | gene body | | | | |
| | (bp) | | | | |
| 14 | Partner 2 | break in HCFC1 | break in EED | break in LMO2 | intergenic break |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A Break in Gene | N/A Break in Gene | N/A |
| | distance to 5′ | | | | |
| | (bp) | | | | |
| 21 | Closest | N/A Break in Gene | N/A Break in Gene | N/A Break in Gene | N/A |
| | distance to | | | | |
| | gene body | | | | |
| | (bp) | | | | |
| 22 | Approx. | chrX: | chr11: | chr11: | chr2: |
| | partner | 153,950001-153,955,000 | 86,246,001-86,247,000 | 33,875,001-33,880,000 | 64,790,001-64,795,000 |
| | breakpoint | | | | |
| | coordinate | | | | |
| | window 2A | | | | |
| 23 | Approx. | chrX: | chr11: | chr11: | chr2: |
| | partner | 153,945,001-153,960,000 | 86,244,001-86,249,000 | 33,870,001-33,885,000 | 64,785,001-64,800,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | FIG. 16 | N/A |
| 26 | NOTES | | | | |
| 1 | VARIANT ID | 85 | 86 | 87 | 88 |
| 2 | SAMPLE | S52 | S53 | S54 | S54 |
| | NUMBER | | | | |
| 3 | Tumor type | Plasmacytoma | Plasmacytoma | High grade | High grade |
| | | | (hx of MM) | adenosarcoma | adenosarcoma |
| | | | | (HG AS) | (HG AS) |
| 4 | Partner 1 | break in WWOX | intergenic break | break in RAD51B | break in ELAVL3 |
| | type | | | | |
| 5 | Approx. | chr16: | chr20: | chr14: | chr19: |
| | breakpoint | 79,170,001-79,175,000 | 40,185,001-40,190,000 | 68,390,001-68,391,000 | 11,470,001-11,471,000 |
| | coordinate | | | | |
| | window 1A | | | | |
| 6 | Approx. | chr16: | chr20: | chr14: | chr19: |
| | breakpoint | 79,165,001-79,180,000 | 40,180,001-40,195,000 | 68,388,001-68,393,000 | 11,468,001-11,473,000 |
| | coordinate | | | | |
| | window 1B | | | | |
| 7 | Relevant | MAF | MAFB | RAD51B | SMARCA4 |
| | cancer | | | | |
| | gene(s) | | | | |
| 8 | Gene 5′ | chr16: 79,600,737 | chr20: 40,689,236 | chr14: 67,865,032 | chr19: 10,961,001 |
| 9 | Gene 3′ | chr16: 79,593,838 | chr20: 40,685,848 | chr14: 68,683,118 | chr19: 11,062,256 |
| 10 | Cancer Gene | Tier 3 | Tier 3 | Tier 1 | Tier 3 |
| | Tier | | | | |
| 11 | HRR GENE | NO | NO | YES | NO |
| 12 | Linear | 425737 | 499236 | N/A Break in Gene | 509000 |
| | distance to 5′ | | | | |
| | (bp) | | | | |
| 13 | Closest | 418838 | 495848 | N/A Break in Gene | 407745 |
| | distance to | | | | |
| | gene body | | | | |
| | (bp) | | | | |
| 14 | Partner 2 | break in MIR4507 | intergenic break | break in ME3 | intergenic break |
| | gene or | | | | |
| | intergenic | | | | |
| 15 | Relevant | IgH locus | IgH locus | N/A | N/A |
| | cancer | | | | |
| | gene(s) | | | | |
| 16 | Gene 5′ | IgH locus | IgH locus | N/A | N/A |
| 17 | Gene 3′ | IgH locus | IgH locus | N/A | N/A |
| 18 | Cancer Gene | Tier 4 | Tier 4 | N/A | N/A |
| | Tier | | | | |
| 19 | HRR GENE | NO | NO | N/A | N/A |
| 20 | Linear | IgH locus | IgH locus | N/A Break in Gene | N/A |
| | distance to 5′ | | | | |
| | (bp) | | | | |
| 21 | Closest | IgH locus | IgH locus | N/A Break in Gene | N/A |
| | distance to | | | | |
| | gene body | | | | |
| | (bp) | | | | |
| 22 | Approx. | chr14: | chr14: | chr11: | chr19: |
| | partner | 105,855,001-105,860,000 | 105,740,001-105,745,000 | 86,463,001-86,464,000 | 13,562,001-13,563,000 |
| | breakpoint | | | | |
| | coordinate | | | | |
| | window 2A | | | | |
| 23 | Approx. | chr14: | chr14: | chr11: | chr19: |
| | partner | 105,850,001-105,865,000 | 105,735,001-105,750,000 | 86,461,001-86,466,000 | 13,560,001-13,565,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 89 | 90 | 91 | 92 |
| 2 | SAMPLE | S54 | S55 | S55 | S55 |
| | NUMBER | | | | |
| 3 | Tumor type | High grade | Undifferentiated | Undifferentiated | Undifferentiated |
| | | adenosarcoma | Uterine Sarcoma | Uterine Sarcoma | Uterine Sarcoma |
| | | (HG AS) | (UUS) - Uterine | (UUS) - Uterine | (UUS) - Uterine |
| 4 | Partner 1 | break in SYN2 | break in CDON | break in AHRR | intergenic break |
| | type | | | | |
| 5 | Approx. | chr3: | chr11: | chr5: | chr5: |
| | breakpoint | 12,075,001-12,080,000 | 126,000,001-126,005,000 | 395,001-400,000 | 1,250,001-1,251,000 |
| | coordinate | | | | |
| | window 1A | | | | |
| 6 | Approx. | chr3: | chr11: | chr5: | chr5: |
| | breakpoint | 12,070,001-12,085,000 | 125,995,001-126,010,000 | 390,001-405,000 | 1,248,001-1,253,000 |
| | coordinate | | | | |
| | window 1B | | | | |
| 7 | Relevant | RAF1 | CHEK1 | SDHA | TERT |
| | cancer | | | | |
| | gene(s) | | | | |
| 8 | Gene 5′ | chr3: 12,664,187 | chr11: 125,625,974 | chr5: 218,320 | chr5: 1,295,068 |
| 9 | Gene 3′ | chr3: 12,582,101 | chr11: 125,676,255 | chr5: 257,082 | chr5: 1,253,167 |
| 10 | Cancer Gene | Tier 1 | Tier 1 | Tier 1 | Tier 3 |
| | Tier | | | | |
| 11 | HRR GENE | NO | YES | NO | NO |
| 12 | Linear | 584187 | 374027 | 176681 | 44068 |
| | distance to 5′ | | | | |
| | (bp) | | | | |
| 13 | Closest | 502101 | 323746 | 137919 | 2167 |
| | distance to | | | | |
| | gene body | | | | |
| | (bp) | | | | |
| 14 | Partner 2 | break in SLC25A26 | break in GAB2 | break in SNX1 | intergenic break |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A Break in Gene | N/A Break in Gene | N/A |
| | distance to 5′ | | | | |
| | (bp) | | | | |
| 21 | Closest | N/A Break in Gene | N/A Break in Gene | N/A Break in Gene | N/A |
| | distance to | | | | |
| | gene body | | | | |
| | (bp) | | | | |
| 22 | Approx. | chr3: | chr11: | chr15: | chr15: |
| | partner | 66,365,001-66,370,000 | 78,415,001-78,420,000 | 64,135,001-64,140,000 | 51,974,001-51,975,000 |
| | breakpoint | | | | |
| | coordinate | | | | |
| | window 2A | | | | |
| 23 | Approx. | chr3: | chr11: | chr15: | chr15: |
| | partner | 66,360,001-66,375,000 | 78,410,001-78,425,000 | 64,130,001-64,145,000 | 51,972,001-51,977,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 93 | 94 | 95 | 96 |
| 2 | SAMPLE | S56 | S56 | S56 | S57 |
| | NUMBER | | | | |
| 3 | Tumor type | High grade (HG) | High grade (HG) | High grade (HG) | HG spindle cell |
| | | spindle cell | spindle cell | spindle cell | an epithelioid |
| | | sarcoma | sarcoma | sarcoma | neoplasm c/w |
| | | | | | UUS |
| 4 | Partner 1 | intergenic break | break in PLEKHG4B | break in PPOX | break in NTRK3 |
| | type | | | | |
| 5 | Approx. | chr5: | chr5: | chr1: | chr15: |
| | breakpoint | 960,001-965,000 | 140,001-145,000 | 161,170,001-161,175,008 | 87,990,001-87,995,000 |
| | coordinate | | | | |
| | window 1A | | | | |
| 6 | Approx. | chr5: | chr5: | chr1: | chr15: |
| | breakpoint | 955,001-970,000 | 135,001-150,000 | 161,165,001-161,180,008 | 87,985,001-88,000,000 |
| | coordinate | | | | |
| | window 1B | | | | |
| 7 | Relevant | TERT | SDHA | SDHC | NTRK3 |
| | cancer | | | | |
| | gene(s) | | | | |
| 8 | Gene 5′ | chr5: 1,295,068 | chr5: 218,320 | chr1: 161,314,381 | chr15: 88,256,747 |
| 9 | Gene 3′ | chr5: 1,253,167 | chr5: 257,082 | chr1: 161,363,206 | chr15: 87,859,751 |
| 10 | Cancer Gene | Tier 3 | Tier 1 | Tier 1 | Tier 1 |
| | Tier | | | | |
| 11 | HRR GENE | NO | NO | NO | NO |
| 12 | Linear | 330068 | 73320 | 139373 | N/A Break in Gene |
| | distance to 5′ | | | | |
| | (bp) | | | | |
| 13 | Closest | 288167 | 73320 | 139373 | N/A Break in Gene |
| | distance to | | | | |
| | gene body | | | | |
| | (bp) | | | | |
| 14 | Partner 2 | intergenic break | break in NR1D2 | intergenic break | break in AKAP13 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A Break in Gene | N/A | N/A Break in Gene |
| | distance to 5′ | | | | |
| | (bp) | | | | |
| 21 | Closest | N/A | N/A Break in Gene | N/A | N/A Break in Gene |
| | distance to | | | | |
| | gene body | | | | |
| | (bp) | | | | |
| 22 | Approx. | chr3: | chr3: | chr1: | chr15: |
| | partner | 31,040,001-31,045,000 | 23,960,001-23,965,000 | 147,740,001-147,745,000 | 85,675,001-85,680,000 |
| | breakpoint | | | | |
| | coordinate | | | | |
| | window 2A | | | | |
| 23 | Approx. | chr3: | chr3: | chr1: | chr15: |
| | partner | 31,035,001-31,050,000 | 23,955,001-23,970,000 | 147,735,001-147,750,000 | 85,670,001-85,685,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 97 | 98 | 99 | 100 |
| 2 | SAMPLE | S57 | S57 | S57 | S57 |
| | NUMBER | | | | |
| 3 | Tumor type | HG spindle cell | HG spindle cell | HG spindle cell | HG spindle cell |
| | | an epithelioid | an epithelioid | an epithelioid | an epithelioid |
| | | neoplasm c/w | neoplasm c/w | neoplasm c/w | neoplasm c/w |
| | | UUS | UUS | UUS | UUS |
| 4 | Partner 1 | break in DEAF1 | break in NF1 | break in ARHGAP12 | break in MME |
| | type | | | | |
| 5 | Approx. | chr11: | chr17: | chr10: | chr3: |
| | breakpoint | 675,001-680,000 | 31,185,001-31,190,000 | 31,905,001-31,910,000 | 155,180,001-155,185,000 |
| | coordinate | | | | |
| | window 1A | | | | |
| 6 | Approx. | chr11: | chr17: | chr10: | chr3: |
| | breakpoint | 670,001-685,000 | 31,180,001-31,195,000 | 31,900,001-31,915,000 | 155,175,001-155,190,000 |
| | coordinate | | | | |
| | window 1B | | | | |
| 7 | Relevant | HRAS | NF1 | EPC1 | MME |
| | cancer | | | | |
| | gene(s) | | | | |
| 8 | Gene 5′ | chr11: 535,576 | chr17: 31,094,977 | chr10: 32,347,158 | chr3: 155,024,124 |
| 9 | Gene 3′ | chr11: 532,242 | chr17: 31,377,675 | chr10: 32,267,751 | chr3: 155,180,849 |
| 10 | Cancer Gene | Tier 1 | Tier 1 | Tier 3 | Tier 3 |
| | Tier | | | | |
| 11 | HRR GENE | NO | NO | NO | NO |
| 12 | Linear | 139425 | N/A Break in Gene | 437158 | N/A Break in Gene |
| | distance to 5′ | | | | |
| | (bp) | | | | |
| 13 | Closest | 139425 | N/A Break in Gene | 357751 | N/A Break in Gene |
| | distance to | | | | |
| | gene body | | | | |
| | (bp) | | | | |
| 14 | Partner 2 | intergenic break | intergenic break | break in ZBTB46 | intergenic break |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A | N/A Break in Gene | N/A |
| | distance to 5′ | | | | |
| | (bp) | | | | |
| 21 | Closest | N/A | N/A | N/A Break in Gene | N/A |
| | distance to | | | | |
| | gene body | | | | |
| | (bp) | | | | |
| 22 | Approx. | chr15: | chr10: | chr20: | chr3: |
| | partner | 87,615,001-87,620,000 | 106,525,001-106,530,000 | 63,765,001-63,770,000 | 166,485,001-166,490,000 |
| | breakpoint | | | | |
| | coordinate | | | | |
| | window 2A | | | | |
| 23 | Approx. | chr15: | chr10: | chr20: | chr3: |
| | partner | 87,610,001-87,625,000 | 106,520,001-106,535,000 | 63,760,001-63,775,000 | 166,480,001-166,495,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 101 | 102 | 103 | 104 |
| 2 | SAMPLE | S58 | S58 | S58 | S58 |
| | NUMBER | | | | |
| 3 | Tumor type | Adenosarcoma | Adenosarcoma | Adenosarcoma | Adenosarcoma |
| | | with sarcoma | with sarcoma | with sarcoma | with sarcoma |
| | | overgrowth | overgrowth | overgrowth | overgrowth |
| | | (ASSO) | (ASSO) | (ASSO) | (ASSO) |
| 4 | Partner 1 | break in KCNMB2 | intergenic break | break in ATM | intergenic break |
| | type | | | | |
| 5 | Approx. | chr3: | chrX: | chr11: | chrX: |
| | breakpoint | 178,735,001-178,740,000 | 67,110,001-67,115,000 | 108,275,001-108,280,000 | 101,755,001-101,760,000 |
| | coordinate | | | | |
| | window 1A | | | | |
| 6 | Approx. | chr3: | chrX: | chr11: | chrX: |
| | breakpoint | 178,730,001-178,745,000 | 67,105,001-67,120,000 | 108,270,001-108,285,000 | 101,750,001-101,765,000 |
| | coordinate | | | | |
| | window 1B | | | | |
| 7 | Relevant | PIK3CA | AR | ATM | BTK |
| | cancer | | | | |
| | gene(s) | | | | |
| 8 | Gene 5′ | chr3: 179,148,357 | chrX: 67,544,021 | chr11: 108,223,067 | chrX: 101,386,182 |
| 9 | Gene 3′ | chr3: 179,240,093 | chrX: 67,730,619 | chr11: 108,369,102 | chrX: 101,349,338 |
| 10 | Cancer Gene | Tier 1 | Tier 1 | Tier 1 | Tier 1 |
| | Tier | | | | |
| 11 | HRR GENE | NO | NO | YES | NO |
| 12 | Linear | 408357 | 429021 | N/A Break in Gene | 368819 |
| | distance to 5′ | | | | |
| | (bp) | | | | |
| 13 | Closest | 408357 | 429021 | N/A Break in Gene | 368819 |
| | distance to | | | | |
| | gene body | | | | |
| | (bp) | | | | |
| 14 | Partner 2 | break in SAMD7 | intergenic break | break in MSANTD2 | intergenic break |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A Break in Gene | N/A | N/A Break in Gene | N/A |
| | distance to 5′ | | | | |
| | (bp) | | | | |
| 21 | Closest | N/A Break in Gene | N/A | N/A Break in Gene | N/A |
| | distance to | | | | |
| | gene body | | | | |
| | (bp) | | | | |
| 22 | Approx. | chr3: | chrX: | chr11: | chrX: |
| | partner | 169,925,001-169,930,000 | 95,255,001-95,260,000 | 124,785,001-124,790,000 | 108,775,001-108,780,000 |
| | breakpoint | | | | |
| | coordinate | | | | |
| | window 2A | | | | |
| 23 | Approx. | chr3: | chrX: | chr11: | chrX: |
| | partner | 169,920,001-169,935,000 | 95,250,001-95,265,000 | 124,780,001-124,795,000 | 108,770,001-108,785,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | FIG. 22C | N/A | N/A |
| 26 | NOTES | | | | |
| 1 | VARIANT ID | 105 | 106 | 107 | 108 |
| 2 | SAMPLE | S58 | S58 | S58 | S58 |
| | NUMBER | | | | |
| 3 | Tumor type | Adenosarcoma | Adenosarcoma | Adenosarcoma | Adenosarcoma |
| | | with sarcoma | with sarcoma | with sarcoma | with sarcoma |
| | | overgrowth | overgrowth | overgrowth | overgrowth |
| | | (ASSO) | (ASSO) | (ASSO) | (ASSO) |
| 4 | Partner 1 | break in NR_038930 | break in AVIL | break in USP34 | break in SPOCD1 |
| | type | | | | |
| 5 | Approx. | chr12: | chr12: | chr2: | chr1: |
| | breakpoint | 68,685,001-68,690,000 | 57,800,001-57,801,000 | 61,260,001-61,265,000 | 31,814,001-31,815,000 |
| | coordinate | | | | |
| | window 1A | | | | |
| 6 | Approx. | chr12: | chr12: | chr2: | chr1: |
| | breakpoint | 68,680,001-68,695,000 | 57,798,001-57,803,000 | 61,255,001-61,270,000 | 31,812,001-31,817,000 |
| | coordinate | | | | |
| | window 1B | | | | |
| 7 | Relevant | MDM2 | CDK4 | XPO1 | HDAC1 |
| | cancer | | | | |
| | gene(s) | | | | |
| 8 | Gene 5′ | chr12: 68,809,002 | chr12: 57,752,310 | chr2: 61,538,741 | chr1: 32,292,083 |
| 9 | Gene 3′ | chr12: 68,840,807 | chr12: 57,747,727 | chr2: 61,477,689 | chr1: 32,333,626 |
| 10 | Cancer Gene | Tier 2 | Tier 2 | Tier 2 | Tier 2 |
| | Tier | | | | |
| 11 | HRR GENE | NO | NO | NO | NO |
| 12 | Linear | 119002 | 47691 | 273741 | 477083 |
| | distance to 5′ | | | | |
| | (bp) | | | | |
| 13 | Closest | 119002 | 47691 | 212689 | 477083 |
| | distance to | | | | |
| | gene body | | | | |
| | (bp) | | | | |
| 14 | Partner 2 | intergenic break | break in SRGAP1 | intergenic break | intergenic break |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A Break in Gene | N/A | N/A |
| | distance to 5′ | | | | |
| | (bp) | | | | |
| 21 | Closest | N/A | N/A Break in Gene | N/A | N/A |
| | distance to | | | | |
| | gene body | | | | |
| | (bp) | | | | |
| 22 | Approx. | chr11: | chr12: | chr2: | chr20: |
| | partner | 57,175,001-57,180,000 | 64,116,001-64,117,000 | 10,540,001-10,545,000 | 58,721,001-58,722,000 |
| | breakpoint | | | | |
| | coordinate | | | | |
| | window 2A | | | | |
| 23 | Approx. | chr11: | chr12: | chr2: | chr20: |
| | partner | 57,170,001-57,185,000 | 64,114,001-64,119,000 | 10,535,001-10,550,000 | 58,719,001-58,724,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | FIG. 22A | FIG. 22B | N/A | N/A |
| 26 | NOTES | | | | |
| 1 | VARIANT ID | 109 | 110 | 111 | 112 |
| 2 | SAMPLE | S58 | S58 | S59 | S59 |
| | NUMBER | | | | |
| 3 | Tumor type | Adenosarcoma | Adenosarcoma | Glioma | Glioma |
| | | with sarcoma | with sarcoma | | |
| | | overgrowth | overgrowth | | |
| | | (ASSO) | (ASSO) | | |
| 4 | Partner 1 | break in CCDC7 | intergenic break | break in CAPZA2 | break in PDIA4 |
| | type | | | | |
| 5 | Approx. | chr10: | chr20: | chr7: | chr7: |
| | breakpoint | 32,753,001-32,754,000 | 47,925,001-47,930,000 | 116,915,001-116,920,000 | 149,005,001-149,010,000 |
| | coordinate | | | | |
| | window 1A | | | | |
| 6 | Approx. | chr10: | chr20: | chr7: | chr7: |
| | breakpoint | 32,751,001-32,756,000 | 47,920,001-47,935,000 | 116,910,001-116,925,000 | 149,000,001-149,015,000 |
| | coordinate | | | | |
| | window 1B | | | | |
| 7 | Relevant | EPC1 | NCOA3 | MET | EZH2 |
| | cancer | | | | |
| | gene(s) | | | | |
| 8 | Gene 5′ | chr10: 32,347,158 | chr20: 47,501,887 | chr7: 116,672,196 | chr7: 148,884,291 |
| 9 | Gene 3′ | chr10: 32,267,751 | chr20: 47,656,872 | chr7: 116,798,377 | chr7: 148,807,383 |
| 10 | Cancer Gene | Tier 3 | Tier 3 | Tier 1 | Tier 1 |
| | Tier | | | | |
| 11 | HRR GENE | NO | NO | NO | NO |
| 12 | Linear | 405843 | 423114 | 242805 | 120710 |
| | distance to 5′ | | | | |
| | (bp) | | | | |
| 13 | Closest | 405843 | 268129 | 116624 | 120710 |
| | distance to | | | | |
| | gene body | | | | |
| | (bp) | | | | |
| 14 | Partner 2 | intergenic break | break in CNTN4 | Intergenic | break in SMARCD3 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer Gene | N/A | N/A | N/A | N/A |
| | Tier | | | | |
| 19 | HRR GENE | N/A | N/A | N/A | N/A |
| 20 | Linear | N/A | N/A Break in Gene | N/A | N/A |
| | distance to 5′ | | | | |
| | (bp) | | | | |
| 21 | Closest | N/A | N/A Break in Gene | N/A | N/A |
| | distance to | | | | |
| | gene body | | | | |
| | (bp) | | | | |
| 22 | Approx. | chr10: | chr3: | chr7: | chr7: |
| | partner | 73,996,001-73,997,000 | 2,460,001-2,465,000 | 148,480,001-148,485,000 | 151,265,001-151,270,000 |
| | breakpoint | | | | |
| | coordinate | | | | |
| | window 2A | | | | |
| 23 | Approx. | chr10: | chr3: | 7: | chr7: |
| | partner | 73,994,001-73,999,000 | 2,455,001-2,470,000 | 148,475,001-148,490,000 | 151,260,001-151,275,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | 1 | 1 |
| 1 | VARIANT ID | 113 | 114 | 115 | 116 |
| 2 | SAMPLE | S60 | S61 | S62 | S62 |
| | NUMBER | | | | |
| 3 | Tumor type | Myxoid | Burkitt lymphoma, | Plasmacytoma | Plasmacytoma |
| | | leiomyosarcoma | HIV, EBV+ | | |
| 4 | Partner 1 | Intergenic break | break in MYC | Intergenic break | break in TENT5C |
| | type | | | | |
| 5 | Approx. | chr2: | chr8: | chr11: | chr1: |
| | breakpoint | 202,590,001-202,595,000 | 127,736,001-127,737,000 | 69,510,001-69,515,000 | 117,613,001-117,614,000 |
| | coordinate | | | | |
| | window 1A | | | | |
| 6 | Approx. | chr2: | chr8: | chr11: | chr1: |
| | breakpoint | 202,585,001-202,600,000 | 127,729,001-127,744,000 | 69,505,001-69,520,000 | 117,608,001-117,619,000 |
| | coordinate | | | | |
| | window 1B | | | | |
| 7 | Relevant | BMPR2 | MYC | CCND1 | TENT5C |
| | cancer | | | FGF19 | |
| | gene(s) | | | FGF4 | |
| | | | | FGF3 | |
| 8 | Gene 5′ | chr2: 202,376,327 | chr8: 127,736,084 | CCND1: | chr1: 117,606,048 |
| | | | | chr11: 69,641,156 | |
| | | | | FGF19: | |
| | | | | chr11: 69,704,022 | |
| | | | | FGF4: | |
| | | | | chr11: 69,775,341 | |
| | | | | FGF3: | |
| | | | | chr11: 69,819,416 | |
| 9 | Gene 3′ | chr2: 202,567,749 | chr8: 127,741,434 | CCND1: | chr1: 117,628,389 |
| | | | | chr11: 69,654,474FGF19: | |
| | | | | chr11: 69,698,238FGF4: | |
| | | | | chr11: 69,771,022FGF3: | |
| | | | | chr11: 69,809,968 | |
| 10 | Cancer Gene | Tier 4 | Tier 3 | CCND1: Tier 3 | Tier 4 |
| | Tier | | | Others: Tier 4 | |
| 11 | HRR GENE | NO | NO | NO | NO |
| 12 | Linear | 213674 | N/A Break in Gene | CCND1: 126,156 | N/A Break in Gene |
| | distance to 5′ | | | FGF19: 189,022 | |
| | (bp) | | | FGF4: 260,341 | |
| | | | | FGF3: 304,416 | |
| 13 | Closest | 22252 | N/A Break in Gene | CCND1: 126,156 | N/A Break in Gene |
| | distance to | | | FGF19: 183,238 | |
| | gene body | | | FGF4: 256,022 | |
| | (bp) | | | FGF3: 294,968 | |
| 14 | Partner 2 | Intergenic break | Intergenic break | Intergenic break | break in TGFBR3 |
| | gene or | | | | |
| | intergenic | | | | |
| 15 | Relevant | N/A | IgH locus | IgH locus | TGFBR3 |
| | cancer | | | | |
| | gene(s) | | | | |
| 16 | Gene 5′ | N/A | IgH locus | IgH locus | chr1: 91,886,151 |
| 17 | Gene 3′ | N/A | IgH locus | IgH locus | chr1: 91,680,343 |
| 18 | Cancer Gene | N/A | Tier 4 | Tier 4 | Tier 4 |
| | Tier | | | | |
| 19 | HRR GENE | N/A | NO | NO | NO |
| 20 | Linear | N/A | IgH locus | IgH locus | N/A Break in Gene |
| | distance to 5′ | | | | |
| | (bp) | | | | |
| 21 | Closest | N/A | IgH locus | IgH locus | N/A Break in Gene |
| | distance to | | | | |
| | gene body | | | | |
| | (bp) | | | | |
| 22 | Approx. | chr10: | chr14: | chr14: | chr1: |
| | partner | 112,060,001-112,065,000 | 105,752,001-105,753,000 | 105,858,001-105,859,000 | 91,844,001-91,845,000 |
| | breakpoint | | | | |
| | coordinate | | | | |
| | window 2A | | | | |
| 23 | Approx. | chr10: | chr14: 14: | chr14: | chr1: |
| | partner | 112,055,001-112,070,000 | 105,749,001-105,756,000 | 105,854,001-105,863,000 | 91,839,001-91,850,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 2 | 3 | |
| 1 | VARIANT ID | 117 | 118 | 119 | 120 |
| 2 | SAMPLE | S62 | S63 | S64 | S64 |
| | NUMBER | | | | |
| 3 | Tumor type | Plasmacytoma | Plasmacytoma | Triple Negative | Triple Negative |
| | | | | Breast Cancer | Breast Cancer |
| 4 | Partner 1 | break in TENT5C | break in LINC01488 | Intergenic break | break in PVT1 |
| | type | | | | |
| 5 | Approx. | chr1: | chr11: | chr10: | chr8: |
| | breakpoint | 117,613,001-117,614,000 | 69,485,001-69,490,000 | 87,214,001-87,215,000 | 128,000,001-128,000,500 |
| | coordinate | | | | |
| | window 1A | | | | |
| 6 | Approx. | chr1: | chr11: | chr10: | chr8: |
| | breakpoint | 117,608,001-117,619,000 | 69,480,001-69,495,000 | 87,212,001-87,217,000 | 127,998,001-128,002,500 |
| | coordinate | | | | |
| | window 1B | | | | |
| 7 | Relevant | TENT5C | CCND1 | NUTM2A | N/A |
| | cancer | | FGF19 | | |
| | gene(s) | | FGF4 | | |
| | | | FGF3 | | |
| 8 | Gene 5′ | chr1: 117,606,048 | CCND1: | chr10: 87,225,448 | N/A |
| | | | chr11: 69,641,156 | | |
| | | | FGF19: | | |
| | | | chr11: 69,704,022 | | |
| | | | FGF4: | | |
| | | | chr11: 69,775,341 | | |
| | | | FGF3: | | |
| | | | chr11: 69,819,416 | | |
| 9 | Gene 3′ | chr1: 117,628,389 | CCND1: | chr10: 87,234,978 | N/A |
| | | | chr11: 69,654,474FGF19: | | |
| | | | chr11: 69,698,238FGF4: | | |
| | | | chr11: 69,771,022FGF3: | | |
| | | | chr11: 69,809,968 | | |
| 10 | Cancer Gene | Tier 4 | CCND1: Tier 3 | Tier 4 | N/A |
| | Tier | | Others: Tier 4 | | |
| 11 | HRR GENE | NO | NO | NO | N/A |
| 12 | Linear | N/A Break in Gene | CCND1: 151,156 | 10448 | N/A |
| | distance to 5′ | | FGF19: 214,022 | | |
| | (bp) | | FGF4: 285,341 | | |
| | | | FGF3: 329,416 | | |
| 13 | Closest | N/A Break in Gene | CCND1: 151,156 | 10448 | N/A |
| | distance to | | FGF19: 208,238 | | |
| | gene body | | FGF4: 281,022 | | |
| | (bp) | | FGF3: 319,968 | | |
| 14 | Partner 2 | Intergenic break | Intergenic break | Intergenic break | Intergenic break |
| | gene or | | | | |
| | intergenic | | | | |
| 15 | Relevant | N/A | IgH locus | N/A | MYC |
| | cancer | | | | |
| | gene(s) | | | | |
| 16 | Gene 5′ | N/A | IgH locus | N/A | chr8: 127,736,084 |
| 17 | Gene 3′ | N/A | IgH locus | N/A | chr8: 127,741,434 |
| 18 | Cancer Gene | N/A | Tier 4 | N/A | Tier 4 |
| | Tier | | | | |
| 19 | HRR GENE | N/A | NO | N/A | NO |
| 20 | Linear | N/A | IgH locus | N/A | 57917 |
| | distance to 5′ | | | | |
| | (bp) | | | | |
| 21 | Closest | N/A | IgH locus | N/A | 52567 |
| | distance to | | | | |
| | gene body | | | | |
| | (bp) | | | | |
| 22 | Approx. | chr1: | chr14: | chr18: | chr8: |
| | partner | 92,793,501-92,794,000 | 105,859,001-105,860,000 | 2,147,001-2,148,000 | 127,794,001-127,794,500 |
| | breakpoint | | | | |
| | coordinate | | | | |
| | window 2A | | | | |
| 23 | Approx. | chr1: | chr14: | chr18: | chr8: |
| | partner | 92,791,001-92,796,000 | 105,855,001-105,864,000 | 2,145,001-2,150,000 | 127,792,001-127,796,500 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 4 | | |
| 1 | VARIANT ID | 121 | 122 | 123 | 124 |
| 2 | SAMPLE | S64 | S64 | S65 | S66 |
| | NUMBER | | | | |
| 3 | Tumor type | Triple Negative | Triple Negative | Glioblastoma | Classic Hodgkins |
| | | Breast Cancer | Breast Cancer | | lymphoma |
| 4 | Partner 1 | break in EPHB1 | Intergenic break | Intergenic break | break in ANKS6 |
| | type | | | | |
| 5 | Approx. | chr3: | chr9: | chr9: | chr9: |
| | breakpoint | 135,155,001-135,160,000 | 10,890,001-10,895,000 | 5,475,001-5,476,000 | 98,795,001-98,800,000 |
| | coordinate | | | | |
| | window 1A | | | | |
| 6 | Approx. | chr3: | chr9: | chr9: | chr9: |
| | breakpoint | 135,150,001-135,165,000 | 10,885,001-10,900,000 | 5,471,000-5,480,000 | 98,790,001-98,805,000 |
| | coordinate | | | | |
| | window 1B | | | | |
| 7 | Relevant | EPHB1 | PTPRD | PD-L1 (CD274) | N/A |
| | cancer | | | PD-L2 (CD273) | |
| | gene(s) | | | | |
| 8 | Gene 5′ | chr3: 134,795,260 | chr9: 10,613,002 | PD-L1 (CD274): | N/A |
| | | | | chr9: 5,450,542 | |
| | | | | PD-L2 (CD273): | |
| | | | | chr9: 5,510,531 | |
| 9 | Gene 3′ | chr3: 135,260,467 | chr9: 8,314,246 | PD-L1 (CD274): | N/A |
| | | | | chr9: 5,470,554PD- | |
| | | | | L2 (CD273): | |
| | | | | chr9: 5,571,282 | |
| 10 | Cancer Gene | Tier 4 | Tier 4 | PD-L1 (CD274): | N/A |
| | Tier | | | Tier 1 | |
| | | | | PD-L2 (CD273): | |
| | | | | Tier 4 | |
| 11 | HRR GENE | NO | NO | NO | N/A |
| 12 | Linear | N/A Break in Gene | 276999 | PD-L1 (CD274): 24,459 | N/A |
| | distance to 5′ | | | PD-L2 (CD273): 34,531 | |
| | (bp) | | | | |
| 13 | Closest | N/A Break in Gene | 276999 | PD-L1 (CD274): 4,447 | N/A |
| | distance to | | | PD-L2 (CD273): 34,531 | |
| | gene body | | | | |
| | (bp) | | | | |
| 14 | Partner 2 | break in SIDT1 | Intergenic | Intergenic | Intergenic |
| | gene or | | | | |
| | intergenic | | | | |
| 15 | Relevant | N/A | PD-L1 (CD274) | N/A | PTPRD |
| | cancer | | PD-L2 (CD273) | | |
| | gene(s) | | | | |
| 16 | Gene 5′ | N/A | PD-L1 (CD274): | N/A | chr9: 10,613,002 |
| | | | chr9: 5,450,542PD- | | |
| | | | L2 (CD273): | | |
| | | | chr9: 5,510,531 | | |
| 17 | Gene 3′ | N/A | PD-L1 (CD274): | N/A | chr9: 8,314,246 |
| | | | chr9: 5,470,554 | | |
| | | | PD-L2 (CD273): | | |
| | | | chr9: 5,571,282 | | |
| 18 | Cancer Gene | N/A | PD-L1 (CD274): | N/A | Tier 4 |
| | Tier | | Tier 1 PD-L2 | | |
| | | | (CD273): Tier 4 | | |
| 19 | HRR GENE | N/A | NO | N/A | NO |
| 20 | Linear | N/A | PD-L1 (CD274): 624,459 | N/A | 1626999 |
| | distance to 5′ | | PD-L2 (CD273): 564,470 | | |
| | (bp) | | | | |
| 21 | Closest | N/A | PD-L1 (CD274): 604,447 | N/A | 1626999 |
| | distance to | | PD-L2 (CD273): 503,719 | | |
| | gene body | | | | |
| | (bp) | | | | |
| 22 | Approx. | chr3: | chr9: | Chr9: | chr9: |
| | partner | 113,575,001-113,580,000 | 6,075,001-6,080,000 | 18,381,001-18,382,000 | 12,240,001-12,245,000 |
| | breakpoint | | | | |
| | coordinate | | | | |
| | window 2A | | | | |
| 23 | Approx. | chr3: | chr9: | Chr9: | chr9: |
| | partner | 113,570,001-113,585,000 | 6,070,001-6,085,000 | 18,377,000-18,386,000 | 12,235,001-12,250,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | FIG. 23 | N/A |
| 26 | NOTES | 5 | | | |
| 1 | VARIANT ID | 125 | 126 | 127 | 128 |
| 2 | SAMPLE | S67 | S68 | S69 | S69 |
| | NUMBER | | | | |
| 3 | Tumor type | Osseous | Plasmacytoma | Diffuse large B | Diffuse large B |
| | | plasmcytoma | | cell lymphoma | cell lymphoma |
| 4 | Partner 1 | Intergenic break | Intergenic break | break in BCL6 | break in BCL6 |
| | type | | | | |
| 5 | Approx. | chr11: | chr14: | chr3: | chr3: |
| | breakpoint | 69,275,001-69,280,000 | 96,017,001-96,018,000 | 187,740,001-187,745,000 | 187,745,001-187,750,000 |
| | coordinate | | | | |
| | window 1A | | | | |
| 6 | Approx. | chr11: | chr14: | chr3: | chr3: |
| | breakpoint | 69,270,001-69,285,000 | 96,015,001-96,020,000 | 187,735,001-187,750,000 | 187,740,001-187,755,000 |
| | coordinate | | | | |
| | window 1B | | | | |
| 7 | Relevant | CCND1 | N/A | BCL6 | BCL6 |
| | cancer | FGF19 | | | |
| | gene(s) | FGF4 | | | |
| | | FGF3 | | | |
| 8 | Gene 5′ | CCND1: | N/A | chr3: 187,745,468 | chr3: 187,745,468 |
| | | chr11: 69,641,156 | | | |
| | | FGF19: | | | |
| | | chr11: 69,704,022 | | | |
| | | FGF4: | | | |
| | | chr11: 69,775,341 | | | |
| | | FGF3: | | | |
| | | chr11: 69,819,416 | | | |
| 9 | Gene 3′ | CCND1: | N/A | chr3: 187,721,381 | chr3: 187,721,381 |
| | | chr11: 69,654,474FGF19: | | | |
| | | chr11: 69,698,238FGF4: | | | |
| | | chr11: 69,771,022FGF3: | | | |
| | | chr11: 69,809,968 | | | |
| 10 | Cancer Gene | CCND1: Tier 3 | N/A | Tier 3 | Tier 3 |
| | Tier | Others: Tier 4 | | | |
| 11 | HRR GENE | NO | N/A | NO | NO |
| 12 | Linear | CCND1: 361156 | N/A | N/A Break in Gene | N/A Break in Gene |
| | distance to 5′ | FGF19: 424,022 | | | |
| | (bp) | FGF4: 495,341 | | | |
| | | FGF3: 539,416 | | | |
| 13 | Closest | CCND1: 361156 | N/A | N/A Break in Gene | N/A Break in Gene |
| | distance to | FGF19: 418,238 | | | |
| | gene body | FGF4: 491,022 | | | |
| | (bp) | FGF3: 529,968 | | | |
| 14 | Partner 2 | break in IGHG3 | break in NIN | Intergenic | Intergenic |
| | gene or | | | | |
| | intergenic | | | | |
| 15 | Relevant | IgH locus | NIN | N/A | N/A |
| | cancer | | | | |
| | gene(s) | | | | |
| 16 | Gene 5′ | IgH locus | chr14: 50,831,121 | N/A | N/A |
| 17 | Gene 3′ | IgH locus | chr14: 50,725,840 | N/A | N/A |
| 18 | Cancer Gene | Tier 4 | Tier 4 | N/A | N/A |
| | Tier | | | | |
| 19 | HRR GENE | NO | NO | N/A | N/A |
| 20 | Linear | IgH locus | N/A Break in Gene | N/A | N/A |
| | distance to 5′ | | | | |
| | (bp) | | | | |
| 21 | Closest | IgH locus | N/A Break in Gene | N/A | N/A |
| | distance to | | | | |
| | gene body | | | | |
| | (bp) | | | | |
| 22 | Approx. | chr14: | chr14: | chr22: | chr22: |
| | partner | 105,765,001-105,770,000 | 50,811,001-50,812,000 | 22,935,001-22,940,000 | 22,695,001-22,700,000 |
| | breakpoint | | | | |
| | coordinate | | | | |
| | window 2A | | | | |
| 23 | Approx. | chr14: | chr14: | chr22: | chr22: |
| | partner | 105,735,001-105,770,000 | 50,809,001-50,814,000 | 22,930,001-22,945,000 | 22,690,001-22,705,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | 6 | | 7 | |
| 1 | VARIANT ID | 129 | 130 | 131 | 132 |
| 2 | SAMPLE | S69 | S70 | S71 | S71 |
| | NUMBER | | | | |
| 3 | Tumor type | Diffuse large B | Chordoma | Diffuse large B | Diffuse large B |
| | | cell lymphoma | | cell lymphoma | cell lymphoma |
| 4 | Partner 1 | break in MIR1291 | break in NSD2 | Intergenic break | break in PTH2 |
| | type | | | | |
| 5 | Approx. | chr12: | chr4: | chr13: | chr19: |
| | breakpoint | 48,655,001-48,660,000 | 1,875,001-1,880,000 | 54,980,001-54,985,000 | 49,420,001-49,430,000 |
| | coordinate | | | | |
| | window 1A | | | | |
| 6 | Approx. | chr12: | chr4: | chr13: | chr19: |
| | breakpoint | 48,645,001-48,670,000 | 1,870,001-1,885,000 | 54,975,001-54,990,000 | 49,410,001-49,440,000 |
| | coordinate | | | | |
| | window 1B | | | | |
| 7 | Relevant | KMT2D | NSD2 | N/A | N/A |
| | cancer | | FGFR3 | | |
| | gene(s) | | | | |
| 8 | Gene 5′ | chr12: 49,060,794 | NSD2: | N/A | N/A |
| | | | chr4: 1,871,393 | | |
| | | | FGFR3: | | |
| | | | chr4: 1,793,293 | | |
| 9 | Gene 3′ | chr12: 49,018,978 | NSD2: | N/A | N/A |
| | | | chr4: 1,982,192FG | | |
| | | | FR3: | | |
| | | | chr4: 1,808,867 | | |
| 10 | Cancer Gene | Tier 4 | NSD2: Tier 4 | N/A | N/A |
| | Tier | | FGFR3: Tier 1 | | |
| 11 | HRR GENE | NO | NO | N/A | N/A |
| 12 | Linear | 400794 | NSD2: N/A Break in Gene | N/A | N/A |
| | distance to 5′ | | | | |
| | (bp) | | FGFR3: 81,708 | | |
| 13 | Closest | 358978 | NSD2: N/A Break in Gene | N/ | N/A |
| | distance to | | | | |
| | gene body | | FGFR3: 66,134 | | |
| | (bp) | | | | |
| 14 | Partner 2 | break in UTY | break in BCR | break in ATP8A2 | break in WDR18 |
| | gene or | | | | |
| | intergenic | | | | |
| 15 | Relevant | N/A | BCR | CDK8 | STK11 |
| | cancer | | | | |
| | gene(s) | | | | |
| 16 | Gene 5′ | N/A | chr22: 23,180,509 | chr13: 26,254,129 | chr19: 1,205,778 |
| 17 | Gene 3′ | N/A | chr22: 23,318,037 | chr13: 26,405,238 | chr19: 1,228,431 |
| 18 | Cancer Gene | N/A | Tier 4 | Tier 4 | Tier 4 |
| | Tier | | | | |
| 19 | HRR GENE | N/A | NO | NO | NO |
| 20 | Linear | N/A | N/A Break in Gene | 499129 | 215778 |
| | distance to 5′ | | | | |
| | (bp) | | | | |
| 21 | Closest | N/A | N/A Break in Gene | 499129 | 215778 |
| | distance to | | | | |
| | gene body | | | | |
| | (bp) | | | | |
| 22 | Approx. | chrY: | chr22: | chr13: | chr19: |
| | partner | 13,360,001-13,365,000 | 23,305,001-23,310,000 | 25,750,001-25,755,000 | 980,001-990,000 |
| | breakpoint | | | | |
| | coordinate | | | | |
| | window 2A | | | | |
| 23 | Approx. | chrY: | chr22: | chr13: | chr19: |
| | partner | 13,350,001-13,375,000 | 23,300,001-23,315,000 | 25,745,001-25,760,000 | 970,001-1,000,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 8 | | |
| 1 | VARIANT ID | 133 | 134 | 135 | 136 |
| 2 | SAMPLE | S71 | S72 | S72 | S73 |
| | NUMBER | | | | |
| 3 | Tumor type | Diffuse large B | Pituitary adenoma | Pituitary adenoma | Myxoid |
| | | cell lymphoma | | | leiomyosarcoma |
| | | | | | (LMS) |
| 4 | Partner 1 | break in NLGN1 | break in FMR1 | Intergenic break | Intergenic break |
| | type | | | | |
| 5 | Approx. | chr3: | chrX: | chr11: | chr8: |
| | breakpoint | 174,220,001-174,230,000 | 147,912,001-147,913,000 | 124,550,001-124,555,000 | 56,129,001-56,130,000 |
| | coordinate | | | | |
| | window 1A | | | | |
| 6 | Approx. | chr3: | chrX: | chr11: | chr8: |
| | breakpoint | 174,210,001-174,240,000 | 147,909,001-147,916,000 | 124,545,001-124,560,000 | 56,127,001-56,132,000 |
| | coordinate | | | | |
| | window 1B | | | | |
| 7 | Relevant | N/A | FMR1 | N/A | PLAG1 |
| | cancer | | | | |
| | gene(s) | | | | |
| 8 | Gene 5′ | N/A | chrX: 147,911,919 | N/A | PLAG1: |
| | | | | | chr8: 56,211,273 |
| 9 | Gene 3′ | N/A | chrX: 147,951,125 | N/A | PLAG1: |
| | | | | | chr8: 56,160,909 |
| 10 | Cancer Gene | N/A | Tier 4 | N/A | Tier 3 |
| | Tier | | | | |
| 11 | HRR GENE | N/A | NO | N/A | NO |
| 12 | Linear | N/A | N/A Break in Gene | N/A | 81273 |
| | distance to 5′ | | | | |
| | (bp) | | | | |
| 13 | Closest | N/A | N/A Break in Gene | N/A | 30909 |
| | distance to | | | | |
| | gene body | | | | |
| | (bp) | | | | |
| 14 | Partner 2 | intergenic break | break in SIN3A | break in PAK1 | break in RAD51B |
| | gene or | | | | |
| | intergenic | | | | |
| 15 | Relevant | MME | SIN3A | PAK1 | RAD51B |
| | cancer | | | | |
| | gene(s) | | | | |
| 16 | Gene 5′ | chr3: 155,024,124 | chr15: 75,455,783 | chr11: 77,474,094 | chr14: 67,865,032 |
| 17 | Gene 3′ | chr3: 155,180,849 | chr15: 75,370,933 | chr11: 77,322,017 | chr14: 68,683,118 |
| 18 | Cancer Gene | Tier 3 | Tier 4 | Tier 4 | Tier 1 |
| | Tier | | | | |
| 19 | HRR GENE | NO | NO | NO | YES |
| 20 | Linear | 164124 | N/A Break in Gene | N/A Break in Gene | N/A Break in Gene |
| | distance to 5′ | | | | |
| | (bp) | | | | |
| 21 | Closest | 164124 | N/A Break in Gene | N/A Break in Gene | N/A Break in Gene |
| | distance to | | | | |
| | gene body | | | | |
| | (bp) | | | | |
| 22 | Approx. | chr3: | chr15: | chr11: | chr14: |
| | partner | 154,850,001-154,860,000 | 75,449,001-75,450,000 | 77,470,001-77,475,000 | 68,523,001-68,524,000 |
| | breakpoint | | | | |
| | coordinate | | | | |
| | window 2A | | | | |
| 23 | Approx. | chr3: | chr15: | chr11: | chr14: |
| | partner | 154,840,001-154,870,000 | 75,446,001-75,453,000 | 77,465,001-77,480,000 | 68,521,001-68,526,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 9 | | 10 |
| 1 | VARIANT ID | 137 | 138 | 139 | 140 |
| 2 | SAMPLE | S73 | S73 | S73 | S73 |
| | NUMBER | | | | |
| 3 | Tumor type | Myxoid | Myxoid | Myxoid | Myxoid |
| | | leiomyosarcoma | leiomyosarcoma | leiomyosarcoma | leiomyosarcoma |
| | | (LMS) | (LMS) | (LMS) | (LMS) |
| 4 | Partner 1 | break in RP1 | Intergenic break | break in NAPSA | break in FAM71E1 |
| | type | | | | |
| 5 | Approx. | chr8: | chr11: | chr19: | chr19: |
| | breakpoint | 54,695,001-54,700,000 | 168,001-169,000 | 50,365,001-50,366,000 | 50,460,001-50,470,000 |
| | coordinate | | | | |
| | window 1A | | | | |
| 6 | Approx. | chr8: | chr11: | chr19: | chr19: |
| | breakpoint | 54,690,001-54,705,000 | 165,001-172,000 | 50,362,001-50,369,000 | 50,455,001-50,475,000 |
| | coordinate | | | | |
| | window 1B | | | | |
| 7 | Relevant | N/A | HRAS | POLD1 | N/A |
| | cancer | | | | |
| | gene(s) | | | | |
| 8 | Gene 5′ | N/A | chr11: 535,576 | chr19: 50,384,323 | N/A |
| 9 | Gene 3′ | N/A | chr11: 532,242 | chr19: 50,418,018 | N/A |
| 10 | Cancer Gene | N/A | Tier 1 | Tier 4 | N/A |
| | Tier | | | | |
| 11 | HRR GENE | N/A | NO | NO | N/A |
| 12 | Linear | N/A | 366576 | 18323 | N/A |
| | distance to 5′ | | | | |
| | (bp) | | | | |
| 13 | Closest | N/A | 363242 | 18323 | N/A |
| | distance to | | | | |
| | gene body | | | | |
| | (bp) | | | | |
| 14 | Partner 2 | break in RAD51B | break in TXNDC16 | Intergenic | break in LINC01480 |
| | gene or | | | | |
| | intergenic | | | | |
| 15 | Relevant | RAD51B | N/A | N/A | TGFB1 |
| | cancer | | | | AXL |
| | gene(s) | | | | |
| 16 | Gene 5′ | chr14: 67,865,032 | N/A | N/A | TGFB1: |
| | | | | | chr19: 41,353,922 |
| | | | | | AXL: |
| | | | | | chr19: 41,219,223 |
| 17 | Gene 3′ | chr14: 68,683,118 | N/A | N/A | TGFB1: |
| | | | | | chr19: 41,330,323 |
| | | | | | AXL: |
| | | | | | chr19: 41,261,766 |
| 18 | Cancer Gene | Tier 1 | N/A | N/A | TGFB1: Tier 4 |
| | Tier | | | | AXL: Tier 2 |
| 19 | HRR GENE | YES | N/A | N/A | NO |
| 20 | Linear | N/A Break in Gene | N/A | N/A | TGFB1: 176,079 |
| | distance to 5′ | | | | AXL: 310,778 |
| | (bp) | | | | |
| 21 | Closest | N/A Break in Gene | N/A | N/A | TGFB1: 176,079 |
| | distance to | | | | AXL: 268,235 |
| | gene body | | | | |
| | (bp) | | | | |
| 22 | Approx. | chr14: | chr14: | chr19: | chr19: |
| | partner | 68,525,001-68,530,000 | 52,505,001-52,506,000 | 36,246,001-36,247,000 | 41,530,001-41,540,000 |
| | breakpoint | | | | |
| | coordinate | | | | |
| | window 2A | | | | |
| 23 | Approx. | chr14: | chr14: | chr19: | chr19: |
| | partner | 68,520,001-68,535,000 | 52,502,001-52,509,000 | 36,243,001-36,250,000 | 41,525,001-41,545,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 141 | 142 | 143 | 144 |
| 2 | SAMPLE | S73 | S74 | S74 | S74 |
| | NUMBER | | | | |
| 3 | Tumor type | Myxoid | Diffuse large B | Diffuse large B | Diffuse large B |
| | | leiomyosarcoma | cell lymphoma | cell lymphoma | cell lymphoma |
| | | (LMS) | | | |
| 4 | Partner 1 | break in LINC01480 | intergenic break | intergenic break | intergenic break |
| | type | | | | |
| 5 | Approx. | chr19: | chr9: | chr1: | chr1: |
| | breakpoint | 41,535,001-41,540,000 | 5,505,001-5,510,000 | 157,117,001-157,118,000 | 157,117,001-157,118,000 |
| | coordinate | | | | |
| | window 1A | | | | |
| 6 | Approx. | chr19: | chr9: | chr1: | chr1: |
| | breakpoint | 41,530,001-41,545,000 | 5,500,001-5,515,000 | 157,114,001-157,121,000 | 157,114,001-157,121,000 |
| | coordinate | | | | |
| | window 1B | | | | |
| 7 | Relevant | TGFB1 | PD-L1 (CD274) | ETV3 | ETV3 |
| | cancer | AXL | PD-L2 (CD273) | | |
| | gene(s) | | JAK2 | | |
| 8 | Gene 5′ | TGFB1: | PD-L1 (CD274): | chr1: 157,138,395 | chr1: 157,138,395 |
| | | chr19: 41,353,922 | chr9: 5,450,542 | | |
| | | AXL: | PD-L2 (CD273): | | |
| | | chr19: 41,219,223 | chr9: 5,510,531 | | |
| | | | JAK2: | | |
| | | | chr9: 4,985,272 | | |
| 9 | Gene 3′ | TGFB1: | PD-L1 (CD274): | chr1: 157,121,191 | chr1: 157,121,191 |
| | | chr19: 41,330,323AXL: | chr9: 5,470,554PD- | | |
| | | chr19: 41,261,766 | L2 (CD273): | | |
| | | | chr9: 5,571,282JAK2: | | |
| | | | chr9: 5,129,948 | | |
| 10 | Cancer Gene | TGFB1: Tier 4 | PD-L1 (CD274); | Tier 4 | Tier 4 |
| | Tier | AXL: Tier 2 | JAK2: Tier 1 | | |
| | | | PD-L2 (CD273): Tier 4 | | |
| 11 | HRR GENE | NO | NO | NO | NO |
| 12 | Linear | TGFB1: 181,079 | PD-L1 (CD274): 54,459 | 20395 | 20395 |
| | distance to 5′ | AXL: 315,778 | PD-L2 (CD273): 531 | | |
| | (bp) | | JAK2: 519,729 | | |
| 13 | Closest | TGFB1: 181,079 | PD-L1 (CD274): 34,447 | 3191 | 3191 |
| | distance to | AXL: 273,235 | PD-L2 (CD273): 531 | | |
| | gene body | | JAK2: 375,053 | | |
| | (bp) | | | | |
| 14 | Partner 2 | break in ZNF565 | break in IGHA1 | intergenic break | intergenic break |
| | gene or | | | | |
| | intergenic | | | | |
| 15 | Relevant | N/A | N/A | ROS1 | VGLL2 |
| | cancer | | | | |
| | gene(s) | | | | |
| 16 | Gene 5′ | N/A | N/A | ROS1: | ROS1: |
| | | | | chr6: 117,425,942V | chr6: 117,425,942 |
| | | | | GLL2: | VGLL2: |
| | | | | chr6: 117,265,558 | chr6: 117,265,558 |
| 17 | Gene 3′ | N/A | N/A | ROS1: | ROS1: |
| | | | | chr6: 117,287,353 | chr6: 117,287,353 |
| | | | | VGLL2: | VGLL2: |
| | | | | chr6: 117,273,565 | chr6: 117,273,565 |
| 18 | Cancer Gene | N/A | N/A | Tier 1 | Tier 4 |
| | Tier | | | | |
| 19 | HRR GENE | N/A | N/A | NO | NO |
| 20 | Linear | N/A | N/A | ROS1: 378,059 | ROS1: 378,059 |
| | distance to 5′ | | | VGLL2: 538,443 | VGLL2: 538,443 |
| | (bp) | | | | |
| 21 | Closest | N/A | N/A | ROS1: 378,059 | ROS1: 378,059 |
| | distance to | | | VGLL2: 530,436 | VGLL2: 530,436 |
| | gene body | | | | |
| | (bp) | | | | |
| 22 | Approx. | chr19: | chr14: | chr6: | chr6: |
| | partner | 36,245,001-36,250,000 | 105,705,001-105,710,000 | 117,804,001-117,805,000 | 117,804,001-117,805,000 |
| | breakpoint | | | | |
| | coordinate | | | | |
| | window 2A | | | | |
| 23 | Approx. | chr19: | chr14: | chr6: | chr6: |
| | partner | 36,240,001-36,255,000 | 105,700,001-105,715,000 | 117,801,001-117,808,000 | 117,801,001-117,808,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 11 | | |
| 1 | VARIANT ID | 145 | 146 | 147 | 148 |
| 2 | SAMPLE | S74 | S74 | S74 | S74 |
| | NUMBER | | | | |
| 3 | Tumor type | Diffuse large B | Diffuse large B | Diffuse large B | Diffuse large B |
| | | cell lymphoma | cell lymphoma | cell lymphoma | cell lymphoma |
| 4 | Partner 1 | break in BCL6 | intergenic break | intergenic break | intergenic break |
| | type | | | | |
| 5 | Approx. | chr3: | chr1: | chr1: | chr2: |
| | breakpoint | 187,740,001-187,745,000 | 155,232,001-155,233,000 | 155,232,001-155,233,000 | 164,860,001-164,865,000 |
| | coordinate | | | | |
| | window 1A | | | | |
| 6 | Approx. | chr3: | chr1: | chr1: | chr2: |
| | breakpoint | 187,735,001-187,750,000 | 155,230,001-155,235,000 | 155,230,001-155,235,000 | 164,855,001-164,870,000 |
| | coordinate | | | | |
| | window 1B | | | | |
| 7 | Relevant | BCL6 | ASH1L | ASH1L | N/A |
| | cancer | | | | |
| | gene(s) | | | | |
| 8 | Gene 5′ | chr3: 187,745,468 | ASH1L: | ASH1L: | N/A |
| | | | chr1: 155,563,162 | chr1: 155,563,162 | |
| 9 | Gene 3′ | chr3: 187,721,381 | ASH1L: | ASH1L: | N/A |
| | | | chr1: 155,335,287 | chr1: 155,335,287 | |
| 10 | Cancer Gene | Tier 3 | Tier 4 | Tier 4 | N/A |
| | Tier | | | | |
| 11 | HRR GENE | NO | NO | NO | N/A |
| 12 | Linear | N/A Break in Gene | ASH1L: 330,162 | ASH1L: 330,162 | N/A |
| | distance to 5′ | | | | |
| | (bp) | | | | |
| 13 | Closest | N/A Break in Gene | ASH1L: 102,287 | ASH1L: 102,287 | N/A |
| | distance to | | | | |
| | gene body | | | | |
| | (bp) | | | | |
| 14 | Partner 2 | intergenic break | break in PPARG | break in PPARG | intergenic break |
| | gene or | | | | |
| | intergenic | | | | |
| 15 | Relevant | N/A | PPARG | RAF1 | ACVR1C |
| | cancer | | | | |
| | gene(s) | | | | |
| 16 | Gene 5′ | N/A | PPARG: | PPARG: | chr2: 157,628,864 |
| | | | chr3: 12,287,368R | chr3: 12,287,368RA | |
| | | | AF1: | F1: | |
| | | | chr3: 12,664,117 | chr3: 12,664,117 | |
| 17 | Gene 3′ | N/A | PPARG: | PPARG: | chr2: 157,526,767 |
| | | | chr3: 12,434,344 | chr3: 12,434,344 | |
| | | | RAF1: | RAF1: | |
| | | | chr3: 12,583,601 | chr3: 12,583,601 | |
| 18 | Cancer Gene | N/A | Tier 4 | Tier 1 | Tier 4 |
| | Tier | | | | |
| 19 | HRR GENE | N/A | NO | NO | NO |
| 20 | Linear | N/A | PPARG: N/A Break in Gene | PPARG: N/A Break in Gene | 6137 |
| | distance to 5′ | | RAF1: 236,117 | RAF1: 236,117 | |
| | (bp) | | PPARG: N/A Break in Gene | PPARG: N/A Break in Gene | |
| 21 | Closest | N/A | RAF1: 155,601 | RAF1: 155,601 | 6137 |
| | distance to | | | | |
| | gene body | | | | |
| | (bp) | | | | |
| 22 | Approx. | chr14: | chr3: | chr3: | 2: |
| | partner | 105,885,001-105,890,000 | 12,427,001-12,428,000 | 12,427,001-12,428,000 | 157,635,001-157,640,000 |
| | breakpoint | | | | |
| | coordinate | | | | |
| | window 2A | | | | |
| 23 | Approx. | chr14: | chr3: | chr3: | 2: |
| | partner | 105,880,001-105,895,000 | 12,425,001-12,430,000 | 12,425,001-12,430,000 | 157,630,001-157,645,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | 12 | | | |
| 1 | VARIANT ID | 149 | 150 | 151 | 152 |
| 2 | SAMPLE | S74 | S74 | S75 | S75 |
| | NUMBER | | | | |
| 3 | Tumor type | Diffuse large B | Diffuse large B | Leiomyosarcoma | Leiomyosarcoma |
| | | cell lymphoma | cell lymphoma | | |
| 4 | Partner 1 | break in TENM3 | intergenic break | break in TATDN2 | intergenic break |
| | type | | | | |
| 5 | Approx. | chr4: | chr1: | chr3: | chr6: |
| | breakpoint | 182,250,001-182,255,000 | 206,030,001-206,035,000 | 10,267,001-10,268,000 | 44,105,001-44,110,000 |
| | coordinate | | | | |
| | window 1A | | | | |
| 6 | Approx. | chr4: | chr1: | chr3: | chr6: |
| | breakpoint | 182,245,001-182,260,000 | 206,025,001-206,040,000 | 10,264,001-10,271,000 | 44,100,001-44,115,000 |
| | coordinate | | | | |
| | window 1B | | | | |
| 7 | Relevant | N/A | RAB29 | VHL | VEGFA |
| | cancer | | SLC45A3 | | |
| | gene(s) | | | | |
| 8 | Gene 5′ | N/A | RAB29: | chr3: 10,141,778 | chr6: 43,771,209 |
| | | | chr1: 205,775,482 | | |
| | | | SLC45A3: | | |
| | | | chr1: 205,680,509 | | |
| 9 | Gene 3′ | N/A | RAB29: | chr3: 10,153,667 | chr6: 43,784,902 |
| | | | chr1: 205,767,986S | | |
| | | | LC45A3: | | |
| | | | chr1: 205,657,851 | | |
| 10 | Cancer Gene | N/A | RAB29; SLC45A3: | Tier 1 | Tier 4 |
| | Tier | | Tier 4 | | |
| 11 | HRR GENE | N/A | NO | NO | NO |
| 12 | Linear | N/A | RAB29: 254,519 | 125223 | 333792 |
| | distance to 5′ | | SLC45A3: 349,492 | | |
| | (bp) | | | | |
| 13 | Closest | N/A | RAB29: 254,519 | 113334 | 320099 |
| | distance to | | SLC45A3: 349,492 | | |
| | gene body | | | | |
| | (bp) | | | | |
| 14 | Partner 2 | break in SLC9A5 | intergenic break | intergenic break | break in EPN2 |
| | gene or | | | | |
| | intergenic | | | | |
| 15 | Relevant | CBFB | N/A | MYC | N/A |
| | cancer | | | | |
| | gene(s) | | | | |
| 16 | Gene 5′ | chr16: 67,029,149 | N/A | chr8: 127,736,084 | N/A |
| 17 | Gene 3′ | chr16: 67,101,058 | N/A | chr8: 127,741,434 | N/A |
| 18 | Cancer Gene | Tier 4 | N/A | Tier 4 | N/A |
| | Tier | | | | |
| 19 | HRR GENE | NO | N/A | NO | N/A |
| 20 | Linear | 220852 | N/A | 1444917 | N/A |
| | distance to 5′ | | | | |
| | (bp) | | | | |
| 21 | Closest | 148943 | V/A | 1439567 | N/A |
| | distance to | | | | |
| | gene body | | | | |
| | (bp) | | | | |
| 22 | Approx. | chr16: | chr18: | chr8: | chr17: |
| | partner | 67,250,001-67,255,000 | 78,025,001-78,030,000 | 129,181,001-129,182,000 | 19,235,001-19,240,000 |
| | breakpoint | | | | |
| | coordinate | | | | |
| | window 2A | | | | |
| 23 | Approx. | chr16: | chr18: | chr8: | chr17: |
| | partner | 67,245,001-67,260,000 | 78,025,001-78,030,000 | 129,178,001-129,185,000 | 19,230,001-19,245,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 153 | 154 | 155 | 156 |
| 2 | SAMPLE | S75 | S75 | S76 | S76 |
| | NUMBER | | | | |
| 3 | Tumor type | Leiomyosarcoma | leiomyosarcoma | Diffuse large B | Diffuse large B |
| | | | (LMS) | cell lymphoma | cell lymphoma |
| 4 | Partner 1 | break in GBA | break in TATDN2 | intergenic break | break in TSPOAP1-AS1 |
| | type | | | | |
| 5 | Approx. | chr1: | chr3: | chr10: | chr17: |
| | breakpoint | 155,240,001-155,245,000 | 10,267,001-10,268,000 | 43,380,001-43,385,000 | 58,332,001-58,333,000 |
| | coordinate | | | | |
| | window 1A | | | | |
| 6 | Approx. | chr1: | chr3: | chr10: | chr17: |
| | breakpoint | 155,235,001-155,250,000 | 10,264,001-10,271,000 | 43,375,001-43,390,000 | 58,329,001-58,337,000 |
| | coordinate | | | | |
| | window 1B | | | | |
| 7 | Relevant | ASH1L | VHL | RET | RAD51C |
| | cancer | | FANCD2 | | RNF43 |
| | gene(s) | | | | |
| 8 | Gene 5′ | chr1: 155,563,162 | VHL: | chr10: 43,077,069 | RAD51C: |
| | | | chr3: 10,141,778 | | chr17: 58,692,602 |
| | | | FANCD2: | | RNF43: |
| | | | chr3: 10,026,437 | | chr17: 58,417,582 |
| 9 | Gene 3′ | chr1: 155,335,287 | VHL: | chr10: 43,130,351 | RAD51C: |
| | | | chr3: 10,153,667FA | | chr17: 58,735,611 |
| | | | NCD2: | | RNF43: |
| | | | chr3: 10,101,932 | | chr17: 58,353,676 |
| 10 | Cancer Gene | Tier 4 | VHL: Tier 1 | Tier 1 | RAD51C: Tier 1 |
| | Tier | | FANCD2: Tier 1 | | RNF43: Tier 4 |
| 11 | HRR GENE | NO | VHL: No | NO | RAD51C: YES |
| | | | FANCD2: Yes | | RNF43: NO |
| 12 | Linear | 318162 | VHL: 125,223 | 302932 | RAD51C: 359,602 |
| | distance to 5′ | | FANCD2: 240,564 | | RNF43: 84,582 |
| | (bp) | | | | |
| 13 | Closest | 90287 | VHL: 113,334 | 249650 | RAD51C: 359,602 |
| | distance to | | FANCD2: 165,069 | | RNF43: 20,676 |
| | gene body | | | | |
| | (bp) | | | | |
| 14 | Partner 2 | intergenic break | intergenic break | break in FAM107B | break in COPE |
| | gene or | | | | |
| | intergenic | | | | |
| 15 | Relevant | N/A | N/A | N/A | MEF2B |
| | cancer | | | | |
| | gene(s) | | | | |
| 16 | Gene 5′ | N/A | N/A | N/A | chr19: 19,192,131 |
| 17 | Gene 3′ | N/A | N/A | N/A | chr19: 19,145,567 |
| 18 | Cancer Gene | N/A | N/A | N/A | Tier 4 |
| | Tier | | | | |
| 19 | HRR GENE | N/A | N/A | N/A | NO |
| 20 | Linear | N/A | N/A | N/A | 288131 |
| | distance to 5′ | | | | |
| | (bp) | | | | |
| 21 | Closest | N/A | N/A | N/A | 241567 |
| | distance to | | | | |
| | gene body | | | | |
| | (bp) | | | | |
| 22 | Approx. | chr7: | chr8: | chr10: | chr19: |
| | partner | 159,220,001-159,225,000 | 129,181,001-129,182,000 | 14,555,001-14,560,000 | 18,903,001-18,904,000 |
| | breakpoint | | | | |
| | coordinate | | | | |
| | window 2A | | | | |
| 23 | Approx. | chr7: | chr8: | chr10: | chr19: |
| | partner | 159,215,001-159,230,000 | 129,178,001-129,185,000 | 14,550,001-14,565,000 | 18,900,001-18,907,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 157 | 158 | 159 | 160 |
| 2 | SAMPLE | S76 | S76 | S76 | S76 |
| | NUMBER | | | | |
| 3 | Tumor type | Diffuse large B | Diffuse large B | Diffuse large B | Diffuse large B |
| | | cell lymphoma | cell lymphoma | cell lymphoma | cell lymphoma |
| 4 | Partner 1 | break in ZNF250 | intergenic break | intergenic break | break in MIR142 |
| | type | | | | |
| 5 | Approx. | chr8: | chr14: | chr10: | chr17: |
| | breakpoint | 144,878,001-144,879,008 | 105,774,001-105,775,000 | 90,640,001-90,645,000 | 58,330,001-58,335,000 |
| | coordinate | | | | |
| | window 1A | | | | |
| 6 | Approx. | chr8: | chr14: | chr10: | chr17: |
| | breakpoint | 144,875,001-144,882,008 | 105,771,001-105,778,000 | 90,635,001-90,650,000 | 58,325,001-58,340,000 |
| | coordinate | | | | |
| | window 1B | | | | |
| 7 | Relevant | RECQL4 | N/A | N/A | N/A |
| | cancer | | | | |
| | gene(s) | | | | |
| 8 | Gene 5′ | chr8: 144,517,833 | N/A | N/A | N/A |
| 9 | Gene 3′ | chr8: 144,511,288 | N/A | N/A | N/A |
| 10 | Cancer Gene | Tier 4 | N/A | N/A | N/A |
| | Tier | | | | |
| 11 | HRR GENE | NO | N/A | N/A | N/A |
| 12 | Linear | 360168 | N/A | N/A | N/A |
| | distance to 5′ | | | | |
| | (bp) | | | | |
| 13 | Closest | 360168 | N/A | N/A | N/A |
| | distance to | | | | |
| | gene body | | | | |
| | (bp) | | | | |
| 14 | Partner 2 | break in CLEC17A | break in JDP2 | break in MINPP1 | break in KLHL26 |
| | gene or | | | | |
| | intergenic | | | | |
| 15 | Relevant | PRKACA | FOS | NUTM2A | PIK3R2 |
| | cancer | PKN1 | MLH3 | | |
| | gene(s) | DNAJB1 | | | |
| 16 | Gene 5′ | PRKACA: | FOS: | chr10: 87,225,448 | chr19: 18,153,163 |
| | | chr19: 14,117,762PKN1: | chr14: 75,278,828 | | |
| | | chr19: 14,433,306DNAJB1: | MLH3: | | |
| | | chr19: 14,529,300 | chr14: 75,051,467 | | |
| 17 | Gene 3′ | PRKACA: | FOS: | chr10: 87,234,978 | chr19: 18,170,532 |
| | | chr19: 14,091,688 | chr14: 75,282,230 | | |
| | | PKN1: | MLH3: | | |
| | | chr19: 14,471,859 | chr14: 75,013,775 | | |
| | | DNAJB1: | | | |
| | | chr19: 14,514,769 | | | |
| 18 | Cancer Gene | Tier 4 | Tier 4 | Tier 4 | Tier 4 |
| | Tier | | | | |
| 19 | HRR GENE | NO | NO | NO | NO |
| 20 | Linear | PRKACA: 480,239 | FOS: 146,173 | 284553 | 496838 |
| | distance to 5′ | PKN1: 164,695 | MLH3: 373,534 | | |
| | (bp) | DNAJB1: 68,701 | | | |
| 21 | Closest | PRKACA: 480,239 | FOS: 142,771 | 275023 | 479469 |
| | distance to | PKN1: 126,142 | MLH3: 373,534 | | |
| | gene body | DNAJB1: 68,701 | | | |
| | (bp) | | | | |
| 22 | Approx. | chr19: | chr14: | chr10: | chr19: |
| | partner | 14,598,001-14,599,000 | 75,425,001-75,430,000 | 87,510,001-87,515,000 | 18,650,001-18,655,000 |
| | breakpoint | | | | |
| | coordinate | | | | |
| | window 2A | | | | |
| 23 | Approx. | chr19: | chr14: | chr10: | chr19: |
| | partner | 14,595,001-14,602,000 | 75,420,001-75,435,000 | 87,505,001-87,520,000 | 18,645,001-18,660,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 13 | | |
| 1 | VARIANT ID | 161 | 162 | 163 | 164 |
| 2 | SAMPLE | S76 | S76 | S76 | S76 |
| | NUMBER | | | | |
| 3 | Tumor type | Diffuse large B | Diffuse large B | Diffuse large B | Diffuse large B |
| | | cell lymphoma | cell lymphoma | cell lymphoma | cell lymphoma |
| 4 | Partner 1 | break in ZNF589 | intergenic break | break in TP63 | break in TP63 |
| | type | | | | |
| 5 | Approx. | chr3: | chr3: | chr3: | chr3: |
| | breakpoint | 48,240,001-48,245,000 | 49,437,001-49,438,000 | 189,715,001-189,720,000 | 189,710,001-189,715,008 |
| | coordinate | | | | |
| | window 1A | | | | |
| 6 | Approx. | chr3: | chr3: | chr3: | chr3: |
| | breakpoint | 48,235,001-48,250,000 | 49,434,001-49,441,000 | 189,710,001-189,725,000 | 189,705,001-189,720,008 |
| | coordinate | | | | |
| | window 1B | | | | |
| 7 | Relevant | N/A | MST1R | TP63 | TP63 |
| | cancer | | | | |
| | gene(s) | | | | |
| 8 | Gene 5′ | N/A | chr3: 49,903,873 | chr3: 189,631,389 | chr3: 189,631,389 |
| 9 | Gene 3′ | N/A | chr3: 49,887,002 | chr3: 189,897,276 | chr3: 189,897,276 |
| 10 | Cancer Gene | N/A | Tier 4 | Tier 3 | Tier 3 |
| | Tier | | | | |
| 11 | HRR GENE | N/A | NO | NO | NO |
| 12 | Linear | N/A | 465873 | N/A break in gene | N/A break in gene |
| | distance to 5′ | | | | |
| | (bp) | | | | |
| 13 | Closest | N/A | 449002 | N/A break in gene | N/A break in gene |
| | distance to | | | | |
| | gene body | | | | |
| | (bp) | | | | |
| 14 | Partner 2 | break in SCAP | break in SCAP | break in P2RY14 | break in GPR87 |
| | gene or | | | | |
| | intergenic | | | | |
| 15 | Relevant | SETD2 | N/A | N/A | N/A |
| | cancer | | | | |
| | gene(s) | | | | |
| 16 | Gene 5′ | chr3: 47,164,113 | N/A | N/A | N/A |
| 17 | Gene 3′ | chr3: 47,016,436 | N/A | N/A | N/A |
| 18 | Cancer Gene | Tier 4 | N/A | N/A | N/A |
| | Tier | | | | |
| 19 | HRR GENE | NO | N/A | N/A | N/A |
| 20 | Linear | 255888 | N/A | N/A | N/A |
| | distance to 5′ | | | | |
| | (bp) | | | | |
| 21 | Closest | 255888 | N/A | N/A | N/A |
| | distance to | | | | |
| | gene body | | | | |
| | (bp) | | | | |
| 22 | Approx. | chr3: | chr3: | chr3: | chr3: |
| | partner | 47,420,001-47,425,000 | 47,422,001-47,423,000 | 151,210,001-151,215,000 | 151,305,001-151,310,000 |
| | breakpoint | | | | |
| | coordinate | | | | |
| | window 2A | | | | |
| 23 | Approx. | chr3: | chr3: | chr3: | chr3: |
| | partner | 47,415,001-47,430,000 | 47,419,001-47,426,000 | 151,205,001-151,220,000 | 151,300,001-151,315,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 165 | 166 | 167 | 168 |
| 2 | SAMPLE | S76 | S76 | S77 | S77 |
| | NUMBER | | | | |
| 3 | Tumor type | Diffuse large B | Diffuse large B | Myxoid | Myxoid |
| | | cell lymphoma | cell lymphoma | leiomyosarcoma | leiomyosarcoma |
| | | | | (LMS) | (LMS) |
| 4 | Partner 1 | break in PIM1 | intergenic break | break in BCL11A | break in GALM |
| | type | | | | |
| 5 | Approx. | chr6: | chr2: | chr2: | chr2: |
| | breakpoint | 37,171,001-37,172,000 | 153,273,001-153,274,000 | 60,479,001-60,480,000 | 38,734,001-38,735,000 |
| | coordinate | | | | |
| | window 1A | | | | |
| 6 | Approx. | chr6: | chr2: | chr2: | chr2: |
| | breakpoint | 37,169,001-37,174,000 | 153,271,001-153,276,000 | 60,477,001-60,482,000 | 38,732,001-38,737,000 |
| | coordinate | | | | |
| | window 1B | | | | |
| 7 | Relevant | PIM1 | N/A | BCL11A | SOS1 |
| | cancer | | | REL | |
| | gene(s) | | | | |
| 8 | Gene 5′ | chr6: 37,170,152 | N/A | BCL11A: | chr2: 39,121,051 |
| | | | | chr2: 60,553,658 | |
| | | | | REL: | |
| | | | | chr2: 60,881,574 | |
| 9 | Gene 3′ | chr6: 37,175,428 | N/A | BCL11A: | chr2: 38,981,549 |
| | | | | chr2: 60,457,679REL: | |
| | | | | chr2: 60,931,612 | |
| 10 | Cancer Gene | Tier 4 | N/A | BCL11A; REL: Tier 4 | Tier 2 |
| | Tier | | | | |
| 11 | HRR GENE | NO | N/A | NO | NO |
| 12 | Linear | N/A break in gene | N/A | BCL11A: N/A Break in Gene | 386051 |
| | distance to 5′ | | | | |
| | (bp) | | | REL: 401,574 | |
| 13 | Closest | N/A break in gene | N/A | BCL11A: N/A Break in Gene | 246549 |
| | distance to | | | REL: 401,574 | |
| | gene body | | | | |
| | (bp) | | | | |
| 14 | Partner 2 | break in H3C7 | break in LRP1B | intergenic break | break in SULF2 |
| | gene or | | | | |
| | intergenic | | | | |
| 15 | Relevant | H3C2 | LRP1B | N/A | NCOA3 |
| | cancer | HI-2 | | | |
| | gene(s) | HI-3 | | | |
| | | H2AC6 | | | |
| 16 | Gene 5′ | H3C2: | chr2: 142,131,016 | N/A | chr20: 47,501,887 |
| | | chr6: 26,032,099H1-2: | | | |
| | | chr6: 26,056,470H1-4: | | | |
| | | chr6: 26,156,329H1-3: | | | |
| | | chr6: 26,234,987H2AC6: | | | |
| | | chr6: 26,124,203 | | | |
| 17 | Gene 3′ | H3C2: | chr2: 140,231,423 | N/A | chr20: 47,656,872 |
| | | chr6: 26,031,589 | | | |
| | | H1-2: | | | |
| | | chr6: 26,055,740 | | | |
| | | H1-4: | | | |
| | | chr6: 26,157,115 | | | |
| | | H1-3: | | | |
| | | chr6: 26,234,212 | | | |
| | | H2AC6: | | | |
| | | chr6: 26,139,084 | | | |
| 18 | Cancer Gene | Tier 4 | Tier 4 | N/A | Tier 3 |
| | Tier | | | | |
| 19 | HRR GENE | NO | NO | N/A | NO |
| 20 | Linear | H3C2: 217,902 | N/A break in gene | N/A | 195114 |
| | distance to 5′ | H1-2: 193,531 | | | |
| | (bp) | H1-4: 93,672 | | | |
| | | H1-3: 15,014 | | | |
| | | H2AC6: 125,798 | | | |
| 21 | Closest | H3C2: 217,902 | N/A break in gene | N/A | 40129 |
| | distance to | H1-2: 193,531 | | | |
| | gene body | H1-4: 92,886 | | | |
| | (bp) | H1-3: 15,014 | | | |
| | | H2AC6: 110,917 | | | |
| 22 | Approx. | chr6: | chr2: | chr21: | chr20: |
| | partner | 26,250,001-26,251,000 | 140,680,001-140,681,000 | 34,219,001-34,220,000 | 47,697,001-47,698,000 |
| | breakpoint | | | | |
| | coordinate | | | | |
| | window 2A | | | | |
| 23 | Approx. | chr6: | chr2: | chr21: | chr20: |
| | partner | 26,247,001-26,254,000 | 140,678,001-140,683,000 | 34,217,001-34,222,000 | 47,695,001-47,700,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 169 | 170 | 171 | 172 |
| 2 | SAMPLE | S77 | S77 | S77 | S78 |
| | NUMBER | | | | |
| 3 | Tumor type | Myxoid | Myxoid | Myxoid | Myxoid |
| | | leiomyosarcoma | leiomyosarcoma | leiomyosarcoma | leiomyosarcoma |
| | | (LMS) | (LMS) | (LMS) | (LMS) |
| 4 | Partner 1 | break in SGPP2 | break in PTGFRN | break in DDAH1 | break in CPS13B |
| | type | | | | |
| 5 | Approx. | chr2: | chr1: | chr1: | chr8: |
| | breakpoint | 222,460,001-222,465,000 | 116,945,001-116,950,000 | 85,460,001-85,465,000 | 99,413,001-99,414,000 |
| | coordinate | | | | |
| | window 1A | | | | |
| 6 | Approx. | chr2: | chr1: | chr1: | chr8: |
| | breakpoint | 222,455,001-222,470,000 | 116,940,001-116,955,000 | 85,455,001-85,470,000 | 99,411,001-99,416,000 |
| | coordinate | | | | |
| | window 1B | | | | |
| 7 | Relevant | PAX3 | N/A | N/A | N/A |
| | cancer | | | | |
| | gene(s) | | | | |
| 8 | Gene 5′ | chr2: 222,298,998 | N/A | N/A | N/A |
| 9 | Gene 3′ | chr2: 222,199,887 | N/A | N/A | N/A |
| 10 | Cancer Gene | Tier 4 | N/A | N/A | N/A |
| | Tier | | | | |
| 11 | HRR GENE | NO | N/A | N/A | N/A |
| 12 | Linear | 161003 | N/A | N/A | N/A |
| | distance to 5′ | | | | |
| | (bp) | | | | |
| 13 | Closest | 161003 | N/A | N/A | N/A |
| | distance to | | | | |
| | gene body | | | | |
| | (bp) | | | | |
| 14 | Partner 2 | break in LTBP1 | intergenic break | break in FUBP1 | intergenic break |
| | gene or | | | | |
| | intergenic | | | | |
| 15 | Relevant | N/A | RBM15 | FUBP1 | PLAG1 |
| | cancer | | | | |
| | gene(s) | | | | |
| 16 | Gene 5′ | N/A | chr1: 110,338,506 | chr1: 77,979,072 | chr8: 56,211,273 |
| 17 | Gene 3′ | N/A | chr1: 110,346,673 | chr1: 77,944,055 | chr8: 56,160,909 |
| 18 | Cancer Gene | N/A | Tier 4 | Tier 4 | Tier 3 |
| | Tier | | | | |
| 19 | HRR GENE | N/A | NO | NO | NO |
| 20 | Linear | N/A | 346495 | N/A break in gene | 107273 |
| | distance to 5′ | | | | |
| | (bp) | | | | |
| 21 | Closest | N/A | 338328 | N/A break in gene | 56909 |
| | distance to | | | | |
| | gene body | | | | |
| | (bp) | | | | |
| 22 | Approx. | chr2: | chr1: | chr1: | chr8: |
| | partner | 33,165,001-33,170,000 | 110,685,001-110,690,000 | 77,955,001-77,960,000 | 56,103,001-56,104,000 |
| | breakpoint | | | | |
| | coordinate | | | | |
| | window 2A | | | | |
| 23 | Approx. | chr2: | chr1: | chr1: | chr8: |
| | partner | 33,160,001-33,175,000 | 110,680,001-110,695,000 | 77,950,001-77,965,000 | 56,101,001-56,106,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 173 | 174 | 175 | 176 |
| 2 | SAMPLE | S78 | S78 | S78 | S78 |
| | NUMBER | | | | |
| 3 | Tumor type | Myxoid | Myxoid | Myxoid | Myxoid |
| | | leiomyosarcoma | leiomyosarcoma | leiomyosarcoma | leiomyosarcoma |
| | | (LMS) | (LMS) | (LMS) | (LMS) |
| 4 | Partner 1 | break in ZMAT4 | break in FAM189A1 | break in NOD2 | intergenic break |
| | type | | | | |
| 5 | Approx. | chr8: | chr15: | chr16: | chr1: |
| | breakpoint | 40,556,001-40,557,000 | 29,555,001-29,560,000 | 50,725,001-50,726,000 | 119,650,001-119,655,000 |
| | coordinate | | | | |
| | window 1A | | | | |
| 6 | Approx. | chr8: | chr15: | chr16: | chr1: |
| | breakpoint | 40,554,001-40,559,000 | 29,550,001-29,565,000 | 50,722,001-50,729,000 | 119,645,001-119,660,000 |
| | coordinate | | | | |
| | window 1B | | | | |
| 7 | Relevant | N/A | N/A | CYLD | HSD3B1 |
| | cancer | | | | |
| | gene(s) | | | | |
| 8 | Gene 5′ | N/A | N/A | chr16: 50,742,050 | chr1: 119,507,210 |
| 9 | Gene 3′ | N/A | N/A | chr16: 50,796,881 | chr1: 119,515,054 |
| 10 | Cancer Gene | N/A | N/A | Tier 4 | Tier 4 |
| | Tier | | | | |
| 11 | HRR GENE | N/A | N/A | NO | NO |
| 12 | Linear | N/A | N/A | 16050 | 142791 |
| | distance to 5′ | | | | |
| | (bp) | | | | |
| 13 | Closest | N/A | N/A | 16050 | 134947 |
| | distance to | | | | |
| | gene body | | | | |
| | (bp) | | | | |
| 14 | Partner 2 | intergenic break | intergenic break | break in PPP4R1 | break in IDO2 |
| | gene or | | | | |
| | intergenic | | | | |
| 15 | Relevant | NRG1 | BMP7 | N/A | N/A |
| | cancer | | | | |
| | gene(s) | | | | |
| 16 | Gene 5′ | chr8: 31,639,222 | chr20: 57,266,641 | N/A | N/A |
| 17 | Gene 3 | chr8: 32,764,405 | chr20: 57,168,753 | N/A | N/A |
| 18 | Cancer Gene | Tier 1 | Tier 4 | N/A | N/A |
| | Tier | | | | |
| 19 | HRR GENE | NO | NO | N/A | N/A |
| 20 | Linear | 322222 | 28360 | N/A | N/A |
| | distance to 5′ | | | | |
| | (bp) | | | | |
| 21 | Closest | 322222 | 28360 | N/A | N/A |
| | distance to | | | | |
| | gene body | | | | |
| | (bp) | | | | |
| 22 | Approx. | chr8: | chr20: | chr18: | chr8: |
| | partner | 31,316,001-31,317,000 | 57,295,001-57,300,000 | 9,614,001-9,615,000 | 40,010,001-40,015,000 |
| | breakpoint | | | | |
| | coordinate | | | | |
| | window 2A | | | | |
| 23 | Approx. | chr8: | chr20: | chr18: | chr8: |
| | partner | 31,314,001-31,319,000 | 57,290,001-57,305,000 | 9,611,001-9,618,000 | 40,005,001-40,020,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 177 | 178 | 179 | 180 |
| 2 | SAMPLE | S78 | S78 | S78 | S79 |
| | NUMBER | | | | |
| 3 | Tumor type | Myxoid | Myxoid | Myxoid | Meningioma |
| | | leiomyosarcoma | leiomyosarcoma | leiomyosarcoma | |
| | | (LMS) | (LMS) | (LMS) | |
| 4 | Partner 1 | intergenic break | break in SCCPDH | intergenic break | break in SMARCC2 |
| | type | | | | |
| 5 | Approx. | chr1: | chr1: | chr8: | chr12: |
| | breakpoint | 30,180,001-30,185,000 | 246,720,001-246,725,000 | 117,622,001-117,623,000 | 56,170,001-56,175,000 |
| | coordinate | | | | |
| | window 1A | | | | |
| 6 | Approx. | chr1: | chr1: | chr8: | chr12: |
| | breakpoint | 30,175,001-30,190,000 | 246,715,001-246,730,000 | 117,620,001-117,625,000 | 56,165,001-56,180,000 |
| | coordinate | | | | |
| | window 1B | | | | |
| 7 | Relevant | N/A | N/A | EXT1 | ERBB3 |
| | cancer | | | | CDK2 |
| | gene(s) | | | | |
| 8 | Gene 5 | N/A | N/A | chr8: 118,111,826 | ERBB3: |
| | | | | | chr12: 56,080,165 |
| | | | | | CDK2: |
| | | | | | chr12: 55,966,830 |
| 9 | Gene 3′ | N/A | N/A | chr8: 117,794,490 | ERBB3: |
| | | | | | chr12: 56,103,505 |
| | | | | | CDK2: |
| | | | | | chr12: 55,972,789 |
| 10 | Cancer Gene | N/A | N/A | Tier 4 | ERBB3; CDK2: Tier 2 |
| | Tier | | | | |
| 11 | HRR GENE | N/A | N/A | NO | NO |
| 12 | Linear | N/A | N/A | 488826 | ERBB3: 89,836 |
| | distance to 5′ | | | | CDK2: 203,171 |
| | (bp) | | | | |
| 13 | Closest | N/A | N/A | 171490 | ERBB3: 66,496 |
| | distance to | | | | CDK2: 197,212 |
| | gene body | | | | |
| | (bp) | | | | |
| 14 | Partner 2 | break in PAX7 | break in RYR2 | break in OSR2 | intergenic break |
| | gene or | | | | |
| | intergenic | | | | |
| 15 | Relevant | PAX7 | MTR | N/A | N/A |
| | cancer | | | | |
| | gene(s) | | | | |
| 16 | Gene 5′ | chr1: 18,630,846 | chr1: 236,795,292 | N/A | N/A |
| 17 | Gene 3′ | chr1: 18,748,866 | chr1: 236,903,981 | N/A | N/A |
| 18 | Cancer Gene | Tier 4 | Tier 4 | N/A | N/A |
| | Tier | | | | |
| 19 | HRR GENE | NO | NO | N/A | N/A |
| 20 | Linear | N/A break in gene | 494709 | N/A | N/A |
| | distance to 5′ | | | | |
| | (bp) | | | | |
| 21 | Closest | N/A break in gene | 386020 | N/A | N/A |
| | distance to | | | | |
| | gene body | | | | |
| | (bp) | | | | |
| 22 | Approx. | chr1: | chr1: | chr8: | chr12: |
| | partner | 18,660,001-18,665,000 | 237,290,001-237,295,000 | 98,945,001-98,946,000 | 47,025,001-47,030,000 |
| | breakpoint | | | | |
| | coordinate | | | | |
| | window 2A | | | | |
| 23 | Approx. | chr1: | chr1: | chr8: | chr12: |
| | partner | 18,655,001-18,670,000 | 237,285,001-237,300,000 | 98,943,001-98,948,000 | 47,020,001-47,035,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 181 | 182 | 183 | 184 |
| 2 | SAMPLE | S79 | S79 | S79 | S79 |
| | NUMBER | | | | |
| 3 | Tumor type | Meningioma | Meningioma | Meningioma | Meningioma |
| 4 | Partner 1 | break in KCNC2 | break in BAZ2A | break in TAMALIN | break in RAI1 |
| | type | | | | |
| 5 | Approx. | chr12: | chr12: | chr12: | chr17: |
| | breakpoint | 75,085,001-75,090,000 | 56,615,001-56,620,000 | 52,015,001-52,020,000 | 17,730,001-17,735,000 |
| | coordinate | | | | |
| | window 1A | | | | |
| 6 | Approx. | chr12: | chr12: | chr12: | chr17: |
| | breakpoint | 75,080,001-75,095,000 | 56,610,001-56,625,000 | 52,010,001-52,025,000 | 17,725,001-17,740,000 |
| | coordinate | | | | |
| | window 1B | | | | |
| 7 | Relevant | N/A | NAB2 | ACVR1B | GID4 |
| | cancer | | STAT6 | | |
| | gene(s) | | | | |
| 8 | Gene 5′ | N/A | NAB2: | chr12: 51,951,699 | chr17: 18,039,408 |
| | | | chr12: 57,089,114 | | |
| | | | STAT6: | | |
| | | | chr12: 57,129,100 | | |
| 9 | Gene 3′ | N/A | NAB2: | chr12: 51,997,078 | chr17: 18,068,405 |
| | | | chr12: 57,095,476STAT6: | | |
| | | | chr12: 57,096,341 | | |
| 10 | Cancer Gene | N/A | NAB2; STAT6: Tier 4 | Tier 4 | Tier 4 |
| | Tier | | | | |
| 11 | HRR GENE | N/A | NO | NO | NO |
| 12 | Linear | N/A | NAB2: 469,114 | 63302 | 304408 |
| | distance to 5′ | | STAT6: 509,100 | | |
| | (bp) | | | | |
| 13 | Closest | N/A | NAB2: 469,114 | 17923 | 304408 |
| | distance to | | STAT6: 476,341 | | |
| | gene body | | | | |
| | (bp) | | | | |
| 14 | Partner 2 | intergenic break | break in LOC339260 | break in TMEM117 | intergenic break |
| | gene or | | | | |
| | intergenic | | | | |
| 15 | Relevant | CDK4 | N/A | ADAMTS20 | N/A |
| | cancer | DDIT3 | | | |
| | gene(s) | | | | |
| 16 | Gene 5′ | CDK4: | N/A | chr12: 43,552,203 | N/A |
| | | chr12: 57,752,310DDIT3: | | | |
| | | chr12: 57,521,737 | | | |
| 17 | Gene 3′ | CDK4: | N/A | chr12: 43,353,866 | N/A |
| | | chr12: 57,747,727 | | | |
| | | DDIT3: | | | |
| | | chr12: 57,516,588 | | | |
| 18 | Cancer Gene | CDK2: Tier 2 | N/A | Tier 4 | N/A |
| | Tier | DDIT3: Tier 4 | | | |
| 19 | HRR GENE | NO | N/A | NO | N/A |
| 20 | Linear | CDK4: 262,691 | N/A | 582798 | N/A |
| | distance to 5′ | DDIT3 493,264 | | | |
| | (bp) | | | | |
| 21 | Closest | CDK4: 262,691 | N/A | 582798 | N/A |
| | distance to | DDIT3 493,264 | | | |
| | gene body | | | | |
| | (bp) | | | | |
| 22 | Approx. | chr12: | chr17: | chr12: | chr17: |
| | partner | 58,015,001-58,020,000 | 20,940,001-20,945,000 | 44,135,001-44,140,000 | 15,210,001-15,215,000 |
| | breakpoint | | | | |
| | coordinate | | | | |
| | window 2A | | | | |
| 23 | Approx. | chr12: | chr17: | chr12: | chr17: |
| | partner | 58,010,001-58,025,000 | 20,935,001-20,950,000 | 44,130,001-44,145,000 | 15,205,001-15,220,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 185 | 186 | 187 | 188 |
| 2 | SAMPLE | S79 | S80 | S80 | S80 |
| | NUMBER | | | | |
| 3 | Tumor type | Meningioma | Chordoma | Chordoma | Chordoma |
| 4 | Partner 1 | intergenic break | break in CCNYL1 | break in XCR1 | intergenic break |
| | type | | | | |
| 5 | Approx. | chr12: | chr2: | chr3: | chr9: |
| | breakpoint | 43,260,001-43,265,000 | 207,735,001-207,740,000 | 46,070,001-46,075,000 | 31,986,001-31,987,000 |
| | coordinate | | | | |
| | window 1A | | | | |
| 6 | Approx. | chr12: | chr2: | chr3: | chr9: |
| | breakpoint | 43,255,001-43,270,000 | 207,730,001-207,745,000 | 46,065,001-46,080,000 | 31,983,001-31,990,000 |
| | coordinate | | | | |
| | window 1B | | | | |
| 7 | Relevant | N/A | IDH1 | LTF | TAF1L |
| | cancer | | CREB1 | LIMD1 | |
| | gene(s) | | | | |
| 8 | Gene 5′ | N/A | IDH1: | LTF: | chr9: 32,635,669 |
| | | | chr2: 208,255,071 | chr3: 46,464,905 | |
| | | | CREB1: | LIMD1: | |
| | | | chr2: 207,529,962 | chr3: 45,594,751 | |
| 9 | Gene 3′ | N/A | IDH1: | LTF: | chr9: 32,629,454 |
| | | | chr2: 208,236,229CREB1: | chr3: 46,435,645LIMD1: | |
| | | | chr2: 207,605,988 | chr3: 45,686,341 | |
| 10 | Cancer Gene | N/A | IDH1: Tier 1 | LTF; LIMD1: Tier 4 | Tier 4 |
| | Tier | | CREB1: Tier 4 | | |
| 11 | HRR GENE | N/A | NO | NO | NO |
| 12 | Linear | N/A | IDH1: 515,071 | LTF: 389,905 | 648669 |
| | distance to 5′ | | CREB1: 205,039 | LIMD1: 475,250 | |
| | (bp) | | | | |
| 13 | Closest | N/A | IDH1: 496,229 | LTF: 360,645 | 642454 |
| | distance to | | CREB1: 129,013 | LIMD1: 383,660 | |
| | gene body | | | | |
| | (bp) | | | | |
| 14 | Partner 2 | intergenic break | intergenic break | break in TRIM9 | break in MIR31HG |
| | gene or | | | | |
| | intergenic | | | | |
| 15 | Relevant | FLCN | N/A | NIN | N/A |
| | cancer | | | | |
| | gene(s) | | | | |
| 16 | Gene 5′ | chr17: 17,237,168 | N/A | chr14: 50,831,121 | N/A |
| 17 | Gene 3′ | chr17: 17,212,212 | N/A | chr14: 50,725,840 | N/A |
| 18 | Cancer Gene | Tier 1 | N/A | Tier 4 | N/A |
| | Tier | | | | |
| 19 | HRR GENE | NO | N/A | NO | N/A |
| 20 | Linear | 402833 | N/A | 263880 | N/A |
| | distance to 5′ | | | | |
| | (bp) | | | | |
| 21 | Closest | 402833 | N/A | 263880 | N/A |
| | distance to | | | | |
| | gene body | | | | |
| | (bp) | | | | |
| 22 | Approx. | chr17: | chr12: | chr14: | chr9: |
| | partner | 17,640,001-17,645,000 | 74,115,001-74,120,000 | 51,095,001-51,100,000 | 21,473,001-21,474,000 |
| | breakpoint | | | | |
| | coordinate | | | | |
| | window 2A | | | | |
| 23 | Approx. | chr17: | chr12: | chr14: | chr9: |
| | partner | 17,635,001-17,650,000 | 74,110,001-74,125,000 | 51,090,001-51,105,000 | 21,470,001-21,477,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 189 | 190 | 191 | 192 |
| 2 | SAMPLE | S80 | S80 | S81 | S81 |
| | NUMBER | | | | |
| 3 | Tumor type | Chordoma | Chordoma | Chordoma | Chordoma |
| 4 | Partner 1 | intergenic break | break in TMEM238L | intergenic break | break in SAMD4B |
| | type | | | | |
| 5 | Approx. | chr7: | chr17: | chr9: | chr19: |
| | breakpoint | 63,205,001-63,210,000 | 10,790,001-10,795,000 | 31,985,001-31,986,000 | 39,345,001-39,350,000 |
| | coordinate | | | | |
| | window 1A | | | | |
| 6 | Approx. | chr7: | chr17: | chr9: | chr19: |
| | breakpoint | 63,200,001-63,215,000 | 10,785,001-10,800,000 | 31,983,001-31,988,000 | 39,340,001-39,355,000 |
| | coordinate | | | | |
| | window 1B | | | | |
| 7 | Relevant | N/A | N/A | TAF1L | N/A |
| | cancer | | | | |
| | gene(s) | | | | |
| 8 | Gene 5′ | N/A | N/A | chr9: 32,635,669 | N/A |
| 9 | Gene 3′ | N/A | N/A | chr9: 32,629,454 | N/A |
| 10 | Cancer Gene | N/A | N/A | Tier 4 | N/A |
| | Tier | | | | |
| 11 | HRR GENE | N/A | N/A | NO | N/A |
| 12 | Linear | N/A | N/A | 649669 | N/A |
| | distance to 5′ | | | | |
| | (bp) | | | | |
| 13 | Closest | N/A | N/A | 643454 | N/A |
| | distance to | | | | |
| | gene body | | | | |
| | (bp) | | | | |
| 14 | Partner 2 | intergenic break | intergenic break | break in MIR31HG | intergenic break |
| | gene or | | | | |
| | intergenic | | | | |
| 15 | Relevant | PTPN1 | NLRP1 | N/A | JAK3 |
| | cancer | | | | |
| | gene(s) | | | | |
| 16 | Gene 5′ | chr20: 50,510,383 | chr17: 5,584,509 | N/A | chr19: 17,847,982 |
| 17 | Gene 3′ | chr20: 50,585,241 | chr17: 5,514,118 | N/A | chr19: 17,824,782 |
| 18 | Cancer Gene | Tier 4 | Tier 4 | N/A | Tier 4 |
| | Tier | | | | |
| 19 | HRR GENE | NO | NO | N/A | NO |
| 20 | Linear | 30383 | 740492 | N/A | 197019 |
| | distance to 5′ | | | | |
| | (bp) | | | | |
| 21 | Closest | 30383 | 740492 | N/A | 197019 |
| | distance to | | | | |
| | gene body | | | | |
| | (bp) | | | | |
| 22 | Approx. | chr20: | chr17: | chr9: | chr19: |
| | partner | 50,475,001-50,480,000 | 6,325,001-6,330,000 | 21,473,001-21,474,000 | 18,045,001-18,050,000 |
| | breakpoint | | | | |
| | coordinate | | | | |
| | window 2A | | | | |
| 23 | Approx. | chr20: | chr17: | chr9: | chr19: |
| | partner | 50,470,001-50,485,000 | 6,320,001-6,335,000 | 21,471,001-21,475,000 | 18,040,001-18,055,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 193 | 194 | 195 | 196 |
| 2 | SAMPLE | S81 | S81 | S81 | S82 |
| | NUMBER | | | | |
| 3 | Tumor type | Chordoma | Chordoma | Chordoma | Chordoma |
| 4 | Partner 1 | break in KIRREL2 | intergenic break | break in OR7C1 | break in LEXM |
| | type | | | | |
| 5 | Approx. | chr19: | chr19: | chr19: | chr1: |
| | breakpoint | 35,860,001-35,865,000 | 18,039,001-18,040,000 | 14,800,001-14,801,000 | 54,805,001-54,810,000 |
| | coordinate | | | | |
| | window 1A | | | | |
| 6 | Approx. | chr19: | chr19: | chr19: | chr1: |
| | breakpoint | 35,855,001-35,870,000 | 18,036,001-18,043,000 | 14,797,001-14,804,000 | 54,800,001-54,815,000 |
| | coordinate | | | | |
| | window 1B | | | | |
| 7 | Relevant | KMT2B | PIK3R2 | DNAJB1 | N/A |
| | cancer | | | PKN1 | |
| | gene(s) | | | | |
| 8 | Gene 5′ | chr19: 35,727,156 | chr19: 18,153,163 | DNAJB1: | N/A |
| | | | | chr19: 14,529,300 | |
| | | | | PKN1: | |
| | | | | chr19: 14,433,306 | |
| 9 | Gene 3′ | chr19: 35,728,171 | chr19: 18,170,532 | DNAJB1: | N/A |
| | | | | chr19: 14,514,769P | |
| | | | | KN1: | |
| | | | | chr19: 14,471,859 | |
| 10 | Cancer Gene | Tier 4 | Tier 4 | DNAJB1; PKN1: Tier 4 | N/A |
| | Tier | | | | |
| 11 | HRR GENE | NO | NO | NO | N/A |
| 12 | Linear | 132845 | 113163 | DNAJB1: 270,701 | N/A |
| | distance to 5′ | | | PKN1: 366,695 | |
| | (bp) | | | | |
| 13 | Closest | 131830 | 113163 | DNAJB1: 270,701 | N/A |
| | distance to | | | PKN1: 328,142 | |
| | gene body | | | | |
| | (bp) | | | | |
| 14 | Partner 2 | intergenic break | break in ZNF266 | intergenic break | break in RAD54L |
| | gene or | | | | |
| | intergenic | | | | |
| 15 | Relevant | N/A | N/A | VAV1 | RAD54L |
| | cancer | | | | MKNK1 |
| | gene(s) | | | | |
| 16 | Gene 5′ | N/A | N/A | chr19: 6,772,708 | RAD54L: |
| | | | | | chr1: 46,247,700MKNK1: |
| | | | | | chr1: 46,604,268 |
| 17 | Gene 3′ | N/A | N/A | chr19: 6,857,361 | RAD54L: |
| | | | | | chr1: 46,278,480 |
| | | | | | MKNK1: |
| | | | | | chr1: 46,557,407 |
| 18 | Cancer Gene | N/A | N/A | Tier 4 | RAD54L: Tier 1 |
| | Tier | | | | MKNK1: Tier 4 |
| 19 | HRR GENE | N/A | N/A | NO | RAD54L: YES |
| | | | | | MKNK1: NO |
| 20 | Linear | N/A | N/A | 96708 | RAD54L: N/A break in gene |
| | distance to 5′ | | | | MKNK1: 344,268 |
| | (bp) | | | | |
| 21 | Closest | N/A | N/A | 96708 | RAD54L: N/A break in gene |
| | distance to | | | | MKNK1: 297,407 |
| | gene body | | | | |
| | (bp) | | | | |
| 22 | Approx. | chr19: | chr19: | chr19: | chr1: |
| | partner | 13,670,001-13,675,000 | 9,435,001-9,436,000 | 6,675,001-6,676,000 | 46,255,001-46,260,000 |
| | breakpoint | | | | |
| | coordinate | | | | |
| | window 2A | | | | |
| 23 | Approx. | chr19: | chr19: | chr19: | chr1: |
| | partner | 13,665,001-13,680,000 | 9,432,001-9,439,000 | 6,672,001-6,679,000 | 46,250,001-46,265,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 197 | 198 | 199 | 200 |
| 2 | SAMPLE | S82 | S82 | S82 | S83 |
| | NUMBER | | | | |
| 3 | Tumor type | Chordoma | Chordoma | Chordoma | Embryonal |
| | | | | | Rhabdomyosarcoma |
| 4 | Partner 1 | break in ECE1 | intergenic break | break in CDKN2A | break in CAAP1 |
| | type | | | | |
| 5 | Approx. | chr1: | chr9: | chr9: | chr9: |
| | breakpoint | 21,215,001-21,220,000 | 22,970,001-22,975,000 | 21,977,001-21,978,000 | 26,880,001-26,885,000 |
| | coordinate | | | | |
| | window 1A | | | | |
| 6 | Approx. | chr1: | chr9: | chr9: | chr9: |
| | breakpoint | 21,210,001-21,225,000 | 22,965,001-22,980,000 | 21,975,001-21,980,000 | 26,875,001-26,890,000 |
| | coordinate | | | | |
| | window 1B | | | | |
| 7 | Relevant | N/A | N/A | CDKN2A | N/A |
| | cancer | | | | |
| | gene(s) | | | | |
| 8 | Gene 5′ | N/A | N/A | chr9: 21,994,392 | N/A |
| 9 | Gene 3′ | N/A | N/A | chr9: 21,967,752 | N/A |
| 10 | Cancer Gene | N/A | N/A | Tier 4 | N/A |
| | Tier | | | | |
| 11 | HRR GENE | N/A | N/A | NO | N/A |
| 12 | Linear | N/A | N/A | N/A break in gene | N/A |
| | distance to 5′ | | | | |
| | (bp) | | | | |
| 13 | Closest | N/A | N/A | N/A break in gene | N/A |
| | distance to | | | | |
| | gene body | | | | |
| | (bp) | | | | |
| 14 | Partner 2 | break in MINPP1 | intergenic break | intergenic break | break in MTAP |
| | gene or | | | | |
| | intergenic | | | | |
| 15 | Relevant | NUTM2A | GATA6 | N/A | MTAP |
| | cancer | | | | |
| | gene(s) | | | | |
| 16 | Gene 5′ | chr10: 87,225,448 | chr18: 22,169,589 | N/A | chr9: 21,802,636 |
| 17 | Gene 3 | chr10: 87,234,978 | chr18: 22,202,528 | N/A | chr9: 21,867,081 |
| 18 | Cancer Gene | Tier 4 | Tier 4 | N/A | Tier 4 |
| | Tier | | | | |
| 19 | HRR GENE | NO | NO | N/A | NO |
| 20 | Linear | 304553 | 380412 | N/A | N/A break in gene |
| | distance to 5′ | | | | |
| | (bp) | | | | |
| 21 | Closest | 295023 | 347473 | N/A | N/A break in gene |
| | distance to | | | | |
| | gene body | | | | |
| | (bp) | | | | |
| 22 | Approx. | chr10: | chr18: | chr18: | chr9: |
| | partner | 87,530,001-87,535,000 | 22,550,001-22,555,000 | 22,582,001-22,583,000 | 21,800,001-21,805,000 |
| | breakpoint | | | | |
| | coordinate | | | | |
| | window 2A | | | | |
| 23 | Approx. | chr10: | chr18: | chr18: | chr9: |
| | partner | 87,525,001-87,540,000 | 22,545,001-22,560,000 | 22,580,001-22,585,000 | 21,795,001-21,810,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 201 | 202 | 203 | 204 |
| 2 | SAMPLE | S84 | S84 | S85 | S86 |
| | NUMBER | | | | |
| 3 | Tumor type | Embryonal | Embryonal | Embryonal | Uterine Myxoid |
| | | Rhabdomyosarcoma | Rhabdomyosarcoma | Rhabdomyosarcoma | Leiomyosarcoma |
| 4 | Partner 1 | intergenic break | break in COL5A1 | break in CFH | Intergenic break |
| | type | | | | |
| 5 | Approx. | chr5: | chr9: | chr1: | chr5: 14,882,001- |
| | breakpoint | 72,715,001-72,720,000 | 134,745,001-134,750,000 | 196,656,001-196,656,992 | chr5: 14,884,000 |
| | coordinate | | | | |
| | window 1A | | | | |
| 6 | Approx. | chr5: | chr9: | chr1: | chr5: 14,880,001- |
| | breakpoint | 72,710,001-72,725,000 | 134,740,001-134,755,000 | 196,654,001-196,658,992 | chr5: 14,886,000 |
| | coordinate | | | | |
| | window 1B | | | | |
| 7 | Relevant | N/A | RXRA | N/A | N/A |
| | cancer | | | | |
| | gene(s) | | | | |
| 8 | Gene 5′ | N/A | chr9: 134,326,455 | N/A | N/A |
| 9 | Gene 3′ | N/A | chr9: 134,440,585 | N/A | N/A |
| 10 | Cancer Gene | N/A | Tier 4 | N/A | N/A |
| | Tier | | | | |
| 11 | HRR GENE | N/A | NO | N/A | N/A |
| 12 | Linear | N/A | 418546 | N/A | N/A |
| | distance to 5′ | | | | |
| | (bp) | | | | |
| 13 | Closest | N/A | 304416 | N/A | N/A |
| | distance to | | | | |
| | gene body | | | | |
| | (bp) | | | | |
| 14 | Partner 2 | intergenic break | intergenic break | break in PBX1 | Intergenic |
| | gene or | | | | |
| | intergenic | | | | |
| 15 | Relevant | PSMB1 | N/A | PBX1 | NUMBL |
| | cancer | | | | |
| | gene(s) | | | | |
| 16 | Gene 5′ | chr6: 170,553,307 | N/A | chr1: 164,559,184 | chr19: 40,690,651 |
| 17 | Gene 3′ | chr6: 170,535,120 | N/A | chr1: 164,851,831 | chr19: 40,665,905 |
| 18 | Cancer Gene | Tier 4 | N/A | Tier 4 | Tier 4 |
| | Tier | | | | |
| 19 | HRR GENE | NO | N/A | NO | NO |
| 20 | Linear | 303307 | N/A | N/A break in gene | 54651 |
| | distance to 5′ | | | | |
| | (bp) | | | | |
| 21 | Closest | 285120 | N/A | N/A break in gene | 29905 |
| | distance to | | | | |
| | gene body | | | | |
| | (bp) | | | | |
| 22 | Approx. | chr6: | chr16: | chr1: | chr19: 40,634,001- |
| | partner | 170,245,001-170,250,000 | 46,640,001-46,645,000 | 164,704,001-164,705,000 | chr19: 40,636,000 |
| | breakpoint | | | | |
| | coordinate | | | | |
| | window 2A | | | | |
| 23 | Approx. | chr6: | chr16: | chr1: | chr19: |
| | partner | 170,240,001-170,255,000 | 46,635,001-46,650,000 | 164,702,001-164,707,000 | 40,632,001-40,638,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | 34 |
| 1 | VARIANT ID | 205 | 206 | 207 | 208 |
| 2 | SAMPLE | S86 | S86 | S86 | S87 |
| | NUMBER | | | | |
| 3 | Tumor type | Uterine Myxoid | Uterine Myxoid | Uterine Myxoid | Uterine Myxoid |
| | | Leiomyosarcoma | Leiomyosarcoma | Leiomyosarcoma | Leiomyosarcoma |
| 4 | Partner 1 | break in LYN | break in SAMD4A | break in GFRA3 | break in LUC7L2 |
| | type | | | | |
| 5 | Approx. | chr8: | Chr14: 54,568,001- | chr5: 138,253,001- | chr7: 139,413,501- |
| | breakpoint | 55,988,001-55,991,000 | chr14: 54,573,000 | chr5: 138,255,008 | chr7: 139,414,000 |
| | coordinate | | | | |
| | window 1A | | | | |
| 6 | Approx. | chr8: | Chr14: 54,566,001- | chr5: | chr7: 139,412,001- |
| | breakpoint | 55,985,001-55,994,000 | chr14: 54,575,000 | 138,253,001-138,257,008 | chr7: 139,416,000 |
| | coordinate | | | | |
| | window 1B | | | | |
| 7 | Relevant | LYN | N/A | N/A | LUC7L2 |
| | cancer | PLAG1 | | | |
| | gene(s) | | | | |
| 8 | Gene 5′ | LYN: | N/A | N/A | chr7: 139,359,894 |
| | | chr8: 55,879,835 | | | |
| | | PLAG1: | | | |
| | | chr8: 56,211,273 | | | |
| 9 | Gene 3′ | LYN: | N/A | N/A | chr7: 139,423,454 |
| | | chr8: 55,879,835PLAG1: | | | |
| | | chr8: 56,160,909 | | | |
| 10 | Cancer Gene | LYN: Tier 4 | N/A | N/A | Tier 4 |
| | Tier | PLAG1: Tier 3 | | | |
| 11 | HRR GENE | NO | N/A | N/A | NO |
| 12 | Linear | LYN: N/A Break in Gene | N/A | N/A | N/A (break in gene) |
| | distance to 5′ | PLAG1: 220,273 | | | |
| | (bp) | | | | |
| 13 | Closest | LYN: N/A Break in Gene | N/A | N /A | N/A (break in gene) |
| | distance to | PLAG1: 169,909 | | | |
| | gene body | | | | |
| | (bp) | | | | |
| 14 | Partner 2 | break in RAD51B | break in PRKD1 | break in AXL | break in SLA |
| | gene or | | | | |
| | intergenic | | | | |
| 15 | Relevant | RAD51B | PKRD1 | AXL | N/A |
| | cancer | | | | |
| | gene(s) | | | | |
| 16 | Gene 5′ | chr14: 67,865,032 | chr14: 29,927,847 | chr19: 41,219,223 | None |
| 17 | Gene 3′ | chr14: 68,683,118 | chr14: 29,576,479 | chr19: 41,261,766 | None |
| 18 | Cancer Gene | Tier 1 | Tier 4 | Tier 2 | N/A |
| | Tier | | | | |
| 19 | HRR GENE | YES | NO | NO | N/A |
| 20 | Linear | N/A Break in Gene | N/A break in gene | N/A (break in gene) | N/A |
| | distance to 5′ | | | | |
| | (bp) | | | | |
| 21 | Closest | N/A Break in Gene | N/A break in gene | N/A (break in gene) | N/A |
| | distance to | | | | |
| | gene body | | | | |
| | (bp) | | | | |
| 22 | Approx. | chr14: 68,632,001- | chr14: 29,713,001- | chr19: | chr8: 133,110,501- |
| | partner | chr14: 68,635,000 | chr14: 29,718,000 | 41,254,001-41,259,000 | chr8: 133,111,000 |
| | breakpoint | | | | |
| | coordinate | | | | |
| | window 2A | | | | |
| 23 | Approx. | chr14: 68,629,001- | chr14: 29,711,001- | chr19: | chr8: 133,110,501- |
| | partner | chr14: 68,638,000 | chr14: 29,720,000 | 41,251,001-41,259,000 | chr8: 133,111,000 |
| | breakpoint | | | | or |
| | coordinate | | | | chr8: 133,109,501- |
| | window 2B | | | | chr8: 133,112,000 |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | 14, 34 | 15, 34 | 16, 34 | 17, 34 |
| 1 | VARIANT ID | 209 | 210 | 211 | 212 |
| 2 | SAMPLE | S87 | S87 | S87 | S87 |
| | NUMBER | | | | |
| 3 | Tumor type | Uterine Myxoid | Uterine Myxoid | Uterine Myxoid | Uterine Myxoid |
| | | Leiomyosarcoma | Leiomyosarcoma | Leiomyosarcoma | Leiomyosarcoma |
| 4 | Partner 1 | break in c8orf34 | break in c11orf45 | break in ADAMTS20 | break in ADAMTS20 |
| | type | | | | |
| 5 | Approx. | chr8: | chr11: | chr12: | chr12: |
| | breakpoint | 68,706,001-68,707,000 | 128,910,001-128,915,000 | 43,381,001-43,382,000 | 43,381,001-43,382,000 |
| | coordinate | | | | |
| | window 1A | | | | |
| 6 | Approx. | chr8: | chr11: | chr12: | chr12: |
| | breakpoint | 68,705,001-68,708,000 | 128,905,001-128,920,000 | 43,379,001-43,384,000 | 43,379,001-43,384,000 |
| | coordinate | | | | |
| | window 1B | | | | |
| 7 | Relevant | N/A | ETS1 | ADAMTS20 | ADAMTS20 |
| | cancer | | FLI1 | | |
| | gene(s) | | | | |
| 8 | Gene 5′ | N/A | ETS1: | chr12: 43,552,203 | chr12: 43,552,203 |
| | | | chr11: 128,522,304; | | |
| | | | FLI1: | | |
| | | | chr11: 128,694,072 | | |
| 9 | Gene 3′ | N/A | ETS1: | chr12: 43,353,866 | chr12: 43,353,866 |
| | | | chr11: 128,461,766; | | |
| | | | FLI1: | | |
| | | | chr11: 128,813,267 | | |
| 10 | Cancer Gene | N/A | ETS1; FLI1: Tier 4 | Tier 4 | Tier 4 |
| | Tier | | | | |
| 11 | HRR GENE | N/A | NO | NO | NO |
| 12 | Linear | N/A | ETS1: 387,697; | N/A (break in gene) | N/A (break in gene) |
| | distance to 5′ | | FLI1: 215,929 | | |
| | (bp) | | | | |
| 13 | Closest | N/A | ETS1: 387,697; | N/A (break in gene) | N/A (break in gene) |
| | distance to | | FLI1: 96,734 | | |
| | gene body | | | | |
| | (bp) | | | | |
| 14 | Partner 2 | break in PRKDC | intergenic | intergenic | intergenic |
| | gene or | | | | |
| | intergenic | | | | |
| 15 | Relevant | PRKDC | N/A | N/A | N/A |
| | cancer | | | | |
| | gene(s) | | | | |
| 16 | Gene 5′ | chr8: 47,960,136 | None | None | None |
| 17 | Gene 3′ | chr8: 47,773,111 | None | None | None |
| 18 | Cancer Gene | Tier 4 | N/A | N/A | N/A |
| | Tier | | | | |
| 19 | HRR GENE | NO | N/A | N/A | N/A |
| 20 | Linear | N/A (break in gene) | N/A | N/A | None |
| | distance to 5′ | | | | |
| | (bp) | | | | |
| 21 | Closest | N/A (break in gene) | N/A | N/A | None |
| | distance to | | | | |
| | gene body | | | | |
| | (bp) | | | | |
| 22 | Approx. | chr8: | chr2: | chr14: | chr14: |
| | partner | 47,877,001-47,878,000 | 65,895,001-65,900,000 | 38,594,001-38,595,000 | 51,446,001-51,447,000 |
| | breakpoint | | | | |
| | coordinate | | | | |
| | window 2A | | | | |
| 23 | Approx. | chr8: | chr2: | 14: | chr14: |
| | partner | 47,876,001-47,879,000 | 65,890,001-65,905,000 | 38,592,001-38,597,000 | 51,444,001-51,449,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | 18, 34 | 19, 34 | 20, 34 | 21, 34 |
| 1 | VARIANT ID | 213 | 214 | 215 | 216 |
| 2 | SAMPLE | S87 | S87 | S88 | S88 |
| | NUMBER | | | | |
| 3 | Tumor type | Uterine Myxoid | Uterine Myxoid | Uterine Myxoid | Uterine Myxoid |
| | | Leiomyosarcoma | Leiomyosarcoma | Leiomyosarcoma | Leiomyosarcoma |
| 4 | Partner 1 | Intergenic Break | Intergenic Break | break in PDS5A | Intergenic Break |
| | type | | | | |
| 5 | Approx. | chr12: | chr12: | chr4: | chr2: |
| | breakpoint | 65,756,001-65,757,000 | 48,190,001-48,195,000 | 39,974,001-39,975,000 | 47,628,001-47,629,000 |
| | coordinate | | | | |
| | window 1A | | | | |
| 6 | Approx. | chr12: | chr12: | chr4: 3 | chr2: |
| | breakpoint | 65,754,001-65,759,000 | 48,190,001-48,195,000 or | 9,972,001-39,977,000 | 47,626,001-47,631,000 |
| | coordinate | | chr12: | | |
| | window 1B | | 48,185,001-48,200,000 | | |
| 7 | Relevant | N/A | N/A | N/A | MSH6 |
| | cancer | | | | |
| | gene(s) | | | | |
| 8 | Gene 5′ | N/A | N/A | N/A | chr2: 47,783,145 |
| 9 | Gene 3′ | N/A | N/A | N/A | chr2: 47,806,953 |
| 10 | Cancer Gene | N/A | N/A | N/A | Tier 4 |
| | Tier | | | | |
| 11 | HRR GENE | N/A | N/A | N/A | NO |
| 12 | Linear | N/A | N/A | N/A | 154145 |
| | distance to 5′ | | | | |
| | (bp) | | | | |
| 13 | Closest | N/A | N/A | N/A | 154145 |
| | distance to | | | | |
| | gene body | | | | |
| | (bp) | | | | |
| 14 | Partner 2 | break in RAD51B | break in RAD51B | Intergenic | break in CNTN4 |
| | gene or | | | | |
| | intergenic | | | | |
| 15 | Relevant | RAD51B | RAD51B | RAD51D | N/A |
| | cancer | | | | |
| | gene(s) | | | | |
| 16 | Gene 5′ | chr14: 67,865,032 | chr14: 67,865,032 | chr17: 35,119,860 | None |
| 17 | Gene 3′ | chr14: 68,683,118 | chr14: 68,683,118 | chr17: 35,092,221 | None |
| 18 | Cancer Gene | Tier 1 | Tier 1 | Tier 1 | N/A |
| | Tier | | | | |
| 19 | HRR GENE | YES | YES | YES | N/A |
| 20 | Linear | N/A (break in gene) | N/A (break in gene) | 25141 | N/A (break in gene) |
| | distance to 5′ | | | | |
| | (bp) | | | | |
| 21 | Closest | N/A (break in gene) | N/A (break in gene) | 25141 | N/A (break in gene) |
| | distance to | | | | |
| | gene body | | | | |
| | (bp) | | | | |
| 22 | Approx. | chr14: | chr14: | chr17: | chr3: |
| | partner | 68,673,001-68,674,000 | 68,675,001-68,680,000 | 35,145,001-35,146,000 | 2,801,001-2,802,000 |
| | breakpoint | | | | |
| | coordinate | | | | |
| | window 2A | | | | |
| 23 | Approx. | chr14: | chr14: | chr17: | chr3: |
| | partner | 68,671,001-68,676,000 | 68,670,001-68,685,000 | 35,143,001-35,148,000 | 2,799,001-2,804,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | 22, 34 | 23, 34 | 34 | 34 |
| 1 | VARIANT ID | 217 | 218 | 219 | 220 |
| 2 | SAMPLE | S88 | S88 | S88 | S88 |
| | NUMBER | | | | |
| 3 | Tumor type | Uterine Myxoid | Uterine Myxoid | Uterine Myxoid | Uterine Myxoid |
| | | Leiomyosarcoma | Leiomyosarcoma | Leiomyosarcoma | Leiomyosarcoma |
| 4 | Partner 1 | break in THADA | break in DNMT3A | Intergenic Break | Intergenic Break |
| | type | | | | |
| 5 | Approx. | chr2: | chr2: | chr22: | chr11: |
| | breakpoint | 43,515,001-43,520,000 | 25,310,001-25,315,000 | 39,005,001-39,010,000 | 67,840,001-67,845,000 |
| | coordinate | | | | |
| | window 1A | | | | |
| 6 | Approx. | chr2: | chr2: | chr22: | chr11: |
| | breakpoint | 43,510,001-43,525,000 | 25,305,001-25,320,000 | 39,000,001-39,015,000 | 67,835,001-67,850,000 |
| | coordinate | | | | |
| | window 1B | | | | |
| 7 | Relevant | THADA | DNMT3A | N/A | N/A |
| | cancer | | | | |
| | gene(s) | | | | |
| 8 | Gene 5′ | chr2: 43,596,038 | chr2: 25,342,590 | N/A | N/A |
| 9 | Gene 3′ | chr2: 43,230,851 | chr2: 25,227,855 | N/A | N/A |
| 10 | Cancer Gene | Tier 4 | Tier 4 | N/A | N/A |
| | Tier | | | | |
| 11 | HRR GENE | NO | NO | N/A | N/A |
| 12 | Linear | N/A (break in gene) | N/A (break in gene) | N/A | N/A |
| | distance to 5′ | | | | |
| | (bp) | | | | |
| 13 | Closest | N/A (break in gene) | N/A (break in gene) | N/A | N/A |
| | distance to | | | | |
| | gene body | | | | |
| | (bp) | | | | |
| 14 | Partner 2 | Intergenic | break in LRRC3B | break in CRKL | Intergenic |
| | gene or | | | | |
| | intergenic | | | | |
| 15 | Relevant | N/A | N/A | CRKL | DKK1 |
| | cancer | | | | |
| | gene(s) | | | | |
| 16 | Gene 5′ | None | None | chr22: 20,917,407 | chr10: 52,314,281 |
| 17 | Gene 3′ | None | None | chr22: 20,953,747 | chr10: 52,317,657 |
| 18 | Cancer Gene | N/A | N/A | Tier 4 | Tier 2 |
| | Tier | | | | |
| 19 | HRR GENE | N/A | N/A | NO | NO |
| 20 | Linear | None | None | N/A (break in gene) | 10720 |
| | distance to 5′ | | | | |
| | (bp) | | | | |
| 21 | Closest | None | None | N/A (break in gene) | 10720 |
| | distance to | | | | |
| | gene body | | | | |
| | (bp) | | | | |
| 22 | Approx. | chr3: | chr3: | chr22: | chr10: |
| | partner | 5,230,001-5,235,000 | 26,625,001-26,630,000 | 20,930,001-20,935,000 | 52,325,001-52,330,000 |
| | breakpoint | | | | |
| | coordinate | | | | |
| | window 2A | | | | |
| 23 | Approx. | chr3: | chr3: | chr22: | chr10: |
| | partner | 5,225,001-5,240,000 | 26,625,001-26,640,000 | 20,925,001-20,940,000 | 52,320,001-52,335,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | 34 | 24,34 | 34 | 34 |
| 1 | VARIANT ID | 221 | 222 | 223 | 224 |
| 2 | SAMPLE | S89 | S89 | S89 | S90 |
| | NUMBER | | | | |
| 3 | Tumor type | Uterine Myxoid | Uterine Myxoid | Uterine Myxoid | Subependymal giant |
| | | Leiomyosarcoma | Leiomyosarcoma | Leiomyosarcoma | cell astrocytoma |
| | | | | | (SEGA), poorly |
| | | | | | classified |
| 4 | Partner 1 | break in DOCK4 | break in CDKAL1 | break in ITGA1 | break in PRCC |
| | type | | | | |
| 5 | Approx. | chr7: | chr6: | chr5: | chr1: |
| | breakpoint | 112,203,001-112,204,000 | 21,127,001-21,130,000 | 52,866,001-52,867,000 | 156,790,001-156,795,008 |
| | coordinate | | | | |
| | window 1A | | | | |
| 6 | Approx. | chr7: | chr6: | chr5: | chr1: |
| | breakpoint | 112,201,001-112,206,000 | 21,126,001-21,131,000 | 52,864,001-52,869,000 | 156,785,001-156,800,008 |
| | coordinate | | | | |
| | window 1B | | | | |
| 7 | Relevant | N/A | N/A | N/A | NTRK1 |
| | cancer | | | | |
| | gene(s) | | | | |
| 8 | Gene 5′ | N/A | N/A | N/A | chr1: 156,860,865 |
| 9 | Gene 3′ | N/A | N/A | N/A | chr1: 156,881,850 |
| 10 | Cancer Gene | N/A | N/A | N/A | Tier 1 |
| | Tier | | | | |
| 11 | HRR GENE | N/A | N/A | N/A | NO |
| 12 | Linear | N/A | N/A | N/A | 65857 |
| | distance to 5′ | | | | |
| | (bp) | | | | |
| 13 | Closest | N/A | N/A | N/A | 65857 |
| | distance to | | | | |
| | gene body | | | | |
| | (bp) | | | | |
| 14 | Partner 2 | break in MRTFA | Intergenic | Intergenic | break in TFE3 |
| | gene or | | (2 breakpoints) | | |
| | intergenic | | | | |
| 15 | Relevant | MRTFA | PRDM1 | PIK3CG | TFE3 |
| | cancer | | | | |
| | gene(s) | | | | |
| 16 | Gene 5′ | chr22: 40,636,685 | chr6: 106,086,336 | chr7: 106,865,282 | chrX: 49,043,357 |
| 17 | Gene 3′ | chr22: 40,410,281 | chr6: 106,109,938 | chr7: 106,908,980 | chrX: 49,028,726 |
| 18 | Cancer Gene | Tier 4 | Tier 4 | Tier 4 | Tier 4 |
| | Tier | | | | |
| 19 | HRR GENE | NO | NO | NO | NO |
| 20 | Linear | N/A (break in gene) | 12336 | 64719 | N/A (break in gene) |
| | distance to 5′ | | | | |
| | (bp) | | | | |
| 21 | Closest | N/A (break in gene) | 12336 | 21021 | N/A (break in gene) |
| | distance to | | | | |
| | gene body | | | | |
| | (bp) | | | | |
| 22 | Approx. | chr22: | chr6: | chr7: | chrX: |
| | partner | 40,633,001-40,634,000 | 106,073,001-106,074,000 and | 106,930,001-106,931,000 | 49,035,001-49,040,000 |
| | breakpoint | | chr6: | | |
| | coordinate | | 106,057,001-106,058,000 | | |
| | window 2A | | | | |
| 23 | Approx. | chr22: | chr6: | chr7: | chrX: |
| | partner | 40,631,001-40,636,000 | 106,071,001-106,076,000 and | 106,928,001-106,933,000 | 49,030,001-49,043,000 |
| | breakpoint | | chr6: | | |
| | coordinate | | 106,055,001-106,060,000 | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | FIG. 11 |
| 26 | NOTES | 34 | 25, 34 | 34 | 26 |
| 1 | VARIANT ID | 225 | 226 | 227 | 228 |
| 2 | SAMPLE | S91 | S92 | S92 | S92 |
| | NUMBER | | | | |
| 3 | Tumor type | Glioma | Malignant Brain | Malignant Brain | Malignant Brain |
| | | | tumor (unclassified) | tumor (unclassified) | Tumor |
| 4 | Partner 1 | break in MYBL1 | break in ERBB4 | break in SPAG16 | break in COMMD1 |
| | type | | | | |
| 5 | Approx. | chr8: | chr2: | chr2: | chr2: |
| | breakpoint | 66,590,001-66,595,000 | 212,430,001-212,440,000 | 214,080,001-214,090,000 | 61,995,001-62,000,000 |
| | coordinate | | | | |
| | window 1A | | | | |
| 6 | Approx. | chr8: | chr2: | chr2: | chr2: |
| | breakpoint | 66,585,001-66,595,000 | 212,425,001-212,445,000 | 214,075,001-214,095,000 | 61,990,001-62,005,000 |
| | coordinate | | | | |
| | window 1B | | | | |
| 7 | Relevant | MYBL1 | ERBB4 | N/A | XPO1 |
| | cancer | | | | |
| | gene(s) | | | | |
| 8 | Gene 5′ | chr8: 66,613,218 | chr2: 212,538,802 | N/A | chr2: 61,538,741 |
| 9 | Gene 3′ | chr8: 66,562,175 | chr2: 211,375,717 | N/A | chr2: 61,477,689 |
| 10 | Cancer Gene | Tier 3 | Tier 4 | N/A | Tier 2 |
| | Tier | | | | |
| 11 | HRR GENE | NO | NO | N/A | NO |
| 12 | Linear | N/A (break in gene) | N/A (break in gene) | N/A | 456260 |
| | distance to 5′ | | | | |
| | (bp) | | | | |
| 13 | Closest | N/A (break in gene) | N/A (break in gene) | N/A | 456260 |
| | distance to | | | | |
| | gene body | | | | |
| | (bp) | | | | |
| 14 | Partner 2 | break in MAML2 | Intergenic | Intergenic | break in CLK1 |
| | gene or | | | | |
| | intergenic | | | | |
| 15 | Relevant | N/A | N/A | STAT4 | N/A |
| | cancer | | | | |
| | gene(s) | | | | |
| 16 | Gene 5′ | None | None | chr2: 191,151,590 | N/A |
| 17 | Gene 3′ | None | None | chr2: 191,029,576 | N/A |
| 18 | Cancer Gene | N/A | N/A | Tier 4 | N/A |
| | Tier | | | | |
| 19 | HRR GENE | N/A | N/A | NO | N/A |
| 20 | Linear | N/A (break in gene) | None | 28411 | N/A Break in Gene |
| | distance to 5′ | | | | |
| | (bp) | | | | |
| 21 | Closest | N/A (break in gene) | None | 28411 | N/A Break in Gene |
| | distance to | | | | |
| | gene body | | | | |
| | (bp) | | | | |
| 22 | Approx. | chr11: | chr2: | chr2: | chr2: |
| | partner | 96,080,001-96,085,000 | 234,810,001-234,820,000 | 191,180,001-191,190,000 | 200,850,001-200,855,000 |
| | breakpoint | | | | |
| | coordinate | | | | |
| | window 2A | | | | |
| 23 | Approx. | chr11: | chr2: | chr2: | chr2: |
| | partner | 96,080,001-96,090,000 | 234,805,001-234,825,000 | 191,175,001-191,195,000 | 200,845,001-200,860,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 27 | 27 | 27 |
| 1 | VARIANT ID | 229 | 230 | 231 | 232 |
| 2 | SAMPLE | S92 | S93 | S94 | S95 |
| | NUMBER | | | | |
| 3 | Tumor type | Malignant Brain | Kidney Primitive | Chordoma | Chordoma |
| | | tumor (unclassified) | Neuroectodermal | | |
| | | | tumor (PNET) | | |
| 4 | Partner 1 | Intergenic break | break in POU5F1 | break in LRIG2 | Intergenic Break |
| | type | | | | |
| 5 | Approx. | chr2: | chr6: | chr1: | chr3: |
| | breakpoint | 239,710,001-239,720,000 | 31,170,001-31,175,000 | 113,126,001-113,128,000 | 51,788,001-51,790,000 |
| | coordinate | | | | |
| | window 1A | | | | |
| 6 | Approx. | chr2: | chr6: | chr1: | chr3: |
| | breakpoint | 239,700,001-239,730,000 | 31,165,001-31,175,000 | 113,124,001-113,130,000 | 51,786,001-51,792,000 |
| | coordinate | | | | |
| | window 1B | | | | |
| 7 | Relevant | N/A | POU5F1 | N/A | PARP3 |
| | cancer | | | | |
| | gene(s) | | | | |
| 8 | Gene 5′ | N/A | chr6: 31,170,682 | N/A | chr3: 51,942,345 |
| 9 | Gene 3′ | N/A | chr6: 31,164,337 | N/A | chr3: 51,948,862 |
| 10 | Cancer Gene | N/A | Tier 4 | N/A | Tier 1 |
| | Tier | | | | |
| 11 | HRR GENE | N/A | NO | N/A | YES |
| 12 | Linear | N/A | N/A (break in gene) | N/A | 152345 |
| | distance to 5′ | | | | |
| | (bp) | | | | |
| 13 | Closest | N/A | N/A (break in gene) | N/A | 152345 |
| | distance to | | | | |
| | gene body | | | | |
| | (bp) | | | | |
| 14 | Partner 2 | Intergenic | break in TAF15 | Intergenic | Intergenic |
| | gene or | | | | |
| | intergenic | | | | |
| 15 | Relevant | LRP1B | N/A | GATA6 | CRBN |
| | cancer | | | | |
| | gene(s) | | | | |
| 16 | Gene 5′ | chr2: 142,131,016 | None | chr18: 22,169,589 | chr3: 3,179,691 |
| 17 | Gene 3′ | chr2: 140,231,423 | None | chr18: 22,202,528 | chr3: 3,150,011 |
| 18 | Cancer Gene | Tier 4 | N/A | Tier 4 | Tier 4 |
| | Tier | | | | |
| 19 | HRR GENE | NO | N/A | NO | NO |
| 20 | Linear | 528985 | N/A (break in gene) | 623412 | 91310 |
| | distance to 5′ | | | | |
| | (bp) | | | | |
| 21 | Closest | 528985 | N/A (break in gene) | 590473 | 91310 |
| | distance to | | | | |
| | gene body | | | | |
| | (bp) | | | | |
| 22 | Approx. | chr2: | chr17: | chr18: | chr3: |
| | partner | 142,660,001-142,670,000 | 35,840,001-35,845,000 | 22,793,001-22,794,000 | 3,271,001-3,273,000 |
| | breakpoint | | | | |
| | coordinate | | | | |
| | window 2A | | | | |
| 23 | Approx. | chr2: | chr17: | chr18: | chr3: |
| | partner | 142,650,001-142,680,000 | 35,835,001-35,850,000 | 22,792,001-22,795,000 | 3,269,001-3,275,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | 27 | | | 28 |
| 1 | VARIANT ID | 233 | 234 | 235 | 236 |
| 2 | SAMPLE | S96 | S96 | S96 | S96 |
| | NUMBER | | | | |
| 3 | Tumor type | Chordoma | Chordoma | Chordoma | Chordoma |
| 4 | Partner 1 | break in ANK1 | break in ASTN1 | break in PBX1 | break in MAST2 |
| | type | | | | |
| 5 | Approx. | chr8: | chr1: | chr1: | chr1: |
| | breakpoint | 41,770,001-41,775,000 | 176,960,001-176,970,000 | 164,822,001-164,823,008 | 45,915,001-45,920,000 |
| | coordinate | | | | |
| | window 1A | | | | |
| 6 | Approx. | chr8: | chr1: | chr1: | chr1: |
| | breakpoint | 41,765,001-41,780,000 | 176,950,001-176,980,000 | 164,820,001-164,825,008 | 45,910,001-45,925,000 |
| | coordinate | | | | |
| | window 1B | | | | |
| 7 | Relevant | N/A | N/A | PBX1 | MAST2 |
| | cancer | | | | |
| | gene(s) | | | | |
| 8 | Gene 5′ | N/A | N/A | chr1: 164,559,184 | chr1: 45,803,612 |
| 9 | Gene 3′ | N/A | N/A | chr1: 164,851,831 | chr1: 46,036,122 |
| 10 | Cancer Gene | N/A | N/A | Tier 4 | Tier 4 |
| | Tier | | | | |
| 11 | HRR GENE | N/A | N/A | NO | NO |
| 12 | Linear | N/A | N/A | N/A (break in gene) | N/A (break in gene) |
| | distance to 5′ | | | | |
| | (bp) | | | | |
| 13 | Closest | N/A | N/A | N/A (break in gene) | N/A (break in gene) |
| | distance to | | | | |
| | gene body | | | | |
| | (bp) | | | | |
| 14 | Partner 2 | break in G2E3-AS1 | break in LOC152048 | Intergenic | Intergenic |
| | gene or | | | | |
| | intergenic | | | | |
| 15 | Relevant | PRKD1 | ITGA9 | N/A | N/A |
| | cancer | | | | |
| | gene(s) | | | | |
| 16 | Gene 5′ | chr14: 29,927,847 | chr3: 37,452,141 | None | None |
| 17 | Gene 3′ | chr14: 29,576,479 | chr3: 37,823,507 | None | None |
| 18 | Cancer Gene | Tier 4 | Tier 4 | N/A | N/A |
| | Tier | | | | |
| 19 | HRR GENE | NO | NO | N/A | N/A |
| 20 | Linear | 557154 | 202141 | None | None |
| | distance to 5′ | | | | |
| | (bp) | | | | |
| 21 | Closest | 557154 | 202141 | None | None |
| | distance to | | | | |
| | gene body | | | | |
| | (bp) | | | | |
| 22 | Approx. | chr14: | chr3: | chr3: | chr1: |
| | partner | 30,485,001-30,490,000 | 37,240,001-37,250,000 | 68,934,001-68,935,000 | 8,065,001-8,070,000 |
| | breakpoint | | | | |
| | coordinate | | | | |
| | window 2A | | | | |
| 23 | Approx. | chr14: | chr3: | chr3: | chr1: |
| | partner | 30,485,001-30,490,000 or | 37,240,001-37,250,000 or | 68,933,001-68,936,000 | 8,060,001-8,075,000 |
| | breakpoint | chr14: | chr3: | | |
| | coordinate | 30,480,001-30,495,000 | 37,235,001-37,255,000 | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 237 | 238 | 239 | 240 |
| 2 | SAMPLE | S96 | S97 | S97 | S97 |
| | NUMBER | | | | |
| 3 | Tumor type | Chrodoma | Meningioma | Meningioma | Meningioma |
| 4 | Partner 1 | Break in MAST2 | Intergenic Break | Intergenic Break | Intergenic Break |
| | type | | | | |
| 5 | Approx. | chr1: | chr7: | chr4: | chr3: |
| | breakpoint | 45,920,001-45,930,000 | 141,036,001-141,037,000 | 119,645,001-119,646,000 | 105,180,001-105,182,000 |
| | coordinate | | | | |
| | window 1A | | | | |
| 6 | Approx. | chr1: | chr7: | chr4: | chr3: |
| | breakpoint | 45,910,001-45,940,000 | 141,034,001-141,039,000 | 119,643,001-119,648,000 | 105,178,001-105,184,000 |
| | coordinate | | | | |
| | window 1B | | | | |
| 7 | Relevant | RAD54L | BRAF | N/A | CBLB |
| | cancer | | | | |
| | gene(s) | | | | |
| 8 | Gene 5′ | chr1: 46,247,700 | chr7: 140,924,928 | N/A | chr3: 105,869,012 |
| 9 | Gene 3′ | chr1: 46,278,480 | chr7: 140,730,665 | N/A | chr3: 105,655,461 |
| 10 | Cancer Gene | Tier 1 | Tier 1 | N/A | Tier 4 |
| | Tier | | | | |
| 11 | HRR GENE | YES | NO | N/A | NO |
| 12 | Linear | 317,700 | 111073 | N/A | 685012 |
| | distance to 5′ | | | | |
| | (bp) | | | | |
| 13 | Closest | 317,700 | 111073 | N/A | 471461 |
| | distance to | | | | |
| | gene body | | | | |
| | (bp) | | | | |
| 14 | Partner 2 | Intergenic | Intergenic | Intergenic | Intergenic |
| | gene or | | | | |
| | intergenic | | | | |
| 15 | Relevant | N/A | N/A | ERBB2 | N/A |
| | cancer | | | | |
| | gene(s) | | | | |
| 16 | Gene 5′ | N/A | None | chr17: 39,700,064 | None |
| 17 | Gene 3′ | N/A | None | chr17: 39,728,658 | None |
| 18 | Cancer Gene | N/A | N/A | Tier 1 | N/A |
| | Tier | | | | |
| 19 | HRR GENE | N/A | N/A | NO | N/A |
| 20 | Linear | N/A | None | 107064 | None |
| | distance to 5′ | | | | |
| | (bp) | | | | |
| 21 | Closest | N/A | None | 107064 | None |
| | distance to | | | | |
| | gene body | | | | |
| | (bp) | | | | |
| 22 | Approx. | chr1: | chrX: | chr17: | chr6: |
| | partner | 164,320,001-164,330,000 | 43,303,001-43,304,000 | 39,592,001-39,593,000 | 120,993,001-120,996,000 |
| | breakpoint | | | | |
| | coordinate | | | | |
| | window 2A | | | | |
| 23 | Approx. | chr1: | chrX: | chr17: | 6: |
| | partner | 164,310,001-164,340,000 | 43,301,001-43,306,000 | 39,590,001-39,595,000 | 120,991,001-120,998,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | | | |
| 1 | VARIANT ID | 241 | 242 | 243 | 244 |
| 2 | SAMPLE | S97 | S97 | S97 | S98 |
| | NUMBER | | | | |
| 3 | Tumor type | Meningioma | Meningioma | Meningioma | Embryonal tumors |
| | | | | | with multilayered |
| | | | | | rosettes (ETMR) |
| 4 | Partner 1 | Intergenic Break | break in ITGA3 | Intergenic break | break in KCNH1 |
| | type | | | | |
| 5 | Approx. | chr14: | chr17: | chr14: | chr1: |
| | breakpoint | 28,970,001-28,975,000 | 50,058,001-50,061,000 | 99,135,001-99,140,000 | 211,085,001-211,090,000 |
| | coordinate | | | | |
| | window 1A | | | | |
| 6 | Approx. | chr14: | chr17: | chr14: | chr1: |
| | breakpoint | 28,965,001-28,980,000 | 50,056,001-50,063,000 | 99,130,001-99,145,000 | 211,080,001-211,095,000 |
| | coordinate | | | | |
| | window 1B | | | | |
| 7 | Relevant | PRKD1 | N/A | BCL11B | RCOR3 |
| | cancer | | | | |
| | gene(s) | | | | |
| 8 | Gene 5′ | chr14: 29,927,847 | N/A | chr14: 99,272,197 | chr1: 211,259,975 |
| 9 | Gene 3′ | chr14: 29,576,479 | N/A | chr14: 99,169,287 | chr1: 211,316,385 |
| 10 | Cancer Gene | Tier 4 | N/A | Tier 4 | Tier 4 |
| | Tier | | | | |
| 11 | HRR GENE | NO | N/A | NO | NO |
| 12 | Linear | 952847 | N/A | 132197 | 169975 |
| | distance to 5′ | | | | |
| | (bp) | | | | |
| 13 | Closest | 601479 | N/A | 29287 | 169975 |
| | distance to | | | | |
| | gene body | | | | |
| | (bp) | | | | |
| 14 | Partner 2 | break in LINC01992 | Intergenic | Intergenic | Intergenic |
| | gene or | | | | |
| | intergenic | | | | |
| 15 | Relevant | N/A | CDK12 | N/A | N/A |
| | cancer | | | | |
| | gene(s) | | | | |
| 16 | Gene 5′ | None | chr17: 39,461,486 | None | None |
| 17 | Gene 3′ | None | chr17: 39,534,544 | None | None |
| 18 | Cancer Gene | N/A | Tier 4 | N/A | N/A |
| | Tier | | | | |
| 19 | HRR GENE | N/A | YES | N/A | N/A |
| 20 | Linear | None | 130515 | None | None |
| | distance to 5′ | | | | |
| | (bp) | | | | |
| 21 | Closest | None | 57457 | None | None |
| | distance to | | | | |
| | gene body | | | | |
| | (bp) | | | | |
| 22 | Approx. | chr17: | chr17: | chr14: | chr4: |
| | partner | 27,975,001-27,980,000 | 39,592,001-39,593,000 | 27,215,001-27,220,000 | 18,080,001-18,085,000 |
| | breakpoint | | | | |
| | coordinate | | | | |
| | window 2A | | | | |
| 23 | Approx. | chr17: | chr17: | chr14: | chr4: |
| | partner | 27,970,001-27,985,000 | 39,590,001-39,595,000 | 27,210,001-27,225,000 | 18,075,001-18,090,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 29 | | |
| 1 | VARIANT ID | 245 | 246 | 247 | 248 |
| 2 | SAMPLE | S99 | S99 | S99 | S100 |
| | NUMBER | | | | |
| 3 | Tumor type | Met high-grade | Met high-grade | Met high-grade | Pleomorphic |
| | | sarcoma, uterine | sarcoma, uterine | sarcoma, uterine | Xanthoastrocytoma |
| | | origin | origin. | origin. | (PXA). |
| 4 | Partner 1 | Intergenic break | Intergenic break | break in PTPRT | break in SYNE1 |
| | type | | | | |
| 5 | Approx. | chr5: | chr12: | chr20: | chr6: |
| | breakpoint | 96,658,001-96,659,000 | 104,448,001-104,450,000 | 42,538,001-42,539,000 | 152,536,001-152,538,000 |
| | coordinate | | | | |
| | window 1A | | | | |
| 6 | Approx. | chr5: | chr12: | chr20: | chr6: |
| | breakpoint | 96,656,001-96,661,000 | 104,446,001-104,452,000 | 42,536,001-42,541,000 | 152,534,001-152,540,000 |
| | coordinate | | | | |
| | window 1B | | | | |
| 7 | Relevant | N/A | N/A | PTPRT | SYNE1 |
| | cancer | | | | |
| | gene(s) | | | | |
| 8 | Gene 5′ | N/A | N/A | chr20: 43,189,906 | chr6: 152,637,362 |
| 9 | Gene 3′ | N/A | N/A | chr20: 42,072,756 | chr6: 152,121,687 |
| 10 | Cancer Gene | N/A | N/A | Tier 4 | Tier 4 |
| | Tier | | | | |
| 11 | HRR GENE | N/A | N/A | NO | NO |
| 12 | Linear | N/A | N/A | N/A (break in gene) | N/A (break in gene) |
| | distance to 5′ | | | | |
| | (bp) | | | | |
| 13 | Closest | N/A | N/A | N/A (break in gene) | N/A (break in gene) |
| | distance to | | | | |
| | gene body | | | | |
| | (bp) | | | | |
| 14 | Partner 2 | Intergenic | break in WRAP53 | Intergenic | Intergenic |
| | gene or | | | | |
| | intergenic | | | | |
| 15 | Relevant | BTK | TP53 | N/A | N/A |
| | cancer | | | | |
| | gene(s) | | | | |
| 16 | Gene 5′ | chrX: 101,386,191 | chr17: 7,687,490 | None | None |
| 17 | Gene 3′ | chrX: 101,349,450 | chr17: 7,668,421 | None | None |
| 18 | Cancer Gene | Tier 1 | Tier 3 | N/A | N/A |
| | Tier | | | | |
| 19 | HRR GENE | NO | NO | N/A | N/A |
| 20 | Linear | 41191 | 11 | None | None |
| | distance to 5′ | | | | |
| | (bp) | | | | |
| 21 | Closest | 4450 | 11 | None | None |
| | distance to | | | | |
| | gene body | | | | |
| | (bp) | | | | |
| 22 | Approx. | chrX: | chr17: | chr20: | chr9: |
| | partner | 101,344,001-101,345,000 | 7,687,501-7,688,000 | 34,684,001-34,685,000 | 22,156,001-22,157,000 |
| | breakpoint | | | | |
| | coordinate | | | | |
| | window 2A | | | | |
| 23 | Approx. | chrX: | chr17: | chr20: | chr9: |
| | partner | 101,342,001-101,347,000 | 7,687,501-7,690,000 | 34,682,001-34,687,000 | 22,154,001-22,159,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | N/A | N/A | N/A | N/A |
| 26 | NOTES | | 30 | | |
| 1 | VARIANT ID | 249 | 250 | 251 | 252 |
| 2 | SAMPLE | S100 | S101 | S101 | S102 |
| | NUMBER | | | | |
| 3 | Tumor type | Pleomorphic | Glioblastoma | Glioblastoma | bile duct tumor |
| | | Xanthoastrocytoma | Multiforme/ | Multiforme/ | |
| | | (PXA). | anaplastic | anaplastic | |
| | | | astrocytoma with | astrocytoma with | |
| | | | piloid features | piloid features | |
| | | | (ANA PA) | (ANA PA) | |
| 4 | Partner 1 | break in SYNE1 | break in XPR1 | break in SETD5 | break in gene |
| | type | | | | |
| 5 | Approx. | chr6: | chr1: | chr3: | chr17: |
| | breakpoint | 152,536,001-152,538,000 | 180,720,001-180,725,000 | 9,475,001-9,480,000 | 30,428,001-30,429,000 |
| | coordinate | | | | |
| | window 1A | | | | |
| 6 | Approx. | chr6: | chr1: | chr3: | chr17: |
| | breakpoint | 152,534,001-152,540,000 | 180,715,001-180,730,000 | 9,470,001-9,480,000 | 30,427,001-30,430,000 |
| | coordinate | | | | |
| | window 1B | | | | |
| 7 | Relevant | ESR1 | N/A | SETD5 | CPD |
| | cancer | | | | |
| | gene(s) | | | | |
| 8 | Gene 5′ | chr6: 151,690,496 | N/A | chr3: 9,397,615 | chr17: 30,378,927 |
| 9 | Gene 3′ | chr6: 152,103,274 | N/A | chr3: 9,478,154 | chr17: 30,469,989 |
| 10 | Cancer Gene | Tier 1 | N/A | Tier 4 | N?A |
| | Tier | | | | |
| 11 | HRR GENE | NO | N/A | NO | NO |
| 12 | Linear | 845505 | N/A | N/A (break in gene) | N/A (break in gene) |
| | distance to 5′ | | | | |
| | (bp) | | | | |
| 13 | Closest | 432727 | N/A | N/A (break in gene) | N/A (break in gene) |
| | distance to | | | | |
| | gene body | | | | |
| | (bp) | | | | |
| 14 | Partner 2 | Intergenic | Intergenic | break in LINC01844 | gene |
| | gene or | | | | |
| | intergenic | | | | |
| 15 | Relevant | N/A | FGF1 | FGF1 | LASP1 |
| | cancer | | | | |
| | gene(s) | | | | |
| 16 | Gene 5′ | None | chr5: 142,698,070 | chr5: 142,698,070 | chr17: 38,870,058 |
| 17 | Gene 3′ | None | chr5: 142,592,179 | chr5: 142,592,179 | chr17: 38,921,770 |
| 18 | Cancer Gene | N/A | Tier 4 | Tier 4 | N/A |
| | Tier | | | | |
| 19 | HRR GENE | N/A | NO | NO | NO |
| 20 | Linear | None | 113070 | 56931 | |
| | distance to 5′ | | | | |
| | (bp) | | | | |
| 21 | Closest | None | 7179 | 56931 | |
| | distance to | | | | |
| | gene body | | | | |
| | (bp) | | | | |
| 22 | Approx. | chr9: | chr5: | chr5: | chr17: |
| | partner | 22,156,001-22,157,000 | 142,580,001-142,585,000 | 142,755,001-142,760,000 | 38,872,001-38,873,000 |
| | breakpoint | | | | |
| | coordinate | | | | |
| | window 2A | | | | |
| 23 | Approx. | chr9: | chr5: | chr5: | chr17: |
| | partner | 22,154,001-22,159,000 | 142,575,001-142,590,000 | 142,750,001-142,765,000 | 38,871,001-38,874,000 |
| | breakpoint | | | | |
| | coordinate | | | | |
| | window 2B | | | | |
| 24 | Genome wide | Genome-wide | Genome-wide | Genome-wide | Genome-wide |
| | or capture | | | | |
| 25 | FIGURE | | N/A | N/A | N/A |
| 26 | NOTES | 31 | | | 39 |
| 1 | VARIANT ID | 253 | 254 | 255 | 256 |
| 2 | SAMPLE | S103 | S104 | S105 | S65 |
| | NUMBER | | | | |
| 3 | Tumor type | ALL | AML | Choroid plexus | Glioblastoma |
| | | | | carcinoma | |
| 4 | Partner 1 | Break in gene | Break in gene | Break in gene | Break in ZCCHC7 |
| | type | | | (NUP107) | |
| 5 | Approx. | chr12: | chr11: | chr12: | chr9: |
| | breakpoint | 6,689,001-6,690,000 | 118,446,318-118,511,511 | 68,730,001-68,735,000 | 37,133,001-37,134,000 |
| | coordinate | | | | |
| | window 1A | | | | |
| 6 | Approx. | chr12: | chr11: | chr12: | chr9: |
| | breakpoint | 6,681,510-6,689,510 | 118,446,318-118,511,511 | 68,720,001-68,745,000 | 37,129,001-37,138,000 |
| | coordinate | | | | |
| | window 1B | | | | |
| 7 | Relevant | ZNF384 | KMT2A | MDM2 | PAX5 |
| | cancer | | | | |
| | gene(s) | | | | |
| 8 | Gene 5′ | chr12: 6,689,510 | chr11: 118,436,490 | chr12: 68,809,002 | chr9: 37,034,268 |
| 9 | Gene 3′ | chr12: 6,666,648 | chr11: 118,523,917 | chr12: 68,840,807 | chr9: 36,833,269 |
| 10 | Cancer Gene | Tier 4 | Tier 1 | Tier 2 | Tier 4 |
| | Tier | | | | |
| 11 | HRR GENE | NO | NO | NO | NO |
| 12 | Linear | N/A (break in gene) | N/A (break in gene) | 137544002 | 99233 |
| | distance to 5′ | | | | |
| | (bp) | | | | |
| 13 | Closest | N/A (break in gene) | N/A (break in gene) | 137544002 | 99233 |
| | distance to | | | | |
| | gene body | | | | |
| | (bp) | | | | |
| 14 | Partner 2 | gene | gene | gene | Intergenic |
| | gene or | | | | |
| | intergenic | | | | |
| 15 | Relevant | EP300 | MLLT10 | LINC01239 | N/A |
| | cancer | | | | |
| | gene(s) | | | | |
| 16 | Gene 5′ | chr22: 41,092,592 | chr10: 21,524,675 | chr9: 22,646,200 | N/A |
| 17 | Gene 3′ | chr22: 41,180,077 | chr10: 21,743,630 | chr9: 22,824,213 | N/A |
| 18 | Cancer Gene | Tier 2 | Tier 4 | Tier 4 | N/A |
| | Tier | | | | |
| 19 | HRR GENE | NO | NO | NO | N/A |
| 20 | Linear | | | | N/A |
| | distance to 5′ | | | | |
| | (bp) | | | | |
| 21 | Closest | | | | N/A |
| | distance to | | | | |
| | gene body | | | | |
| | (bp) | | | | |
| 22 | Approx. | chr22: | chr10: | chr9: | chr9: |
| | partner | 41,133,001-41,134,00 | 21,655,001-21,660,00 | 22,780,001-22,785,000 | 34,915,001-34,916,000 |
| | breakpoint | | | | |
| | coordinate | | | | |
| | window 2A | | | | |
| 23 | Approx. | chr22: | chr10: | chr9: | chr9: |
| | partner | 41,129,001-41,138,00 | 21,650,001-21,665,00 | 22,775,001-22,790,000 | 34,911,001-34,920,000 |
| | breakpoint | | | | |
| | coordinate | | | | |
| | window 2B | | | | |
| 24 | Genome wide | Genome-wide | capture | Genome-wide | Genome-wide |
| | or capture | | | | |
| 25 | FIGURE | N/A | FIG. 4; FIG. 5 | N/A | N/A |
| 26 | NOTES | 36 | 37 | 38 | |
| 1 | VARIANT ID | 257 | 258 | 259 | 260 |
| 2 | SAMPLE | S65 | S106 | S106 | S107 |
| | NUMBER | | | | |
| 3 | Tumor type | Glioblastoma | Chordoma | Chordoma | Chordoma |
| 4 | Partner 1 | Break in ZCCHC7 | Intergenic break | Intergenic break | Intergenic break |
| | type | | | | |
| 5 | Approx. | chr9: | chr3: | chr1: | chr5: |
| | breakpoint | 37,133,001-37,134,000 | 89,070,001-89,075,000 | 115,205,001-115,210,000 | 1,248,001-1,250,000 |
| | coordinate | | | | |
| | window 1A | | | | |
| 6 | Approx. | chr9: | chr3: | chr1: | chr5: |
| | breakpoint | 37,129,001-37,138,000 | 89,065,001-89,080,000 | 115,200,001-115,215,000 | 1,247,001-1,251,000 |
| | coordinate | | | | |
| | window 1B | | | | |
| 7 | Relevant | ZCCHC7 | EPHA3 | NRAS | TERT |
| | cancer | | | | |
| | gene(s) | | | | |
| 8 | Gene 5′ | chr9: 37,120,574 | chr3: 89,107,621 | chr1: 114,716,771 | chr5: 1,295,068 |
| 9 | Gene 3′ | chr9: 37,358,149 | chr3: 89,482,134 | chr1: 114,704,469 | chr5: 1,253,167 |
| 10 | Cancer Gene | Tier 4 | Tier 4 | Tier 1 | Tier 3 |
| | Tier | | | | |
| 11 | HRR GENE | NO | NO | NO | NO |
| 12 | Linear | N/A (break in gene) | 32621 | 488230 | 45068 |
| | distance to 5′ | | | | |
| | (bp) | | | | |
| 13 | Closest | N/A (break in gene) | 32621 | 488230 | 3167 |
| | distance to | | | | |
| | gene body | | | | |
| | (bp) | | | | |
| 14 | Partner 2 | Intergenic | Intergenic | break in SVIL2P | break in CAV1 |
| | gene or | | | | |
| | intergenic | | | | |
| 15 | Relevant | N/A | N/A | N/A | MET |
| | cancer | | | | |
| | gene(s) | | | | |
| 16 | Gene 5′ | N/A | N/A | none | chr7: 116,672,196 |
| 17 | Gene 3′ | N/A | N/A | none | chr7: 116,798,377 |
| 18 | Cancer Gene | N/A | N/A | N/A | Tier 1 |
| | Tier | | | | |
| 19 | HRR GENE | N/A | N/A | N/A | NO |
| 20 | Linear | N/A | N/A | N/A | 125196 |
| | distance to 5′ | | | | |
| | (bp) | | | | |
| 21 | Closest | N/A | N/A | N/A | 125196 |
| | distance to | | | | |
| | gene body | | | | |
| | (bp) | | | | |
| 22 | Approx. | chr9: | chr3: | chr10: | chr7: |
| | partner | 34,915,001-34,916,000 | 1,425,001-1,430,000 | 30,715,001-30,716,000 | 116,546,001-116,547,000 |
| | breakpoint | | | | |
| | coordinate | | | | |
| | window 2A | | | | |
| 23 | Approx. | chr9: | chr3: | chr10: | chr7: |
| | partner | 34,911,001-34,920,000 | 1,420,001-1,435,000 | 30,714,001-30,717,000 | 116,545,001-116,548,000 |
| | breakpoint | | | | |
| | coordinate | | | | |
| | window 2B | | | | |
| 24 | Genome wide | Genome-wide | Genome-wide | Genome-wide | capture |
| | or capture | | | | |
| 25 | FIGURE | N/A | N/A | N/A | FIG. 7; FIG. 8 |
| 26 | NOTES | | 35 | 35 | |
| 1 | VARIANT ID | 261 | 262 | 263 | |
| 2 | SAMPLE | S108 | S109 | S110 | |
| | NUMBER | | | | |
| 3 | Tumor type | Chordoma | Glioma | Chordoma | |
| 4 | Partner 1 | Intergenic break | break in MYBL1 | Break in MAST2 | |
| | type | and/or break in | | | |
| | | NR_155748 | | | |
| 5 | Approx. | chr10: | chr8: | chr1: | |
| | breakpoint | 52,560,001-52,565,000 | 66,610,000-66611,000 and | 45,920,001-45,930,000 | |
| | coordinate | | chr8: | | |
| | window 1A | | 66,586,000-66,587,000 | | |
| 6 | Approx. | chr10: | | chr1: | |
| | breakpoint | 52,555,001-52,570,000 | | 45,910,001-45,940,000 | |
| | coordinate | | | | |
| | window 1B | | | | |
| 7 | Relevant | DKK1 | MYBL1 | RAD54L | |
| | cancer | | | | |
| | gene(s) | | | | |
| 8 | Gene 5′ | chr10: 52,314,281 | chr8: 66,613,218 | chr1: 46,247,700 | |
| 9 | Gene 3′ | chr10: 52,317,657 | chr8: 66,562,175 | chr1: 46,278,480 | |
| 10 | Cancer Gene | Tier 2 | Tier 3 | Tier 1 | |
| | Tier | | | | |
| 11 | HRR GENE | NO | NO | YES | |
| 12 | Linear | 245720 | N/A (break in gene) | 317,700 | |
| | distance to 5′ | | | | |
| | (bp) | | | | |
| 13 | Closest | 242344 | N/A (break in gene) | 317,700 | |
| | distance to | | | | |
| | gene body | | | | |
| | (bp) | | | | |
| 14 | Partner 2 | intergenic and/or | break in CHD7 | Intergenic | |
| | gene or | break in | | | |
| | intergenic | NR_110304 | | | |
| 15 | Relevant | N/A | CHD7 | N/A | |
| | cancer | | | | |
| | gene(s) | | | | |
| 16 | Gene 5′ | none | chr8: 60,678,740 | N/A | |
| 17 | Gene 3′ | none | chr8: 60,868,028 | N/A | |
| 18 | Cancer Gene | N/A | Tier 4 | N/A | |
| | Tier | | | | |
| 19 | HRR GENE | N/A | NO | N/A | |
| 20 | Linear | N/A | See notes | N/A | |
| | distance to 5′ | | | | |
| | (bp) | | | | |
| 21 | Closest | N/A | See notes | N/A | |
| | distance to | | | | |
| | gene body | | | | |
| | (bp) | | | | |
| 22 | Approx. | chr10: | chr8: | chr1: | |
| | partner | 75,405,001-75,410,000 | 60,790,000-60,795,000 and | 164,320,001-164,330,000 | |
| | breakpoint | | chr8: | | |
| | coordinate | | 60,820,000-60,825,000 | | |
| | window 2A | | | | |
| 23 | Approx. | chr10: 10: | | chr1: | |
| | partner | 75,400,001-75,415,000 | | 164,310,001-164,340,000 | |
| | breakpoint | | | | |
| | coordinate | | | | |
| | window 2B | | | | |
| 24 | Genome wide | Genome-wide | Genome-wide | Genome-wide | |
| | or capture | | | | |
| 25 | FIGURE | N/A | N/A | N/A | |
| 26 | NOTES | 32 | 32 | | |

NOTES (from row 26 of Table 10):
1. This tumor also had 3 known fusions, that were previously detected by targeted RNA-seq: TNS3-ETV1; EGFR-IMPP2L; GNAI1-BRAF. The two novel neighborhood fusions found in this sample, plus the 3 known fusions are all byproducts of an isolated chr7 chromothripsis.
2. The intergenic breakpoint on chr14 is located in a cluster of IgH genes. This locus is known to rearrange with MYC in lymphoma and other hematological cancers.
3. The intergenic breakpoint on chr14 is located in a cluster of IgH genes. This locus is known to rearrange with oncogene loci in hematological cancers.
4. The intergenic breakpoint on chr14 is located in a cluster of IgH genes. This locus is known to rearrange with oncogene loci in hematological cancers.
5. Produces SIDT1-EPHB1 fusion gene.
6. The intergenic breakpoint on chr14 is located in a cluster of IgH genes. This locus is known to rearrange with oncogene loci in hematological cancers.
7. The intergenic breakpoint on chr22 is located in a cluster of IgL genes. This locus is known to rearrange with oncogene loci in hematological cancers.
8. The BCR-NSD2 fusion is a “head to head” fusion, fusing the 5′ ends of both genes. Also, the breakpoint on chr22 is just downstream of the IgL locus, which is known to rearrange with oncogenes. For e.g. in myeloma, immunoglobulin rearrangements with NSD2 also increase expression of nearby FGFR3.
9. The FMR1-SIN3A fusion is a “tail to tail” fusion, fusing the 3′ ends of both genes. Literature suggests cancer implications (i.e. Tier 4).
10. Translocation forms RP1-RAD51B gene fusion.
11. The intergenic breakpoint on chr14 is located in a cluster of IgH genes. This locus is known to rearrange with oncogene loci, such as programmed cell death ligands, in hematological cancers such as lymphomas (https://pubmed.ncbi.nlm.nih.gov/24497532/).
12. The intergenic breakpoint on chr14 is located in a cluster of IgH genes. This locus is known to rearrange with oncogene loci in hematological cancers.
13. The intergenic breakpoint on chr14 is located in a cluster of IgH genes. This locus is known to rearrange with oncogene loci in hematological cancers.
14. translocation, resulting in an in-frame gene fusion with RAD51B as the 5′ partner and LYN as the 3′ partner. As far as I can tell, Lyn is a tyrosine kinase and a known 3′ fusion partner in hematologic cancers. The tyrosine kinas domain is in the 3′ portion of LYN. Not aware of any reports of Lyn fusions in sarcomas. LYN is also involved in a complex rearrangement involving ZFPM2 on chr8 and ARFGEF1 also on chr8.
15. Inversion, resulting in a in-frame gene fusion where SAMD4A is the 5′ partner and PRDK1 is the 3′ partner. PRKD1 is a serine/threonine-protein kinase, with the kinas domain in the 3′ portion of the gene.
16. Translocation, resulting in an in-frame gene fusion with AXL as the 5′ partner and GFRA3 as the 3′ partner.
17. Translocation, resulting in a gene fusion where LUC7L2 is the 5′ partner and SLA is the 3′ partner.
18. Intra-chromosomal rearrangement creating an in-frame gene fusion with c8orf34 as the 5′ partner, and PRKDC as the 3′ partner.
19. Translocation, where the breakpoint on chr11 is in linear proximity to the 2 oncogenes, FLI1 and ETS1.
20. Translocation, with a breakpoint in ADAMTS20, but the other partner in an intergenic region.
21. Translocation, with the same breakpoint in ADAMTS20 as above, but the partner here has an intergenic break and the rearrangement extends into the 3′ of the FRMD6-AS2, which is an antisense transcript for the gene FRMD6.
22. This translocation has a breakpoint in RAD51B, and the 5′ portion of RAD51B is involved in the rearrangement.
23. This translocation has a breakpoint in RAD51B, and the 3′ portion of RAD51B is involved in the rearrangement. This could be a complex rearrangement with variant 213.
24. This translocation appears to create a fusion between DNMT3A and LRRC3B, however, the gene fusion does not appear to be in the correct orientation since the fusion involves the 3′ ends of both genes.
25. This structural variant is an inversion, and one end of the inverted sequence also had a deletion. So technically, there are 3 total breakpoints. The sequence between the two breakpoints in partner #2 has been deleted. The distance to PRDM1 is the closets distance to one of the breakpoints.
26. Reciprocal translocation that creates the fusion genes PRCC-TFE3, and, TFE3-PRCC. Essentially the reciprocal nature of the translocation produces fusion genes where each gene is either the 5′ or 3′ partner.
27. A segment of ERBB4, ranging from chr2: 212,250,001-212,440,000 is involved in a rearrangement with a segment from chr2: 212,440,000-234,820,000. This also appears to be in complex rearrangement with another segment on chr2, from chr2: 2: 225,560,001-2: 225,560,001, which is entirely contained with the gene NYAP2. Note that chr2 in this sample has massive chromothripsis of chr2.
28. This SV is an inversion.
29. This structural variant is a deletion - the segment between the breakpoints has been deleted.
30. This one is interesting because the disruption is in the promoter region of TP53. There are other reports of translocation involving the 5′ end of TP53 in osteosarcoma, and those result in reduced expression of the TP53 gene, which makes sense because it′s a tumor suppressor gene. (https://www.ncbi.nlm.nih.gov/pmc/articles/PMC4480712/)
31. This variant (variant 249) is the same set of breakpoints as for variant 248, except, the first breakpoint is near an oncogene called ESR1, and this row describes the distance of ESR1 to the breakpoint in SYNE1.
32. The “genes” in sample S108 are non-coding uncharacterized loci with the nomenclature in RefSeq as “NR_”.
33. The fusion of MYBL1 with CHD7 is complex, and involves an inversion and at least 2 breakpoints within each gene. The breakpoints in MYBL1 are: chr8: 66,610,000-66,611,000 and chr8: 66,586,000-66,587,000. The breakpoints in CHD7 are chr8: 60,790,000-60,795,000 and chr8: 60,820,000-60,825,000. The HiC signal indicates an inversion, which would be necessary to create an “in frame” fusion between MYBL1 and CHD7 because their gene orientations (before the inversion) are on different strands. The portion of MYBL1 between the breakpoints has fused to the 5′ portion of CHD7. Therefore the fusion point is MYBL1: chr8: 66,610,000-66611,000 and the fusion point for CHD7 is: chr8: 60,790,000-60,795,000. This would create an in-frame CHD7-MYBL1 fusion. Because this is an inversion, the reciprocal fusion also occurs but where MYBL1 is the 5′ partner in the fusion, and CHD7 is the 3′ partner. In this case the MYBL1 breakpoint is chr8: 66,610,000-66611,000 and the CHD7 breakpoint is chr8: 60,820,000-60,825,000. Also based on the HiC signal for this fusion, the sequence between the two breakpoints in CHD7 have been deleted. There is also involvement with 2 other genes, CDH17 and AGTPBP1, based on the spatial proximity signal from HiC. The breakpoint in CDH17 is chr8: 94,130,000-94,140,000, however, the specific connectivity to MYBL1, AGTPBP1 and CHD7 is not clear. The breakpoint in AGTPBP1 is chr9: 85,570,000-85,580,000, however, the specific connectivity to MYBL1, CDH17 and CHD7 is not clear.
34. Notable trends in the 4 uterine myxoid LMS tumors: RAD51 alterations were found in 3/4 tumors, with 2 involving RAD51B and 1 with RAD51D. Two with breakpoints within RAD51 genes, and one with breakpoint adjacent to the gene. PRKD gene fusions observed in 2/4 samples. One was PRKD1 and the other PRKDC. Highly rearranged chr8 (with numerous intra-and inter-chromosomal rearrangements) in 2/4 samples (S86 and S87)
35. Part of a complex rearrangement between chr1, chr3, chr10.
36. This sample had no clear/known tumor driver by standard cyto/molecular testing (e.g. chromosomal karyotyping, a FISH panel, DNA microarray, and a cancer NGS panel).
37. This sample had no clear/known tumor driver by standard cyto/molecular testing (e.g. chromosomal karyotyping, a FISH panel, DNA microarray, and a cancer NGS panel). Prior testing via FISH for KMT2A rearrangement was negative. FISH was also negative for other AML translocations (RUNX1, NUP98, CBFB). Applicants have identified the fusion as KMT2A-MLLT10, however, sample was tested for KMT2A via FISH and it came back negative, thereby showing the inventive technology disclosed herein can identify SVs not able to be found by standard techniques.
38. This sample had no clear/known tumor driver by standard cyto/molecular testing (e.g. chromosomal karyotyping, a FISH panel, DNA microarray, methylation array, and a cancer NGS panel).
39. This SV is a deletion.

The entirety of each patent, patent application, publication and document referenced herein is incorporated by reference, to the extent permitted by law. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein. Unless otherwise noted, technical terms are used according to conventional usage. Definitions of common terms in molecular biology may be found in Benjamin Lewin's Genes XII, published by Jones & Bartlett Learning, 2017 (ISBN-10:1284104494) and Joseph Jez (ed), Encyclopedia of Biological Chemistry, published by Elsevier, 2021 (ISBN 9780128194607).

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. A method for detecting the presence or absence of a structural variant in a sample, the method comprising:
a) performing a nucleic acid analysis on the selected sample, wherein the analysis comprises a method that preserves spatial-proximal contiguity information; and
b) detecting whether a structural variant is present or absent in the selected sample according to the nucleic acid analysis in (b), wherein a breakpoint of the structural variant is not within the one or more cancer genes analyzed in (a).

2. The method of claim 1, wherein the structural variant breakpoint is associated with and/or adjacent to a Tier 1, Tier 2, or Tier 3 gene.

3. The method of claim 2, wherein the structural variant breakpoint is adjacent to a Tier 1, Tier 2, or Tier 3 gene.

4. The method of claim 3, further comprising administering a treatment to the subject when the structural variant is present.

5. The method of claim 1, wherein the sample is a patient-derived sample.

6. The method of claim 5, wherein the sample is an FFPE sample,

7. A method for detecting the presence or absence of a structural variant in a sample, the method comprising:
a) selecting a sample from a subject, wherein one or more oncogenes in the sample were analyzed for one or more genetic variations associated with cancer, and the one or more oncogenes comprise no detectable genetic variation associated with cancer;
b) performing a nucleic acid analysis on the selected sample, wherein the analysis comprises a method that preserves spatial-proximal contiguity information; and
c) detecting whether a structural variant is present or absent in the selected sample according to the nucleic acid analysis in (b), wherein a breakpoint of the structural variant is not within the one or more cancer genes analyzed in (a).

8. The method of claim 7, wherein the structural variant breakpoint is associated with and/or adjacent to a Tier 1, Tier 2, or Tier 3 gene.

9. The method of claim 8, wherein the structural variant breakpoint is adjacent to a Tier 1, Tier 2, or Tier 3 gene.

10. The method of claim 9, further comprising administering a treatment to the subject when the structural variant is present.

11. The method of claim 7, wherein the sample is a patient-derived sample.

12. The method of claim 11, wherein the sample is an FFPE sample,

13. A method for detecting the presence or absence of a structural variant in a sample, the method comprising:
a) performing a nucleic acid analysis on a sample from a subject, wherein the analysis comprises i) generating proximity ligated nucleic acid molecules and ii) contacting the proximity ligated nucleic acid molecules with one or more capture probe species, thereby generating enriched proximity ligated nucleic acid molecules, wherein the one or more capture probe species each comprise a polynucleotide identical to or complementary to a subsequence of a cancer gene; and
b) detecting whether a structural variant is present or absent in the selected sample according to the nucleic acid analysis in (a).

14. The method of claim 13, wherein the sample is a patient-derived sample.

15. The method of claim 14, wherein the sample is an FFPE sample,

16. A composition comprising a set of synthetic oligonucleotide species, wherein:
a) each oligonucleotide species is 10 to 500 consecutive nucleotides in length;
b) each oligonucleotide species comprises a polynucleotide identical to or complementary to a subsequence in an exon of an oncogene; and
c) the polynucleotide maps to coordinates that are within 300-400 bp of one or more sites targeted by one or more restriction enzymes.

17. A method for detecting the presence or absence of a structural variant in a sample, the method comprising:
a) obtaining a sample from a subject over a plurality of time points;
b) for the sample obtained at each of the time points, performing a nucleic acid analysis on the sample, wherein the analysis comprises a method that preserves spatial-proximal contiguity information; and
c) detecting whether a structural variant is present or absent in the selected sample according to the nucleic acid analysis in (b).
